(19) United States
(12) Patent Application Publication
(10) Pub. No.: US 2024/0417767 A1
(43) Pub. Date: Dec. 19, 2024

(54) PRODUCTION OF ALPHA-1,3-FUCOSYLATED COMPOUNDS

(71) Applicant: INBIOSE N.V., Zwijnaarde (BE)

(72) Inventors: Joeri Beauprez, Zwijnaarde (BE); Kristof Vandewalle, Zwijnaarde (BE); Annelies Vercauteren, Zwijnaarde (BE)

(21) Appl. No.: 18/718,677

(22) PCT Filed: Dec. 14, 2022
(86) PCT No.: PCT/EP2022/085811

§ 371 Date: Jun. 11, 2024

(30) Foreign Application Priority Data
Dec. 14, 2021 (EP) 21214480.2
Dec. 14, 2021 (LU) 501009

(51) Int. Cl. C12P 19/04; C07H 1/08; C12N 9/10; C12P 19/18

(52) U.S. Cl. CPC C12P 19/04; C07H 1/08; C12N 9/1051; C12P 19/18; C12Y 204/01065

(57) ABSTRACT

This disclosure is in the technical field of synthetic biology, metabolic engineering and cell cultivation. The disclosure describes methods for the production of a fucosylated compound using a fucosyltransferase as well as the purification of the fucosylated compound, the fucosyltransferase having alpha-1,3-fucosyltransferase activity on the N-acetylglucosamine (GlcNAc) and/or the glucose (Glc) residue of Gal-β1,m-GlcNAc-β1,n-Gal-β1,4-Glc of a saccharide substrate comprising Gal-β1,m-GlcNAc-β1,n-Gal-β1,4-Glc wherein the m is 3 or 4 and the n is 3 or 6. The disclosure also provides a cell for production of a fucosylated compound. Next, the disclosure describes methods for the production of 3-fucosyllactose (3-FL) using a fucosyltransferase having alpha-1,3-fucosyltransferase activity on the Glc residue of lactose, as well as the purification of the 3-FL. The disclosure also provides a cell for production of 3-FL.

### FIELD OF THE INVENTION

The present invention is in the technical field of synthetic biology, metabolic engineering and cell cultivation. The present invention describes methods for the production of a fucosylated compound using a fucosyltransferase as well as the purification of said fucosylated compound, said fucosyltransferase having alpha-1,3-fucosyltransferase activity on the N-acetylglucosamine (GlcNAc) and/or the glucose (Glc) residue of Gal-β1,m-GlcNAc-β1,n-Gal-β1,4-Glc of a saccharide substrate comprising Gal-β1,m-GlcNAc-β1,n-Gal-β1,4-Glc wherein said m is 3 or 4 and said n is 3 or 6. The present invention also provides a cell for production of a fucosylated compound. Next, the present invention describes methods for the production of 3-fucosyllactose (3-FL) using a fucosyltransferase having alpha-1,3-fucosyltransferase activity on the Glc residue of lactose, as well as the purification of said 3-FL. The present invention also provides a cell for production of 3-FL.

### BACKGROUND

Oligosaccharides, often present as glyco-conjugated forms to proteins and lipids, are involved in many vital phenomena such as differentiation, development and biological recognition processes related to the development and progress of fertilization, embryogenesis, inflammation, metastasis, and host pathogen adhesion. Oligosaccharides can also be present as unconjugated glycans in body fluids and mammalian milk, comprising human milk, wherein they also modulate important developmental and immunological processes (Bode, Early Hum. Dev. 1-4 (2015); Reily et al., Nat. Rev. Nephrol. 15, 346-366 (2019); Varki, Glycobiology 27, 3-49 (2017)). Human milk oligosaccharides (HMOs) are complex oligosaccharides derived from the elongation of a lactose backbone (Gal-β1,4-Glc) at the reducing glucose (Glc) by a fucose (Fuc) residue and/or at the non-reducing galactose (Gal) by a monosaccharide residue like e.g. fucose, a sialic acid, N-acetylglucosamine. Additional elongation can occur, and more than 150 HMOs have been described to date (Bode, Early Hum. Dev. 91, 619-622 (2015); Hill et al., Nutrients 13, 3364 (2021); Rousseaux et al., Front. Immunol. 2021, https://doi.org/10.3389/fimmu.2021.680911). The fucosylated oligosaccharides Gal-β1,4-[Fuc-a1,3]-Glc, (3-FL, 3-fucosyllactose), Gal-β1,4-[Fuc-a1,3]-GlcNAc-β1,3-Gal-β1,4-Glc (lacto-N-fucopentaose III, LNFP-III), Gal-β1,4-GlcNAc-β1,3-Gal-β1,4-[Fuc-a1,3]-Glc (lacto-N-neofucopentaose V, LNFP-VI), Gal-β1,4-[Fuc-a1,3]-GlcNAc-β1,3-Gal-β1,4-[Fuc-a1,3]-Glc (lacto-N-neodifucohexaose II, LNnDFH II), Gal-β1,3-GlcNAc-β1,3-Gal-β1,4-[Fuc-a1,3]-Glc (lacto-N-fucopentaose V, LNFP-V) and Gal-β1,3-[Fuc-a1,4]-GlcNAc-β1,3-Gal-β1,4-[Fuc-a1,3]-Glc (lacto-N-difucohexaose II, LNDFH-II) are part of the group of neutral HMOs that do not share a negatively charged monosaccharide residue. Amongst other HMOs, these fucosylated oligosaccharides play essential roles in promoting digestive health and the development of the microbiome and provide both direct and indirect immune protection Hill et al., Nutrients 13, 3364 (2021). There is large scientific and commercial interest in these structures or compounds, yet the availability is limited.

### SUMMARY OF THE INVENTION

It is an object of the present invention to provide for tools and methods by means of which these structures can be produced, preferably in an efficient, time and cost-effective way and which yields high amounts of the desired compound. According to the invention, this and other objects are achieved by providing methods and a cell for the production of a fucosylated compound, wherein said fucosylated compound is a saccharide comprising Gal-β1,m-[Fuc-α1,3]-GlcNAc-β1,n-Gal-β1,4-Glc, Gal-β1,m-GlcNAc-β1,n-Gal-β1,4-[Fuc-α1,3]-Glc or Gal-β1,m-[Fuc-α1,3]-GlcNAc-β1,n-Gal-β1,4-[Fuc-α1,3]-Glc, wherein said m is 3 or 4 and said n is 3 or 6. Optionally, said saccharide is linked to a peptide, a protein and/or a lipid. The present invention also provides methods for the purification of said fucosylated compound. Furthermore, the present invention provides a cell metabolically engineered for the production of said fucosylated compound. Next, the invention provides methods and a cell for the production of Gal-β1,4-[Fuc-α1,3]-Glc (3-fucosyllactose, 3-FL). The present invention also provides methods for the purification of 3-FL. Furthermore, the present invention provides a cell metabolically engineered for the production of said 3-FL.

Surprisingly, it has now been found that it is possible to produce said fucosylated compound. The present invention describes methods for the production of said fucosylated compound. The methods comprise the steps of providing 1) GDP-fucose, 2) a saccharide substrate comprising Gal-β1,m-GlcNAc-β1,n-Gal-β1,4-Glc wherein said m is 3 or 4 and said n is 3 or 6, optionally said saccharide is linked to a peptide, a protein and/or a lipid and 3) a fucosyltransferase that has alpha-1,3-fucosyltransferase activity on the N-acetylglucosamine (GlcNAc) and/or the glucose (Glc) residue of Gal-β1,m-GlcNAc-β1,n-Gal-β1,4-Glc of said saccharide substrate, and contacting said fucosyltransferase and GDP-fucose with said saccharide substrate under conditions where the fucosyltransferase catalyses the transfer of a fucose residue from said GDP-fucose to the GlcNAc and/or Glc residue of said Gal-β1,m-GlcNAc-β1,n-Gal-β1,4-Glc of said saccharide substrate in an alpha-1,3-glycosidic linkage resulting in the production of said fucosylated compound. The present invention also describes a method wherein said fucosylated compound is produced by a cell. Herein, the cell expresses a fucosyltransferase that has alpha-1,3-fucosyltransferase activity on the GlcNAc and/or the Glc residue of Gal-β1,m-GlcNAc-β1,n-Gal-β1,4-Glc of a saccharide substrate comprising Gal-β1,m-GlcNAc-β1,n-Gal-β1,4-Glc wherein said m is 3 or 4 and said n is 3 or 6. One method of present invention comprises the steps of providing a cell which expresses said fucosyltransferase capable of transferring a fucose residue from GDP-fucose to the GlcNAc and/or Glc residue of Gal-β1,m-GlcNAc-β1,n-Gal-β1,4-Glc of said saccharide substrate in an alpha-1,3-glycosidic linkage, and cultivating said cell under conditions permissive for production of said fucosylated compound. The present invention also provides a cell metabolically engineered for the production of said fucosylated compound. Furthermore, the present invention provides methods for the purification of said fucosylated compound. In the present invention, said saccharide substrate comprising Gal-β1,m-GlcNAc-β1,n-Gal-β1,4-Glc may be linked to a peptide, a protein and/or a lipid. The present invention also describes methods for the production of 3-FL. The methods comprise the steps of providing 1) GDP-fucose, 2) lactose and 3) a fucosyltransferase that has alpha-1,3-fucosyltransferase activity on the Glc residue of lactose, and contacting said fucosyltransferase and GDP-fucose with said lactose under conditions where the fucosyltransferase catalyses the transfer of a fucose residue from said GDP-fucose to the Glc residue of said lactose in an alpha-1,3-glycosidic linkage resulting in the production of 3-FL. The present invention also describes a method wherein 3-FL is produced by a cell. Herein, the cell expresses a fucosyltransferase that has alpha-1,3-fucosyltransferase activity on the Glc residue of lactose. One method of present invention comprises the steps of providing a cell which expresses said fucosyltransferase capable of transferring a fucose residue from GDP-fucose to the Glc residue of lactose in an alpha-1,3-glycosidic linkage, and cultivating said cell under conditions permissive for production of said 3-FL. The present invention also provides a cell metabolically engineered for the production of 3-FL. Furthermore, the present invention provides methods for the purification of 3-FL.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the invention disclosed herein are to be understood not only in the order and context specifically described in this specification, but to include any order and any combination thereof. Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference in its entirety. Whenever the context requires, unless specifically stated otherwise, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications. In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only and are not intended to imply any particular order for performing the steps, unless specifically stated otherwise. Throughout the application, unless explicitly stated otherwise, the features “synthesize”, “synthesized” and “synthesis” are interchangeably used with the features “produce”, “produced” and “production”, respectively. Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e., “expresses” is preferably replaced with “capable of expressing”. In this document and in its claims, the verb “to comprise”, “to have” and “to contain” and their conjugations are used in their non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, said additional component(s) not altering the unique characteristic of the invention. In this document and in its claims, unless specifically stated otherwise, the verb “to comprise”, “to have” and “to contain”, and their conjugations, may be replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one”. Throughout the application, unless explicitly stated otherwise, the articles “a” and “an” are preferably replaced by “at least two”, more preferably by “at least three”, even more preferably by “at least four”, even more preferably by “at least five”, even more preferably by “at least six”, most preferably by “at least two”. The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y”, wherein x and y represent numerical values, refers to a range of numerical values wherein x is the lower value of the range and y is the upper value of the range. Herein, x and y are also included in the range.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

The terms “recombinant” or “transgenic” or “metabolically engineered” or “genetically engineered”, as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one which has been produced by a recombinant cell. The terms also encompass cells that have been modified by removing a nucleic acid endogenous to the cell by means of common well-known technologies for a skilled person (like e.g., knocking-out genes).

A “heterologous sequence” or a “heterologous nucleic acid”, as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the present invention refers to a cell or microorganism which is genetically engineered.

The term “endogenous,” within the context of the present invention refers to any polynucleotide, polypeptide or protein sequence which is a natural part of a cell and is occurring at its natural location in the cell chromosome and of which the control of expression has not been altered compared to the natural control mechanism acting on its expression. The term “exogenous” refers to any polynucleotide, polypeptide or protein sequence which originates from outside the cell under study and not a natural part of the cell or which is not occurring at its natural location in the cell chromosome or plasmid. The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild type expression of said gene in any phase of the production process of the desired fucosylated compound. Said modified expression is either a lower or higher expression compared to the wild type, wherein the term “higher expression” is also defined as “overexpression” of said gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, etc.) which are used to change the genes in such a way that they are “less-able” (i.e., statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild type or an inducible promoter which result in regulated expression or a repressible promoter which results in regulated expression. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein said gene is part of an “expression cassette” which relates to any sequence in which a promoter sequence, untranslated region sequence (containing either a ribosome binding sequence, Shine Dalgarno or Kozak sequence), a coding sequence and optionally a transcription terminator is present, and leading to the expression of a functional active protein. Said expression is either constitutive, conditional, tuneable or regulated.

The term “constitutive expression” is defined as expression that is not regulated by transcription factors other than the subunits of RNA polymerase (e.g., bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, Lacl, ArcA, Cra, IcIR in E. coli, or Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed. The term “inducible expression by a natural inducer” is defined as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. The term “inducible expression upon chemical treatment” is defined as a facultative or regulatory expression of a gene that is only expressed upon treatment with a chemical inducer or repressor, wherein said inducer and repressor comprise but are not limited to an alcohol (e.g. ethanol, methanol), a carbohydrate (e.g. glucose, galactose, glycerol, lactose, arabinose, rhamnose, fucose, allo-lactose), metal ions (e.g. aluminium, copper, zinc), nitrogen, phosphates, IPTG, acetate, formate, xylene.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous. The term “wild type” refers to the commonly known genetic or phenotypical situation as it occurs in nature.

The term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native in the expression host) protein, iv) reduced expression of an endogenous protein or v) expression and/or overexpression of a variant protein that has a reduced activity compared to the wild-type (i.e., native in the expression host) protein. Preferably, the term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native in the expression host) protein.

The term “modified activity” of a protein relates to a non-native activity of the protein in any phase of the production process of the fucosylated compound. The term “non-native”, as used herein with reference to the activity of a protein indicates that the protein has been modified to have an abolished, impaired, reduced, delayed, higher, accelerated or improved activity compared to the native activity of said protein. A modified activity of a protein is obtained by modified expression of said protein or is obtained by expression of a modified, i.e., mutant form of the protein. A mutant form of the protein can be obtained by expression of a mutant form of the gene encoding the protein, e.g., comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis. The term “non-native”, as used herein with reference to a cell producing a fucosylated compound, indicates that the fucosylated compound is i) not naturally produced or ii) when naturally produced not in the same amounts by the cell; and that the cell has been genetically engineered to be able to produce said fucosylated compound or to have a higher production of said fucosylated compound.

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g., derived from a mammary cell lineage or a non-mammary cell lineage) that exhibits either naturally, or has been engineered to, be capable of expressing at least one milk component.

As used herein, the term “non-mammary cell(s)” may generally include any mammalian cell of non-mammary lineage. In the context of the invention, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present invention contemplates making functional variants by modifying the structure of an enzyme as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the invention results in a functional homolog can be readily determined by assessing the ability of the variant polypeptide to produce a response in cells in a fashion similar to the wild-type polypeptide.

“Fragment”, with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, comprising or consisting of at least about 9, 10, 11, 12 consecutive nucleotides from said polynucleotide SEQ ID NO, for example at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO preferably means a nucleotide sequence which comprises or consists of said polynucleotide SEQ ID NO wherein no more than about 200, 150, 100, 50 or 25 consecutive nucleotides are missing, preferably no more than about 50 consecutive nucleotides are missing, and which retains a usable, functional characteristic (e.g. activity) of the full-length polynucleotide molecule which can be assessed by the skilled person through routine experimentation. Alternatively, a fragment of a polynucleotide SEQ ID NO preferably means a nucleotide sequence which comprises or consists of an amount of consecutive nucleotides from said polynucleotide SEQ ID NO and wherein said amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0%, of the full-length of said polynucleotide SEQ ID NO and retains a usable, functional characteristic (e.g. activity) of the full-length polynucleotide molecule. As such, a fragment of a polynucleotide SEQ ID NO preferably means a nucleotide sequence which comprises or consists of said polynucleotide SEQ ID NO, wherein an amount of consecutive nucleotides is missing and wherein said amount is no more than 50.0%, 40.0%, 30.0% of the full-length of said polynucleotide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polynucleotide SEQ ID NO and wherein said fragment retains a usable, functional characteristic (e.g. activity) of the full-length polynucleotide molecule which can be routinely assessed by the skilled person.

“Fragment”, with respect to a polypeptide, refers to a subsequence of the polypeptide which performs at least one biological function of the intact polypeptide in substantially the same manner, or to a similar extent, as does the intact polypeptide. A “subsequence of the polypeptide” or “a stretch of amino acid residues” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 10 amino acid residues in length, for example at least about 20 amino acid residues in length, for example at least about 30 amino acid residues in length, for example at least about 100 amino acid residues in length, for example at least about 150 amino acid residues in length, for example at least about 200 amino acid residues in length. As such, a fragment of a polypeptide SEQ ID NO (or UniProt ID) preferably means a polypeptide sequence which comprises or consists of said polypeptide SEQ ID NO (or UniProt ID) wherein no more than about 200, 150, 125, 100, 80, 60, 50, 40, 30, 20 or 15 consecutive amino acid residues are missing, preferably no more than about 100 consecutive amino acid residues are missing, more preferably no more than about 50 consecutive amino acid residues are missing, even more preferably no more than about 40 consecutive amino acid residues are missing, and performs at least one biological function of the intact polypeptide in substantially the same manner, preferably to a similar or greater extent, as does the intact polypeptide which can be routinely assessed by the skilled person. Alternatively, a fragment of a polypeptide SEQ ID NO (or UniProt ID) preferably means a polypeptide sequence which comprises or consists of an amount of consecutive amino acid residues from said polypeptide SEQ ID NO (or UniProt ID) and wherein said amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0% of the full-length of said polypeptide SEQ ID NO (or UniProt ID) and which performs at least one biological function of the intact polypeptide in substantially the same manner, preferably to a similar or greater extent, as does the intact polypeptide which can be routinely assessed by the skilled person. As such, a fragment of a polypeptide SEQ ID NO (or UniProt ID) preferably means a polypeptide sequence which comprises or consists of said polypeptide SEQ ID NO (or UniProt ID), wherein an amount of consecutive amino acid residues is missing and wherein said amount is no more than 50.0%, 40.0%, 30.0% of the full-length of said polypeptide SEQ ID NO (or UniProt ID), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polypeptide SEQ ID NO (or UniProt ID) and which performs at least one biological function of the intact polypeptide in substantially the same manner, preferably to a similar or greater extent, as does the intact polypeptide which can be routinely assessed by the skilled person.

Throughout the application, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the polypeptide of interest. Sequence analysis can involve BLAST, Reciprocal BLAST, or PSI-BLAST analysis of non-redundant databases using the amino acid sequence of a reference polypeptide sequence. The amino acid sequence is, in some instances, deduced from the nucleotide sequence. Typically, those polypeptides in the database that have greater than 40 percent sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (EI-Gebali et al., Nucleic Acids Res. 47 (2019) D427-D432), an IPR (InterPro domain) (http://ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48(D1) (2020) D606-D612). Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the application, the sequence of a polypeptide is represented by a SEQ ID NO or an UniProt ID. Unless stated otherwise, the UniProt IDs of the proteins described correspond to their sequence version 01 as present in the UniProt Database (www.uniprot.org) version release 2021_03 and consulted on 9 Jun. 2021. InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

It should be understood for those skilled in the art that for the databases used herein, comprising Pfam 34.0 (released March 2021), CDD v3.19 (released 8 Mar. 2021), EggNOG 5.0.0 (released November 2018), InterPro 86.0 (released 3 Jun. 2021) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “identical” or “percent identity” or “% identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global percent identity score. Alternatively, percent identity may be calculated over a partial sequence of the reference sequence, resulting in a local percent identity score. A partial sequence preferably means at least about 25%, 30%, 35%, 40%, 45%, 50%, 55%, 60%, 65%, 70%, 75%, 80%, 85%, 87.5%, 90%, 91%, 92%, 93%, 94% or 95% of the full-length reference sequence. In another preferred embodiment, a partial sequence of a reference polypeptide sequence means a stretch of at least 150 amino acid residues up to the total number of amino acid residues of a reference polypeptide sequence. In another more preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence.

Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

As used herein, a polypeptide comprising or consisting of an amino acid sequence having 50.0% or more sequence identity to the full-length sequence of a reference polypeptide sequence is to be understood as that the amino acid sequence has 50.0%, 50.50% or more sequence identity to the full-length of the amino acid sequence of the reference polypeptide sequence. A polypeptide comprising or consisting of an amino acid sequence having 50.50% or more sequence identity to the full-length sequence of a reference polypeptide sequence is to be understood as that the amino acid sequence has 50.50%, 51.0%, 52.0%, 52.50% or more sequence identity to the full-length of the amino acid sequence of the reference polypeptide sequence. A polypeptide comprising or consisting of an amino acid sequence having 52.50% or more sequence identity to the full-length sequence of a reference polypeptide sequence is to be understood as that the amino acid sequence has 52.50%, 55.0%, 57.50%, 60.0%, 62.50%, 65.0%, 67.50%, 70.0%, 72.50% or more sequence identity to the full-length of the amino acid sequence of the reference polypeptide sequence. A polypeptide comprising or consisting of an amino acid sequence having 72.50% or more sequence identity to the full-length sequence of a reference polypeptide sequence is to be understood as that the amino acid sequence has 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of the amino acid sequence of the reference polypeptide sequence. Throughout the application, unless explicitly specified otherwise, a polypeptide comprising, consisting or having an amino acid sequence having 50.0% or more sequence identity to the full-length amino acid sequence of a reference polypeptide, usually indicated with a SEQ ID NO or UniProt ID, preferably has 50.0%, 50.50% or more, more preferably has 50.50% or more, even more preferably has 52.50% or more sequence identity to the full-length reference sequence. Additionally, a polypeptide comprising, consisting or having an amino acid sequence having 50.50% or more sequence identity to the full-length amino acid sequence of a reference polypeptide, usually indicated with a SEQ ID NO or UniProt ID, preferably has 50.50%, 51.0%, 52.0%, 52.50% or more, more preferably has 52.50%, even more preferably has 55.0% or more sequence identity to the full-length reference sequence. Additionally, a polypeptide comprising, consisting or having an amino acid sequence having 52.50% or more sequence identity to the full-length amino acid sequence of a reference polypeptide, usually indicated with a SEQ ID NO or UniProt ID, preferably has at least 52.50%, 55.0%, 57.50%, 60.0%, 62.50%, 65.0%, 67.50%, 70.0%, 72.50% or more, more preferably has 72.50%, even more preferably has 75.0% or more sequence identity to the full-length reference sequence. Additionally, a polypeptide comprising, consisting or having an amino acid sequence having 72.50% or more sequence identity to the full-length amino acid sequence of a reference polypeptide, usually indicated with a SEQ ID NO or UniProt ID, preferably has at least 72.50%, 75.0%, 77.50%, 80.0%, 82.50%, 85.0%, 90.0%, 91.0%, 92.0%, 93.0%, 94.0%, 95.0%, 96.0%, 97.0%, 98.0% or 99.0%, more preferably has at least 75.0%, even more preferably has at least 80.0%, most preferably has at least 82.50%, sequence identity to the full length reference sequence.

Additionally, unless explicitly specified otherwise, a polynucleotide comprising, consisting or having a nucleotide sequence having 50.0% or more sequence identity to the full-length nucleotide sequence of a reference polynucleotide sequence, usually indicated with a SEQ ID NO, preferably has 50.0%, 50.50% or more, more preferably has 50.50% or more, even more preferably has 52.50% or more sequence identity to the full-length reference sequence. Additionally, a polynucleotide comprising, consisting or having a nucleotide sequence having 50.50% or more sequence identity to the full-length nucleotide sequence of a reference polynucleotide sequence, usually indicated with a SEQ ID NO, preferably has 50.50%, 51.0%, 52.0%, 52.50% or more, more preferably has 52.50%, even more preferably has 55.0% or more sequence identity to the full-length reference sequence. Additionally, a polynucleotide comprising, consisting or having a nucleotide sequence having 52.50% or more sequence identity to the full-length nucleotide sequence of a reference polynucleotide sequence, usually indicated with a SEQ ID NO, preferably has at least 52.50%, 55.0%, 57.50%, 60.0%, 62.50%, 65.0%, 67.50%, 70.0%, 72.50% or more, more preferably has 72.50%, even more preferably has 75.0% or more sequence identity to the full-length reference sequence. Additionally, a polynucleotide comprising, consisting or having a nucleotide sequence having 72.50% or more sequence identity to the full-length nucleotide sequence of a reference polynucleotide sequence usually indicated with a SEQ ID NO, preferably has at least 72.50%, 75.0%, 77.50%, 80.0%, 625 82.50%, 85.0%, 90.0%, 91.0%, 92.0%, 93.0%, 94.0%, 95.0%, 96.0%, 97.0%, 98.0% or 99.0%, more preferably has at least 75.0%, even more preferably has at least 80.0%, most preferably has at least 82.50%, sequence identity to the full-length reference sequence.

For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50. In a preferred embodiment, sequence identity is calculated based on the full-length sequence of a given SEQ ID NO, i.e. the reference sequence, or a part thereof. Part thereof preferably means at least 50%, 60%, 70%, 80%, 90% or 95% of the complete reference sequence.

The term “glycosyltransferase” as used herein refers to an enzyme capable to catalyse the transfer of a sugar moiety from an activated donor molecule to a specific substrate molecule, forming glycosidic bonds. Said activated donor molecule can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-(1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto a substrate. Fucosyltransferases comprise alpha-1,2-fucosyltransferases, alpha-1,3-fucosyltransferases, alpha-1,3/4-fucosyltransferases, alpha-1,4-fucosyltransferases and alpha-1,6-fucosyltransferases that catalyse the transfer of a Fuc residue from GDP-Fuc onto a substrate via alpha-glycosidic bonds. Fucosyltransferases can be found but are not limited to the GT10, GT11, GT23, GT65, GT68 and GT74 CAZy families.

The wording “a fucosyltransferase that has alpha-1,3-fucosyltransferase activity on the N-acetylglucosamine (GlcNAc) residue and/or the glucose (Glc) residue of Gal-β1,m-GlcNAc-β1,n-Gal-β1,4-Glc, wherein said m is 3 or 4 and said n is 3 or 6, of a saccharide substrate comprising said Gal-β1,m-GlcNAc-β1,n-Gal-β1,4-Glc” refers to a fucosyltransferase that catalyses the transfer of fucose (Fuc) from the donor GDP-L-fucose to the GlcNAc residue and/or the Glc residue of Gal-β1,m-GlcNAc-β1,n-Gal-β1,4-Glc of a saccharide substrate comprising said Gal-β1,m-GlcNAc-β1,n-Gal-β1,4-Glc in an alpha-1,3-linkage producing a fucosylated compound as described herein.

The wording “a fucosyltransferase that has alpha-1,3-fucosyltransferase activity on the Glc residue of lactose” refers to a fucosyltransferase that catalyses the transfer of fucose (Fuc) from the donor GDP-L-fucose to the Glc residue of lactose in an alpha-1,3-linkage producing Gal-β1,4-[Fuc-α1,3]-Glc (3-fucosyllactose, 3-FL)”.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides comprise UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose or UDP-xylose. Nucleotide-sugars act as glycosyl donors in glycosylation reactions. Those reactions are catalysed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar.

The term “disaccharide” as used herein refers to a saccharide polymer containing two simple sugars, i.e., monosaccharides. Examples of disaccharides comprise lactose (Gal-β1,4-Glc), lacto-N-biose (Gal-β1,3-GlcNAc) (LNB), N-acetyllactosamine (Gal-β1,4-GlcNAc) (LacNAc), LacDiNAc (GalNAc-β1,4-GlcNAc), N-acetylgalactosaminylglucose (GalNAc-β1,4-Glc), Neu5Ac-α2,3-Gal, Neu5Ac-α2,6-Gal, fucopyranosyl-(1-4)-N-glycolylneuraminic acid (Fuc-(1-4)-Neu5Gc), sucrose (Glc-α1,2-Fru), maltose (Glc-α1,4-Glc) and melibiose (Gal-α1,6-Glc).

The terms “LNB”, “lacto-N-biose”, “lacto-N-biose I”, “LacNAc type 1”, “LacNAc type I” and “2-Acetamido-2-deoxy-3-O-(b-D-galactopyranosyl)-D-glucopyranose” are used interchangeably and refer to the disaccharide Gal-β1,3-GlcNAc.

The terms “LacNAc”, “N-acetyllactosamine”, “LacNAc type 2”, “LacNAc type II”, “Galactopyranosyl-b-1,4-N-acetyl-D-glucosamine”, “beta-D-galactosyl-1,4-N-acetyl-D-glucosamine” and “2-Acetamido-2-deoxy-D-lactose” are used interchangeably and refer to the disaccharide Gal-β1,4-GlcNAc.

“Oligosaccharide” as the term is used herein and as generally understood in the state of the art, refers to a saccharide polymer containing a small number, typically three to twenty, preferably three to fifteen, more preferably three to thirteen, even more preferably three to twelve, even more preferably three to eleven, most preferably three to ten, of simple sugars, i.e., monosaccharides. The oligosaccharide as used in the present invention can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “Gal-β1,4-Glc”, “b-Gal-(1->4)-Glc”, “β-Gal-(1->4)-Glc”, “Galbeta1-4-Glc”, “Gal-b(1-4)-Glc” and “Gal-β(1-4)-Glc” have the same meaning, i.e. a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars, antigens of the human ABO blood group system, neutral (non-charged) oligosaccharides, negatively charged oligosaccharides, fucosylated oligosaccharides, sialylated oligosaccharides, N-acetylglucosamine containing oligosaccharides, N-acetyllactosamine containing oligosaccharides, lacto-N-biose containing oligosaccharides, lactose containing oligosaccharides, non-fucosylated neutral (non-charged) oligosaccharides, N-acetyllactosamine containing fucosylated oligosaccharides, N-acetyllactosamine non-fucosylated oligosaccharides, lacto-N-biose containing fucosylated oligosaccharides, lacto-N-biose containing non-fucosylated oligosaccharides, N-acetyllactosamine containing negatively charged oligosaccharides, lacto-N-biose containing negatively charged oligosaccharides, animal oligosaccharides, preferably selected from the group consisting of N-glycans and O-glycans, plant oligosaccharides, preferably selected from the group consisting of N-glycans and O-glycans.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of said monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages. Examples comprise 2′-fucosyllactose (2′FL), 3-fucosyllactose (3FL), 4-fucosyllactose (4FL), 6-fucosyllactose (6FL), difucosyllactose (diFL), Lacto-N-fucopentaose I (LNF I), Lacto-N-fucopentaose II (LNF II), Lacto-N-fucopentaose II (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose II (MFLNH III), Difucosyllacto-N-hexaose (DFLNH a, DFLNH c), difucosyl-lacto-N-neohexaose, GlcNAc-LNFP II, GlcNAc-LNFP V, GlcNAc-LNDFH-II, lacto-N-difucohexaose II (LNDFH-II), lacto-N-neodifucohexaose II (LNnDFH II), 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose.

As used herein, a ‘sialylated oligosaccharide’ is to be understood as a negatively charged sialic acid containing oligosaccharide, i.e., an oligosaccharide having a sialic acid residue. It has an acidic nature.

A ‘neutral oligosaccharide’ or ‘a non-charged oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2,3-difucosyllactose (diFL), lacto-N-triose II (LN3), lacto-N-tetraose (LNT), lacto-N-neotetraose (LNnT), lacto-N-fucopentaose I (LNFP I), lacto-N-neofucopentaose I (LNnFP I), lacto-N-fucopentaose II (LNFP II), lacto-N-fucopentaose II (LNFP III), lacto-N-fucopentaose V (LNFP V), lacto-N-neofucopentaose V (LNnFP V), lacto-N-fucopentaose VI, lacto-N-difucohexaose I (LNDFH I), lacto-N-difucohexaose II (LNDFH-II), 6′-galactosyllactose, 3′-galactosyllactose, lacto-N-hexaose, lacto-N-neohexaose, para-lacto-N-hexaose, para-lacto-N-neohexaose, fucosyl-lacto-N-hexaose, difucosyl-lacto-N-hexaose, difucosyl-lacto-N-neohexaose (LNnDFH II), difucosyl-para-lacto-N-neohexaose, trifucosyllacto-N-hexaose, para-lacto-N-fucohexaose and lacto-N-trifucoheptaose.

Mammalian milk oligosaccharides or MMOs comprise oligosaccharides present in milk found in any phase during lactation including colostrum milk from humans (i.e. human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). Human milk oligosaccharides are also known as human identical milk oligosaccharides which are chemically identical to the human milk oligosaccharides found in human breast milk, but which are biotechnologically produced (e.g., using cell free systems or cells and organisms comprising a bacterium, a fungus, a yeast, a plant, animal, or protozoan cell, preferably metabolically engineered cells and organisms). As used herein, “mammalian milk oligosaccharide” or MMO refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides. HMOs comprise fucosylated oligosaccharides, non-fucosylated neutral oligosaccharides and sialylated oligosaccharides (see e.g., Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

As used herein the term “Lewis-type antigens” comprise the following oligosaccharides: H1 antigen, which is Fucα1-2Galβ1-3GlcNAc, or in short 2FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, an antigen of the human ABO blood group system is an oligosaccharide. Such antigens of the human ABO blood group system are not restricted to human structures. Said structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal-that are present on disaccharide core structures comprising Gal-beta1,3-GlcNAc, Gal-beta1,4-GlcNAc, Gal-beta1,3-GalNAc and Gal-beta1,4-Glc.

The terms “LNT II”, “LNT-II”, “LN3”, “lacto-N-triose II”, “lacto-N-triose II”, “lacto-N-triose”, “lacto-N-triose” or “GlcNAcβ1-3Galβ1-4Glc” as used in the present invention, are used interchangeably. The terms “LNT”, “lacto-N-tetraose”, “lacto-N-tetraose” or “Galβ1-3GlcNAcβ1-3Galβ1-4Glc” as used in the present invention, are used interchangeably. The terms “LNnT”, “lacto-N-neotetraose”, “lacto-N-neotetraose”, “lacto-N-neotetraose”, “neo-LNT” or “Galβ1-4GlcNAcβ1-3Galγ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GlcNAc-b1,3-Gal-b1,4-Glc” as used in the present invention, are used interchangeably. The terms “LSTb”, “LS-Tetrasaccharide b”, “Sialyl-lacto-N-tetraose b”, “sialyllacto-N-tetraose b” or “Gal-β1,3-(Neu5Ac-α2,6)-GlcNAc-β1,3-Gal-β1,4-Glc” as used in the present invention, are used interchangeably. The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-α2,6-Gal-β1,4-GlcNAc-β1,3-Gal-β1,4-Glc” as used in the present invention, are used interchangeably. The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-α2,3-Gal-β1,4-GlcNAc-β1,3-Gal-β1,4-Glc” as used in the present invention, are used interchangeably. The terms “DSLNnT” and “Disialyllacto-N-neotetraose” are used interchangeably and refer to Neu5Ac-α2,6-Gal-β1,4-GlcNAc-β1,3-[Neu5Ac-α2,6]-Gal-β1,4-Glc. The terms “DSLNT”, “DS-LNT” and “Disialyllacto-N-tetraose” are used interchangeably and refer to Neu5Ac-α2,3-Gal-β1,3-[Neu5Ac-α2,6]-GlcNAc-β1,3-Gal-β1,4-Glc.

The terms “LNFP-1”, “lacto-N-fucopentaose I”, “LNFP I”, “LNFPI”, “LNF I OH type I determinant”, “LNF I”, “LNF1”, “LNF 1” and “Blood group H antigen pentaose type 1” are used interchangeably and refer to Fuc-α1,2-Gal-β1,3-GlcNAc-β1,3-Gal-β1,4-Glc. The terms “GalNAc-LNFP-1” and “blood group A antigen hexaose type I” are used interchangeably and refer to GalNAc-α1,3-(Fuc-α1,2)-Gal-β1,3-GlcNAc-β1,3-Gal-β1,4-Glc. The terms “LNFP-II” and “lacto-N-fucopentaose II” are used interchangeably and refer to Gal-β1,3-[Fuc-α1,4]-GlcNAc-β1,3-Gal-β1,4-Glc. The terms “LNFP-III”, “LNFP III”, “LNFPIII” and “lacto-N-fucopentaose III” are used interchangeably and refer to Gal-β1,4-(Fuc-α1,3)-GlcNAc-β1,3-Gal-β1,4-Glc. The terms “LNFP-V”, “LNFP V”, “LNFPV” and “lacto-N-fucopentaose V” are used interchangeably and refer to Gal-β1,3-GlcNAc-β1,3-Gal-β1,4-(Fuc-α1,3)-Glc. The terms “LNFP-VI”, “LNFP VI”, “LNnFP V” and “lacto-N-neofucopentaose V” are used interchangeably and refer to Gal-β1,4-GlcNAc-β1,3-Gal-β1,4-(Fuc-α1,3)-Glc. The terms “LNnFP I” and “Lacto-N-neofucopentaose I” are used interchangeably and refer to Fuc-α1,2-Gal-β1,4-GlcNAc-β1,3-Gal-β1,4-Glc. The terms “LNDFH I”, “Lacto-N-difucohexaose I”, “LNDFH-1”, “LDFH I”, “Le^{b}-lactose” and “Lewis-b hexasaccharide” are used interchangeably and refer to Fuc-α1,2-Gal-β1,3-[Fuc-α1,4]-GlcNAc-β1,3-Gal-β1,4-Glc. The terms “LNDFH II”, “Lacto-N-difucohexaose II”, “LNDFH-II”, “Lewis a-Lewis x” and “LDFH II” are used interchangeably and refer to Gal-β1,3-[Fuc-α1,4]-GlcNAc-β1,3-Gal-β1,4-(Fuc-α1,3)-Glc. The terms “LNnDFH”, “LNnDFH II”, “LNnDFH-II”, “Lacto-N-neodifucohexaose II”, “LNDFH III”, “Lewis x hexaose” and “LeX hexaose” are used interchangeably and refer to Gal-β1,4-(Fuc-α1,3)-GlcNAc-β1,3-Gal-β1,4-(Fuc-α1,3)-Glc.

The terms “LNH” and “lacto-N-hexaose” are used interchangeably and refer to Gal-β1,3-GlcNAc-β1,3-(Gal-β1,4-GlcNAc-β1,6)-Gal-β1,4-Glc. The terms “para-LNH”, “pLNH” and “para-lacto-N-hexaose” are used interchangeably and refer to Gal-β1,3-GlcNAc-β1,3-Gal-β1,4-GlcNAc-β1,3-Gal-β1,4-Glc. The terms “LNnH” and “lacto-N-neohexaose” are used interchangeably and refer to Gal-β1,4-GlcNAc-β1,3-[Gal-β1,4-GlcNAc-β1,6]-Gal-β1,4-Glc. The terms “para-LNnH”, “pLNnH” and “para-lacto-N-neohexaose” are used interchangeably and refer to Gal-β1,4-GlcNAc-β1,3-Gal-β1,4-GlcNAc-β1,3-Gal-β1,4-Glc.

The terms “F-LNH I”, “FLNH I” and “fucosyllacto-N-hexaose I” are used interchangeably and refer to Fuc-α1,2-Gal-β1,3-GlcNAc-β1,3-[Gal-β1,4-GcNAc-β1,6]-Gal-β1,4-Glc. The terms “F-LNH-II”, “FLNH II” and “fucosyllacto-N-hexaose II” are used interchangeably and refer to Gal-β1,3-GlcNAc-β1,3-[Gal-β1,4-[Fuc-α1,3]-GlcNAc-β1,6]-Gal-β1,4-Glc. The terms “DF-LNH I”, “difucosyllacto-N-hexaose I”, “DF-LNH a”, “DFLNH a”, “difucosyllacto-N-hexaose a” and “2,3-Difucosyllacto-N-hexaose” are used interchangeably and refer to Fuc-α1,2-Gal-β1,3-GlcNAc-β1,3-[Gal-β1,4-[Fuc-α1,3]-GlcNAc-β1,6]-Gal-β1,4-Glc. The terms “DF-LNH II”, “DF-LNH b”, “DFLNH b” and “difucosyllacto-N-hexaose II” are used interchangeably and refer to Gal-β1,3-[Fuc-α1,4]-GlcNAc-β1,3-[Gal-β1,4-[Fuc-α1,3]-GlcNAc-β1,6]-Gal-β1,4-Glc. The terms “DFLNH c”, “DF-LNH c” and “difucosyllacto-N-hexaose c” are used interchangeably and refer to Fuc-α1,2-Gal-β1,3-[Fuc-α1,4]-GlcNAc-β1,3-[Gal-β1,4-GlcNAc-β1,6]-Gal-β1,4-Glc. The terms “DF-LNnH” and “difucosyllacto-N-neohexaose” are used interchangeably and refer to Gal-β1,4-[Fuc-α1,3]-GlcNAc-β1,3-[Gal-β1,4-[Fuc-α1,3]-GlcNAc-β1,6]-Gal-β1,4-Glc.

The terms “DF-para-LNH”, “DF-p-LNH”, “DF-pLNH” and “difucosyl-para-lacto-N-hexaose” are used interchangeably and refer to Gal-β1,3-[Fuc-α1,4]-GlcNAc-β1,3-Gal-β1,4-[Fuc-α1,3]-GlcNAc-β1,3-Gal-β1,4-Glc. The terms “DF-para-LNnH”, “DF-p-LNnH” and “difucosyl-para-lacto-N-neohexaose” are used interchangeably and refer to Gal-β1,4-[Fuc-α1,3]-GlcNAc-β1,3-Gal-β1,4-[Fuc-α1,3]-GlcNAc-β1,3-Gal-β1,4-Glc. The terms “TF-LNH” and “trifucosyllacto-N-hexaose” are used interchangeably and refer to Fuc-α1,2-Gal-β1,3-[Fuc-α1,4]-GlcNAc-β1,3-[Gal-β1,4-[Fuc-α1,3]-GlcNAc-β1,6]-Gal-β1,4-Glc.

The terms “F-LST a”, “F-LSTa”, “S-LNF II” and “fucosyl-sialyllacto-N-tetraose a” are used interchangeably and refer to Neu5Ac-α2,3-Gal-β1,3-[Fuc-α1,4]-GlcNAc-β1,3-Gal-β1,4-Glc. The terms “F-LST b”, “F-LSTb”, “S-LNF I” and “fucosyl-sialyllacto-N-tetraose b” are used interchangeably and refer to Fuc-α1,2-Gal-β1,3-(Neu5Ac-α2,6)-GlcNAc-β1,3-Gal-β1,4-Glc. The terms “F-LST C”, “F-LSTc” and “fucosyl-sialyllacto-N-neotetraose” are used interchangeably and refer to Neu5Ac-α2,6-Gal-β1,4-GlcNAc-β1,3-Gal-β1,4-[Fuc-α1,3]-Glc.

The terms “FS-LNH” and “fucosyl-sialyllacto-N-hexaose” are used interchangeably and refer to Fuc-α1,2-Gal-β1,3-GlcNAc-β1,3-(Neu5Ac-α2,6-Gal-β1,4-GlcNAc-β1,6)-Gal-β1,4-Glc.

The terms “FS-LNnH I” and “fucosyl-sialyllacto-N-neohexaose I” are used interchangeably and refer to Neu5Ac-α2,6-Gal-β1,4-GlcNAc-β1,3-[Gal-β1,4-[Fuc-α1,3]-GlcNAc-β1,6]-Gal-β1,4-Glc.

The terms “FDS-LNH II” and “fucosyldisialyllacto-N-hexaose II” are used interchangeably and refer to Neu5Ac-α2,3-Gal-β1,3-[Neu5Ac-α2,6]-GlcNAc-β1,3-[Gal-β1,4-[Fuc-α1,3]-GlcNAc-β1,6]-Gal-β1,4-Glc.

The terms “alpha-tetrasaccharide” and “A-tetrasaccharide” are used interchangeably and refer to GalNAc-α1,3-(Fuc-α1,2)-Gal-β1,4-Glc.

The terms “Fuc-α1,2-Gal-β1,3-GlcNAc”, “2-fucosyllacto-N-biose”, “2FLNB”, “2 FLNB”, “2-FLNB”, “2′-FLNB” and “2′FLNB” are used interchangeably and refer to a trisaccharide wherein a fucose residue is linked to the galactose residue of lacto-N-biose (LNB, Gal-β1,3-GlcNAc) in an alpha-1,2 linkage.

The terms “Gal-β1,3-[Fuc-α1,4]-GlcNAc”, “4-fucosyllacto-N-biose”, “4FLNB”, “4 FLNB” and “4-FLNB” are used interchangeably and refer to a trisaccharide wherein a fucose residue is linked to the N-acetylglucosamine residue of lacto-N-biose (LNB, Gal-β1,3-GlcNAc) in an alpha-1,4 linkage.

The terms “Gal-β1,4-[Fuc-α1,3]-GlcNAc”, “3-fucosyl-N-acetyllactosamine”, “3-FLacNAc”, “3FLacNAc” and “3 FLacNAc” are used interchangeably and refer to a trisaccharide wherein a fucose residue is linked to the GlcNAc residue of N-acetyllactosamine (LacNAc, Gal-β1,4-GlcNAc) in an alpha-1,3-linkage.

The term “glycopeptide” as used herein refers to a peptide that contains one or more saccharide groups, being mono-, di-, oligo-, polysaccharides and/or glycans, that is/are covalently attached to the side chains of the amino acid residues of the peptide. Glycopeptides comprise natural glycopeptide antibiotics such as e.g., the glycosylated non-ribosomal peptides produced by a diverse group of soil actinomycetes that target Gram-positive bacteria by binding to the acyl-D-alanyl-D-alanine (D-Ala-D-Ala) terminus of the growing peptidoglycan on the outer surface of the cytoplasmatic membrane, and synthetic glycopeptide antibiotics. The common core of natural glycopeptides is made of a cyclic peptide consisting in 7 amino acids, to which are bound 2 sugars. Examples of glycopeptides comprise vancomycin, teicoplanin, oritavancin, chloroeremomycin, telavancin and dalbavancin.

The terms “glycoprotein” and “glycopolypeptide” are used interchangeably and refer to a polypeptide that contains one or more saccharide groups, being mono-, di-, oligo-, polysaccharides and/or glycans, that is/are covalently attached to the side chains of the amino acid residues of the polypeptide.

The term “lipids” as used herein refers to hydrophobic or amphiphilic small biomolecules that are soluble in nonpolar solvents. Lipids range in structure from simple short hydrocarbon chains to more complex molecules, including triacylglycerols, phospholipids and sterols and their esters. Lipids comprise fatty acids, acyl groups and ceramides. A fatty acid is a carboxylic acid with an aliphatic chain, which is either saturated or unsaturated. An acyl group is a functional group used in organic chemistry which is generally known in the art and refers to any RCO-group, wherein the ‘R’ is any carbon chain from 1 CH3 up to hundreds of CH2 ending in a CH3. The ‘R’ chain of said RCO-group can also have other substituents, functional groups or double or triple bonds. The carbon ‘C’ in the acyl group is double bonded to the oxygen ‘O’. A ceramide is a specific class of lipids that refers to a fatty acid that is linked to sphingosine. As used herein, the term “glycolipid” refers to any of the glycolipids which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like for example glycerol (glycoglycerolipids), peptide (glycopeptidolipids), acylated-sphingosine (glycosphingolipids), or other residues (lipopolysaccharides, phenolic glycolipids, nucleoside lipids).

The term “membrane transporter proteins” as used herein refers to proteins that are part of or interact with the cell membrane and control the flow of molecules and information across the cell. The membrane proteins are thus involved in transport, be it import into or export out of the cell.

Such membrane transporter proteins can be but are not limited to porters, P—P-bond-hydrolysis-driven transporters, β-Barrel Porins, auxiliary transport proteins and phosphotransfer-driven group translocators.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Membrane transporter proteins are included in this class when they utilize a carrier-mediated process to catalyse uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy, of secondary carriers (Forrest et al., Biochim. Biophys. Acta 1807 (2011) 167-188). These systems are usually stereospecific. Solute:solute countertransport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolytes, siderophores. Membrane transporter proteins are included in the class of P—P-bond hydrolysis-driven transporters if they hydrolyse the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379). The membrane transporter protein may or may not be transiently phosphorylated, but the substrate is not phosphorylated. Substrates that are transported via the class of P—P-bond hydrolysis-driven transporters include but are not limited to cations, heavy metals, beta-glucan, UDP-glucose, lipopolysaccharides, teichoic acid.

The β-Barrel porins membrane transporter proteins form transmembrane pores that usually allow the energy independent passage of solutes across a membrane. The transmembrane portions of these proteins consist exclusively of β-strands which form a β-barrel (Saier et al., Nucleic Acids Res. 44 (2016) D372-D379). These porin-type proteins are found in the outer membranes of Gram-negative bacteria, mitochondria, plastids, and possibly acid-fast Gram-positive bacteria. Solutes that are transported via these β-Barrel porins include but are not limited to nucleosides, raffinose, glucose, beta-glucosides, oligosaccharides.

The auxiliary transport proteins are defined to be proteins that facilitate transport across one or more biological membranes but do not themselves participate directly in transport. These membrane transporter proteins always function in conjunction with one or more established transport systems such as but not limited to outer membrane factors (OMFs), polysaccharide (PST) porters, the ATP-binding cassette (ABC)-type transporters. They may provide a function connected with energy coupling to transport, play a structural role in complex formation, serve a biogenic or stability function or function in regulation (Saier et al., Nucleic Acids Res. 44 (2016) D372-D379). Examples of auxiliary transport proteins include but are not limited to the polysaccharide copolymerase family involved in polysaccharide transport, the membrane fusion protein family involved in bacteriocin and chemical toxin transport.

The phosphotransfer-driven group translocators are also known as the PEP-dependent phosphoryl transfer-driven group translocators of the bacterial phosphoenolpyruvate:sugar phosphotransferase system (PTS). The product of the reaction, derived from extracellular sugar, is a cytoplasmic sugar-phosphate. The enzymatic constituents, catalysing sugar phosphorylation, are superimposed on the transport process in a tightly coupled process. The PTS system is involved in many different aspects comprising in regulation and chemotaxis, biofilm formation, and pathogenesis (Lengeler, J. Mol. Microbiol. Biotechnol. 25 (2015) 79-93; Saier, J. Mol. Microbiol. Biotechnol. 25 (2015) 73-78). Membrane transporter protein families classified within the phosphotransfer-driven group translocators comprise PTS systems linked to transport of glucose-glucosides, fructose-mannitol, lactose-N,N′-diacetylchitobiose-beta-glucoside, glucitol, galactitol, mannose-fructose-sorbose and ascorbate.

The major facilitator superfamily (MFS) is a superfamily of membrane transporter proteins that are single-polypeptide secondary carriers with InterPro domain IPR036259, capable of transporting small solutes in response to chemiosmotic ion gradients (Pao et al., J. Microbiol. Mol. Biol. Rev. 62 (1998) 1-34; Walmsley et al., Trends Biochem. Sci. 23 (1998) 476-481; Wang et al., Jr. Biochim. Biophys. Acta Biomembr. 1862 (2020) 183277; Teelucksingh et al. 202 (2020) e00367-20). MFS transporters catalyse uniport, solute:cation (H+, but seldom Na+) symport and/or solute:H+ or solute:solute antiport. Most are of 400-amino acyl residues in length and possess either 12, 14, or occasionally, 24 transmembrane α-helical spanners (TMSs).

“SET” or “Sugar Efflux Transporter” as used herein is part of the MFS superfamily and refers to membrane proteins of the SET family which are proteins with InterPro domain IPR004750 and/or are proteins that belong to the eggNOGv5.0.0 family ENOG410XTE9. Identification of the InterPro domain can be done by using the online tool on https://www.ebi.ac.uk/interpro/ or a standalone version of InterProScan (https://www.ebi.ac.uk/interpro/download.html) using the default values. Identification of the orthology family in eggNOGv4.5 can be done using the online version or a standalone version of eggNOG-mapperv2 (http://eggnog-mapper.embl.de). This family of proteins is an efflux system for lactose, glucose, aromatic glucosides and galactosides, cellobiose, maltose, a-methyl glucoside and other sugar compounds. They are found in both Gram-negative and Gram-positive bacteria (Liu et al., Mol. Microbiol. 31 (1999) 1845-1851; Liu et al., J. Biol. Chem. 274 (1999) 22977-22984; Sun and Vanderpool, J. Bacteriol. 193 (2011) 143-153).

The term “Siderophore” as used herein is referring to the secondary metabolite of various microorganisms which are mainly ferric ion specific chelators (Neilands, J. Biol. Chem. 270 (1995) 26723-26726). These molecules have been classified as catecholate, hydroxamate, carboxylate and mixed types. Siderophores are in general synthesized by a nonribosomal peptide synthetase (NRPS) dependent pathway or an NRPS independent pathway (NIS) (Barry and Challis, Curr. Opin. Chem. Biol. 13 (2009) 205-215). The most important precursor in NRPS-dependent siderophore biosynthetic pathway is chorismate. 2, 3-DHBA could be formed from chorismate by a three-step reaction catalysed by isochorismate synthase, isochorismatase, and 2,3-dihydroxybenzoate-2,3-dehydrogenase. Siderophores can also be formed from salicylate which is formed from isochorismate by isochorismate pyruvate lyase. When ornithine is used as precursor for siderophores, biosynthesis depends on the hydroxylation of ornithine catalysed by L-ornithine N5-monooxygenase. In the NIS pathway, an important step in siderophore biosynthesis is N(6)-hydroxylysine synthase.

A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily (Teelucksingh et al. 202 (2020) e00367-20). In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two groups generally cluster loosely together. ATP hydrolysis without protein phosphorylation energizes transport. There are dozens of families within the ABC superfamily, and family generally correlates with substrate specificity (Davidson et al. Microbiol. Mol. Biol. Rev. 72 (2008) 317-364; Goffeau et al. (2013). “ABC Transporters”. In Lane W J, Lennarz M D (eds.). Encyclopedia of Biological Chemistry (Second ed.). London: Academic Press. pp. 7-11).

It should be understood for those skilled in the art that for the databases used herein, comprising EggNOG 5.0.0 (released November 2018) and InterPro 86.0 (released 3 Jun. 2021), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to α 1,2; α 1,3; α 1,4 and/or α 1,6 fucosylated oligosaccharides.

The term “enabled efflux” means to introduce the activity of transport of a solute over the cytoplasm membrane and/or the cell wall. Said transport may be enabled by introducing and/or increasing the expression of a membrane transporter protein as described in the present invention. The term “enhanced efflux” means to improve the activity of transport of a solute over the cytoplasm membrane and/or the cell wall. Transport of a solute over the cytoplasm membrane and/or cell wall may be enhanced by introducing and/or increasing the expression of a membrane transporter protein as described in the present invention. “Expression” of a membrane transporter protein is defined as “overexpression” of the gene encoding said membrane transporter protein in the case said gene is an endogenous gene or “expression” in the case the gene encoding said membrane transporter protein is a heterologous gene that is not present in the wild-type strain or cell.

The term “purified” refers to material that is substantially or essentially free from components which interfere with the activity of the biological molecule. For cells, saccharides, nucleic acids, polypeptides, peptides, glycoproteins, glycopeptides, lipids and glycolipids the term “purified” refers to material that is substantially or essentially free from components which normally accompany the material as found in its native state. Typically, purified saccharides, oligosaccharides, peptides, glycopeptides, proteins, glycoproteins, lipids, glycolipids or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins which can be present in an aqueous medium like e.g., a cultivation or an incubation. The term “clarifying” as used herein refers to the act of treating an aqueous medium like e.g., a cultivation or an incubation, to remove suspended particulates and contaminants from the production process, like e.g., cells, cell components, insoluble metabolites and debris, that could interfere with the eventual purification of the one or more bioproduct(s). Such treatment can be carried out in a conventional manner by centrifugation, flocculation, flocculation with optional ultrasonic treatment, gravity filtration, microfiltration, foam separation or vacuum filtration (e.g., through a ceramic filter which can include a Celite™ filter aid).

The term “cultivation” refers to the culture medium wherein the cell is cultivated or fermented, the cell itself, and a fucosylated compound and/or 3-FL of present invention that is produced by the cell in whole broth, i.e. inside (intracellularly) as well as outside (extracellularly) of the cell.

The terms “culture medium” and “cultivation medium” as used herein are used interchangeably and refer to the medium wherein the cell is cultivated.

The term “incubation” refers to a mixture wherein a fucosylated compound of present invention and/or 3-FL is produced. Said mixture can comprise one or more enzyme(s) and one or more precursor(s) as defined herein present in a buffered solution and incubated for a certain time at a certain temperature enabling production of a fucosylated compound of present invention and/or 3-FL, catalysed by said one or more enzyme(s) using said one or more precursor(s) in said mixture. Said mixture can also comprise i) the cell obtained after cultivation or incubation, optionally said cell is subjected to cell lysis, ii) a buffered solution or the cultivation or incubation medium wherein the cell was cultivated or fermented, and iii) a fucosylated compound of present invention and/or 3-FL that is/are produced by the cell in whole broth, i.e., inside (intracellularly) as well as outside (extracellularly) of the cell. Said incubation can also be the cultivation as defined herein.

The terms “reactor” and “incubator” refer to the recipient filled with the cultivation or incubation. Examples of reactors and incubators comprise but are not limited to microfluidic devices, well plates, tubes, shake flasks, fermenters, bioreactors, process vessels, cell culture incubators, CO2 incubators. Said reactor and incubator can each vary from lab-scale dimensions to large-scale industrial dimensions. As used herein, the term “cell productivity index (CPI)” refers to the mass of the product produced by the cells divided by the mass of the cells produced in the culture.

The term “precursor” as used herein refers to substances which are taken up and/or synthetized by the cell for the specific production of a fucosylated compound and/or 3-FL according to the present invention. In this sense a precursor can be a saccharide substrate comprising Gal-β1,m-GlcNAc-β1,n-Gal-β1,4-Glc as described herein, but can also be another substance, metabolite, a mono-, di- or oligosaccharide, a protein, a glycoprotein, a peptide, a glycopeptide, a lipid or glycolipid which is first modified within the cell as part of the biochemical synthesis route of a fucosylated compound as described herein and/or 3-FL. The term “precursor” as used herein is also to be understood as a chemical compound that participates in a chemical or enzymatic reaction to produce another compound like e.g., an intermediate, as part in the metabolic pathway of a fucosylated compound of present invention and/or 3-FL according to the present invention. The term “precursor” as used herein is also to be understood as a donor that is used by a glycosyltransferase to modify an acceptor substrate with a sugar moiety in a glycosidic bond, as part in the metabolic pathway of a fucosylated compound of present invention and/or 3-FL according to the present invention. Examples of such precursors comprise glucose, galactose, fructose, glycerol, sialic acid, fucose, mannose, maltose, sucrose, lactose, dihydroxyacetone, glucosamine, N-acetyl-glucosamine, mannosamine, N-acetyl-mannosamine, galactosamine, N-acetylgalactosamine, galactosyllactose, N-acetyl-lactosamine (LacNAc) and oligosaccharide containing 1 or more N-acetyllactosamine units or an intermediate into oligosaccharide, fucosylated and sialylated versions thereof, lacto-N-triose, a substrate comprising Gal-β1,3-GlcNAc, lacto-N-biose (LNB), lacto-N-tetose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and/or 1 or more lacto-N-biose units, peptides, polypeptides, lipids, sphingolipids, cerebrosides, ceramide lipids, phosphatidylinositol lipids, and glycosylated versions of peptides, polypeptides, lipids, sphingolipids, cerebrosides, ceramide lipids, phosphatidylinositol lipids, phosphorylated sugars like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetyl-glucosamine-6-phosphate, N-acetylmannosamine-6-phosphate, N-acetylglucosamine-1-phosphate, N-acetyl-neuraminic acid-9-phosphate and nucleotide-activated sugars as defined herein like e.g. UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, fucose transporter, transporter for a nucleotide-activated sugar wherein said transporter internalizes a to the medium added precursor for the production of a fucosylated compound of present invention and/or 3-FL.

### DETAILED DESCRIPTION OF THE INVENTION

According to a first aspect, the present invention provides a method for the production of a fucosylated compound. The method comprises the steps of a) providing i) GDP-fucose, ii) a saccharide substrate comprising Gal-β1,m-GlcNAc-β1,n-Gal-β1,4-Glc wherein said m is 3 or 4 and said n is 3 or 6, and iii) a fucosyltransferase, and b) contacting said fucosyltransferase and GDP-fucose with said saccharide substrate under conditions where the fucosyltransferase catalyses the transfer of a fucose residue from said GDP-fucose to the N-acetylglucosamine (GlcNAc) and/or the glucose (Glc) residue of Gal-β1,m-GlcNAc-β1,n-Gal-β1,4-Glc of said saccharide substrate in an alpha-1,3-glycosidic linkage resulting in the production of said fucosylated compound, c) preferably, separating said produced fucosylated compound. Optionally, said saccharide substrate is linked to a peptide, a protein and/or a lipid.

Throughout the application, the term “saccharide substrate comprising Gal-β1,m-GlcNAc-β1,n-Gal-β1,4-Glc” may be replaced by the term “saccharide substrate” or by the term “substrate comprising “Gal-β1,m-GlcNAc-β1,n-Gal-β1,4-Glc” and vice versa.

In a preferred embodiment of the method and/or cell of present invention, the fucosylated compound is a saccharide comprising Gal-β1,m-[Fuc-α1,3]-GlcNAc-β1,n-Gal-β1,4-Glc, wherein said m is 3 or 4 and said n is 3 or 6. Said fucosylated compound may be a saccharide comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc. Optionally, said saccharide is linked to a peptide, a protein and/or a lipid. In a more preferred embodiment, said fucosylated compound is a saccharide chosen from the list comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc. Optionally, said saccharide is linked to a peptide, a protein and/or a lipid.

In an alternative more preferred embodiment, said fucosylated compound is a saccharide comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc, wherein said Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc is glycosidically linked to one or more monosaccharide residues. Said one or more monosaccharide residues is/are chosen from the list of monosaccharide residues as defined herein, comprising N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), a sialic acid, Neu5Ac, Neu5Gc, N-acetylgalactosamine (GalNAc), galactosamine (Galn), fucose (Fuc), rhamnose (Rha), glucuronic acid, gluconic acid, fructose (Fru) and polyols. Said one or more monosaccharide residue(s) may be glycosidically linked to said non-reducing Gal, internal Gal and/or reducing Glc residue of said Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc of said saccharide. Said glycosidic linkages comprise alpha- and beta-glycosidic linkages. Said fucosylated compound may be a saccharide with a linear structure. Alternatively, said fucosylated compound may be a saccharide with a branched structure. Said saccharide may be an oligosaccharide, a polysaccharide or a glycan as defined herein. Optionally, said saccharide is linked to a peptide, a protein and/or a lipid.

In another preferred embodiment, the fucosylated compound is an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation (DP) of at least six. Said fucosylated compound is an oligosaccharide 1) comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc and 2) having a DP of at least six. The degree of polymerization of an oligosaccharide refers to the number of monosaccharide units present in the oligosaccharide structure. An oligosaccharide with a DP of at least six refers to a hexasaccharide, a heptasaccharide or saccharide structures comprising eight or more monosaccharide residues. Said fucosylated compound may be an oligosaccharide comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc wherein said Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc is glycosidically linked to at least one monosaccharide residue chosen from the list of monosaccharide residues as defined herein resulting in a fucosylated compound being an oligosaccharide with a DP of at least 6. Said at least one monosaccharide residue may be glycosidically linked to said non-reducing Gal, internal Gal and/or reducing Glc residue of said Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc of said oligosaccharide. Said glycosidic linkages comprise alpha- and beta-glycosidic linkages. In an even more preferred embodiment, the fucosylated compound is an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation of at least seven. In other words, said fucosylated compound may be an oligosaccharide comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc wherein said Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc or Gal-β1,4-[Fuc-α1,3]-GlcNAc-β1,6-Gal-β1,4-Glc is glycosidically linked to at least two monosaccharide residues chosen from the list of monosaccharide residues as defined herein resulting in a fucosylated compound being an oligosaccharide with a DP of at least 7. Said fucosylated compound may be an oligosaccharide with a linear structure. Alternatively, said fucosylated compound may be an oligosaccharide with a branched structure. Optionally, said oligosaccharide is linked to a peptide, a protein and/or a lipid.

In another preferred embodiment of the method and/or cell of the present invention, the fucosylated compound is a saccharide comprising a formula Ra-[Rb]-[Re]-[Rf]-Gal-β1,m-[Fuc-α1,3]-GlcNAc-β1,n-[Rc]-Gal-β1,4-Glc-[Rd], wherein said m is 3 or 4 and said n is 3 or 6 and i) wherein the non-reducing Gal residue of said Ra-[Rb]-[Re]-[Rf]-Gal-β1,m-[Fuc-α1,3]-GlcNAc-β1,n-[Rc]-Gal-β1,4-Glc-[Rd] is glycosidically linked to an Ra, Rb, Re and/or an Rf group, and/or ii) wherein the internal Gal residue of said Ra-[Rb]-[Re]-[Rf]-Gal-β1,m-[Fuc-α1,3]-GlcNAc-β1,n-[Rc]-Gal-β1,4-Glc-[Rd] is glycosidically linked to an Rc group and/or iii) wherein the reducing Glc residue of said Ra-[Rb]-[Re]-[Rf]-Gal-β1,m-[Fuc-α1,3]-GlcNAc-β1,n-[Rc]-Gal-β1,4-Glc-[Rd] is glycosidically linked to an Rd group. In the scope of the invention, any one of said Ra, Rb, Rc, Rd, Re and Rf groups is chosen from the list comprising a monosaccharide, a disaccharide and an oligosaccharide, as described herein. Optionally, said saccharide is linked to a peptide, a protein and/or a lipid.

In a more preferred embodiment of the method and/or cell of the present invention, the fucosylated compound is a saccharide comprising the formula Gal-β1,m-[Fuc-α1,3]-GlcNAc-β1,n-Gal-β1,4-Glc-[Rd] wherein said m is 3 or 4 and said n is 3 or 6, wherein the reducing Glc residue of said Gal-β1,m-[Fuc-α1,3]-GlcNAc-β1,n-Gal-β1,4-Glc-[Rd] is linked to an Rd group via an alpha-glycosidic or beta-glycosidic linkage and wherein said Rd group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Gal-β1,m-[Fuc-α1,3]-GlcNAc-β1,n-[Rc]-Gal-β1,4-Glc, wherein said m is 3 or 4 and said n is 3 or 6, wherein the internal Gal residue of said Gal-β1,m-[Fuc-α1,3]-GlcNAc-β1,n-[Rc]-Gal-β1,4-Glc is linked to an Rc group via an alpha-glycosidic or beta-glycosidic linkage and wherein said Rc group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Gal-β1,m-[Fuc-α1,3]-GlcNAc-β1,n-[Rc]-Gal-β1,4-Glc-[Rd] wherein said m is 3 or 4 and said n is 3 or 6, wherein the reducing Glc residue of said Gal-β1,m-[Fuc-α1,3]-GlcNAc-β1,n-[Rc]-Gal-β1,4-Glc-[Rd] is linked to an Rd group via an alpha-glycosidic or beta-glycosidic linkage and wherein the internal Gal residue of said Gal-β1,m-[Fuc-α1,3]-GlcNAc-β1,n-[Rc]-Gal-β1,4-Glc-[Rd] is linked to an Rc group via an alpha-glycosidic or beta-glycosidic linkage and wherein said Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-Gal-β1,4-Glc wherein said m is 3 or 4 and said n is 3 or 6, wherein the non-reducing Gal residue of said Ra-Gal-β1,m-[Fuc-α1,3]-GlcNAc-β1,n-Gal-β1,4-Glc is linked to an Ra group via an alpha-glycosidic or beta-glycosidic linkage and wherein said Ra group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-Gal-β1,4-Glc wherein said m is 3 or 4 and said n is 3 or 6, wherein the non-reducing Gal residue of said Ra-[Rb]-Gal-β1,m-[Fuc-α1,3]-GlcNAc-β1,n-Gal-β1,4-Glc is linked to 1) an Ra group via an alpha-glycosidic or beta-glycosidic linkage and 2) an Rb group via an alpha-glycosidic or beta-glycosidic linkage, and wherein said Ra group and Rb group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-Gal-β1,4-Glc wherein said m is 3 or 4 and said n is 3 or 6, wherein the non-reducing Gal residue of said Ra-[Rb]-[Re]-Gal-β1,m-[Fuc-α1,3]-GlcNAc-β1,n-Gal-β1,4-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, and wherein said Ra group, Rb group and Re group are chosen from the list comprising a monosaccharide, a disaccharide and an oligosaccharide. Optionally, said saccharide is linked to a peptide, protein or lipid. In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-Gal-β1,4-Glc wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-Gal-β1,4-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, and wherein said Ra group, Rb group, Re group and Rf group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-Gal-β1,4-Glc-[Rd] wherein said m is 3 or 4 and said n is 3 or 6, wherein the non-reducing Gal residue of said Ra-Gal-β1,m-[Fuc-α1,3]-GlcNAc-β1,n-Gal-β1,4-Glc-[Rd] is linked to an Ra group via an alpha-glycosidic or beta-glycosidic linkage, wherein the reducing Glc residue of said Ra-Gal-β1,m-[Fuc-α1,3]-GlcNAc-β1,n-Gal-β1,4-Glc-[Rd] is linked to an Rd group via an alpha-glycosidic or beta-glycosidic linkage, and wherein said Ra group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-[Rc]-Gal-β1,4-Glc wherein said m is 3 or 4 and said n is 3 or 6, wherein the non-reducing Gal residue of said Ra-Gal-β1,m-[Fuc-α1,3]-GlcNAc-β1,n-[Rc]-Gal-β1,4-Glc] is linked to an Ra group via an alpha-glycosidic or beta-glycosidic linkage, wherein the internal Gal residue of said Ra-Gal-β1,m-[Fuc-α1,3]-GlcNAc-β1,n-[Rc]-Gal-β1,4-Glc is linked to an Rc group via an alpha-glycosidic or beta-glycosidic linkage, and wherein said Ra group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-[Rc]-Gal-β1,4-Glc-[Rd] wherein said m is 3 or 4 and said n is 3 or 6, wherein the non-reducing Gal residue of said Ra-Gal-β1,m-[Fuc-α1,3]-GlcNAc-β1,n-[Rc]-Gal-β1,4-Glc-[Rd] is linked to an Ra group via an alpha-glycosidic or beta-glycosidic linkage, wherein the internal Gal residue of said Ra-Gal-β1,m-[Fuc-α1,3]-GlcNAc-β1,n-[Rc]-Gal-β1,4-Glc-[Rd] is linked to an Rc group via an alpha-glycosidic or beta-glycosidic linkage, wherein the reducing Glc residue of said Ra-Gal-β1,m-[Fuc-α1,3]-GlcNAc-β1,n-[Rc]-Gal-β1,4-Glc-[Rd] is linked to an Rd group via an alpha-glycosidic or beta-glycosidic linkage, and wherein said Ra group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-Gal-β1,4-Glc-[Rd] wherein said m is 3 or 4 and said n is 3 or 6, wherein the non-reducing Gal residue of said Ra-[Rb]-Gal-β1,m-[Fuc-α1,3]-GlcNAc-β1,n-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the reducing Glc residue of said Ra-[Rb]-Gal-β1,m-[Fuc-α1,3]-GlcNAc-β1,n-Gal-β1,4-Glc-[Rd] is linked to an Rd group via an alpha-glycosidic or beta-glycosidic linkage, and wherein said Ra group, Rb group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-[Rc]-Gal-β1,4-Glc wherein said m is 3 or 4 and said n is 3 or 6, wherein the non-reducing Gal residue of said Ra-[Rb]-Gal-β1,m-[Fuc-α1,3]-GlcNAc-β1,n-[Rc]-Gal-β1,4-Glc is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-[Fuc-α1,3]-GlcNAc-β1,n-[Rc]-Gal-β1,4-Glc is linked to an Rc group via an alpha-glycosidic or beta-glycosidic linkage, and wherein said Ra group, Rb group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-[Rc]-Gal-β1,4-Glc-[Rd] wherein said m is 3 or 4 and said n is 3 or 6, wherein the non-reducing Gal residue of said Ra-[Rb]-Gal-β1,m-[Fuc-α1,3]-GlcNAc-β1,n-[Rc]-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-[Fuc-α1,3]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-Gal-β1,m-[Fuc-α1,3]-GlcNAc-β1,n-[Rc]-Gal-β1,4-Glc-[Rd] is linked to an Rd group via an alpha-glycosidic or beta-glycosidic linkage, and wherein said Ra group, Rb group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-Gal-β1,4-Glc-[Rd] wherein said m is 3 or 4 and said n is 3 or 6, wherein the non-reducing Gal residue of said Ra-[Rb]-[Re]-Gal-β1,m-[Fuc-α1,3]-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the reducing Glc residue of said Ra-[Rb]-[Re]-Gal-β1,m-[Fuc-α1,3]-GlcNAc-β1,n-Gal-β1,4-Glc-[Rd] is linked to an Rd group via an alpha-glycosidic or beta-glycosidic linkage, and wherein said Ra group, Rb group, Re group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-[Rc]-Gal-β1,4-Glc wherein said m is 3 or 4 and said n is 3 or 6, wherein the non-reducing Gal residue of said Ra-[Rb]-[Re]-Gal-β1,m-[Fuc-α1,3]-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-[Fuc-α1,3]-GlcNAc-β1,n-[Rc]-Gal-β1,4-Glc is linked to an Rc group via an alpha-glycosidic or beta-glycosidic linkage, and wherein said Ra group, Rb group, Re group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-[Rc]-Gal-β1,4-Glc-[Rd] wherein said m is 3 or 4 and said n is 3 or 6, wherein the non-reducing Gal residue of said Ra-[Rb]-[Re]-Gal-β1,m-[Fuc-α1,3]-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-[Fuc-α1,3]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-Gal-β1,m-[Fuc-α1,3]-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-Gal-β1,4-Glc-[Rd] wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the reducing Glc residue of said Ra-[Rb]-[Re]-[Rf]-Gal-β1,m-[Fuc-α1,3]-GlcNAc-β1,n-Gal-β1,4-Glc-[Rd] is linked to an Rd group via an alpha-glycosidic or beta-glycosidic linkage, and wherein said Ra group, Rb group, Re group, Rf group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-[Rc]-Gal-β1,4-Glc wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-[Fuc-α1,3]-GlcNAc-β1,n-[Rc]-Gal-β1,4-Glc is linked to an Rc group via an alpha-glycosidic or beta-glycosidic linkage, and wherein said Ra group, Rb group, Re group, Rf group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-[Rc]-Gal-β1,4-Glc-[Rd] wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-[Fuc-α1,3]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-[Rf]-Gal-β1,m-[Fuc-α1,3]-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rf group, Rc group and Rd group are chosen from the list comprising a monosaccharide, a disaccharide and an oligosaccharide. Optionally, said saccharide is linked to a peptide, protein or lipid.

In another preferred embodiment of the method and/or cell of present invention, the fucosylated compound is a saccharide comprising Gal-β1,m-GlcNAc-β1,n-Gal-β1,4-[Fuc-α1,3]-Glc, wherein said m is 3 or 4 and said n is 3 or 6. Said fucosylated compound may be a saccharide comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc. Optionally, said saccharide is linked to a peptide, a protein and/or a lipid. In a more preferred embodiment, said fucosylated compound is a saccharide chosen from the list comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc. Optionally, said saccharide is linked to a peptide, a protein and/or a lipid.

In an alternative more preferred embodiment, said fucosylated compound is a saccharide comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc, wherein said Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc is glycosidically linked to one or more monosaccharide residues. Said one or more monosaccharide residues is/are chosen from the list of monosaccharide residues as defined herein, comprising N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), a sialic acid, Neu5Ac, Neu5Gc, N-acetylgalactosamine (GalNAc), galactosamine (Galn), fucose (Fuc), rhamnose (Rha), glucuronic acid, gluconic acid, fructose (Fru) and polyols. Said one or more monosaccharide residue(s) may be glycosidically linked to said non-reducing Gal, internal Gal and/or GlcNAc residue of said Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc of said saccharide. Said glycosidic linkages comprise alpha- and beta-glycosidic linkages. Said fucosylated compound may be a saccharide with a linear structure. Alternatively, said fucosylated compound may be a saccharide with a branched structure. Said saccharide may be an oligosaccharide, a polysaccharide or a glycan as defined herein. Optionally, said saccharide is linked to a peptide, a protein and/or a lipid.

In another preferred embodiment, the fucosylated compound is an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation (DP) of at least six. Said fucosylated compound is an oligosaccharide 1) comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc and 2) having a DP of at least six. The degree of polymerization of an oligosaccharide refers to the number of monosaccharide units present in the oligosaccharide structure. An oligosaccharide with a DP of at least six refers to a hexasaccharide, a heptasaccharide or saccharide structures comprising eight or more monosaccharide residues. Said fucosylated compound may be an oligosaccharide comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc wherein said Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc is glycosidically linked to at least one monosaccharide residue chosen from the list of monosaccharide residues as defined herein resulting in a fucosylated compound being an oligosaccharide with a DP of at least 6. Said at least one monosaccharide residue may be glycosidically linked to said non-reducing Gal, internal Gal and/or GlcNAc residue of said Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc of said oligosaccharide. Said glycosidic linkages comprise alpha- and beta-glycosidic linkages. In an even more preferred embodiment, the fucosylated compound is an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation of at least seven. In other words, said fucosylated compound may be an oligosaccharide comprising Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc wherein said Gal-β1,3-GlcNAc-β1,3-Gal-β1,4-[Fuc-α1,3]-Glc, Gal-β1,3-GlcNAc-β1,6-Gal-β1,4-[Fuc-α1,3]-Glc, Gal-β1,4-GlcNAc-β1,3-Gal-β1,4-[Fuc-α1,3]-Glc or Gal-β1,4-GlcNAc-β1,6-Gal-β1,4-[Fuc-α1,3]-Glc is glycosidically linked to at least two monosaccharide residues chosen from the list of monosaccharide residues as defined herein resulting in a fucosylated compound being an oligosaccharide with a DP of at least 7. Said fucosylated compound may be an oligosaccharide with a linear structure. Alternatively, said fucosylated compound may be an oligosaccharide with a branched structure. Optionally, said oligosaccharide is linked to a peptide, a protein and/or a lipid.

In another preferred embodiment of the method and/or cell of the present invention, the fucosylated compound is a saccharide comprising a formula Ra-[Rb]-[Re]-[Rf]-Gal-β1,m-[Rg]-GlcNAc-β1,n-[Rc]-Gal-β1,4-[Fuc-α1,3]-Glc, wherein said m is 3 or 4 and said n is 3 or 6 and i) wherein the non-reducing Gal residue of said Ra-[Rb]-[Re]-[Rf]-Gal-β1,m-[Rg]-GlcNAc-β1,n-[Rc]-Gal-β1,4-[Fuc-α1,3]-Glc is glycosidically linked to an Ra, Rb, Re and/or an Rf group, and/or ii) wherein the GlcNAc residue of said Ra-[Rb]-[Re]-[Rf]-Gal-β1,m-[Rg]-GlcNAc-β1,n-[Rc]-Gal-β1,4-[Fuc-α1,3]-Glc is glycosidically linked to an Rg group and/or iii) wherein the internal Gal residue of said Ra-[Rb]-[Re]-[Rf]-Gal-β1,m-[Rg]-GlcNAc-β1,n-[Rc]-Gal-β1,4-[Fuc-α1,3]-Glc is glycosidically linked to an Rc group. In the scope of the invention, any one of said Ra, Rb, Rc, Re, Rf and Rg groups is chosen from the list comprising a monosaccharide, a disaccharide and an oligosaccharide, as described herein. Optionally, said saccharide is linked to a peptide, a protein and/or a lipid.

In a more preferred embodiment of the method and/or cell of the present invention, the fucosylated compound is a saccharide comprising the formula Gal-β1,m-[Rg]-GlcNAc-β1,n-Gal-β1,4-[Fuc-α1,3]-Glc wherein said m is 3 or 4 and said n is 3 or 6, wherein the reducing GlcNAc residue of said Gal-β1,m-[Rg]-GlcNAc-β1,n-Gal-β1,4-[Fuc-α1,3]-Glc is linked to an Rg group via an alpha-glycosidic or beta-glycosidic linkage and wherein said Rg group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Gal-β1,m-GlcNAc-β1,n-[Rc]-Gal-β1,4-[Fuc-α1,3]-Glc, wherein said m is 3 or 4 and said n is 3 or 6, wherein the internal Gal residue of said Gal-β1,m-GlcNAc-β1,n-[Rc]-Gal-β1,4-[Fuc-α1,3]-Glc is linked to an Rc group via an alpha-glycosidic or beta-glycosidic linkage and wherein said Rc group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Gal-β1,m-[Rg]-GlcNAc-β1,n-[Rc]-Gal-β1,4-[Fuc-α1,3]-Glc wherein said m is 3 or 4 and said n is 3 or 6, wherein the GlcNAc residue of said Gal-β1,m-[Rg]-GlcNAc-β1,n-[Rc]-Gal-β1,4-[Fuc-α1,3]-Glc is linked to an Rg group via an alpha-glycosidic or beta-glycosidic linkage and wherein the internal Gal residue of said Gal-β1,m-[Rg]-GlcNAc-β1,n-[Rc]-Gal-β1,4-[Fuc-α1,3]-Glc is linked to an Rc group via an alpha-glycosidic or beta-glycosidic linkage and wherein said Rc group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-GlcNAc-β1,n-Gal-β1,4-[Fuc-α1,3]-Glc wherein said m is 3 or 4 and said n is 3 or 6, wherein the non-reducing Gal residue of said Ra-Gal-β1,m-GlcNAc-β1,n-Gal-β1,4-[Fuc-α1,3]-Glc is linked to an Ra group via an alpha-glycosidic or beta-glycosidic linkage and wherein said Ra group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-GlcNAc-β1,n-Gal-β1,4-[Fuc-α1,3]-Glc wherein said m is 3 or 4 and said n is 3 or 6, wherein the non-reducing Gal residue of said Ra-[Rb]-Gal-β1,m-GlcNAc-β1,n-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, and wherein said Ra group and Rb group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-Gal-β1,m-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, and wherein said Ra group, Rb group and Re group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, and wherein said Ra group, Rb group, Re group and Rf group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Rg]-GlcNAc-β1,n-Gal-β1,4-[Fuc-α1,3]-Glc wherein said m is 3 or 4 and said n is 3 or 6, wherein the non-reducing Gal residue of said Ra-Gal-β1,m-[Rg]-GlcNAc-β1,n-Gal-β1,4-[Fuc-α1,3]-Glc is linked to an Ra group via an alpha-glycosidic or beta-glycosidic linkage, wherein the GlcNAc residue of said Ra-Gal-β1,m-[Rg]-GlcNAc-β1,n-Gal-β1,4-[Fuc-α1,3]-Glc is linked to an Rg group via an alpha-glycosidic or beta-glycosidic linkage, and wherein said Ra group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-GlcNAc-β1,n-[Rc]-Gal-β1,4-[Fuc-α1,3]-Glc wherein said m is 3 or 4 and said n is 3 or 6, wherein the non-reducing Gal residue of said Ra-Gal-β1,m-GlcNAc-β1,n-[Rc]-Gal-β1,4-[Fuc-α1,3]-Glc is linked to an Ra group via an alpha-glycosidic or beta-glycosidic linkage, wherein the internal Gal residue of said Ra-Gal-β1,m-GlcNAc-β1,n-[Rc]-Gal-β1,4-[Fuc-α1,3]-Glc is linked to an Rc group via an alpha-glycosidic or beta-glycosidic linkage, and wherein said Ra group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Rg]-GlcNAc-β1,n-[Rc]-Gal-β1,4-[Fuc-α1,3]-Glc wherein said m is 3 or 4 and said n is 3 or 6, wherein the non-reducing Gal residue of said Ra-Gal-β1,m-[Rg]-GlcNAc-β1,n-[Rc]-Gal-β1,4-[Fuc-α1,3]-Glc is linked to an Ra group via an alpha-glycosidic or beta-glycosidic linkage, wherein the GlcNAc residue of said Ra-Gal-β1,m-[Rg]-GlcNAc-β1,n-[Rc]-Gal-β1,4-[Fuc-α1,3]-Glc is linked to an Rg group via an alpha-glycosidic or beta-glycosidic linkage, wherein the internal Gal residue of said Ra-Gal-β1,m-[Rg]-GlcNAc-β1,n-[Rc]-Gal-β1,4-[Fuc-α1,3]-Glc is linked to an Rc group via an alpha-glycosidic or beta-glycosidic linkage, and wherein said Ra group, Rc group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Rg]-GlcNAc-β1,n-Gal-β1,4-[Fuc-α1,3]-Glc wherein said m is 3 or 4 and said n is 3 or 6, wherein the non-reducing Gal residue of said Ra-[Rb]-Gal-β1,m-[Rg]-GlcNAc-β1,n-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-Gal-β1,4-[Fuc-α1,3]-Glc is linked to an Rg group via an alpha-glycosidic or beta-glycosidic linkage, and wherein said Ra group, Rb group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-Gal-β1,m-GlcNAc-β1,n-[Rc]-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Rg]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-Gal-β1,m-[Rg]-GlcNAc-β1,n-[Rc]-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-[Rc]-Gal-β1,4-[Fuc-α1,3]-Glc is linked to an Rg group via an alpha-glycosidic or beta-glycosidic linkage, wherein the internal Gal residue of said Ra-[Rb]-Gal-β1,m-[Rg]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Rc group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Rg]-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-Gal-β1,m-[Rg]-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said Ra-[Rb]-[Re]-Gal-β1,m-[Rg]-GlcNAc-β1,n-Gal-β1,4-[Fuc-α1,3]-Glc is linked to an Rg group via an alpha-glycosidic or beta-glycosidic linkage, and wherein said Ra group, Rb group, Re group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-Gal-β1,m-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Rg]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-Gal-β1,m-[Rg]-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said Ra-[Rb]-[Re]-Gal-β1,m-[Rg]-GlcNAc-β1,n-[Rc]-Gal-β1,4-[Fuc-α1,3]-Glc is linked to an Rg group via an alpha-glycosidic or beta-glycosidic linkage, wherein the internal Gal residue of said Ra-[Rb]-[Re]-Gal-β1,m-[Rg]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group, Rc group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Rg]-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-[Rg]-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said Ra-[Rb]-[Re]-[Rf]-Gal-β1,m-[Rg]-GlcNAc-β1,n-Gal-β1,4-[Fuc-α1,3]-Glc is linked to an Rg group via an alpha-glycosidic or beta-glycosidic linkage, and wherein said Ra group, Rb group, Re group, Rf group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group, Rf group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Rg]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-[Rg]-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said Ra-[Rb]-[Re]-[Rf]-Gal-β1,m-[Rg]-GlcNAc-β1,n-[Rc]-Gal-β1,4-[Fuc-α1,3]-Glc is linked to an Rg group via an alpha-glycosidic or beta-glycosidic linkage, wherein the internal Gal residue of said Ra-[Rb]-[Re]-[Rf]-Gal-β1,m-[Rg]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group, Rf group, Rc group and Rg group are chosen from the list comprising a monosaccharide, a disaccharide and an oligosaccharide. Optionally, said saccharide is linked to a peptide, protein or lipid.

In another preferred embodiment of the method and/or cell of present invention, the fucosylated compound is a saccharide comprising Gal-β1,m-[Fuc-α1,3]-GlcNAc-β1,n-Gal-β1,4-[Fuc-α1,3]-Glc, wherein said m is 3 or 4 and said n is 3 or 6. Said fucosylated compound may be a saccharide comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc. Optionally, said saccharide is linked to a peptide, a protein and/or a lipid. In a more preferred embodiment, said fucosylated compound is a saccharide chosen from the list comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc. Optionally, said saccharide is linked to a peptide, a protein and/or a lipid. In an alternative more preferred embodiment, said fucosylated compound is a saccharide comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc, wherein said Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc is glycosidically linked to one or more monosaccharide residues. Said one or more monosaccharide residues is/are chosen from the list of monosaccharide residues as defined herein, comprising N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), a sialic acid, Neu5Ac, Neu5Gc, N-acetylgalactosamine (GalNAc), galactosamine (Galn), fucose (Fuc), rhamnose (Rha), glucuronic acid, gluconic acid, fructose (Fru) and polyols. Said one or more monosaccharide residue(s) may be glycosidically linked to said non-reducing Gal and/or internal Gal residue of said Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc of said saccharide. Said glycosidic linkages comprise alpha- and beta-glycosidic linkages. Said fucosylated compound may be a saccharide with a linear structure. Alternatively, said fucosylated compound may be a saccharide with a branched structure. Said saccharide may be an oligosaccharide, a polysaccharide or a glycan as defined herein. Optionally, said saccharide is linked to a peptide, a protein and/or a lipid.

In another preferred embodiment, the fucosylated compound is an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation (DP) of at least seven. Said fucosylated compound is an oligosaccharide 1) comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc and 2) having a DP of at least seven. The degree of polymerization of an oligosaccharide refers to the number of monosaccharide units present in the oligosaccharide structure. An oligosaccharide with a DP of at least seven refers to a heptasaccharide or saccharide structures comprising eight or more monosaccharide residues. Said fucosylated compound may be an oligosaccharide comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc wherein said Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-(1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc is glycosidically linked to at least one monosaccharide residue chosen from the list of monosaccharide residues as defined herein resulting in a fucosylated compound being an oligosaccharide with a DP of at least 7. Said at least one monosaccharide residue may be glycosidically linked to said non-reducing Gal and/or internal Gal residue of said Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc of said oligosaccharide. Said glycosidic linkages comprise alpha- and beta-glycosidic linkages. In an even more preferred embodiment, the fucosylated compound is an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation of at least eighth. In other words, said fucosylated compound may be an oligosaccharide comprising Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc wherein said Gal-β1,3-[Fuc-α1,3]-GlcNAc-β1,3-Gal-β1,4-[Fuc-α1,3]-Glc, Gal-β1,3-[Fuc-α1,3]-GlcNAc-β1,3-Gal-β1,6-[Fuc-α1,3]-Glc, Gal-β1,4-[Fuc-α1,3]-GlcNAc-β1,3-Gal-β1,4-[Fuc-α1,3]-Glc or Gal-β1,4-[Fuc-α1,3]-GlcNAc-β1,3-Gal-β1,6-[Fuc-α1,3]-Glc is glycosidically linked to at least two monosaccharide residues chosen from the list of monosaccharide residues as defined herein resulting in a fucosylated compound being an oligosaccharide with a DP of at least 8. Said fucosylated compound may be an oligosaccharide with a linear structure. Alternatively, said fucosylated compound may be an oligosaccharide with a branched structure. Optionally, said oligosaccharide is linked to a peptide, a protein and/or a lipid.

In another preferred embodiment of the method and/or cell of the present invention, the fucosylated compound is a saccharide comprising a formula Ra-[Rb]-[Re]-[Rf]-Gal-β1,m-[Fuc-α1,3]-GlcNAc-β1,n-[Rc]-Gal-β1,4-[Fuc-α1,3]-Glc, wherein said m is 3 or 4 and said n is 3 or 6 and i) wherein the non-reducing Gal residue of said Ra-[Rb]-[Re]-[Rf]-Gal-β1,m-[Fuc-α1,3]-GlcNAc-β1,n-[Rc]-Gal-β1,4-[Fuc-α1,3]-Glc is glycosidically linked to an Ra, Rb, Re and/or an Rf group, and/or ii) wherein the internal Gal residue of said Ra-[Rb]-[Re]-[Rf]-Gal-β1,m-[Fuc-α1,3]-GlcNAc-β1,n-[Rc]-Gal-β1,4-[Fuc-α1,3]-Glc is glycosidically linked to an Rc group. In the scope of the invention, any one of said Ra, Rb, Rc, Re and Rf groups is chosen from the list comprising a monosaccharide, a disaccharide and an oligosaccharide, as described herein. Optionally, said saccharide is linked to a peptide, a protein and/or a lipid.

In a more preferred embodiment of the method and/or cell of the present invention, the fucosylated compound is a saccharide comprising the formula Gal-β1,m-[Fuc-α1,3]-GlcNAc-β1,n-[Rc]-Gal-β1,4-[Fuc-α1,3]-Glc wherein said m is 3 or 4 and said n is 3 or 6, wherein the internal Gal residue of said Ra-[Rb]-[Re]-[Rf]-Gal-β1,m-[Fuc-α1,3]-GlcNAc-β1,n-[Rc]-Gal-β1,4-[Fuc-α1,3]-Glc is linked to an Rc group via an alpha-glycosidic or beta-glycosidic linkage and wherein said Rc group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-Gal-β1,4-[Fuc-α1,3]-Glc wherein said m is 3 or 4 and said n is 3 or 6, wherein the non-reducing Gal residue of said Ra-Gal-β1,m-[Fuc-α1,3]-GlcNAc-β1,n-Gal-β1,4-[Fuc-α1,3]-Glc is linked to an Ra group via an alpha-glycosidic or beta-glycosidic linkage and wherein said Ra group is chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-Gal-β1,4-[Fuc-α1,3]-Glc wherein said m is 3 or 4 and said n is 3 or 6, wherein the non-reducing Gal residue of said Ra-[Rb]-Gal-β1,m-[Fuc-α1,3]-GlcNAc-β1,n-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, and wherein said Ra group and Rb group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-Gal-β1,m-[Fuc-α1,3]-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, and wherein said Ra group, Rb group and Re group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, and wherein said Ra group, Rb group, Re group and Rf group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-Gal-β1,m-[Fuc-α1,3]-GlcNAc-β1,n-[Rc]-Gal-β1,4-[Fuc-α1,3]-Glc wherein said m is 3 or 4 and said n is 3 or 6, wherein the non-reducing Gal residue of said Ra-Gal-β1,m-[Fuc-α1,3]-GlcNAc-β1,n-[Rc]-Gal-β1,4-[Fuc-α1,3]-Glc is linked to an Ra group via an alpha-glycosidic or beta-glycosidic linkage, wherein the internal Gal residue of said Ra-Gal-β1,m-[Fuc-α1,3]-GlcNAc-β1,n-[Rc]-Gal-β1,4-[Fuc-α1,3]-Glc is linked to an Rc group via an alpha-glycosidic or beta-glycosidic linkage, and wherein said Ra group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-Gal-β1,m-[Fuc-α1,3]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-Gal-β1,m-[Fuc-α1,3]-GlcNAc-β1,n-[Rc]-Gal-β1,4-[Fuc-α1,3]-Glc is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-[Fuc-α1,3]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-Gal-β1,m-[Fuc-α1,3]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-Gal-β1,m-[Fuc-α1,3]-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-[Fuc-α1,3]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the fucosylated compound is a saccharide comprising the formula Ra-[Rb]-[Re]-[Rf]-Gal-β1,m-[Fuc-α1,3]-GlcNAc-β1,n-[Rc]-Gal-β1,4-[Fuc-α1,3]-Glc wherein said m is 3 or 4 and said n is 3 or 6, wherein the non-reducing Gal residue of said Ra-[Rb]-[Re]-[Rf]-Gal-β1,m-[Fuc-α1,3]-GlcNAc-β1,n-[Rc]-Gal-β1,4-[Fuc-α1,3]-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-[Fuc-α1,3]-GlcNAc-β1,n-[Rc]-Gal-β1,4-[Fuc-α1,3]-Glc is linked to an Rc group via an alpha-glycosidic or beta-glycosidic linkage, and wherein said Ra group, Rb group, Re group, Rf group and Rc group are chosen from the list comprising a monosaccharide, a disaccharide and an oligosaccharide. Optionally, said saccharide is linked to a peptide, protein or lipid.

In another preferred embodiment of the method and/or cell of present invention, said fucosylated compound is an oligosaccharide. In a more preferred embodiment, said fucosylated compound is a mammalian milk oligosaccharide (MMO) as defined herein. In an even more preferred embodiment, said fucosylated compound is a human milk oligosaccharide (HMO).

In another preferred embodiment of the method and/or cell of present invention, said fucosylated compound is a negatively charged or a neutral molecule. In a more preferred embodiment, said fucosylated compound is a sialylated molecule.

In another preferred embodiment of the method and/or cell of present invention, said fucosylated compound is a negatively charged or a neutral oligosaccharide. In a more preferred embodiment, said fucosylated compound is a sialylated oligosaccharide.

In another more preferred embodiment of the method and/or cell of the present invention, said fucosylated compound is an oligosaccharide chosen from the list comprising: Gal-β1,4-[Fuc-α1,3]-GlcNAc-β1,3-Gal-β1,4-Glc (lacto-N-fucopentaose III, LNFP-III); Gal-β1,4-GlcNAc-β1,3-Gal-β1,4-[Fuc-α1,3]-Glc (lacto-N-neofucopentaose V, LNFP-VI); Gal-β1,4-[Fuc-α1,3]-GlcNAc-β1,3-Gal-β1,4-[Fuc-α1,3]-Glc (lacto-N-neodifucohexaose II, LNnDFH II); Gal-β1,3-GlcNAc-β1,3-Gal-β1,4-[Fuc-α1,3]-Glc (lacto-N-fucopentaose V, LNFP-V); Gal-β1,3-[Fuc-α1,4]-GlcNAc-β1,3-Gal-β1,4-[Fuc-α1,3]-Glc (lacto-N-difucohexaose II, LNDFH-II); Neu5Ac-α2,3-Gal-β1,3-[Neu5Ac-α2,6]-GlcNAc-β1,3-Gal-β1,4-[Fuc-α1,3]-Glc (disialylfucosyllacto-N-tetraose, DS-LNF V, FDS-LNT II); Neu5Ac-α2,6-Gal-β1,4-GlcNAc-β1,3-Gal-β1,4-[Fuc-α1,3]-Glc (fusocylsialyllacto-N-neotetraose, F-LSTc); Neu5Ac-α2,6-Gal-β1,4-[Fuc-α1,3]-GlcNAc-β1,3-[Neu5Ac-α2,6-Gal-β1,4-GlcNAc-β1,6]-Gal-β1,4-Glc (fucosyldisialyllacto-N-neohexaose, FDS-LNnH); Neu5Ac-α2,3-Gal-β1,4-[Fuc-α1,3]-GlcNAc-β1,3-[Neu5Ac-α2,3-Gal-1,4-GlcNAc-β1,6]-Gal-β1,4-Glc (fucosyldisialyllacto-N-neohexaose, FDS-LNnH); Gal-β1,4-[Fuc-α1,3]-GlcNAc-β1,3-[Gal-β1,4-[Fuc-α1,3]-GlcNAc-β1,6]-Gal-β1,4-Glc (difucosyllacto-N-neohexaose, DF-LNnH); Gal-β1,3-GlcNAc-β1,3-Gal-β1,4-[Fuc-α1,3]-GlcNAc-β1,3-Gal-β1,4-Glc (fucosyl-para-lacto-N-hexaose IV, F-pLNH IV); Gal-β1,3-[Fuc-α1,4]-GlcNAc-β1,3-Gal-β1,4-[Fuc-α1,3]-GlcNAc-β1,3-Gal-β1,4-Glc (difucosyl-para-lacto-N-hexaose, DF-pLNH); Fuc-α1,2-Gal-β1,3-[Fuc-α1,4]-GlcNAc-β1,3-Gal-β1,4-[Fuc-α1,3]-GlcNAc-β1,3-Gal-β1,4-Glc (trifucosyl-para-lacto-N-hexaose I, TF-pLNH 1); Gal-1,3-[Fuc-α1,4]-GlcNAc-β1,3-Gal-1,4-[Fuc-α1,3]-GlcNAc-β1,3-Gal-β1,4-[Fuc-α1,3]-Glc (trifucosyl-para-lacto-N-hexaose II, TF-pLNH II); Fuc-α1,2-Gal-β1,3-GlcNAc-β1,3-Gal-β1,4-[Fuc-α1,3]-GlcNAc[6S]-β1,3-Gal-β1,4-Glc (difucosyl-para-lacto-N-hexaose sulfate I, DF-pLNH sulfate I); Gal-β1,3-[Fuc-α1,4]-GlcNAc-β1,3-Gal-β1,4-[Fuc-α1,3]-GcNAc[6S]-γ1,3-Gal-β1,4-Glc (difucosyl-para-lacto-N-hexaose sulfate II, DF-pLNH sulfate II); Fuc-α1,2-Gal-β1,3-[Fuc-α1,4]-GlcNAc-β1,3-Gal-β1,4-[Fuc-α1,3]-GlcNAc[6S]-β1,3-Gal-β1,4-Glc (difucosyl-para-lacto-N-hexaose sulfate III, DF-pLNH sulfate III); Gal-β1,3-GlcNAc-β1,3-Gal-β1,4-[Fuc-1,3]-GlcNAc-1,6-[Gal-1,4-[Fuc-α1,3]-GlcNAc-β1,3]-Gal-1,4-Glc; Gal-β1,3-[Fuc-α1,4]-GlcNAc-β1,3-Gal-1,4-[Fuc-α1,3]-GlcNAc-β1,6-[Gal-1,4-[Fuc-α1,3]-GlcNAc-β1,3]-Gal-1,4-Glc; Fuc-α1,2-Gal-1,3-[Fuc-α1,4]-GlcNAc-β1,3-Gal-1,4-[Fuc-α1,3]-GlcNAc-β1,3-Gal-1,4-[Fuc-α1,3]-GlcNAc-β1,3-Gal-β1,4-Glc; Gal-β1,4-[Fuc-α1,3]-GcNAc-β1,6-[Gal-β1,3-GlcNAc-β1,3]-Gal-β1,4-Glc; Gal-β1,4-[Fuc-α1,3]-GlcNAc-β1,6-[Neu5Ac-α2,6-Gal-β1,4-GcNAc-β1,3]-Gal-β1,4-Glc; Fuc-α1,2-Gal-β1,4-[Fuc-α1,3]-GlcNAc-β1,6-[Neu5Ac-α2,6-Gal-β1,4-GlcNAc-β1,3]-Gal-β1,4-Glc; Neu5Ac-α2,6-Gal-β1,4-GlcNAc-β1,3-Gal-β1,4-[Fuc-α1,3]-GlcNAc-β1,6-[Fuc-α1,2-Gal-1,3-[Fuc-α1,4]-GlcNAc-β1,3]-Gal-β1,4-Glc; Gal-β1,3-[Fuc-α1,4]-GlcNAc-β1,3-Gal-β1,4-[Fuc-α1,3]-GcNAc-β1,6-[Gal-β1,4-GcNAc-β1,3]-Gal-β1,4-Glc; Fuc-α1,2-Gal-1,3-GlcNAc-β1,3-Gal-1,4-[Fuc-α1,3]-GlcNAc-β1,6-[Fuc-α1,2-Gal-1,4-GlcNAc-β1,3]-Gal-β1,4-Glc; Gal-β1,3-GlcNAc-β1,3-Gal-β1,4-[Fuc-α1,3]-GcNAc-β1,6-[Gal-β1,3-GlcNAc-β1,3]-Gal-β1,4-Glc; Gal-(1,3-[Fuc-α1,4]-GlcNAc-β1,3-Gal-β1,4-[Fuc-α1,3]-GcNAc-1,6-[Gal-β1,3-GcNAc-β1,3]-Gal-β1,4-Glc; Gal-(1,3-GlcNAc-β1,3-Gal-β1,4-[Fuc-α1,3]-GcNAc-β1,6-[Fuc-α1,2-Gal-β1,3-GcNAc-β1,3]-Gal-β1,4-Glc; Fuc-α1,2-Gal-1,3-GlcNAc-β1,3-Gal-1,4-[Fuc-α1,3]-GlcNAc-β1,6-[Gal-1,3-GlcNAc-β1,3]-Gal-1,4-Glc; Fuc-α1,2-Gal-1,3-GlcNAc-β1,3-Gal-1,4-[Fuc-α1,3]-GlcNAc-β1,6-[Fuc-α1,2-Gal-1,3-GlcNAc-β1,3]-Gal-β1,4-Glc; Gal-1,3-[Fuc-α1,4]-GlcNAc-β1,3-Gal-1,4-[Fuc-α1,3]-GlcNAc-β1,6-[Gal-1,3-[Fuc-α1,4]-GlcNAc-β1,3]-Gal-β1,4-Glc; Fuc-α1,2-Ga-β1,3-GcNAc-α1,3-Gal-β1,4-[Fuc-β1,3]-GlcNAc-β1,6-[Fuc-α1,2-Gal-(1,3-[Fuc-α1,4]-GlcNAc-β1,3]-Gal-1,4-Glc; Fuc-α1,2-Gal-1,3-[Fuc-α1,4]-GlcNAc-β1,3-Gal-1,4-[Fuc-α1,3]-GlcNAc-β1.6-[Fuc-α1,2-Gal-β1,3-[Fuc-α1,4]-GlcNAc-β1,3]-Gal-β1,4-Glc: Gal-β1,3-GlcNAc-β1,3-Gal-β1,4-[Fuc-α1,3]-GlcNAc-β1,6-[Neu5Ac-α2,3-Gal-β1,3-[Fuc-α1,4]-GlcNAc-β1,3]-Gal-β1,4-Glc; Fuc-α1,2-Gal-β1,3-GlcNAc-β1,3-Gal-β1,4-[Fuc-α1,3]-GlcNAc-β1,6-[Neu5Ac-α2,3-Gal-β1,3-[Fuc-α1,4]-GlcNAc-β1,3]-Gal-β1,4-Glc; Gal-β1,4-GlcNAc-β1,6-[Gal-β1,3-GlcNAc-β1,3]-Gal-β1,4-[Fuc-α1,3]-GlcNAc-β1,6-[Fuc-α1,2-Gal-β1,3-[Fuc-α1,4]-GlcNAc-β1,3]-Gal-β1,4-Glc and Gal-β1,4-[Fuc-α1,3]-GlcNAc-β1,6-[Neu5Ac-α2,3-Gal-β1,3-GlcNAc-β1,3]-Gal-β1,4-Glc. In a most preferred embodiment of the method and/or cell of the present invention, said fucosylated compound is an oligosaccharide chosen from the list comprising: Gal-β1,4-[Fuc-α1,3]-GlcNAc-β1,3-Gal-β1,4-Glc (lacto-N-fucopentaose III, LNFP-III); Gal-β1,4-GlcNAc-β1,3-Gal-β1,4-[Fuc-α1,3]-Glc (lacto-N-neofucopentaose V, LNFP-VI); Gal-β1,4-[Fuc-α1,3]-GlcNAc-β1,3-Gal-β1,4-[Fuc-α1,3]-Glc (lacto-N-neodifucohexaose II, LNnDFH II); Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II).

In another preferred embodiment of the method and/or cell of present invention, the saccharide substrate is a saccharide comprising Gal-β1,m-GlcNAc-β1,n-Gal-β1,4-Glc, wherein said m is 3 or 4 and said n is 3 or 6. Said saccharide substrate may be a saccharide comprising Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc. Optionally, said saccharide is linked to a peptide, a protein and/or a lipid. In a more preferred embodiment, said saccharide substrate is an oligosaccharide chosen from the list comprising Gal-β1,3-[Fuc-α1,3]-GlcNAc-β1,3-Gal-β1,4-Glc, Gal-β1,3-[Fuc-α1,3]-GlcNAc-β1,6-Gal-β1,4-Glc, Gal-β1,4-[Fuc-α1,3]-GlcNAc-β1,3-Gal-β1,4-Glc and Gal-β1,4-[Fuc-α1,3]-GlcNAc-β1,6-Gal-β1,4-Glc. Optionally, said oligosaccharide is linked to a peptide, a protein and/or a lipid.

In an alternative more preferred embodiment, said saccharide substrate is a saccharide comprising Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc, wherein said Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc is glycosidically linked to one or more monosaccharide residues. Said one or more monosaccharide residues is/are chosen from the list of monosaccharide residues as defined herein, comprising N-acetyl-L-rhamnosamine, N-acetyl-D-fucosamine, N-acetyl-L-pneumosamine, N-acetylmuramic acid, N-acetyl-L-quinovosamine, Glc, Gal, GlcNAc, glucosamine, mannose, xylose, N-acetylmannosamine, a sialic acid, Neu5Ac, Neu5Gc, N-acetylgalactosamine, galactosamine, fucose, rhamnose, glucuronic acid, gluconic acid, fructose and polyols. Said one or more monosaccharide residue(s) may be glycosidically linked to said non-reducing Gal, GlcNAc, internal Gal and/or reducing Glc residue of said Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc of said saccharide. Said glycosidic linkages comprise alpha- and beta-glycosidic linkages. In the context of present invention, said saccharide substrate is not Gal-β1,m-[Fuc-α1,3]-GlcNAc-β1,n-Gal-β1,4-[Fuc-α1,3]-Glc or is not a saccharide comprising Gal-β1,m-[Fuc-α1,3]-GlcNAc-β1,n-Gal-β1,4-[Fuc-α1,3]-Glc. Said saccharide substrate may be a saccharide with a linear structure. Alternatively, said saccharide substrate may be a saccharide with a branched structure. Said saccharide substrate may be an oligosaccharide, a polysaccharide or a glycan as defined herein. Optionally, said saccharide substrate is linked to a peptide, a protein and/or a lipid.

In another preferred embodiment of the method and/or cell of present invention, the saccharide substrate is an oligosaccharide comprising Gal-β1,m-GlcNAc-β1,n-Gal-β1,4-Glc wherein said m is 3 or 4 and said n is 3 or 6 and having a degree of polymerisation (DP) of at least five. Said saccharide substrate is an oligosaccharide 1) comprising Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc and 2) having a DP of at least five. An oligosaccharide with a DP of at least five refers to a pentasaccharide, a hexasaccharide, a heptasaccharide or saccharide structures comprising eight or more monosaccharide residues. Said saccharide substrate may be an oligosaccharide comprising Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc wherein said Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc is glycosidically linked to at least one monosaccharide residue chosen from the list of monosaccharide residues as defined herein resulting in a fucosylated compound being an oligosaccharide with a DP of at least 5. Said at least one monosaccharide residue may be glycosidically linked to said non-reducing Gal, GlcNAc, internal Gal and/or reducing Glc residue of said Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc of said oligosaccharide. Said glycosidic linkages comprise alpha- and beta-glycosidic linkages. In the context of present invention, said saccharide substrate is not Gal-β1,m-[Fuc-α1,3]-GlcNAc-β1,n-Gal-β1,4-[Fuc-α1,3]-Glc or is not an oligosaccharide comprising Gal-β1,m-[Fuc-α1,3]-GlcNAc-β1,n-Gal-β1,4-[Fuc-α1,3]-Glc. In an even more preferred embodiment, the saccharide substrate is an oligosaccharide comprising Gal-β1,m-GlcNAc-β1,n-Gal-β1,4-Glc wherein said m is 3 or 4 and said n is 3 or 6 and having a degree of polymerisation of at least six. In other words, said saccharide substrate may be an oligosaccharide comprising Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc wherein said Gal-β1,3-GlcNAc-β1,3-Gal-β1,4-Glc, Gal-β1,3-GlcNAc-β1,6-Gal-β1,4-Glc, Gal-β1,4-GlcNAc-β1,3-Gal-β1,4-Glc or Gal-β1,4-GlcNAc-β1,6-Gal-β1,4-Glc is glycosidically linked to at least two monosaccharide residues chosen from the list of monosaccharide residues as defined herein resulting in a fucosylated compound being an oligosaccharide with a DP of at least 6. In the context of present invention, said saccharide substrate is not Gal-β1,m-[Fuc-α1,3]-GlcNAc-β1,n-Gal-β1,4-[Fuc-α1,3]-Glc or is not an oligosaccharide comprising Gal-β1,m-[Fuc-α1,3]-GlcNAc-β1,n-Gal-β1,4-[Fuc-α1,3]-Glc. Said fucosylated compound may be an oligosaccharide with a linear structure. Alternatively, said fucosylated compound may be an oligosaccharide with a branched structure. Optionally, said oligosaccharide is linked to a peptide, a protein and/or a lipid.

In another preferred embodiment of the method and/or cell of the present invention, the saccharide substrate is a saccharide comprising a formula Ra-[Rb]-[Re]-[Rf]-Gal-β1,m-[Rg]-GlcNAc-β1,n-[Rc]-Gal-β1,4-Glc-[Rd], wherein said m is 3 or 4 and said n is 3 or 6 and i) wherein the non-reducing Gal residue of said Ra-[Rb]-[Re]-[Rf]-Gal-β1,m-[Rg]-GlcNAc-β1,n-[Rc]-Gal-β1,4-Glc-[Rd] is glycosidically linked to an Ra, Rb, Re and/or an Rf group, and/or ii) wherein the GlcNAc residue of said Ra-[Rb]-[Re]-[Rf]-Gal-β1,m-[Rg]-GlcNAc-β1,n-[Rc]-Gal-β1,4-Glc-[Rd] is glycosidically linked to an Rg group, and/or iii) wherein the internal Gal residue of said Ra-[Rb]-[Re]-[Rf]-Gal-β1,m-[Rg]-GlcNAc-β1,n-[Rc]-Gal-β1,4-Glc-[Rd] is glycosidically linked to an Rc group and/or iv) wherein the reducing Glc residue of said Ra-[Rb]-[Re]-[Rf]-Gal-β1,m-[Rg]-GlcNAc-β1,n-[Rc]-Gal-β1,4-Glc-[Rd] is glycosidically linked to an Rd group. In the scope of the invention, any one of said Ra, Rb, Rc, Rd, Re, Rf and Rg groups is chosen from the list comprising a monosaccharide, a disaccharide and an oligosaccharide, as described herein. It should be understood that both the Rg group and the Rd group are not a fucose residue when both are present in said Ra-[Rb]-[Re]-[Rf]-Gal-β1,m-[Rg]-GlcNAc-β1,n-[Rc]-Gal-β1,4-Glc-[Rd] of said saccharide substrate. Said Rg group of said Ra-[Rb]-[Re]-[Rf]-Gal-β1,m-[Rg]-GlcNAc-β1,n-[Rc]-Gal-β1,4-Glc-[Rd] of said saccharide substrate may be a fucose residue when said Rd group is not present in said Ra-[Rb]-[Re]-[Rf]-Gal-β1,m-[Rg]-GlcNAc-β1,n-[Rc]-Gal-β1,4-Glc-[Rd] of said saccharide substrate. Alternatively, said Rd group of said Ra-[Rb]-[Re]-[Rf]-Gal-β1,m-[Rg]-GlcNAc-β1,n-[Rc]-Gal-β1,4-Glc-[Rd] of said saccharide substrate may be a fucose residue when said Rg group is not present in said Ra-[Rb]-[Re]-[Rf]-Gal-β1,m-[Rg]-GlcNAc-β1,n-[Rc]-Gal-β1,4-Glc-[Rd] of said saccharide substrate. Optionally, said saccharide is linked to a peptide, a protein and/or a lipid.

In a more preferred embodiment of the method and/or cell of the present invention, the saccharide substrate is a saccharide comprising the formula Gal-β1,m-GlcNAc-β1,n-Gal-β1,4-Glc-[Rd], wherein said m is 3 or 4 and said n is 3 or 6, wherein the reducing Glc residue of said Gal-β1,m-GlcNAc-β1,n-Gal-β1,4-Glc-[Rd] is linked to an Rd group via an alpha-glycosidic or beta-glycosidic linkage and wherein said Rd group is chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-GlcNAc-β1,n-[Rc]-Gal-β1,4-Glc wherein said m is 3 or 4 and said n is 3 or 6, wherein the internal Gal residue of said Gal-β1,m-GlcNAc-β1,n-[Rc]-Gal-β1,4-Glc is linked to an Rc group via an alpha-glycosidic or beta-glycosidic linkage and wherein said Rc group is chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-[Rg]-GlcNAc-β1,n-Gal-β1,4-Glc wherein said m is 3 or 4 and said n is 3 or 6, wherein the GlcNAc residue of said formula Gal-β1,m-[Rg]-GlcNAc-β1,n-Gal-β1,4-Glc is linked to an Rg group via an alpha-glycosidic or beta-glycosidic linkage and wherein said Rc group is chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-GlcNAc-β1,n-[Rc]-Gal-β1,4-Glc-[Rd], wherein said m is 3 or 4 and said n is 3 or 6, wherein the reducing Glc residue of said Gal-β1,m-GlcNAc-β1,n-[Rc]-Gal-β1,4-Glc-[Rd] is linked to an Rd group via an alpha-glycosidic or beta-glycosidic linkage, wherein the internal Gal residue of said Gal-β1,m-GlcNAc-β1,n-[Rc]-Gal-β1,4-Glc-[Rd] is linked to an Rc group via an alpha-glycosidic or beta-glycosidic linkage and wherein said Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-[Rg]-GlcNAc-β1,n-Gal-β1,4-Glc-[Rd], wherein said m is 3 or 4 and said n is 3 or 6, wherein the reducing Glc residue of said Gal-β1,m-GlcNAc-β1,n-[Rc]-Gal-β1,4-Glc-[Rd] is linked to an Rd group via an alpha-glycosidic or beta-glycosidic linkage, wherein the GlcNAc residue of said Gal-β1,m-[Rg]-GlcNAc-β1,n-Gal-β1,4-Glc-[Rd] is linked to an Rg group via an alpha-glycosidic or beta-glycosidic linkage and wherein said Rg group and Rd group are chosen from the list comprising a monosaccharide except a fucose residue, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-[Rg]-GlcNAc-β1,n-[Rc]-Gal-β1,4-Glc, wherein said m is 3 or 4 and said n is 3 or 6, wherein the GlcNAc residue of said Gal-β1,m-[Rg]-GlcNAc-β1,n-[Rc]-Gal-β1,4-Glc is linked to an Rg group via an alpha-glycosidic or beta-glycosidic linkage, wherein the internal Gal residue of said Gal-β1,m-[Rg]-GlcNAc-β1,n-[Rc]-Gal-β1,4-Glc is linked to an Rc group via an alpha-glycosidic or beta-glycosidic linkage, and wherein said Rc group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Gal-β1,m-[Rg]-GlcNAc-β1,n-[Rc]-Gal-β1,4-Glc-[Rd], wherein said m is 3 or 4 and said n is 3 or 6, wherein the GlcNAc residue of said Gal-β1,m-[Rg]-GlcNAc-β1,n-[Rc]-Gal-β1,4-Glc-[Rd] is linked to an Rg group via an alpha-glycosidic or beta-glycosidic linkage, wherein the internal Gal residue of said Gal-β1,m-[Rg]-GlcNAc-β1,n-[Rc]-Gal-β1,4-Glc-[Rd] is linked to an Rc group via an alpha-glycosidic or beta-glycosidic linkage, wherein the reducing Glc residue of said Gal-β1,m-[Rg]-GlcNAc-β1,n-[Rc]-Gal-β1,4-Glc-[Rd] is linked to an Rd group via an alpha-glycosidic or beta-glycosidic linkage, and wherein said Rc group and Rg group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-GlcNAc-β1,n-Gal-β1,4-Glc, wherein said m is 3 or 4 and said n is 3 or 6, wherein the non-reducing Gal residue of said Ra-Gal-β1,m-GlcNAc-β1,n-Gal-β1,4-Glc is linked to an Ra group via an alpha-glycosidic or beta-glycosidic linkage and wherein said Ra group is chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-GlcNAc-β1,n-Gal-β1,4-Glc, wherein said m is 3 or 4 and said n is 3 or 6, wherein the non-reducing Gal residue of said Ra-[Rb]-Gal-β1,m-GlcNAc-β1,n-Gal-β1,4-Glc is linked to i) an Ra group via an alpha-glycosidic or beta-glycosidic linkage and ii) an Rb group via an alpha-glycosidic or beta-glycosidic linkage, and wherein said Ra group and Rb group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-GlcNAc-β1,n-Gal-β1,4-Glc, wherein said m is 3 or 4 and said n is 3 or 6, wherein the non-reducing Gal residue of said Ra-[Rb]-[Re]-Gal-β1,m-GlcNAc-β1,n-Gal-β1,4-Glc is linked to i) an Ra group via an alpha-glycosidic or beta-glycosidic linkage, ii) an Rb group via an alpha-glycosidic or beta-glycosidic linkage and iii) an Re group via an alpha-glycosidic or beta-glycosidic linkage, and wherein said Ra group, Rb group and Re group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-GlcNAc-β1,n-Gal-β1,4-Glc, wherein said m is 3 or 4 and said n is 3 or 6, wherein the non-reducing Gal residue of said Ra-[Rb]-[Re]-[Rf]-Gal-β1,m-GlcNAc-β1,n-Gal-β1,4-Glc is linked to i) an Ra group via an alpha-glycosidic or beta-glycosidic linkage, ii) an Rb group via an alpha-glycosidic or beta-glycosidic linkage, iii) an Re group via an alpha-glycosidic or beta-glycosidic linkage and iv) an Rf group via an alpha-glycosidic or beta-glycosidic linkage, and wherein said Ra group, Rb group, Re and Rf group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-GlcNAc-β1,n-Gal-β1,4-Glc-[Rd], wherein said m is 3 or 4 and said n is 3 or 6, wherein the non-reducing Gal residue of said Ra-Gal-β1,m-GlcNAc-β1,n-Gal-β1,4-Glc-[Rd] is linked to an Ra group via an alpha-glycosidic or beta-glycosidic linkage, wherein the reducing Glc residue of said Ra-Gal-β1,m-GlcNAc-β1,n-Gal-β1,4-Glc-[Rd] is linked to an Rd group via an alpha-glycosidic or beta-glycosidic linkage, and wherein said Ra group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-GlcNAc-β1,n-[Rc]-Gal-β1,4-Glc, wherein said m is 3 or 4 and said n is 3 or 6, wherein the non-reducing Gal residue of said Ra-Gal-β1,m-GlcNAc-β1,n-[Rc]-Gal-β1,4-Glc is linked to an Ra group via an alpha-glycosidic or beta-glycosidic linkage, wherein the internal Gal residue of said Ra-Gal-β1,m-GlcNAc-β1,n-[Rc]-Gal-β1,4-Glc is linked to an Rc group via an alpha-glycosidic or beta-glycosidic linkage, and wherein said Ra group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-[Rg]-GlcNAc-β1,n-Gal-β1,4-Glc, wherein said m is 3 or 4 and said n is 3 or 6, wherein the non-reducing Gal residue of said Ra-Gal-β1,m-[Rg]-GlcNAc-β1,n-Gal-β1,4-Glc is linked to an Ra group via an alpha-glycosidic or beta-glycosidic linkage, wherein the GlcNAc residue of said Ra-Gal-β1,m-[Rg]-GlcNAc-β1,n-Gal-β1,4-Glc is linked to an Rg group via an alpha-glycosidic or beta-glycosidic linkage and wherein said Ra group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-GlcNAc-β1,n-[Rc]-Gal-β1,4-Glc-[Rd], wherein said m is 3 or 4 and said n is 3 or 6, wherein the non-reducing Gal residue of said Ra-Gal-β1,m-GlcNAc-β1,n-[Rc]-Gal-β1,4-Glc-[Rd] is linked to an Ra group via an alpha-glycosidic or beta-glycosidic linkage, wherein the internal Gal residue of said Ra-Gal-β1,m-GlcNAc-β1,n-[Rc]-Gal-β1,4-Glc-[Rd] is linked to an Rc group via an alpha-glycosidic or beta-glycosidic linkage, wherein the reducing Glc residue of said Ra-Gal-β1,m-GlcNAc-β1,n-[Rc]-Gal-β1,4-Glc-[Rd] is linked to an Rd group via an alpha-glycosidic or beta-glycosidic linkage, and wherein said Ra group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-[Rg]-GlcNAc-β1,n-Gal-β1,4-Glc-[Rd], wherein said m is 3 or 4 and said n is 3 or 6, wherein the non-reducing Gal residue of said Ra-Gal-β1,m-[Rg]-GlcNAc-β1,n-Gal-β1,4-Glc-[Rd] is linked to an Ra group via an alpha-glycosidic or beta-glycosidic linkage, wherein the GlcNAc residue of said Ra-Gal-β1,m-[Rg]-GlcNAc-β1,n-Gal-β1,4-Glc-[Rd] is linked to an Rg group via an alpha-glycosidic or beta-glycosidic linkage, wherein the reducing Glc residue of said Ra-Gal-β1,m-[Rg]-GlcNAc-β1,n-Gal-β1,4-Glc-[Rd] is linked to an Rd group via an alpha-glycosidic or beta-glycosidic linkage, and wherein said Ra group, Rg group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-[Rg]-GlcNAc-β1,n-[Rc]-Gal-β1,4-Glc, wherein said m is 3 or 4 and said n is 3 or 6, wherein the non-reducing Gal residue of said Ra-Gal-β1,m-[Rg]-GlcNAc-β1,n-[Rc]-Gal-β1,4-Glc is linked to an Ra group via an alpha-glycosidic or beta-glycosidic linkage, wherein the GlcNAc residue of said Ra-Gal-β1,m-[Rg]-GlcNAc-β1,n-[Rc]-Gal-β1,4-Glc is linked to an Rg group via an alpha-glycosidic or beta-glycosidic linkage, wherein the internal Gal residue of said Ra-Gal-β1,m-[Rg]-GlcNAc-β1,n-[Rc]-Gal-β1,4-Glc is linked to an Rc group via an alpha-glycosidic or beta-glycosidic linkage, and wherein said Ra group, Rg group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-Gal-β1,m-[Rg]-GlcNAc-β1,n-[Rc]-Gal-β1,4-Glc-[Rd], wherein said m is 3 or 4 and said n is 3 or 6, wherein the non-reducing Gal residue of said Ra-Gal-β1,m-[Rg]-GlcNAc-β1,n-[Rc]-Gal-β1,4-Glc-[Rd] is linked to an Ra group via an alpha-glycosidic or beta-glycosidic linkage, wherein the GlcNAc residue of said Ra-Gal-β1,m-[Rg]-GlcNAc-β1,n-[Rc]-Gal-β1,4-Glc-[Rd] is linked to an Rg group via an alpha-glycosidic or beta-glycosidic linkage, wherein the internal Gal residue of said Ra-Gal-β1,m-[Rg]-GlcNAc-β1,n-[Rc]-Gal-β1,4-Glc-[Rd] is linked to an Rc group via an alpha-glycosidic or beta-glycosidic linkage, wherein the reducing Glc residue of said Ra-Gal-β1,m-[Rg]-GlcNAc-β1,n-[Rc]-Gal-β1,4-Glc-[Rd] is linked to an Rd group via an alpha-glycosidic or beta-glycosidic linkage, and wherein said Ra group, Rg group, Rc group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-GlcNAc-β1,n-Gal-β1,4-Glc-[Rd], wherein said m is 3 or 4 and said n is 3 or 6, wherein the non-reducing Gal residue of said Ra-[Rb]-Gal-β1,m-GlcNAc-β1,n-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the reducing Glc residue of said Ra-[Rb]-Gal-β1,m-GlcNAc-β1,n-Gal-β1,4-Glc-[Rd] is linked to an Rd group via an alpha-glycosidic or beta-glycosidic linkage, and wherein said Ra group, Rb group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-GlcNAc-β1,n-[Rc]-Gal-β1,4-Glc, wherein said m is 3 or 4 and said n is 3 or 6, wherein the non-reducing Gal residue of said Ra-[Rb]-Gal-β1,m-GlcNAc-β1,n-[Rc]-Gal-β1,4-Glc is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-GlcNAc-β1,n-[Rc]-Gal-β1,4-Glc is linked to an Rc group via an alpha-glycosidic or beta-glycosidic linkage, and wherein said Ra group, Rb group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-[Rg]-GlcNAc-β1,n-Gal-β1,4-Glc, wherein said m is 3 or 4 and said n is 3 or 6, wherein the non-reducing Gal residue of said Ra-[Rb]-Gal-β1,m-[Rg]-GlcNAc-β1,n-Gal-β1,4-Glc is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-Gal-β1,4-Glc is linked to an Rg group via an alpha-glycosidic or beta-glycosidic linkage and wherein said Ra group, Rb group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-GlcNAc-β1,n-[Rc]-Gal-β1,4-Glc-[Rd], wherein said m is 3 or 4 and said n is 3 or 6, wherein the non-reducing Gal residue of said Ra-[Rb]-Gal-β1,m-GlcNAc-β1,n-[Rc]-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the internal Gal residue of said Ra-[Rb]-Gal-β1,m-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-Gal-β1,m-GlcNAc-β1,n-[Rc]-Gal-β1,4-Glc-[Rd] is linked to an Rd group via an alpha-glycosidic or beta-glycosidic linkage, and wherein said Ra group, Rb group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-[Rg]-GlcNAc-β1,n-Gal-β1,4-Glc-[Rd], wherein said m is 3 or 4 and said n is 3 or 6, wherein the non-reducing Gal residue of said Ra-[Rb]-Gal-β1,m-[Rg]-GlcNAc-β1,n-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-Gal-β1,4-Glc-[Rd] is linked to an Rg group via an alpha-glycosidic or beta-glycosidic linkage, wherein the reducing Glc residue of said Ra-[Rb]-Gal-β1,m-[Rg]-GlcNAc-β1,n-Gal-β1,4-Glc-[Rd] is linked to an Rd group via an alpha-glycosidic or beta-glycosidic linkage, and wherein said Ra group, Rb group, Rg group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-[Rg]-GlcNAc-β1,n-[Rc]-Gal-β1,4-Glc, wherein said m is 3 or 4 and said n is 3 or 6, wherein the non-reducing Gal residue of said Ra-[Rb]-Gal-β1,m-[Rg]-GlcNAc-β1,n-[Rc]-Gal-β1,4-Glc is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-[Rc]-Gal-β1,4-Glc is linked to an Rg group via an alpha-glycosidic or beta-glycosidic linkage, wherein the internal Gal residue of said Ra-[Rb]-Gal-β1,m-[Rg]-GlcNAc-β1,n-[Rc]-Gal-β1,4-Glc is linked to an Rc group via an alpha-glycosidic or beta-glycosidic linkage, and wherein said Ra group, Rb group, Rg group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-Gal-β1,m-[Rg]-GlcNAc-β1,n-[Rc]-Gal-β1,4-Glc-[Rd], wherein said m is 3 or 4 and said n is 3 or 6, wherein the non-reducing Gal residue of said Ra-[Rb]-Gal-β1,m-[Rg]-GlcNAc-β1,n-[Rc]-Gal-β1,4-Glc-[Rd] is linked to 1) an Ra group via an alpha-glycosidic or beta-glycosidic linkage and 2) an Rb group via an alpha-glycosidic or beta-glycosidic linkage, wherein the GlcNAc residue of said Ra-[Rb]-Gal-β1,m-[Rg]-GlcNAc-β1,n-[Rc]-Gal-β1,4-Glc-[Rd] is linked to an Rg group via an alpha-glycosidic or beta-glycosidic linkage, wherein the internal Gal residue of said Ra-[Rb]-Gal-β1,m-[Rg]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-Gal-β1,m-[Rg]-GlcNAc-β1,n-[Rc]-Gal-β1,4-Glc-[Rd] is linked to an Rd group via an alpha-glycosidic or beta-glycosidic linkage, and wherein said Ra group, Rb group, Rg group, Rc group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-GlcNAc-β1,n-Gal-β1,4-Glc-[Rd], wherein said m is 3 or 4 and said n is 3 or 6, wherein the non-reducing Gal residue of said Ra-[Rb]-[Re]-Gal-β1,m-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the reducing Glc residue of said Ra-[Rb]-[Re]-Gal-β1,m-GlcNAc-β1,n-Gal-β1,4-Glc-[Rd] is linked to an Rd group via an alpha-glycosidic or beta-glycosidic linkage, and wherein said Ra group, Rb group, Re group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-GlcNAc-β1,n-[Rc]-Gal-β1,4-Glc, wherein said m is 3 or 4 and said n is 3 or 6, wherein the non-reducing Gal residue of said Ra-[Rb]-[Re]-Gal-β1,m-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-GlcNAc-β1,n-[Rc]-Gal-β1,4-Glc is linked to an Rc group via an alpha-glycosidic or beta-glycosidic linkage, and wherein said Ra group, Rb group, Re group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-[Rg]-GlcNAc-β1,n-Gal-β1,4-Glc, wherein said m is 3 or 4 and said n is 3 or 6, wherein the non-reducing Gal residue of said formula Ra-[Rb]-[Re]-Gal-β1,m-[Rg]-GlcNAc-β1,n-Gal-β1,4-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said formula Ra-[Rb]-[Re]-Gal-β1,m-[Rg]-GlcNAc-β1,n-Gal-β1,4-Glc is linked to an Rg group via an alpha-glycosidic or beta-glycosidic linkage and wherein said Ra group, Rb group, Re group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-GlcNAc-β1,n-[Rc]-Gal-β1,4-Glc-[Rd], wherein said m is 3 or 4 and said n is 3 or 6, wherein the non-reducing Gal residue of said Ra-[Rb]-[Re]-Gal-β1,m-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the internal Gal residue of said Ra-[Rb]-[Re]-Gal-β1,m-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-Gal-β1,m-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-[Rg]-GlcNAc-β1,n-Gal-β1,4-Glc-[Rd], wherein said m is 3 or 4 and said n is 3 or 6, wherein the non-reducing Gal residue of said Ra-[Rb]-[Re]-Gal-β1,m-[Rg]-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said Ra-[Rb]-[Re]-Gal-β1,m-[Rg]-GlcNAc-β1,n-Gal-β1,4-Glc-[Rd] is linked to an Rg group via an alpha-glycosidic or beta-glycosidic linkage, wherein the reducing Glc residue of said Ra-[Rb]-[Re]-Gal-β1,m-[Rg]-GlcNAc-β1,n-Gal-β1,4-Glc-[Rd] is linked to an Rd group via an alpha-glycosidic or beta-glycosidic linkage, and wherein said Ra group, Rb group, Re group, Rg group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-[Rg]-GlcNAc-β1,n-[Rc]-Gal-β1,4-Glc, wherein said m is 3 or 4 and said n is 3 or 6, wherein the non-reducing Gal residue of said Ra-[Rb]-[Re]-Gal-β1,m-[Rg]-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said Ra-[Rb]-[Re]-Gal-β1,m-[Rg]-GlcNAc-β1,n-[Rc]-Gal-β1,4-Glc is linked to an Rg group via an alpha-glycosidic or beta-glycosidic linkage, wherein the internal Gal residue of said Ra-[Rb]-[Re]-Gal-β1,m-[Rg]-GlcNAc-β1,n-[Rc]-Gal-β1,4-Glc is linked to an Rc group via an alpha-glycosidic or beta-glycosidic linkage, and wherein said Ra group, Rb group, Re group, Rg group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-Gal-β1,m-[Rg]-GlcNAc-β1,n-[Rc]-Gal-β1,4-Glc-[Rd], wherein said m is 3 or 4 and said n is 3 or 6, wherein the non-reducing Gal residue of said Ra-[Rb]-[Re]-Gal-β1,m-[Rg]-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage and 3) an Re group via an alpha-glycosidic or beta-glycosidic linkage, wherein the GlcNAc residue of said Ra-[Rb]-[Re]-Gal-β1,m-[Rg]-GlcNAc-β1,n-[Rc]-Gal-β1,4-Glc-[Rd] is linked to an Rg group via an alpha-glycosidic or beta-glycosidic linkage, wherein the internal Gal residue of said Ra-[Rb]-[Re]-Gal-β1,m-[Rg]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-Gal-β1,m-[Rg]-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rg group, Rc group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-GlcNAc-β1,n-Gal-β1,4-Glc-[Rd], wherein said m is 3 or 4 and said n is 3 or 6, wherein the non-reducing Gal residue of said Ra-[Rb]-[Re]-[Rf]-Gal-β1,m-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the reducing Glc residue of said Ra-[Rb]-[Re]-[Rf]-Gal-β1,m-GlcNAc-β1,n-Gal-β1,4-Glc-[Rd] is linked to an Rd group via an alpha-glycosidic or beta-glycosidic linkage, and wherein said Ra group, Rb group, Re group, Rf group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-GlcNAc-β1,n-[Rc]-Gal-β1,4-Glc, wherein said m is 3 or 4 and said n is 3 or 6, wherein the non-reducing Gal residue of said Ra-[Rb]-[Re]-[Rf]-Gal-β1,m-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-GlcNAc-β1,n-[Rc]-Gal-β1,4-Glc is linked to an Rc group via an alpha-glycosidic or beta-glycosidic linkage, and wherein said Ra group, Rb group, Re group, Rf group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-[Rg]-GlcNAc-β1,n-Gal-β1,4-Glc, wherein said m is 3 or 4 and said n is 3 or 6, wherein the non-reducing Gal residue of said formula Ra-[Rb]-[Re]-[Rf]-Gal-β1,m-[Rg]-GlcNAc-β1,n-Gal-β1,4-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said formula Ra-[Rb]-[Re]-[Rf]-Gal-β1,m-[Rg]-GlcNAc-β1,n-Gal-β1,4-Glc is linked to an Rg group via an alpha-glycosidic or beta-glycosidic linkage and wherein said Ra group, Rb group, Re group, Rf group and Rg group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-GlcNAc-β1,n-[Rc]-Gal-β1,4-Glc-[Rd], wherein said m is 3 or 4 and said n is 3 or 6, wherein the non-reducing Gal residue of said Ra-[Rb]-[Re]-[Rf]-Gal-β1,m-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the internal Gal residue of said Ra-[Rb]-[Re]-[Rf]-Gal-β1,m-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-[Rf]-Gal-β1,m-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rf group, Rc group and Rd group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-[Rg]-GlcNAc-β1,n-Gal-β1,4-Glc-[Rd], wherein said m is 3 or 4 and said n is 3 or 6, wherein the non-reducing Gal residue of said Ra-[Rb]-[Re]-[Rf]-Gal-β1,m-[Rg]-GlcNAc-β1,n-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said Ra-[Rb]-[Re]-[Rf]-Gal-β1,m-[Rg]-GlcNAc-β1,n-Gal-β1,4-Glc-[Rd] is linked to an Rg group via an alpha-glycosidic or beta-glycosidic linkage, wherein the reducing Glc residue of said Ra-[Rb]-[Re]-[Rf]-Gal-β1,m-[Rg]-GlcNAc-β1,n-Gal-β1,4-Glc-[Rd] is linked to an Rd group via an alpha-glycosidic or beta-glycosidic linkage, and wherein said Ra group, Rb group, Re group, Rf group, Rg group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-[Rg]-GlcNAc-β1,n-[Rc]-Gal-β1,4-Glc, wherein said m is 3 or 4 and said n is 3 or 6, wherein the non-reducing Gal residue of said Ra-[Rb]-[Re]-[Rf]-Gal-β1,m-[Rg]-GlcNAc-β1,n-[Rc]-Gal-β1,4-Glc is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said Ra-[Rb]-[Re]-[Rf]-Gal-β1,m-[Rg]-GlcNAc-β1,n-[Rc]-Gal-β1,4-Glc is linked to an Rg group via an alpha-glycosidic or beta-glycosidic linkage, wherein the internal Gal residue of said Ra-[Rb]-[Re]-[Rf]-Gal-β1,m-[Rg]-GlcNAc-β1,n-[Rc]-Gal-β1,4-Glc is linked to an Rc group via an alpha-glycosidic or beta-glycosidic linkage, and wherein said Ra group, Rb group, Re group, Rf group, Rg group and Rc group are chosen from the list comprising a monosaccharide, a disaccharide and an oligosaccharide. Optionally, said saccharide is linked to a peptide, protein or lipid.

In another more preferred embodiment of the method and/or cell of the present invention, the saccharide substrate is a saccharide comprising the formula Ra-[Rb]-[Re]-[Rf]-Gal-β1,m-[Rg]-GlcNAc-β1,n-[Rc]-Gal-β1,4-Glc-[Rd], wherein said m is 3 or 4 and said n is 3 or 6, wherein the non-reducing Gal residue of said Ra-[Rb]-[Re]-[Rf]-Gal-β1,m-[Rg]-GlcNAc-β1,n-[Rc]-Gal-β1,4-Glc-[Rd] is linked to 1) an Ra group via an alpha-glycosidic or beta-glycosidic linkage, 2) an Rb group via an alpha-glycosidic or beta-glycosidic linkage, 3) an Re group via an alpha-glycosidic or beta-glycosidic linkage and 4) an Rf group via an alpha-glycosidic or beta-glycosidic linkage, wherein the GlcNAc residue of said Ra-[Rb]-[Re]-[Rf]-Gal-β1,m-[Rg]-GlcNAc-β1,n-[Rc]-Gal-β1,4-Glc-[Rd] is linked to an Rg group via an alpha-glycosidic or beta-glycosidic linkage, wherein the internal Gal residue of said Ra-[Rb]-[Re]-[Rf]-Gal-β1,m-[Rg]-GlcNAc-β1,n-[Rc]-Gal-β1,4-Glc-[Rd] is linked to an Rc group via an alpha-glycosidic or beta-glycosidic linkage, wherein the reducing Glc residue of said Ra-[Rb]-[Re]-[Rf]-Gal-β1,m-[Rg]-GlcNAc-β1,n-[Rc]-Gal-β1,4-Glc-[Rd] is linked to an Rd group via an alpha-glycosidic or beta-glycosidic linkage, and wherein said Ra group, Rb group, Re group, Rf group, Rg group, Rc group and Rd group are chosen from the list comprising a monosaccharide except the Rg group and the Rd group being a fucose residue at the same time, a disaccharide and an oligosaccharide. Optionally, said saccharide is linked to a peptide, protein or lipid.

In another preferred embodiment of the method and/or cell of present invention, said saccharide substrate is an oligosaccharide. In a more preferred embodiment, said saccharide substrate is a mammalian milk oligosaccharide (MMO) as defined herein. In an even more preferred embodiment, said saccharide substrate is a human milk oligosaccharide (HMO).

In another preferred embodiment of the method and/or cell of present invention, said saccharide substrate is a negatively charged or a neutral molecule. In a more preferred embodiment, said saccharide substrate is a sialylated molecule.

In another preferred embodiment of the method and/or cell of present invention, said saccharide substrate is a negatively charged or a neutral oligosaccharide. In a more preferred embodiment, said saccharide substrate is a sialylated oligosaccharide.

In another preferred embodiment of the method and/or cell of present invention, said saccharide substrate is chosen from the list comprising Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).

In the context of the present invention, the fucosyltransferase used in the methods and/or cell of present invention for the production of a fucosylated compound as described herein has alpha-1,3-fucosyltransferase activity on the GlcNAc and/or the Glc residue of Gal-β1,m-GlcNAc-β1,n-Gal-β1,4-Glc of a saccharide substrate as described herein and i) comprises a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or ii) is a polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or iii) comprises a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or iv) comprises a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51. It is to be understood that said fucosyltransferase is capable to catalyse, preferably catalyses, the transfer of a fucose residue from GDP-fucose to the GlcNAc and/or the Glc residue of Gal-β1,m-GlcNAc-β1,n-Gal-β1,4-Glc of a saccharide substrate as described herein in an alpha-1,3-glycosidic linkage resulting in the production of a fucosylated compound as described herein.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. A polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 is to be understood as that the polypeptide comprises or consists of an amino acid sequence that has 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, respectively, as given herein. A polypeptide comprising a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 is to be understood as a polypeptide comprising an amino acid sequence that shares at least one property or activity of any one of the polypeptides with SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, respectively, as given herein, from which it is derived, preferably to a similar or greater extent.

A polypeptide comprising a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, respectively, wherein said fragment shares at least one property or activity of any one of the polypeptides with SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, respectively, as given herein, from which it is derived, preferably to a similar or greater extent.

According to an embodiment of the method and/or cell of the invention, the fucosyltransferase of present invention solely has alpha-1,3-fucosyltransferase activity. According to an alternative embodiment of the method and/or cell of the invention, the fucosyltransferase of present invention has both alpha-1,3-fucosyltransferase activity and alpha-1,4-fucosyltransferase activity. Said fucosyltransferase having both alpha-1,3- and alpha-1,4-fucosyltransferase activity may have alpha-1,3-fucosyltransferase activity on a substrate transferring fucose from GDP-fucose to said substrate in an alpha-1,3-glycosidic linkage and alpha-1,4-fucosyltransferase activity on another substrate transferring fucose from GDP-fucose to said another substrate in an alpha-1,4-glycosidic linkage. Alternatively, said fucosyltransferase having both alpha-1,3- and alpha-1,4-fucosyltransferase activity may exert alpha-1,3-fucosyltransferase activity on a monosaccharide A and alpha-1,4-fucosyltransferase activity on a monosaccharide B, wherein both monosaccharide A and B are part of one and the same substrate.

In another preferred embodiment of the method and/or cell of present invention, the fucosyltransferase having alpha-1,3-fucosyltransferase activity on the GlcNAc and/or the Glc residue of Gal-β1,m-GlcNAc-β1,n-Gal-β1,4-Glc of a saccharide substrate as described herein has additional alpha-1,3-fucosyltransferase activity on a) a monosaccharide residue of said saccharide substrate excluding the GlcNAc and Glc residues of said Gal-β1,m-GlcNAc-β1,n-Gal-β1,4-Glc of said saccharide substrate, and/or b) a compound that is different from said saccharide substrate, said compound being chosen from the list comprising a monosaccharide like e.g. galactose, glucose, GlcNAc, a disaccharide like e.g. lactose, lactulose, N-acetyllactosamine (LacNAc), lacto-N-biose (LNB) and an oligosaccharide, like e.g. 2′fucosyllactose (2′FL), lacto-N-triose (LN3), optionally said compound is linked to a peptide, a protein and/or a lipid.

In another preferred embodiment of the method and/or cell of present invention, the fucosyltransferase having alpha-1,3-fucosyltransferase activity on the GlcNAc and/or the Glc residue of Gal-β1,m-GlcNAc-β1,n-Gal-β1,4-Glc of a saccharide substrate as described herein has alpha-1,4-fucosyltransferase activity on a) a saccharide substrate as described herein and/or b) a compound that is different from said saccharide substrate, said compound being chosen from the list comprising a monosaccharide, like e.g. galactose, glucose, GlcNAc, a disaccharide like e.g. lactose, lactulose, N-acetyllactosamine (LacNAc), lacto-N-biose (LNB) and an oligosaccharide, like e.g. 2′fucosyllactose (2′FL), lacto-N-triose (LN3), optionally said compound is linked to a peptide, a protein and/or a lipid

In another preferred embodiment of the method and/or cell of present invention, the fucosyltransferase a) has alpha-1,3-fucosyltransferase activity on the GlcNAc residue of LNnT and b) comprises a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or is a polypeptide comprising or consisting of an amino acid sequence having 72.50% or more sequence identity to the full-length amino acid sequence of any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or comprises a functional fragment of a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or comprises a functional fragment comprising an amino acid sequence of at least 10 consecutive amino acid residues from any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50. A polypeptide comprising or consisting of an amino acid sequence having 72.50% or more sequence identity to the full-length amino acid sequence of any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50 is to be understood as that the polypeptide comprises or consists of an amino acid sequence that has 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, respectively, as given herein. A polypeptide comprising a functional fragment of a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50 is to be understood as a polypeptide comprising an amino acid sequence that shares at least one property or activity of any one of the polypeptides with SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, respectively, as given herein, from which it is derived, preferably to a similar or greater extent. A polypeptide comprising a functional fragment comprising an amino acid sequence of at least 10 consecutive amino acid residues from any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, respectively, wherein said fragment shares at least one property or activity of any one of the polypeptides with SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, respectively, as given herein, from which it is derived, preferably to a similar or greater extent.

In another preferred embodiment of the method and/or cell of present invention, the fucosyltransferase a) has alpha-1,3-fucosyltransferase activity on the Glc residue of LNnT and b) comprises a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or is a polypeptide comprising or consisting of an amino acid sequence having 50.50% or more sequence identity to the full-length amino acid sequence of any one of SEQ ID NO 03, 01, 12, 18, 02, 50, 20, 09, 48, 10, 36, 08, 33, 32, 49, 35, 34, 07, 31, 28, 05, 19 or 26, or comprises a functional fragment of a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or comprises a functional fragment comprising an amino acid sequence of at least 10 consecutive amino acid residues from any one of SEQ ID NO 03, 01, 12, 18, 02, 50, 20, 09, 48, 10, 36, 08, 33, 32, 49, 35, 34, 07, 31, 28, 05, 19 or 26. A polypeptide comprising or consisting of an amino acid sequence having 50.50% or more sequence identity to the full-length amino acid sequence of any one of SEQ ID NO 03, 01, 12, 18, 02, 50, 20, 09, 48, 10, 36, 08, 33, 32, 49, 35, 34, 07, 31, 28, 05, 19 or 26 is to be understood as that the polypeptide comprises or consists of an amino acid sequence that has 50.50%, 51.0%, 52.0%, 52.50%, 55.0%, 57.50%, 60.0%, 62.50%, 65.0%, 67.50%, 70.0%, 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 03, 01, 12, 18, 02, 50, 20, 09, 48, 10, 36, 08, 33, 32, 49, 35, 34, 07, 31, 28, 05, 19 or 26, respectively, as given herein. A polypeptide comprising a functional fragment of a polypeptide according to any one of SEQ ID NO 03, 01, 12, 18, 02, 50, 20, 09, 48, 10, 36, 08, 33, 32, 49, 35, 34, 07, 31, 28, 05, 19 or 26 is to be understood as a polypeptide comprising an amino acid sequence that shares at least one property or activity of any one of the polypeptides with SEQ ID NO 03, 01, 12, 18, 02, 50, 20, 09, 48, 10, 36, 08, 33, 32, 49, 35, 34, 07, 31, 28, 05, 19 or 26, respectively, as given herein, from which it is derived, preferably to a similar or greater extent. A polypeptide comprising a functional fragment comprising an amino acid sequence of at least 10 consecutive amino acid residues from any one of SEQ ID NO 03, 01, 12, 18, 02, 50, 20, 09, 48, 10, 36, 08, 33, 32, 49, 35, 34, 07, 31, 28, 05, 19 or 26 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 03, 01, 12, 18, 02, 50, 20, 09, 48, 10, 36, 08, 33, 32, 49, 35, 34, 07, 31, 28, 05, 19 or 26, respectively, wherein said fragment shares at least one property or activity of any one of the polypeptides with SEQ ID NO 03, 01, 12, 18, 02, 50, 20, 09, 48, 10, 36, 08, 33, 32, 49, 35, 34, 07, 31, 28, 05, 19 or 26, respectively, as given herein, from which it is derived, preferably to a similar or greater extent.

In another preferred embodiment of the method and/or cell of present invention, the fucosyltransferase a) has alpha-1,3-fucosyltransferase activity on the GlcNAc residue and on the Glc residue of LNnT and b) comprises a polypeptide according to any one of SEQ ID NO 10, 09, 34, 36, 08, 07, 28, 31, 48, 35, 32, 19, 49, 26, 05 or 33, or is a polypeptide comprising or consisting of an amino acid sequence having 50.50% or more sequence identity to the full-length amino acid sequence of any one of SEQ ID NO 10, 09, 34, 36, 08, 07, 28, 31, 48, 35, 32, 19, 49, 26, 05 or 33, or comprises a functional fragment of a polypeptide according to any one of SEQ ID NO 10, 09, 34, 36, 08, 07, 28, 31, 48, 35, 32, 19, 49, 26, 05 or 33, or comprises a functional fragment comprising an amino acid sequence of at least 10 consecutive amino acid residues from any one of SEQ ID NO 10, 09, 34, 36, 08, 07, 28, 31, 48, 35, 32, 19, 49, 26, 05 or 33. A polypeptide comprising or consisting of an amino acid sequence having 50.50% or more sequence identity to the full-length amino acid sequence of any one of SEQ ID NO 10, 09, 34, 36, 08, 07, 28, 31, 48, 35, 32, 19, 49, 26, 05 or 33 is to be understood as that the polypeptide comprises or consists of an amino acid sequence that has 50.50%, 51.0%, 52.0%, 52.50%, 55.0%, 57.50%, 60.0%, 62.50%, 65.0%, 67.50%, 70.0%, 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 10, 09, 34, 36, 08, 07, 28, 31, 48, 35, 32, 19, 49, 26, 05 or 33, respectively, as given herein. A polypeptide comprising a functional fragment of a polypeptide according to any one of SEQ ID NO 10, 09, 34, 36, 08, 07, 28, 31, 48, 35, 32, 19, 49, 26, 05 or 33 is to be understood as a polypeptide comprising an amino acid sequence that shares at least one property or activity of any one of the polypeptides with SEQ ID NO 10, 09, 34, 36, 08, 07, 28, 31, 48, 35, 32, 19, 49, 26, 05 or 33, respectively, as given herein, from which it is derived, preferably to a similar or greater extent. A polypeptide comprising a functional fragment comprising an amino acid sequence of at least 10 consecutive amino acid residues from any one of SEQ ID NO 10, 09, 34, 36, 08, 07, 28, 31, 48, 35, 32, 19, 49, 26, 05 or 33 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 10, 09, 34, 36, 08, 07, 28, 31, 48, 35, 32, 19, 49, 26, 05 or 33, respectively, wherein said fragment shares at least one property or activity of any one of the polypeptides with SEQ ID NO 10, 09, 34, 36, 08, 07, 28, 31, 48, 35, 32, 19, 49, 26, 05 or 33, respectively, as given herein, from which it is derived, preferably to a similar or greater extent.

In another preferred embodiment of the method and/or cell of present invention, the fucosyltransferase a) has alpha-1,3-fucosyltransferase activity on the Glc residue of LNT and b) comprises a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or is a polypeptide comprising or consisting of an amino acid sequence having 50.50% or more sequence identity to the full-length amino acid sequence of any one of SEQ ID NO 48, 34, 32, 50, 03, 09, 33, 35, 01, 31, 28, 07, 49, 10, 18, 26, 02, 05, 08, 30, 06, 04, 27 or 11, or comprises a functional fragment of a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or comprises a functional fragment comprising an amino acid sequence of at least 10 consecutive amino acid residues from any one of SEQ ID NO 48, 34, 32, 50, 03, 09, 33, 35, 01, 31, 28, 07, 49, 10, 18, 26, 02, 05, 08, 30, 06, 04, 27 or 11. A polypeptide comprising or consisting of an amino acid sequence having 50.50% or more sequence identity to the full-length amino acid sequence of any one of SEQ ID NO 48, 34, 32, 50, 03, 09, 33, 35, 01, 31, 28, 07, 49, 10, 18, 26, 02, 05, 08, 30, 06, 04, 27 or 11 is to be understood as that the polypeptide comprises or consists of an amino acid sequence that has 50.50%, 51.0%, 52.0%, 52.50%, 55.0%, 57.50%, 60.0%, 62.50%, 65.0%, 67.50%, 70.0%, 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 48, 34, 32, 50, 03, 09, 33, 35, 01, 31, 28, 07, 49, 10, 18, 26, 02, 05, 08, 30, 06, 04, 27 or 11, respectively, as given herein. A polypeptide comprising a functional fragment of a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11 is to be understood as a polypeptide comprising an amino acid sequence that shares at least one property or activity of any one of the polypeptides with SEQ ID NO 48, 34, 32, 50, 03, 09, 33, 35, 01, 31, 28, 07, 49, 10, 18, 26, 02, 05, 08, 30, 06, 04, 27 or 11, respectively, as given herein, from which it is derived, preferably to a similar or greater extent. A polypeptide comprising a functional fragment comprising an amino acid sequence of at least 10 consecutive amino acid residues from any one of SEQ ID NO 48, 34, 32, 50, 03, 09, 33, 35, 01, 31, 28, 07, 49, 10, 18, 26, 02, 05, 08, 30, 06, 04, 27 or 11 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 48, 34, 32, 50, 03, 09, 33, 35, 01, 31, 28, 07, 49, 10, 18, 26, 02, 05, 08, 30, 06, 04, 27 or 11, respectively, wherein said fragment shares at least one property or activity of any one of the polypeptides with SEQ ID NO 48, 34, 32, 50, 03, 09, 33, 35, 01, 31, 28, 07, 49, 10, 18, 26, 02, 05, 08, 30, 06, 04, 27 or 11, respectively, as given herein, from which it is derived, preferably to a similar or greater extent.

In another preferred embodiment of the method and/or cell of present invention, the fucosyltransferase a) has alpha-1,3-fucosyltransferase activity on the Glc residue of LNT and alpha-1,4-fucosyltransferase activity on the GlcNAc residue of LNT and b) comprises a polypeptide according to any one of SEQ ID NO 03, 09, 33, 35, 08, 01, 10, 31, 28, 07, 18, 26, 02, 05, 30, 06, 04, 27 or 11, or is a polypeptide comprising or consisting of an amino acid sequence having 50.50% or more sequence identity to the full-length amino acid sequence of any one of SEQ ID NO 03, 09, 33, 35, 08, 01, 10, 31, 28, 07, 18, 26, 02, 05, 30, 06, 04, 27 or 11, or comprises a functional fragment of a polypeptide according to any one of SEQ ID NO 03, 09, 33, 35, 08, 01, 10, 31, 28, 07, 18, 26, 02, 05, 30, 06, 04, 27 or 11, or comprises a functional fragment comprising an amino acid sequence of at least 10 consecutive amino acid residues from any one of SEQ ID NO 03, 09, 33, 35, 08, 01, 10, 31, 28, 07, 18, 26, 02, 05, 30, 06, 04, 27 or 11. A polypeptide comprising or consisting of an amino acid sequence having 50.50% or more sequence identity to the full-length amino acid sequence of any one of SEQ ID NO 03, 09, 33, 35, 08, 01, 10, 31, 28, 07, 18, 26, 02, 05, 30, 06, 04, 27 or 11 is to be understood as that the polypeptide comprises or consists of an amino acid sequence that has 50.50%, 51.0%, 52.0%, 52.50%, 55.0%, 57.50%, 60.0%, 62.50%, 65.0%, 67.50%, 70.0%, 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 03, 09, 33, 35, 08, 01, 10, 31, 28, 07, 18, 26, 02, 05, 30, 06, 04, 27 or 11, respectively, as given herein. A polypeptide comprising a functional fragment of a polypeptide according to any one of SEQ ID NO 03, 09, 33, 35, 08, 01, 10, 31, 28, 07, 18, 26, 02, 05, 30, 06, 04, 27 or 11 is to be understood as a polypeptide comprising an amino acid sequence that shares at least one property or activity of any one of the polypeptides with SEQ ID NO 03, 09, 33, 35, 08, 01, 10, 31, 28, 07, 18, 26, 02, 05, 30, 06, 04, 27 or 11, respectively, as given herein, from which it is derived, preferably to a similar or greater extent. A polypeptide comprising a functional fragment comprising an amino acid sequence of at least 10 consecutive amino acid residues from any one of SEQ ID NO 03, 09, 33, 35, 08, 01, 10, 31, 28, 07, 18, 26, 02, 05, 30, 06, 04, 27 or 11 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 03, 09, 33, 35, 08, 01, 10, 31, 28, 07, 18, 26, 02, 05, 30, 06, 04, 27 or 11, respectively, wherein said fragment shares at least one property or activity of any one of the polypeptides with SEQ ID NO 03, 09, 33, 35, 08, 01, 10, 31, 28, 07, 18, 26, 02, 05, 30, 06, 04, 27 or 11, respectively, as given herein, from which it is derived, preferably to a similar or greater extent.

In a preferred embodiment of the method of present invention, the fucosylated compound is produced in a cell-free system. In an alternative preferred embodiment of the method of present invention, the fucosylated compound is produced by a cell. In a more preferred embodiment of the method of present invention, the fucosylated compound is produced by a single cell.

In a preferred embodiment of the method of present invention, the method comprises the steps of i) providing a cell expressing a fucosyltransferase having alpha-1,3-fucosyltransferase activity on the GlcNAc and/or the Glc residue of Gal-β1,m-GlcNAc-β1,n-Gal-β1,4-Glc of a saccharide substrate as described herein as described herein, ii) providing GDP-fucose, iii) providing a saccharide substrate comprising Gal-β1,m-GlcNAc-β1,n-Gal-β1,4-Glc as described herein, iv) cultivating and/or incubating said cell under conditions permissive to express said fucosyltransferase resulting in the production of said fucosylated compound, v) preferably, separating said fucosylated compound from said cultivation. Optionally, the cell is capable to produce GDP-fucose and/or said saccharide substrate Gal-β1,m-GlcNAc-β1,n-Gal-β1,4-Glc as described herein. Additionally, the cell is optionally cultivated and/or incubated under conditions permissive to produce said GDP-fucose and/or said saccharide substrate.

According to the invention, said method for the production of said fucosylated compound can make use of a non-metabolically engineered cell or can make use of a metabolically engineered cell as disclosed herein. In another preferred embodiment of the method of present invention, the fucosylated compound is produced by a metabolically engineered cell.

In a second aspect, the present invention provides a cell metabolically engineered for the production of a fucosylated compound as described herein. In the context of the invention, said fucosylated compound preferably does not occur in the wild-type progenitor of said cell. A metabolically engineered cell, preferably a single cell, is provided which is capable to express, preferably expresses a fucosyltransferase having alpha-1,3-fucosyltransferase activity on the GlcNAc and/or the Glc residue of Gal-β1,m-GlcNAc-β1,n-Gal-β1,4-Glc of a saccharide substrate as described herein.

According to another aspect, the present invention provides a method for the production of a 3-fucosyllactose (3-FL). The method comprises the steps of a) providing GDP-fucose, lactose and a fucosyltransferase having alpha-1,3-fucosyltransferase activity on the Glc residue of lactose, and b) contacting said fucosyltransferase and GDP-fucose with said lactose under conditions where the fucosyltransferase catalyses the transfer of a fucose residue from said GDP-fucose to the Glc residue of said lactose in an alpha-1,3-glycosidic linkage resulting in the production of said 3-FL, c) preferably, separating said produced 3-FL.

The fucosyltransferase used in the methods and/or cell of present invention for the production of 3-FL has alpha-1,3-fucosyltransferase activity on the Glc residue of lactose and i) comprises a polypeptide according to any one of SEQ ID NO 36, 32, 34, 28, 31, 37, 50, 27, 38, 29, 41, 49, 35, 39, 45, 43, 40 or 51, or ii) is a polypeptide comprising or consisting of an amino acid sequence having 50.0% or more sequence identity to the full-length amino acid sequence of any one of SEQ ID NO 36, 32, 34, 28, 31, 37, 50, 27, 38, 29, 41, 49, 35, 39, 45, 43, 40 or 51, or iii) comprises a functional fragment of a polypeptide according to any one of SEQ ID NO 36, 32, 34, 28, 31, 37, 50, 27, 38, 29, 41, 49, 35, 39, 45, 43, 40 or 51, or iv) comprises a functional fragment comprising an amino acid sequence of at least 10 consecutive amino acid residues from any one of SEQ ID NO 36, 32, 34, 28, 31, 37, 50, 27, 38, 29, 41, 49, 35, 39, 45, 43, 40 or 51. It is to be understood that said fucosyltransferase is capable to catalyse, preferably catalyses, the transfer of a fucose residue from GDP-fucose to the Glc residue of lactose in an alpha-1,3-glycosidic linkage resulting in the production of 3-FL. A polypeptide comprising or consisting of an amino acid sequence having 50.0% or more sequence identity to the full-length amino acid sequence of any one of SEQ ID NO 36, 32, 34, 28, 31, 37, 50, 27, 38, 29, 41, 49, 35, 39, 45, 43, 40 or 51 is to be understood as that the polypeptide comprises or consists of an amino acid sequence that has 50.0%, 50.50%, 51.0%, 52.0%, 52.50%, 55.0%, 57.50%, 60.0%, 62.50%, 65.0%, 67.50%, 70.0%, 72.50%, 75.0%, 77.50%, 80.0%, 81.0%, 82.0%, 82.50%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of any one of SEQ ID NO 36, 32, 34, 28, 31, 37, 50, 27, 38, 29, 41, 49, 35, 39, 45, 43, 40 or 51, respectively, as given herein. A polypeptide comprising a functional fragment of a polypeptide according to any one of SEQ ID NO 36, 32, 34, 28, 31, 37, 50, 27, 38, 29, 41, 49, 35, 39, 45, 43, 40 or 51 is to be understood as a polypeptide comprising an amino acid sequence that shares at least one property or activity of any one of the polypeptides with SEQ ID NO 36, 32, 34, 28, 31, 37, 50, 27, 38, 29, 41, 49, 35, 39, 45, 43, 40 or 51, respectively, as given herein, from which it is derived, preferably to a similar or greater extent. A polypeptide comprising a functional fragment comprising an amino acid sequence of at least 10 consecutive amino acid residues from any one of SEQ ID NO 36, 32, 34, 28, 31, 37, 50, 27, 38, 29, 41, 49, 35, 39, 45, 43, 40 or 51 is to be understood as a polypeptide comprising a functional fragment comprising an amino acid sequence of at least 10, 11, 12, 13, 14, 15, 16, 17, 18, 19, 20 up to the total number of consecutive amino acid residues from any one of the polypeptides with SEQ ID NO 36, 32, 34, 28, 31, 37, 50, 27, 38, 29, 41, 49, 35, 39, 45, 43, 40 or 51, respectively, wherein said fragment shares at least one property or activity of any one of the polypeptides with SEQ ID NO 36, 32, 34, 28, 31, 37, 50, 27, 38, 29, 41, 49, 35, 39, 45, 43, 40 or 51, respectively, as given herein, from which it is derived, preferably to a similar or greater extent.

In a preferred embodiment of the method of present invention, the 3-FL is produced in a cell-free system. In an alternative preferred embodiment of the method of present invention, the 3-FL is produced by a cell. In a more preferred embodiment of the method of present invention, the 3-FL is produced by a single cell.

In a preferred embodiment of the method of present invention, the method comprises the steps of i) providing a cell expressing a fucosyltransferase that has alpha-1,3-fucosyltransferase activity on the Glc residue of lactose as described herein, ii) providing GDP-fucose, iii) providing lactose, iv) cultivating and/or incubating said cell under conditions permissive to express said fucosyltransferase resulting in the production of said 3-FL, v) preferably, separating said 3-FL from said cultivation. Optionally, the cell is capable to produce GDP-fucose and/or lactose as described herein. Additionally, the cell is optionally cultivated and/or incubated under conditions permissive to produce said GDP-fucose and/or lactose. According to the invention, said method for the production of said 3-FL can make use of a non-metabolically engineered cell or can make use of a metabolically engineered cell as disclosed herein. In another preferred embodiment of the method of present invention, the 3-FL is produced by a metabolically engineered cell.

Throughout the application, unless explicitly stated otherwise, a “genetically engineered cell” or “metabolically engineered cell” preferably means a cell which is genetically engineered or metabolically engineered, respectively, for the production of said fucosylated compound and/or 3-FL according to the invention.

In another aspect, the present invention provides a cell metabolically engineered for the production of 3-FL as described herein. In the context of the invention, said 3-FL preferably does not occur in the wild-type progenitor of said cell. A metabolically engineered cell, preferably a single cell, is provided which is capable to express, preferably expresses a fucosyltransferase that has alpha-1,3-fucosyltransferase activity on the Glc residue of lactose as described herein.

In the scope of the present invention, the wording “permissive conditions to produce said fucosylated compound” or “permissive conditions to produce said 3-FL” is to be understood to be conditions relating to physical or chemical parameters including but not limited to temperature, pH, pressure, osmotic pressure and product/precursor concentration. In a particular embodiment, such conditions may include a temperature-range of 30+/−20 degrees centigrade, a pH-range of 7.0+/−3.0.

In a preferred embodiment of the method, the permissive conditions comprise use of a culture medium comprising at least one precursor as defined herein for the production of said fucosylated compound and/or 3-FL. In an alternative and/or additional preferred embodiment of the method, the permissive conditions comprise adding to the culture medium at least one precursor feed for the production of said fucosylated compound and/or 3-FL.

According to a preferred embodiment of the present invention, the cell is modified with one or more expression modules. Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

The expression of each of said expression modules can be constitutive or is created by a natural or chemical inducer. As used herein, constitutive expression should be understood as expression of a gene that is transcribed continuously in an organism. Expression that is created by a natural inducer should be understood as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. Expression that is created by a chemical inducer should be understood as a facultative or regulatory expression of a gene that is only expressed upon sensing of external chemicals (e.g. IPTG, arabinose, lactose, allo-lactose, rhamnose or fucose) via an inducible promoter or via a genetic circuit that either induces or represses the transcription or translation of said polynucleotide to a polypeptide.

The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the production of a fucosylated compound and/or 3-FL as described herein; or said recombinant gene is linked to other pathways in said host cell that are not involved in the production of said fucosylated compound and/or 3-FL. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

In a preferred embodiment of the method and/or cell of the invention, the cell is modified in the expression or activity of any one of the fucosyltransferases described herein. Preferably, said cell is capable to produce GDP-fucose which is donor for said fucosyltransferase(s).

In a preferred embodiment of the method and/or cell of the present invention, the cell expresses a fucosyltransferase that preferably uses LNnT as substrate for alpha-1,3-fucosylation of the GlcNAc residue within said LNnT over other substrates like e.g. galactose, glucose, GlcNAc, lactose, lactulose, LacNAc, LNB, 2′FL, LN3 and LNT. In a more preferred embodiment, at least 50% of the fucosylated compound obtained in a mixture by the fucosyltransferase expressed in the cell is derived from alpha-1,3-fucosylation of the GlcNAc residue of LNnT. In other words, at least 50% of the fucosylated compound obtained in a mixture by the fucosyltransferase expressed in the cell is Gal-β1,4-[Fuc-α1,3]-GlcNAc-β1,3-Gal-β1,4-Glc (lacto-N-fucopentaose III, LNFP-III). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNFP-III. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNFP-III.

In another preferred embodiment of the method and/or cell of the present invention, the cell expresses a fucosyltransferase that preferably uses LNnT as substrate for alpha-1,3-fucosylation of the Glc residue within said LNnT over other substrates like e.g. galactose, glucose, GlcNAc, lactose, lactulose, LacNAc, LNB, 2′FL, LN3 and LNT. In a more preferred embodiment, at least 50% of the fucosylated compound obtained in a mixture by the fucosyltransferase expressed in the cell is derived from alpha-1,3-fucosylation of the Glc residue of LNnT. In other words, at least 50% of the fucosylated compound obtained in a mixture by the fucosyltransferase expressed in the cell is Gal-β1,4-GlcNAc-β1,3-Gal-β1,4-[Fuc-α1,3]-Glc (lacto-N-neofucopentaose V, LNFP-VI). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNFP-VI. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNFP-VI.

In another preferred embodiment of the method and/or cell of the present invention, the cell expresses a fucosyltransferase that preferably uses LNnT as substrate for alpha-1,3-fucosylation of the GlcNAc residue and the Glc residue within said LNnT over other substrates like e.g. galactose, glucose, GlcNAc, lactose, lactulose, LacNAc, LNB, 2′FL, LN3 and LNT. In a more preferred embodiment, at least 50% of the fucosylated compound obtained in a mixture by the fucosyltransferase expressed in the cell is derived from alpha-1,3-fucosylation of the GlcNAc residue and the Glc residue of LNnT. In other words, at least 50% of the fucosylated compound obtained in a mixture by the fucosyltransferase expressed in the cell is Gal-β1,4-[Fuc-α1,3]-GlcNAc-β1,3-Gal-β1,4-[Fuc-α1,3]-Glc (lacto-N-neodifucohexaose II, LNnDFH II). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNnDFH II. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNnDFH II.

In another preferred embodiment of the method and/or cell of the present invention, the cell expresses a fucosyltransferase that preferably uses LNFP-III as substrate for alpha-1,3-fucosylation of the Glc residue within said LNFP-III over other substrates like e.g. galactose, glucose, GlcNAc, lactose, lactulose, LacNAc, LNB, 2′FL, LN3 and LNT. In a more preferred embodiment, at least 50% of the fucosylated compound obtained in a mixture by the fucosyltransferase expressed in the cell is derived from alpha-1,3-fucosylation of the Glc residue of LNFP-III. In other words, at least 50% of the fucosylated compound obtained in a mixture by the fucosyltransferase expressed in the cell is Gal-β1,4-[Fuc-α1,3]-GlcNAc-β1,3-Gal-β1,4-[Fuc-α1,3]-Glc (lacto-N-neodifucohexaose II, LNnDFH II). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNnDFH II. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNnDFH II.

In another preferred embodiment of the method and/or cell of the present invention, the cell expresses a fucosyltransferase that preferably uses LNFP-VI as substrate for alpha-1,3-fucosylation of the GlcNAc residue within said LNFP-VI over other substrates like e.g. galactose, glucose, GlcNAc, lactose, lactulose, LacNAc, LNB, 2′FL, LN3 and LNT. In a more preferred embodiment, at least 50% of the fucosylated compound obtained in a mixture by the fucosyltransferase expressed in the cell is derived from alpha-1,3-fucosylation of the GlcNAc residue of LNFP-VI. In other words, at least 50% of the fucosylated compound obtained in a mixture by the fucosyltransferase expressed in the cell is Gal-β1,4-[Fuc-α1,3]-GlcNAc-β1,3-Gal-β1,4-[Fuc-α1,3]-Glc (lacto-N-neodifucohexaose II, LNnDFH II). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNnDFH II. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNnDFH II. In another preferred embodiment of the method and/or cell of the present invention, the cell expresses a fucosyltransferase that preferably uses LNT as substrate for alpha-1,3-fucosylation of the Glc residue within said LNT over other substrates like e.g. galactose, glucose, GlcNAc, lactose, lactulose, LacNAc, LNB, 2′FL, LN3 and LNnT. In a more preferred embodiment, at least 50% of the fucosylated compound obtained in a mixture by the fucosyltransferase expressed in the cell is derived from alpha-1,3-fucosylation of the Glc residue of LNT. In other words, at least 50% of the fucosylated compound obtained in a mixture by the fucosyltransferase expressed in the cell is Gal-β1,3-GlcNAc-β1,3-Gal-β1,4-[Fuc-α1,3]-Glc (lacto-N-fucopentaose V, LNFP-V). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNFP-V. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNFP-V.

In another preferred embodiment of the method and/or cell of the present invention, the cell expresses a fucosyltransferase that preferably uses Gal-β1,3-[Fuc-α1,4]-GlcNAc-β1,3-Gal-β1,4-Glc (lacto-N-fucopentaose II, LNFP-II) as substrate for alpha-1,3-fucosylation of the Glc residue within said LNFP-II over other substrates like e.g. galactose, glucose, GlcNAc, lactose, lactulose, LacNAc, LNB, 2′FL, LN3 and LNnT. In a more preferred embodiment, at least 50% of the fucosylated compound obtained in a mixture by the fucosyltransferase expressed in the cell is derived from alpha-1,3-fucosylation of the Glc residue of LNFP-II. In other words, at least 50% of the fucosylated compound obtained in a mixture by the fucosyltransferase expressed in the cell is Gal-β1,3-[Fuc-α1,4]-GlcNAc-β1,3-Gal-β1,4-[Fuc-α1,3]-Glc (lacto-N-difucohexaose II, LNDFH-III). At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is LNDFH-II. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is LNDFH-II.

In another preferred embodiment of the method and/or cell of the present invention, the cell expresses a fucosyltransferase that preferably uses lactose as substrate for alpha-1,3-fucosylation of the Glc residue within said lactose over other substrates like e.g. galactose, glucose, GlcNAc, lactulose, LacNAc, LNB, 2′FL, LN3, LNT, LNnT. In a more preferred embodiment, at least 50% of the fucosylated compound obtained in a mixture by the fucosyltransferase expressed in the cell is derived from alpha-1,3-fucosylation of the Glc residue of lactose. In other words, at least 50% of the fucosylated compound obtained in a mixture by the fucosyltransferase expressed in the cell is 3-FL. At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% of the fucosylated compound in a mixture is 3-FL. Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the fucosyltransferase expressed in the cell is 3-FL.

In another preferred embodiment of the method and/or cell of present invention, the cell produces an oligosaccharide mixture comprising a fucosylated compound as described herein. In a more preferred embodiment, a cell expressing a fucosyltransferase of present invention produces an oligosaccharide mixture that comprises at least 50% of a fucosylated compound as described herein, wherein said fucosylated compound is obtained by alpha-1,3 fucosylation of the GlcNAc and/or the Glc residue of Gal-β1,m-GlcNAc-β1,n-Gal-β1,4-Glc of a saccharide substrate as described herein. Preferably, at least 60%, more preferably at least 70%, even more preferably at least 75%, even more preferably at least 80%, even more preferably at least 85%, even more preferably at least 90%, most preferably at least 95% of the oligosaccharides obtained in an oligosaccharide mixture by a cell of present invention is a fucosylated compound as described herein.

In another preferred embodiment of the method and/or cell of present invention, the cell produces an oligosaccharide mixture comprising 3-FL. In a more preferred embodiment, a cell expressing a fucosyltransferase of present invention produces an oligosaccharide mixture that comprises at least 50% of 3-FL, wherein said 3-FL is obtained by alpha-1,3 fucosylation of the Glc residue of lactose. Preferably, at least 60%, more preferably at least 70%, even more preferably at least 75%, even more preferably at least 80%, even more preferably at least 85%, even more preferably at least 90%, most preferably at least 95% of the oligosaccharides obtained in an oligosaccharide mixture by a cell of present invention is 3-FL.

In another preferred embodiment of the method and/or cell of present invention, the cell produces an oligosaccharide mixture comprising negatively charged (preferably sialylated) and neutral oligosaccharides. Said neutral oligosaccharides can be fucosylated non-charged oligosaccharides. Alternatively, said neutral oligosaccharides are non-fucosylated non-charged oligosaccharides. Alternatively, said neutral oligosaccharides comprise fucosylated and non-fucosylated non-charged oligosaccharides. Preferably, said negatively charged oligosaccharides comprise a fucosylated oligosaccharide as described herein and one or more negatively charged monosaccharide residues like e.g., a sialic acid. Alternatively, and/or additionally, said neutral oligosaccharides preferably comprise a non-charged fucosylated oligosaccharide as described herein and/or 3-FL.

In an additional preferred embodiment of the method and/or cell, the relative abundance of said negatively charged (preferably sialylated) oligosaccharides in said oligosaccharide mixture is at least 5%, preferably at least 7%, more preferably at least 10%. Preferably, the relative abundance of said negatively charged oligosaccharides in said oligosaccharide mixture is less than 20%, preferably less than 15%. As such, the relative abundance of said negatively charged oligosaccharides in said oligosaccharide mixture is preferably 5-20%, preferably 5-15%, more preferably 10-15%, even more preferably 12-14%, most preferably reflecting the relative abundance of negatively charged oligosaccharides in the oligosaccharide fraction of human breast milk and/or colostrum. The skilled person will further understand that if the relative abundance of the negatively charged oligosaccharides in the oligosaccharide mixture is defined, inevitably the remainder fraction of oligosaccharides in the oligosaccharide mixture are neutral oligosaccharides.

In an additional preferred embodiment of the method and/or cell, the relative abundance of fucosylated oligosaccharides in the neutral oligosaccharides fraction of said oligosaccharide mixture comprising negatively charged and neutral oligosaccharides is at least 10%, preferably at least 20%, more preferably at least 30%, most preferably at least 35%. Preferably, the relative abundance of fucosylated oligosaccharides in the neutral oligosaccharides fraction of said oligosaccharide mixture comprising negatively charged and neutral oligosaccharides is 10-60%, preferably 20-60%, more preferably 30-60%, even more preferably 30-50%, even more preferably 35-50%, most preferably reflecting the relative abundance of fucosylated oligosaccharides in the neutral oligosaccharides fraction in human breast milk and/or colostrum.

In an additional and/or alternative embodiment of the method and/or cell according to the invention, the oligosaccharide mixture comprising negatively charged and neutral oligosaccharides comprises fucosylated oligosaccharide(s) with a relative abundance in said oligosaccharide mixture of at least 10%, preferably at least 20%, more preferably at least 30%, even more preferably at least 35%, even more preferably at least 40%, most preferably at least 50%. Preferably, the relative abundance of said fucosylated oligosaccharides in said oligosaccharide mixture is less than 90%, preferably less than 80%, more preferably less than 70%, even more preferably less than 60, even more preferably less than 55%, most preferably less than 50%. As such, the relative abundance of said fucosylated oligosaccharides in said oligosaccharide mixture is preferably 10-90%, preferably 20-80%, more preferably 30-60%, even more preferably 35-50%, most preferably reflecting the relative abundance of fucosylated oligosaccharides in the oligosaccharide fraction human breast milk and/or colostrum. In a more preferred embodiment, said fucosylated oligosaccharide(s) present in said oligosaccharide mixture comprise a non-charged fucosylated oligosaccharide as described herein.

In another preferred embodiment, the cell is modified to produce an oligosaccharide mixture comprising at least 50% of a fucosylated compound wherein said fucosylated compound is an oligosaccharide obtained by alpha-1,3 fucosylation of the GlcNAc and/or the Glc residue of Gal-β1,m-GlcNAc-β1,n-Gal-β1,4-Glc of a saccharide substrate being an oligosaccharide as described herein. In a more preferred embodiment, the cell is modified to produce an oligosaccharide mixture comprising at least 60%, more preferably at least 70%, even more preferably at least 75%, even more preferably at least 80%, even more preferably at least 85%, even more preferably at least 90%, most preferably at least 95% of a fucosylated compound wherein said fucosylated compound is an oligosaccharide obtained by alpha-1,3 fucosylation of the GlcNAc and/or the Glc residue of Gal-β1,m-GlcNAc-β1,n-Gal-β1,4-Glc of a saccharide substrate being an oligosaccharide as described herein.

In another preferred embodiment, the cell is modified to produce an oligosaccharide mixture comprising at least 50% of 3-FL. In a more preferred embodiment, the cell is modified to produce an oligosaccharide mixture comprising at least 60%, more preferably at least 70%, even more preferably at least 75%, even more preferably at least 80%, even more preferably at least 85%, even more preferably at least 90%, most preferably at least 95% of 3-FL wherein said 3-FL is obtained by alpha-1,3 fucosylation of the Glc residue of lactose as described herein.

In a preferred embodiment of the method and/or cell of the present invention, the cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose.

In another preferred embodiment of the method and/or cell of the invention, the cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.

In a more preferred embodiment of the method and/or cell, the cell is modified in the expression or activity of any one of said polypeptides. Any one of said polypeptides is an endogenous protein of the cell with a modified expression or activity, preferably said endogenous polypeptide is overexpressed; alternatively, any one of said polypeptides is a heterologous protein that is heterogeneously introduced and expressed in said cell, preferably overexpressed. Said endogenous polypeptide can have a modified expression in the cell which also expresses a heterologous polypeptide of said list.

GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

CMP-Neu5Ac can be provided by an enzyme expressed in the cell or by the metabolism of the cell. Such cell producing CMP-Neu5Ac can express an enzyme converting, e.g., sialic acid, which is to be added to the cell, to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of a glucosamine-6-phosphate deaminase, over-expression of a sialate synthase encoding gene, and over-expression of an N-acetyl-D-glucosamine-2-epimerase encoding gene.

UDP-GalNAc can be synthesized from UDP-GlcNAc by the action of a single-step reaction using an UDP-N-acetylglucosamine 4-epimerase like e.g. wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production. UDP-ManNAc can be synthesized directly from UDP-GlcNAc via an epimerization reaction performed by an UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production. According to a preferred embodiment of the method and/or cell of the present invention, the cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a more preferred embodiment of the method and/or cell, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase.

In another more preferred embodiment of the method and/or cell, the sialyltransferase is chosen from the list comprising alpha-2,3-sialyltransferase, alpha-2,6-sialyltransferase and alpha-2,8-sialyltransferase.

In another more preferred embodiment of the method and/or cell, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In another more preferred embodiment of the method and/or cell, the glucosyltransferase is chosen from the list comprising alpha-glucosyltransferase, beta-1,2-glucosyltransferase, beta-1,3-glucosyltransferase and beta-1,4-glucosyltransferase.

In another more preferred embodiment of the method and/or cell, the mannosyltransferase is chosen from the list comprising alpha-1,2-mannosyltransferase, alpha-1,3-mannosyltransferase and alpha-1,6-mannosyltransferase.

In another more preferred embodiment of the method and/or cell, the N-acetylglucosaminyltransferase is chosen from the list comprising galactoside beta-1,3-N-acetylglucosaminyltransferase and beta-1,6-N-acetylglucosaminyltransferase.

In another more preferred embodiment of the method and/or cell, the N-acetylgalactosaminyltransferase is an alpha-1,3-N-acetylgalactosaminyltransferase.

In another more preferred embodiment of the method and/or cell of the invention, the cell is modified in the expression or activity of at least one of said glycosyltransferases. Said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to a preferred embodiment of the method and/or cell of the invention, the cell is using a precursor as defined herein for the production of said fucosylated compound and/or 3-FL, preferably said precursor being fed to the cell from the cultivation medium. According to a more preferred embodiment of the method and/or cell, the cell is using at least two precursors for the production of said fucosylated compound and/or 3-FL, preferably said precursors being fed to the cell from the cultivation medium. According to another preferred embodiment of the method and/or cell of the invention, the cell is producing at least one precursor, preferably at least two precursors, for the production of said fucosylated compound and/or 3-FL. In a preferred embodiment of the method and/or cell, the precursor that is used by the cell for the production of said fucosylated compound or 3-FL is completely converted into said fucosylated compound or 3-FL.

In a preferred embodiment of the method and/or cell according to the invention, the cell is capable to produce said saccharide substrate.

In a more preferred embodiment of the method and/or cell according to the invention, the cell is capable to produce GlcNAc-β1,3-Gal-β1,4-Glc or lacto-N-triose (LN3). LN3 production in a cell can be obtained by expression of a galactoside beta-1,3-N-acetylglucosaminyltransferase gene which transfers a GlcNAc residue from UDP-GlcNAc to lactose to form LN3. The UDP-GlcNAc and lactose that are needed in said reaction can be fed to the cultivation and/or can be produced by the metabolism of the cell and/or can be provided by enzymes expressed in the cell.

In an alternative and/or additional more preferred embodiment of the method and/or cell according to the invention, the cell is capable to produce Gal-β1,3-GlcNAc-β1,3-Gal-β1,4-Glc or lacto-N-tetraose (LNT). LNT production in a cell can be obtained by expression of a galactoside beta-1,3-N-acetylglucosaminyltransferase gene and an N-acetylglucosamine beta-1,3-galactosyltransferase gene which respectively transfers a GlcNAc residue from UDP-GlcNAc to lactose to form LN3 and that transfers a Gal residue from UDP-Gal to LN3 to form LNT. The UDP-GlcNAc, UDP-Gal and lactose that is/are needed in said reaction can be fed to the cultivation and/or can be produced by the metabolism of the cell and/or can be provided by enzymes expressed in the cell.

In an alternative and/or additional more preferred embodiment of the method and/or cell according to the invention, the cell is capable to produce Gal-β1,3-[Fuc-α1,4]-GlcNAc-β1,3-Gal-β1,4-Glc (LNFP-II). LNFP-II production in a cell can be obtained by expression of a galactoside beta-1,3-N-acetylglucosaminyltransferase gene, an N-acetylglucosamine beta-1,3-galactosyltransferase gene and an alpha-1,2-fucosyltransferase gene, which respectively transfers a GlcNAc residue from UDP-GlcNAc to lactose to form LN3 and that transfers a Gal residue from UDP-Gal to LN3 to form LNT and that transfers a fucose residue from GDP-fucose to the GlcNAc residue of LNT to form LNFP-II. The UDP-GlcNAc, UDP-Gal and lactose that is/are needed in said reaction can be fed to the cultivation and/or can be produced by the metabolism of the cell and/or can be provided by enzymes expressed in the cell.

In an alternative and/or additional more preferred embodiment of the method and/or cell according to the invention, the cell is capable to produce Gal-β1,4-GlcNAc-β1,3-Gal-β1,4-Glc or lacto-N-neotetraose (LNnT). LNnT production in a cell can be obtained by expression of a galactoside beta-1,3-N-acetylglucosaminyltransferase gene and an N-acetylglucosamine beta-1,4-galactosyltransferase gene which respectively transfers a GlcNAc residue from UDP-GlcNAc to lactose to form LN3 and that transfers a Gal residue from UDP-Gal to LN3 to form LNnT. The UDP-GlcNAc, UDP-Gal and lactose that is/are needed in said reaction can be fed to the cultivation and/or can be produced by the metabolism of the cell and/or can be provided by enzymes expressed in the cell.

In an alternative and/or additional more preferred embodiment of the method and/or cell according to the invention, the cell is capable to produce Gal-β1,4-[Fuc-α1,3]-GlcNAc-β1,3-Gal-β1,4-Glc (lacto-N-fucopentaose III, LNFP-III). LNFP-III production in a cell can be obtained by expression of a galactoside beta-1,3-N-acetylglucosaminyltransferase gene, an N-acetylglucosamine beta-1,4-galactosyltransferase gene and an alpha-1,3-fucosyltransferase gene, which respectively transfers a GlcNAc residue from UDP-GlcNAc to lactose to form LN3 and that transfers a Gal residue from UDP-Gal to LN3 to form LNnT and that transfers a fucose residue from GDP-fucose to the GlcNAc residue of LNnT to form LNFP-III. The UDP-GlcNAc, UDP-Gal and lactose that is/are needed in said reaction can be fed to the cultivation and/or can be produced by the metabolism of the cell and/or can be provided by enzymes expressed in the cell.

In an alternative and/or additional more preferred embodiment of the method and/or cell according to the invention, the cell is capable to produce Gal-β1,4-GlcNAc-β1,3-Gal-β1,4-[Fuc-α1,3]-Glc (lacto-N-neofucopentaose V, LNFP-VI). LNFP-VI production in a cell can be obtained by expression of a galactoside beta-1,3-N-acetylglucosaminyltransferase gene, an N-acetylglucosamine beta-1,4-galactosyltransferase gene and an alpha-1,3-fucosyltransferase gene, which respectively transfers a GlcNAc residue from UDP-GlcNAc to lactose to form LN3 and that transfers a Gal residue from UDP-Gal to LN3 to form LNnT and that transfers a fucose residue from GDP-fucose to the Glc residue of LNnT to form LNFP-VI. The UDP-GlcNAc, UDP-Gal and lactose that is/are needed in said reaction can be fed to the cultivation and/or can be produced by the metabolism of the cell and/or can be provided by enzymes expressed in the cell.

In another and/or additional more preferred embodiment of the method and/or cell according to the invention, the cell is capable to produce lactose. A cell producing UDP-Gal can express an enzyme converting, e.g. UDP-glucose, to UDP-Gal. This enzyme may be, e.g., the UDP-glucose 4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of an UDP-glucose 4-epimerase encoding gene.

A cell producing UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, and Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

A cell producing lactose can express a beta-1,4-galactosyltransferase which transfers a Gal residue from UDP-Gal to glucose in a beta-1,4-linkage, wherein said glucose can be fed to the cultivation and/or can be produced by the metabolism of the cell and/or can be provided by enzymes expressed in the cell like e.g. an UDP-glucose 4-epimerase. Preferably, the cell using lactose for LN3, LNT and/or derivatives thereof does not have an active galactosidase like e.g., lacZ that degrades lactose into glucose and galactose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

Preferably, the cell is modified to produce GDP-fucose. More preferably, the cell is modified for enhanced GDP-fucose production. Said modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

In the context of the invention, it should be understood that said fucosylated compound as described herein and/or 3-FL is preferably produced intracellularly. The skilled person will further understand that a fraction or substantially all of said produced fucosylated compound and/or 3-FL remains intracellularly and/or is excreted outside the cell either passively or through active transport.

In a preferred embodiment of the method and/or cell of the invention, the cell expresses a membrane transporter protein or a polypeptide having transport activity hereby transporting compounds across the outer membrane of the cell wall. In another preferred embodiment of the method and/or cell of the invention, the cell expresses more than one membrane transporter protein or polypeptide having transport activity hereby transporting compounds across the outer membrane of the cell wall. In a more preferred embodiment of the method and/or cell of the invention, the cell is modified in the expression or activity of said membrane transporter protein or polypeptide having transport activity. Said membrane transporter protein or polypeptide having transport activity is an endogenous protein of the cell with a modified expression or activity, preferably said endogenous membrane transporter protein or polypeptide having transport activity is overexpressed; alternatively said membrane transporter protein or polypeptide having transport activity is a heterologous protein that is heterogeneously introduced and expressed in said cell, preferably overexpressed. Said endogenous membrane transporter protein or polypeptide having transport activity can have a modified expression in the cell which also expresses a heterologous membrane transporter protein or polypeptide having transport activity.

In a more preferred embodiment of the method and/or cell of the invention, the membrane transporter protein or polypeptide having transport activity is chosen from the list comprising porters, P—P-bond-hydrolysis-driven transporters, β-barrel porins, auxiliary transport proteins and phosphotransfer-driven group translocators. In an even more preferred embodiment of the method and/or cell of the invention, the porters comprise MFS transporters, sugar efflux transporters and siderophore exporters. In another more preferred embodiment of the method and/or cell of the invention, the P—P-bond-hydrolysis-driven transporters comprise ABC transporters and siderophore exporters.

In another preferred embodiment of the method and/or cell of the invention, the membrane transporter protein or polypeptide having transport activity controls the flow over the outer membrane of the cell wall of said fucosylated compound and/or 3-FL. In an alternative and/or additional preferred embodiment of the method and/or cell of the invention, the membrane transporter protein or polypeptide having transport activity controls the flow over the outer membrane of the cell wall of one or more precursor(s) to be used in said production of said fucosylated compound and/or 3-FL.

In another preferred embodiment of the method and/or cell of the invention, the membrane transporter protein or polypeptide having transport activity provides improved production of said fucosylated compound and/or 3-FL. In an alternative and/or additional preferred embodiment of the method and/or cell of the invention, the membrane transporter protein or polypeptide having transport activity provides enabled efflux of said fucosylated compound and/or 3-FL. In an alternative and/or additional preferred embodiment of the method and/or cell of the invention, the membrane transporter protein or polypeptide having transport activity provides enhanced efflux of said fucosylated compound and/or 3-FL.

In another preferred embodiment of the method and/or cell of the invention, the cell expresses a polypeptide selected from the group comprising a lactose transporter like e.g., the LacY or lac12 permease, a fucose transporter, a glucose transporter, a galactose transporter, a transporter for a nucleotide-activated sugar like for example a transporter for UDP-GlcNAc, UDP-Gal and/or GDP-Fuc.

In another preferred embodiment of the method and/or cell of the present invention, the cell expresses a membrane transporter protein belonging to the family of MFS transporters like e.g., an MdfA polypeptide of the multidrug transporter MdfA family from species comprising E. coli (UniProt ID POAEY8), Cronobacter muytjensii (UniProt ID A0A2T7ANQ9), Citrobacter youngae (UniProt ID D4BC23) and Yokenella regensburgei (UniProt ID G9Z5F4). In another preferred embodiment of the method and/or cell of the present invention, the cell expresses a membrane transporter protein belonging to the family of sugar efflux transporters like e.g., a SetA polypeptide of the SetA family from species comprising E. coli (UniProt ID P31675) and Citrobacter koseri (UniProt ID A0A078LM16). In another preferred embodiment of the method and/or cell of the present invention, the cell expresses a membrane transporter protein belonging to the family of siderophore exporters like e.g., the E. coli entS (UniProt ID P24077) and the E. coli iceT (UniProt ID A0A024L207). In another preferred embodiment of the method and/or cell of the present invention, the cell expresses a membrane transporter protein belonging to the family of ABC transporters like e.g., oppF from E. coli (UniProt ID P77737), ImrA from Lactococcus lactis subsp. lactis bv. diacetylactis (UniProt ID A0A1V0NEL4) and Blon_2475 from Bifidobacterium longum subsp. infantis (UniProt ID B7GPD4). In a more preferred embodiment of the method and/or cell of the present invention, the cell expresses more than one membrane transporter protein chosen from the list comprising a lactose transporter like e.g. the LacY or lac12 permease, a fucose transporter, a glucose transporter, a galactose transporter, a transporter for a nucleotide-activated sugar like for example a transporter for UDP-GlcNAc, UDP-Gal and/or GDP-Fuc, the MdfA protein from E. coli (UniProt ID POAEY8), the MdfA protein from Cronobacter muytjensii (UniProt ID A0A2T7ANQ9), the MdfA protein from Citrobacter youngae (UniProt ID D4BC23), the MdfA protein from Yokenella regensburgei (UniProt ID G9Z5F4), the SetA protein from E. coli (UniProt ID P31675), the SetA protein from Citrobacter koseri (UniProt ID A0A078LM16), the entS protein from E. coli (UniProt ID P24077), the iceT protein from E. coli (UniProt ID A0A024L207), the oppF protein from E. coli (UniProt ID P77737), the ImrA protein from Lactococcus lactis subsp. lactis bv. diacetylactis (UniProt ID A0A1V0NEL4) and Blon_2475 from Bifidobacterium longum subsp. infantis (UniProt ID B7GPD4).

According to another preferred embodiment of the method and/or cell of the invention, the cell comprises a catabolic pathway for selected mono-, di- or oligosaccharides which is at least partially inactivated, the mono-, di-, or oligosaccharides being involved in and/or required for the production of said fucosylated compound and/or 3-FL.

According to another preferred embodiment of the method and/or cell of the invention, the cell produces 90 g/L or more of said fucosylated compound and/or 3-FL in the whole broth and/or supernatant and/or wherein said fucosylated compound in the whole broth and/or supernatant has a purity of at least 80% measured on the total amount of said fucosylated compound and its precursor(s) in the whole broth and/or supernatant, respectively, and/or wherein said 3-FL in the whole broth and/or supernatant has a purity of at least 80% measured on the total amount of said 3-FL and its precursor(s) in the whole broth and/or supernatant, respectively.

Another embodiment of the invention provides for a method and a cell wherein said fucosylated compound and/or 3-FL is produced in and/or by a fungal, yeast, bacterial, insect, plant, animal or protozoan cell as described herein.

The cell is chosen from the list comprising a bacterium, a yeast, or a fungus, or refers to a plant, animal, or protozoan cell. The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g. Saccharomyces cerevisiae, S. bayanus, S. boulardii), Zygosaccharomyces, Pichia (with members like e.g. Pichia pastoris, P. anomala, P. kluyveri), Komagataella, Hansenula, Kluyveromyces (with members like e.g. Kluyveromyces lactis, K. marxianus, K. thermotolerans), Debaromyces, Candida, Schizosaccharomyces, Schwanniomyces, Torulaspora, Yarrowia (like e.g. Yarrowia lipolytica) or Starmerella (like e.g. Starmerella bombicola). The latter yeast is preferably selected from Pichia pastoris, Yarrowia lipolitica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. The latter fungus belongs preferably to the genus Rhizopus, Dictyostelium, Penicillium, Mucor or Aspergillus. Plant cells include cells of flowering and non-flowering plants, as well as algal cells, for example Chlamydomonas, Chlorella, etc. Preferably, said plant is a tobacco, alfalfa, rice, tomato, cotton, rapeseed, soy, maize, or corn plant. The latter animal cell is preferably derived from non-human mammals (e.g. cattle, buffalo, pig, sheep, mouse, rat, primate (e.g., chimpanzee, orangutan, gorilla, monkey (e.g., Old World, New World), lemur), dog, cat, rabbit, horse, cow, goat, ox, deer, musk deer, bovid, whale, dolphin, hippopotamus, elephant, rhinoceros, giraffe, zebra, lion, cheetah, tiger, panda, red panda, otter), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a genetically modified or engineered cell line derived from human cells excluding embryonic stem cells. Both human and non-human mammalian cells are preferably chosen from the list comprising an epithelial cell like e.g., a mammary epithelial cell, an embryonic kidney cell (e.g., HEK293 or HEK 293T cell), a fibroblast cell, a COS cell, a Chinese hamster ovary (CHO) cell, a murine myeloma cell like e.g. an N20, SP2/0 or YB2/0 cell, an NIH-3T3 cell, a non-mammary adult stem cell or derivatives thereof such as described in WO21067641, a lactocyte derived from mammalian induced pluripotent stem cells, preferably human induced pluripotent stem cells, a lactocyte as part of mammary-like gland organoids, a post-parturition mammary epithelium cell, a polarized mammary cell, preferably a polarized mammary cell selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof as well-known to the person skilled in the art from e.g., WO2021/219634, WO 2022/054053, WO 2021/141762, WO 2021/142241, WO 2021/067641 and WO2021/242866. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell. More preferably, the cell is selected from the group consisting of prokaryotic cells and eukaryotic cells, preferably from the group consisting of yeast cells, bacterial cells, archaebacterial cells, algae cells, and fungal cells as described herein.

Another embodiment provides for a cell to be stably cultured in a medium, wherein said medium can be any type of growth medium as well-known to the skilled person comprising minimal medium, complex medium or growth medium enriched in certain compounds like for example but not limited to vitamins, trace elements, amino acids and/or, precursors as defined herein.

The cell as used herein is capable to grow on a monosaccharide, disaccharide, oligosaccharide, polysaccharide, polyol, glycerol, a complex medium including molasses, corn steep liquor, peptone, tryptone, yeast extract or a mixture thereof like e.g. a mixed feedstock, preferably a mixed monosaccharide feedstock like e.g. hydrolysed sucrose as the main carbon source. With the term “complex medium” is meant a medium for which the exact constitution is not determined. With the term “main” is meant the most important carbon source for the cell for the production of a fucosylated compound of interest and/or 3-FL, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. In one embodiment of the invention, said carbon source is the sole carbon source for said organism, i.e., 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of said fucosylated compound and/or 3-FL.

According to another embodiment of the method of the invention, the conditions permissive to produce said fucosylated compound and/or 3-FL comprise the use of a culture medium comprising at least one precursor for the production of said fucosylated compound and/or 3-FL, respectively. Preferably, the culture medium contains at least one precursor selected from the group comprising lactose, galactose, fucose, sialic acid, GlcNAc, GalNAc, lacto-N-biose (LNB), N-acetyllactosamine (LacNAc).

According to an alternative and/or additional embodiment of the method of the invention, the conditions permissive to produce said fucosylated compound and/or 3-FL comprise adding to the culture medium at least one precursor feed for the production of said fucosylated compound and/or 3-FL.

According to an alternative embodiment of the method of the invention, the conditions permissive to produce said fucosylated compound and/or 3-FL comprise the use of a culture medium to cultivate a cell of present invention for the production of said fucosylated compound and/or 3-FL wherein said culture medium lacks any precursor for the production of said fucosylated compound and/or 3-FL, respectively and is combined with a further addition to said culture medium of at least one precursor feed for the production of said fucosylated compound and/or 3-FL.

In a preferred embodiment, the method for the production of said fucosylated compound as described herein comprises at least one of the following steps:
i) Use of a culture medium comprising at least one precursor;
ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
iv) Adding at least one precursor feed in a continuous manner to the culture medium over the course of 1 day, 2 days, 3 days, 4 days, 5 days by means of a feeding solution;
v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least g/L, more preferably at least 150 g/L, more preferably at least 175 g/L, more preferably at least 200 g/L in the final cultivation.

In another and/or additional preferred embodiment, the method for the production of said fucosylated compound as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
ii) Adding to the culture medium at least one precursor in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than two-fold of the volume of the culture medium before the addition of said precursor feed pulse(s);
iii) Adding to the culture medium in a reactor at least one precursor feed in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than two-fold of the volume of the culture medium before the addition of said precursor feed pulse(s) and wherein preferably, the pH of said precursor feed pulse(s) is set between 3.0 and 7.0 and wherein preferably, the temperature of said precursor feed pulse(s) is kept between 20° C. and 80° C.;
iv) Adding at least one precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least g/L, more preferably at least 150 g/L, more preferably at least 175 g/L, more preferably at least 200 g/L in the final cultivation.

In a further, more preferred embodiment, the method for the production of said fucosylated compound as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least g/L, more preferably at least 150 g/L, more preferably at least 175 g/L, more preferably at least 200 g/L in the final cultivation.

In a preferred embodiment, the method for the production of said 3-FL as described herein comprises at least one of the following steps:
i) Use of a culture medium comprising at least one precursor;
ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
iv) Adding at least one precursor feed in a continuous manner to the culture medium over the course of 1 day, 2 days, 3 days, 4 days, 5 days by means of a feeding solution;
v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In another and/or additional preferred embodiment, the method for the production of said fucosylated compound as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
ii) Adding to the culture medium at least one precursor in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than two-fold of the volume of the culture medium before the addition of said precursor feed pulse(s);
iii) Adding to the culture medium in a reactor at least one precursor feed in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than two-fold of the volume of the culture medium before the addition of said precursor feed pulse(s) and wherein preferably, the pH of said precursor feed pulse(s) is set between 3.0 and 7.0 and wherein preferably, the temperature of said precursor feed pulse(s) is kept between 20° C. and 80° C.;
iv) Adding at least one precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In a further, more preferred embodiment, the method for the production of said 3-FL as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

Preferably the lactose feed is accomplished by adding lactose from the beginning of the cultivation at a concentration of at least 5 mM, preferably in a concentration of 30, 40, 50, 60, 70, 80, 90, 100, 150 mM, more preferably at a concentration >300 mM.

In another embodiment the lactose feed is accomplished by adding lactose to the cultivation in a concentration, such that throughout the production phase of the cultivation a lactose concentration of at least 5 mM, preferably 10 mM or 30 mM is obtained.

In a further embodiment of the methods described herein the cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the culture medium in a second phase.

In another preferred embodiment of the method of present invention, a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.

In another preferred embodiment of the method of present invention, a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.

In an alternative preferable embodiment, in the method as described herein, the lactose is added already in the first phase of exponential growth together with the carbon-based substrate.

According to the present invention, the methods as described herein preferably comprises a step of separating said fucosylated compound and/or said 3-FL from said cultivation.

The terms “separating from said cultivation” means harvesting, collecting, or retrieving said fucosylated compound and/or 3-FL from the cell and/or the medium of its growth.

Said fucosylated compound and/or 3-FL can be separated in a conventional manner from the aqueous culture medium, in which the cell was grown. In case said fucosylated compound and/or 3-FL is still present in the cells producing said fucosylated compound and/or 3-FL, conventional manners to free or to extract said fucosylated compound and/or 3-FL out of the cells can be used, such as cell destruction using high pH, heat shock, sonication, French press, homogenization, enzymatic hydrolysis, chemical hydrolysis, solvent hydrolysis, detergent, hydrolysis, etc. The culture medium and/or cell extract together and separately can then be further used for separating said fucosylated compound and/or 3-FL.

This preferably involves clarifying said fucosylated compound and/or 3-FL to remove suspended particulates and contaminants, particularly cells, cell components, insoluble metabolites and debris produced by culturing the metabolically engineered cell. In this step, said fucosylated compound and/or 3-FL can be clarified in a conventional manner. Preferably, said fucosylated compound and/or 3-FL is clarified by centrifugation, flocculation, decantation and/or filtration. Another step of separating said fucosylated compound and/or 3-FL preferably involves removing substantially all the proteins, peptides, amino acids, RNA and DNA, and any endotoxins and glycolipids that could interfere with the subsequent separation step, from said fucosylated compound and/or 3-FL, preferably after it has been clarified. In this step, proteins and related impurities can be removed from said fucosylated compound and/or 3-FL in a conventional manner. Preferably, proteins, salts, by-products, colour, endotoxins and other related impurities are removed from said fucosylated compound and/or 3-FL by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography or electrodialysis. With the exception of size exclusion chromatography, remaining proteins and related impurities are retained by a chromatography medium or a selected membrane.

In a further preferred embodiment, the methods as described herein also provide for a further purification of said fucosylated compound and/or 3-FL as produced according to a method of present invention. A further purification of said fucosylated compound and/or 3-FL may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of said fucosylated compound and/or 3-FL. Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced fucosylated compound and/or 3-FL.

In an exemplary embodiment, the separation and purification of said fucosylated compound and/or 3-FL is made in a process, comprising the following steps in any order:
a) contacting the cultivation or a clarified version thereof with a nanofiltration membrane with a molecular weight cut-off (MWCO) of 600-3500 Da ensuring the retention of the produced fucosylated compound and/or 3-FL and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said fucosylated compound and/or 3-FL in the form of a salt from the cation of said electrolyte.

In an alternative exemplary embodiment, the separation and purification of said fucosylated compound and/or 3-FL is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said fucosylated compound and/or 3-FL is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of said fucosylated compound and/or 3-FL is made in the following way. The cultivation comprising the produced fucosylated compound and/or 3-FL, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
   wherein a purified solution comprising the produced fucosylated compound and/or 3-FL at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In a specific embodiment, the present invention provides the produced fucosylated compound and/or 3-FL which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

The invention furthermore provides a spray-dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. In an embodiment, the present invention provides a spray-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the methods described herein. In a preferred embodiment, said at least fucosylated compound present in said spray-dried powder is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II). In another preferred embodiment, said spray-dried powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH-II). In another embodiment, the present invention provides a spray-dried powder comprising a mixture of mammalian milk oligosaccharides (MMOs). In a preferred embodiment, a spray-dried powder is provided that comprises at least one negatively charged and/or at least one neutral MMO. In a more preferred embodiment, a spray-dried powder is provided that comprises, consists of or consists essentially of at least one of LNFP-III, LNFP-VI, LNnDFH II, LNFP-V and LNDFH-II. In an even more preferred embodiment, a spray-dried powder is provided that comprises, consists of or consists essentially of at least one of LNFP-III, LNFP-VI, LNnDFH II, LNFP-V and LNDFH-II that is/are obtainable, preferably obtained, by the methods as described herein. In another more preferred embodiment, a spray-dried powder is provided that comprises a mixture of MMOs comprising, consisting of or consisting essentially of at least one MMO chosen from the group comprising LNFP-1, LNFP-II, LNFP-III, LNFP-V, LNFP-VI, LNDFH-1, LNDFH-II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.

The invention furthermore provides a drum-dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. In an embodiment, the present invention provides a drum-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the methods described herein. In a preferred embodiment, said at least fucosylated compound present in said drum-dried powder is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II). In another preferred embodiment, said drum-dried powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH-II). In another embodiment, the present invention provides a drum-dried powder comprising a mixture of mammalian milk oligosaccharides (MMOs). In a preferred embodiment, a drum-dried powder is provided that comprises at least one negatively charged and/or at least one neutral MMO. In a more preferred embodiment, a drum-dried powder is provided that comprises, consists of or consists essentially of at least one of LNFP-III, LNFP-VI, LNnDFH II, LNFP-V and LNDFH-II. In an even more preferred embodiment, a drum-dried powder is provided that comprises, consists of or consists essentially of at least one of LNFP-III, LNFP-VI, LNnDFH II, LNFP-V and LNDFH-II that is/are obtainable, preferably obtained, by the methods as described herein. In another more preferred embodiment, a drum-dried powder is provided that comprises a mixture of MMOs comprising, consisting of or consisting essentially of at least one MMO chosen from the group comprising LNFP-1, LNFP-II, LNFP-III, LNFP-V, LNFP-VI, LNDFH-1, LNDFH-II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.

The invention furthermore provides a roller-dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. In an embodiment, the present invention provides a roller-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the methods described herein. In a preferred embodiment, said at least fucosylated compound present in said roller-dried powder is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II). In another preferred embodiment, said roller-dried powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH-II). In another embodiment, the present invention provides a roller-dried powder comprising a mixture of mammalian milk oligosaccharides (MMOs). In a preferred embodiment, a roller-dried powder is provided that comprises at least one negatively charged and/or at least one neutral MMO. In a more preferred embodiment, a roller-dried powder is provided that comprises, consists of or consists essentially of at least one of LNFP-III, LNFP-VI, LNnDFH II, LNFP-V and LNDFH-II. In an even more preferred embodiment, a roller-dried powder is provided that comprises, consists of or consists essentially of at least one of LNFP-III, LNFP-VI, LNnDFH II, LNFP-V and LNDFH-II that is/are obtainable, preferably obtained, by the methods as described herein. In another more preferred embodiment, a roller-dried powder is provided that comprises a mixture of MMOs comprising, consisting of or consisting essentially of at least one MMO chosen from the group comprising LNFP-1, LNFP-II, LNFP-III, LNFP-V, LNFP-VI, LNDFH-1, LNDFH-II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.

The invention furthermore provides a dried powder comprising at least 50% w/w of a fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. The invention also provides a dried powder comprising at least 50% w/w of a fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, wherein said fucosylated compound is obtainable, preferably obtained, by a method of present invention. In a preferred embodiment, the powder is dried by any one of spray-drying, drum-drying or roller-drying.

The invention also provides a dried powder comprising, consisting of, or consisting essentially of a mixture of MMOs, wherein said mixture comprises 0.1 to 30% w/w, preferably 0.1 to 20% w/w, more preferably 1 to 10% w/w, of one or more fucosylated compound(s) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. The invention also provides a dried powder comprising, consisting of, or consisting essentially of a mixture of MMOs, wherein said mixture comprises 0.1 to 30% w/w, preferably 0.1 to 20% w/w, more preferably 1 to 10% w/w, of one or more fucosylated compound(s) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid wherein said fucosylated compound is obtainable, preferably obtained, by a method of present invention. In a preferred embodiment, the powder is dried by any one of spray-drying, drum-drying or roller-drying.

Another aspect of the present invention provides the use of a cell as defined herein, in a method for the production of said fucosylated compound, preferably in a method for the production of said fucosylated compound according to the invention. An alternative and/or additional aspect of the present invention provides the use of a cell as defined herein, in a method for the production of a mixture of di- and oligosaccharides comprising at least one fucosylated compound as described herein. An alternative and/or additional aspect of the present invention provides the use of a cell as defined herein, in a method for the production of a mixture of negatively charged and/or neutral di- and oligosaccharides comprising at least one fucosylated compound as described herein. A preferred aspect of the present invention provides the use of a cell as defined herein, in a method for the production of a mixture of sialylated and/or neutral di- and oligosaccharides comprising at least one fucosylated compound.

An alternative and/or additional aspect of the present invention provides the use of a cell as defined herein, in a method for the production of a mixture of oligosaccharides comprising at least one fucosylated compound according to the invention. An alternative and/or additional aspect of the present invention provides the use of a cell as defined herein, in a method for the production of a mixture of negatively charged and/or neutral oligosaccharides comprising at least one fucosylated compound as described herein. A preferred aspect of the present invention provides the use of a cell as defined herein, in a method for the production of a mixture of sialylated and/or neutral oligosaccharides comprising at least one fucosylated compound as described herein. A preferred aspect provides the use of a cell of present invention in a method for the production of a mixture of mammalian milk oligosaccharides (MMOs) comprising at least one fucosylated compound as described herein. A further aspect of the present invention provides the use of a method as defined herein for the production of said fucosylated compound.

Another aspect of the present invention provides the use of a cell as defined herein, in a method for the production of 3-FL, preferably in a method for the production of said 3-FL. An alternative and/or additional aspect of the present invention provides the use of a cell as defined herein, in a method for the production of a mixture of di- and oligosaccharides comprising 3-FL. An alternative and/or additional aspect of the present invention provides the use of a cell as defined herein, in a method for the production of a mixture of negatively charged and/or neutral di- and oligosaccharides comprising 3-FL. A preferred aspect of the present invention provides the use of a cell as defined herein, in a method for the production of a mixture of sialylated and/or neutral di- and oligosaccharides comprising 3-FL.

An alternative and/or additional aspect of the present invention provides the use of a cell as defined herein, in a method for the production of a mixture of oligosaccharides comprising 3-FL. An alternative and/or additional aspect of the present invention provides the use of a cell as defined herein, in a method for the production of a mixture of negatively charged and/or neutral oligosaccharides comprising 3-FL. A preferred aspect of the present invention provides the use of a cell as defined herein, in a method for the production of a mixture of sialylated and/or neutral oligosaccharides comprising 3-FL. A preferred aspect provides the use of a cell of present invention in a method for the production of a mixture of mammalian milk oligosaccharides (MMOs) comprising 3-FL. A further aspect of the present invention provides the use of a method as defined herein for the production of said 3-FL.

Furthermore, the invention also relates to said fucosylated compound and/or said 3-FL obtained by the methods according to the invention, as well as to the use of a polynucleotide, the vector, host cells or the polypeptide as described above for the production of said fucosylated compound and/or 3-FL. Said fucosylated compound may be used for the manufacture of a preparation, as food additive, prebiotic, symbiotic, for the supplementation of baby food, adult food, infant animal feed, adult animal feed, or as either therapeutically or pharmaceutically active compound or in cosmetic applications.

In a preferred embodiment, said preparation comprises, consists of or consists essentially of at least one fucosylated compound that is obtainable, preferably obtained, by the methods as described herein. In another preferred embodiment, said preparation comprises, consists of or consists essentially of a spray-dried powder that comprises at least one fucosylated compound as described in present invention. In another preferred embodiment, said preparation comprises, consists of or consists essentially of a drum-dried powder that comprises at least one fucosylated compound as described in present invention. In another preferred embodiment, said preparation comprises, consists of or consists essentially of a roller-dried powder that comprises at least one fucosylated compound as described in present invention. In another preferred embodiment, said preparation comprises, consists of or consists essentially of a mixture of mammalian milk oligosaccharides (MMOs) wherein said mixture comprises at least one of LNFP-III, LNFP-VI, LNnDFH II, LNFP-V and LNDFH-II, that is/are obtainable, preferably obtained, by the methods as described herein. In a more preferred embodiment, said preparation comprises, consists of or consists essentially of at least one negatively charged MMO and/or at least one neutral MMO. In an even more preferred embodiment, said at least one negatively charged MMO is a sialylated MMO. In a most preferred embodiment, said at least one negatively charged MMO is chosen from the group comprising 3′-sialyllactose, 6′-sialyllactose, sialyllacto-N-tetraose (LST-a, LST-b, LST-c, LSTd) and disialyllacto-N-tetraose. In another even more preferred embodiment, said at least one neutral MMO is chosen from the list comprising fucosylated neutral MMOs and non-fucosylated neutral MMOs. In a most preferred embodiment, said at least one neutral MMO is chosen from the group comprising 2′-fucosyllactose, 3-fucosyllactose, lacto-N-tetraose, lacto-N-neotetraose, lacto-N-fucopentaose I, LNFP-II, LNFP-III, LNFP-V, LNFP-VI, LNDFH-I, LNDFH-II and LNnDFH II. In another even more preferred embodiment, said preparation comprises, consists of or consists essentially of a mixture of MMOs comprising LNFP-III, LNFP-VI, LNnDFH II, LNFP-V and LNDFH-II.

In another preferred embodiment, a preparation is provided that comprises, consists of or consists essentially of a dried powder that comprises at least 50% w/w of a fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. In a more preferred embodiment, said fucosylated compound is obtainable, preferably obtained, by the methods as described herein. In another more preferred embodiment, the powder is dried by any one of spray-drying, drum-drying or roller-drying.

In another preferred embodiment, a preparation is provided that comprises, consists of or consists essentially of a dried powder that comprises, consists of or essentially consists of a mixture of mammalian milk oligosaccharides (MMOs) wherein said mixture comprises 0.1 to 30% w/w, preferably 0.1 to 20% w/w, more preferably 1 to 10% w/w, of one or more fucosylated compound(s) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid. In a more preferred embodiment, said fucosylated compound is obtainable, preferably obtained, by the methods as described herein. In another more preferred embodiment, the powder is dried by any one of spray-drying, drum-drying or roller-drying.

In another preferred embodiment, a preparation is provided that further comprises at least one probiotic microorganism. In another preferred embodiment of present invention, said preparation is a nutritional composition. In a more preferred embodiment, said preparation is a medicinal formulation, a dietary supplement, a dairy drink or an infant formula.

With the novel methods, said fucosylated compound and/or 3-FL can easily and effectively be provided, without the need for complicated, time and cost consuming synthetic processes.

For identification of said fucosylated compound and/or 3-FL produced as described herein, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the fucosylated compound and/or 3-FL methods such as e.g., acid-catalysed hydrolysis, HPLC (high performance liquid chromatography) or GLC (gas-liquid chromatography) (after conversion to alditol acetates) may be used. To determine the glycosidic linkages, said fucosylated compound and/or 3-FL is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, said fucosylated compound and/or 3-FL is subjected to enzymatic analysis, e.g., it is contacted with an enzyme that is specific for a particular type of linkage, e.g., beta-galactosidase, or alpha-glucosidase, etc., and NMR may be used to analyse the products.

The separated and preferably also purified fucosylated compound and/or 3-FL as described herein is incorporated into a food (e.g., human food or feed), dietary supplement, pharmaceutical ingredient, cosmetic ingredient or medicine. In some embodiments, said fucosylated compound and/or 3-FL is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including said fucosylated compound and/or 3-FL being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, said fucosylated compound and/or 3-FL produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, said fucosylated compound and/or 3-FL is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, said fucosylated compound and/or 3-FL produced and/or purified by a process in this specification is included in infant formula to provide nutritional benefits similar to those provided by the oligosaccharides in human breast milk. In some embodiments, said fucosylated compound and/or 3-FL is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the oligosaccharide in the infant formula is approximately the same concentration as the concentration of the oligosaccharide generally present in human breast milk.

In some embodiments, a fucosylated compound and/or 3-FL is added to the infant formula with a concentration that is approximately the same concentration as the concentration of the compound generally present in human breast milk.

In some embodiments, said fucosylated compound and/or 3-FL is incorporated into a feed preparation, wherein said feed is chosen from the list comprising pet food, animal milk replacer, veterinary product, veterinary feed supplement, nutrition supplement, post weaning feed, or creep feed.

As will be shown in the examples herein, the methods and the cell of the invention preferably provide at least one of the following surprising advantages:
Higher titres of said fucosylated compound (g/L),
Higher purity of said fucosylated compound (g/L),
Higher production rate r (g of said fucosylated compound/L/h),
Higher cell performance index CPI (g of said fucosylated compound/g X),
Higher specific productivity Qp (g of said fucosylated compound/g X/h),
Higher yield on the carbon source used Y (g of said fucosylated compound/g carbon source used),
Higher yield on sucrose Ys (g of said fucosylated compound/g sucrose),
Higher uptake/conversion rate of the carbon source used Q (g carbon source/g X/h),
Higher sucrose uptake/conversion rate Qs (g sucrose/g X/h),
Higher lactose conversion/consumption rate rs (g lactose/h),
Higher secretion or excretion or extracellular transport of said fucosylated compound, and/or
Higher growth speed of the production host,
   when compared to a method and a host for production of said fucosylated compound lacking a fucosyltransferase of present invention having alpha-1,3-fucosyltransferase activity on the GlcNAc and/or Glc residue of Gal-β1,m-GlcNAc-β1,n-Gal-β1,4-Glc of a saccharide substrate comprising said Gal-β1,m-GlcNAc-β1,n-Gal-β1,4-Glc as described herein.

As will also be shown in the examples herein, the methods and the cell of the invention preferably provide at least one of the following surprising advantages:
Higher titres of 3-FL (g/L),
Higher purity of 3-FL (g/L),
Higher production rate r (g 3-FL/L/h),
Higher cell performance index CPI (g 3-FL/g X),
Higher specific productivity Qp (g 3-FL/g X/h),
Higher yield on the carbon source used Y (g 3-FL/g carbon source used),
Higher yield on sucrose Ys (g 3-FL/g sucrose),
Higher uptake/conversion rate of the carbon source used Q (g carbon source/g X/h),
Higher sucrose uptake/conversion rate Qs (g sucrose/g X/h),
Higher lactose conversion/consumption rate rs (g lactose/h),
Higher secretion or excretion or extracellular transport of said 3-FL, and/or
Higher growth speed of the production host,
   when compared to a method and a host for production of 3-FL lacking a fucosyltransferase of present invention having alpha-1,3-fucosyltransferase activity on the Glc residue of lactose as described herein.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. A method for the production of a fucosylated compound, said method comprising the steps of:
   a) providing
      i) GDP-fucose,
      ii) a saccharide substrate comprising Gal-β1,m-GlcNAc-β1,n-Gal-β1,4-Glc wherein said m is 3 or 4 and said n is 3 or 6, optionally said saccharide is linked to a peptide, a protein and/or a lipid, and
      iii) a fucosyltransferase, wherein said fucosyltransferase has alpha-1,3-fucosyltransferase activity on the N-acetylglucosamine (GlcNAc) and/or the glucose (Glc) residue of said Gal-β1,m-GlcNAc-β1,n-Gal-β1,4-Glc of said saccharide substrate, and:
         comprises a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
         is a polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
         comprises a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
         comprises a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31,
   b) contacting said fucosyltransferase and GDP-fucose with said saccharide substrate under conditions where the fucosyltransferase catalyses the transfer of a fucose residue from said GDP-fucose to the GlcNAc and/or Glc residue of said Gal-β1,m-GlcNAc-β1,n-Gal-β1,4-Glc of said saccharide substrate in an alpha-1,3-glycosidic linkage resulting in the production of said fucosylated compound,
   c) preferably, separating said produced fucosylated compound.
2. Method according to embodiment 1, wherein said fucosylated compound is a saccharide comprising Gal-β1,m-[Fuc-α1,3]-GlcNAc-β1,n-Gal-β1,4-Glc, wherein said m is 3 or 4 and said n is 3 or 6, optionally said saccharide is linked to a peptide, a protein and/or a lipid.
3. Method according to embodiment 1, wherein said fucosylated compound is a saccharide comprising Gal-β1,m-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
4. Method according to embodiment 1, wherein said fucosylated compound is a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
5. Method according to any one of embodiment 1 or 2, wherein said fucosylated compound is an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation of at least six, preferably at least seven, optionally said oligosaccharide is linked to a peptide, a protein and/or a lipid.
6. Method according to any one of embodiment 1 or 3, wherein said fucosylated compound is an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation of at least six, preferably at least seven, optionally said oligosaccharide is linked to a peptide, a protein and/or a lipid.
7. Method according to any one of embodiment 1 or 4, wherein said fucosylated compound is an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation of at least seven, preferably at least eight, optionally said oligosaccharide is linked to a peptide, a protein and/or a lipid.
8. Method according to any one of previous embodiments, wherein said fucosylated compound is an oligosaccharide, preferably said oligosaccharide is a mammalian milk oligosaccharide (MMO), more preferably a human milk oligosaccharide (HMO).
9. Method according to any one of previous embodiments, wherein said fucosylated compound is a negatively charged, preferably sialylated, molecule or a neutral molecule, preferably, wherein said fucosylated compound is a negatively charged, preferably sialylated, oligosaccharide or a neutral oligosaccharide.
10. Method according to any one of previous embodiments, wherein said fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH II).
11. Method according to any one of previous embodiments, wherein said saccharide substrate is an oligosaccharide comprising Gal-β1,m-GlcNAc-β1,n-Gal-β1,4-Glc wherein said m is 3 or 4 and said n is 3 or 6 and having a degree of polymerisation of at least five, preferably at least six, optionally said oligosaccharide is linked to a peptide, a protein and/or a lipid.
12. Method according to any one of previous embodiments, wherein said saccharide substrate is an oligosaccharide, preferably said oligosaccharide is a mammalian milk oligosaccharide (MMO), more preferably a human milk oligosaccharide (HMO).
13. Method according to any one of previous embodiments, wherein said saccharide substrate is a negatively charged, preferably sialylated, molecule or a neutral molecule, preferably, wherein said fucosylated compound is a negatively charged, preferably sialylated, oligosaccharide or a neutral oligosaccharide.
14. Method according to any one of previous embodiments, wherein said saccharide substrate is chosen from the list comprising Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).
15. Method according to any one of previous embodiments, wherein said fucosyltransferase has additional alpha-1,3-fucosyltransferase activity on
   a) a monosaccharide residue of said saccharide substrate excluding the GlcNAc and Glc residues of said Gal-β1,m-GlcNAc-β1,n-Gal-β1,4-Glc of said saccharide substrate, and/or
   b) a compound that is different from said saccharide substrate, said compound being chosen from the list comprising a monosaccharide, a disaccharide and an oligosaccharide, optionally said compound is linked to a peptide, a protein and/or a lipid.
16. Method according to any one of previous embodiments, wherein said fucosyltransferase has alpha-1,4-fucosyltransferase activity on said saccharide substrate and/or on a compound that is different from said saccharide substrate, said compound being chosen from the list comprising a monosaccharide, a disaccharide and an oligosaccharide, optionally said compound is linked to a peptide, a protein and/or a lipid.
17. Method according to any one of previous embodiments, wherein said fucosyltransferase has alpha-1,3-fucosyltransferase activity on the GlcNAc residue of LNnT and
   comprises a polypeptide according to any one of SEQ ID NO 06, 19, 23, 14, 22, 21, 16, 04, 13, 15, 05, 07, 26, 31, 11, 29, 17, 25, 24, 28, 08, 09 or 10, or
   is a polypeptide comprising or consisting of an amino acid sequence having 72.50% or more sequence identity to the full-length amino acid sequence of any one of SEQ ID NO 06, 19, 23, 14, 22, 21, 16, 04, 13, 15, 05, 07, 26, 31, 11, 29, 17, 25, 24, 28, 08, 09 or 10, or
   comprises a functional fragment of a polypeptide according to any one of SEQ ID NO 06, 19, 23, 14, 22, 21, 16, 04, 13, 15, 05, 07, 26, 31, 11, 29, 17, 25, 24, 28, 08, 09 or 10, or
   comprises a functional fragment comprising an amino acid sequence of at least 10 consecutive amino acid residues from any one of SEQ ID NO 06, 19, 23, 14, 22, 21, 16, 04, 13, 15, 05, 07, 26, 31, 11, 29, 17, 25, 24, 28, 08, 09 or 10.
18. Method according to any one of embodiments 1 to 16, wherein said fucosyltransferase has alpha-1,3-fucosyltransferase activity on the Glc residue of LNnT and
   comprises a polypeptide according to any one of SEQ ID NO 03, 01, 12, 18, 02, 20, 09, 10, 08, 07, 31, 28, 05, 19 or 26, or
   is a polypeptide comprising or consisting of an amino acid sequence having 50.50% or more sequence identity to the full-length amino acid sequence of any one of SEQ ID NO 03, 01, 12, 18, 02, 20, 09, 10, 08, 07, 31, 28, 05, 19 or 26, or
   comprises a functional fragment of a polypeptide according to any one of SEQ ID NO 03, 01, 12, 18, 02, 20, 09, 10, 08, 07, 31, 28, 05, 19 or 26, or
   comprises a functional fragment comprising an amino acid sequence of at least 10 consecutive amino acid residues from any one of SEQ ID NO 03, 01, 12, 18, 02, 20, 09, 10, 08, 07, 31, 28, 05, 19 or 26.
19. Method according to any one of embodiments 1 to 16, wherein said fucosyltransferase has alpha-1,3-fucosyltransferase activity on the GlcNAc residue and on the Glc residue of LNnT and
   comprises a polypeptide according to any one of SEQ ID NO 10, 09, 08, 07, 28, 31, 19, 26 or 05, or
   is a polypeptide comprising or consisting of an amino acid sequence having 50.50% or more sequence identity to the full-length amino acid sequence of any one of SEQ ID NO 10, 09, 08, 07, 28, 31, 19, 26 or 05, or
   comprises a functional fragment of a polypeptide according to any one of SEQ ID NO 10, 09, 08, 07, 28, 31, 19, 26 or 05, or
   comprises a functional fragment comprising an amino acid sequence of at least 10 consecutive amino acid residues from any one of SEQ ID NO 10, 09, 08, 07, 28, 31, 19, 26 or 05.
20. Method according to any one of embodiments 1 to 16, wherein said fucosyltransferase has alpha-1,3-fucosyltransferase activity on the Glc residue of LNT and
   comprises a polypeptide according to any one of SEQ ID NO 03, 09, 01, 31, 28, 07, 10, 18, 26, 02, 05, 08, 30, 06, 04, 27 or 11, or
   is a polypeptide comprising or consisting of an amino acid sequence having 50.50% or more sequence identity to the full-length amino acid sequence of any one of SEQ ID NO 03, 09, 01, 31, 28, 07, 10, 18, 26, 02, 05, 08, 30, 06, 04, 27 or 11, or
   comprises a functional fragment of a polypeptide according to any one of SEQ ID NO 03, 09, 01, 31, 28, 07, 10, 18, 26, 02, 05, 08, 30, 06, 04, 27 or 11, or
   comprises a functional fragment comprising an amino acid sequence of at least 10 consecutive amino acid residues from any one of SEQ ID NO 03, 09, 01, 31, 28, 07, 10, 18, 26, 02, 05, 08, 30, 06, 04, 27 or 11.
21. Method according to any one of embodiments 1 to 16, wherein said fucosyltransferase has alpha-1,3-fucosyltransferase activity on the Glc residue of LNT and alpha-1,4-fucosyltransferase activity on the GlcNAc residue of LNT, and
   comprises a polypeptide according to any one of SEQ ID NO 03, 09, 08, 01, 10, 31, 28, 07, 18, 26, 02, 05, 30, 06, 04, 27 or 11, or
   is a polypeptide comprising or consisting of an amino acid sequence having 50.50% or more sequence identity to the full-length amino acid sequence of any one of SEQ ID NO 03, 09, 08, 01, 10, 31, 28, 07, 18, 26, 02, 05, 30, 06, 04, 27 or 11, or
   comprises a functional fragment of a polypeptide according to any one of SEQ ID NO 03, 09, 08, 01, 10, 31, 28, 07, 18, 26, 02, 05, 30, 06, 04, 27 or 11, or
   comprises a functional fragment comprising an amino acid sequence of at least 10 consecutive amino acid residues from any one of SEQ ID NO 03, 09, 08, 01, 10, 31, 28, 07, 18, 26, 02, 05, 30, 06, 04, 27 or 11.
22. Method according to any one of previous embodiments, wherein said fucosylated compound is produced in a cell-free system.
23. Method according to any one of embodiments 1 to 22, wherein said fucosylated compound is produced by a cell, preferably a single cell.
24. Method according to embodiment 23, wherein said cell is a metabolically engineered cell, preferably wherein said cell is metabolically engineered for the production of said fucosylated compound.
25. Method according to any one of embodiment 23 or 24, wherein said method comprises the steps of:
   i. providing a cell expressing said fucosyltransferase, and
   ii. providing GDP-fucose, optionally said GDP-fucose is produced by said cell, and
   iii. providing said saccharide substrate comprising Gal-β1,m-GlcNAc-β1,n-Gal-β1,4-Glc, optionally said saccharide substrate is produced by said cell, and
   iv. cultivating and/or incubating said cell under conditions permissive to express said fucosyltransferase, optionally permissive to produce said GDP-fucose and/or said saccharide substrate,
   v. preferably, separating said fucosylated compound from said cultivation.
26. A method for the production of a 3-fucosyllactose (3-FL), said method comprising the steps of:
   a) providing GDP-fucose, lactose and a fucosyltransferase, wherein said fucosyltransferase has alpha-1,3-fucosyltransferase activity on the glucose (Glc) residue of said lactose, and:
      comprises a polypeptide according to any one of SEQ ID NO 27, 28, 29 or 31, or
      is a polypeptide comprising or consisting of an amino acid sequence having 50.0% or more sequence identity to the full-length amino acid sequence of any one of SEQ ID NO 27, 28, 29 or 31, or
      comprises a functional fragment of a polypeptide according to any one of SEQ ID NO 27, 28, 29 or 31, or
      comprises a functional fragment comprising an amino acid sequence of at least 10 consecutive amino acid residues from any one of SEQ ID NO 27, 28, 29 or 31,
   b) contacting said fucosyltransferase and GDP-fucose with said lactose under conditions where the fucosyltransferase catalyses the transfer of a fucose residue from said GDP-fucose to the Glc residue of said lactose in an alpha-1,3-glycosidic linkage resulting in the production of said 3-FL,
   c) preferably, separating said produced 3-FL.
27. Method according to embodiment 26, wherein said 3-FL is produced in a cell-free system.
28. Method according to embodiment 26, wherein said 3-FL is produced by a cell, preferably a single cell.
29. Method according to embodiment 28, wherein said cell is a metabolically engineered cell, preferably wherein said cell is metabolically engineered for the production of said 3-FL.
30. Method according to any one of embodiment 28 or 29, wherein said method comprises the steps of:
   i. providing a cell expressing said fucosyltransferase, and
   ii. providing GDP-fucose, optionally said GDP-fucose is produced by said cell, and
   iii. providing lactose, optionally said lactose is produced by said cell, and
   iv. cultivating and/or incubating said cell under conditions permissive to express said fucosyltransferase, optionally permissive to produce said GDP-fucose and/or said lactose,
   v. preferably, separating said 3-FL from said cultivation.
31. Method according to any one of embodiments 23 to 25, 28 to 30, wherein said cell is modified in the expression or activity of any one of said fucosyltransferases.
32. Method according to any one of embodiment 23 to 25, 28 to 31 wherein said cell is modified with one or more gene expression modules, characterized in that the expression from any of said expression modules is either constitutive or is created by a natural inducer.
33. Method according to any one of embodiment 23 to 25, 28 to 32, wherein said cell comprises multiple copies of the same coding DNA sequence encoding for one protein.
34. Method according to any one of embodiments 23 to 25, 28 to 33, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose.
35. Method according to any one of embodiments 23 to 25, 28 to 34, wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.
36. Method according to any one of embodiments 23 to 25, 28 to 35, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, said fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is an alpha-1,3-N-acetylgalactosaminyltransferase,
   preferably, said cell is modified in the expression or activity of any one of said glycosyltransferases.
37. Method according to any one of embodiments 23 to 25, 28 to 36, wherein said cell is using one or more precursor(s) for the production of said fucosylated compound and/or said 3-FL, said precursor(s) being fed to the cell from the cultivation medium.
38. Method according to any one of embodiments 23 to 25, 28 to 37, wherein said cell is producing one or more precursor(s) for the production of said fucosylated compound and/or said 3-FL.
39. Method according to any one of embodiment 37 or 38, wherein said precursor for the production of said fucosylated compound or 3-FL is completely converted into said fucosylated compound or 3-FL, respectively.
40. Method according to any one of embodiments 23 to 25, 31 to 39, wherein said cell is capable to produce said saccharide substrate.
41. Method according to any one of embodiments 23 to 25, 28 to 40, wherein said cell is capable to produce lactose.
42. Method according to any one of embodiments 23 to 25, 28 to 41, wherein said cell produces said fucosylated compound and/or 3-FL intracellularly and wherein a fraction or substantially all of said produced fucosylated compound and/or 3-FL, respectively, remains intracellularly and/or is excreted outside said cell via passive or active transport.
43. Method according to any one of embodiments 23 to 25, 28 to 42, wherein said cell expresses a membrane transporter protein or a polypeptide having transport activity hereby transporting compounds across the outer membrane of the cell wall,
   preferably, said cell is modified in the expression or activity of said membrane transporter protein or polypeptide having transport activity.
44. Method according to embodiment 43, wherein said membrane transporter protein or polypeptide having transport activity is chosen from the list comprising porters, P—P-bond-hydrolysis-driven transporters, β-barrel porins, auxiliary transport proteins, and phosphotransfer-driven group translocators,
   preferably, said porters comprise MFS transporters, sugar efflux transporters and siderophore exporters,
   preferably, said P—P-bond-hydrolysis-driven transporters comprise ABC transporters and siderophore exporters.
45. Method according to any one of embodiment 43 or 44, wherein said membrane transporter protein or polypeptide having transport activity controls the flow over the outer membrane of the cell wall of said fucosylated compound and/or of one or more precursor(s) to be used in said production of said fucosylated compound and/or said 3-FL and/or of one or more precursor(s) to be used in said production of 3-FL.
46. Method according to any one of embodiments 43 to 45, wherein said membrane transporter protein or polypeptide having transport activity provides improved production and/or enabled and/or enhanced efflux of said fucosylated compound and/or said 3-FL.
47. Method according to any one of embodiments 23 to 25, 28 to 46, wherein the cell comprises a catabolic pathway for selected mono-, di- or oligosaccharides which is at least partially inactivated, the mono-, di-, or oligosaccharides being involved in and/or required for said production of said fucosylated compound and/or said 3-FL.
48. Method according to any one of embodiments 23 to 25, 28 to 47, wherein said cell resists the phenomenon of lactose killing when grown in an environment in which lactose is combined with one or more other carbon source(s).
49. Method according to any one of embodiments 23 to 25, 28 to 48, wherein said cell produces 90 g/L or more of said fucosylated compound and/or 3-FL in the whole broth and/or supernatant and/or wherein said fucosylated compound in the whole broth and/or supernatant has a purity of at least 80% measured on the total amount of said fucosylated compound and its precursor(s) in the whole broth and/or supernatant, respectively, and/or wherein said 3-FL in the whole broth and/or supernatant has a purity of at least 80% measured on the total amount of said 3-FL and its precursor(s) in the whole broth and/or supernatant, respectively.
50. Method according to any one of embodiments 23 to 25, 28 to 49, wherein said cell is a bacterium, fungus, yeast, a plant cell, an animal cell, or a protozoan cell,
   preferably said bacterium is an Escherichia coli strain, more preferably an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
51. Method according to embodiment 50, wherein said cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.
52. Method according to any one of embodiments 23 to 25, 28 to 51, wherein said cell is stably cultured in a medium.
53. Method according to any one of embodiments 23 to 25, 28 to 52, wherein said conditions comprise:
   use of a culture medium comprising at least one precursor for the production of said fucosylated compound and/or said 3-FL, and/or
   adding to the culture medium at least one precursor feed for the production of said fucosylated compound and/or said 3-FL.
54. Method according to any one of embodiments 23 to 25, 28 to 53, the method comprising at least one of the following steps:
   i) Use of a culture medium comprising at least one precursor;
   ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
   iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
   iv) Adding at least one precursor feed in a continuous manner to the culture medium over the course of 1 day, 2 days, 3 days, 4 days, 5 days by means of a feeding solution;
   v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.
55. Method according to any one of embodiments 28 to 53, the method comprising at least one of the following steps:
   i) Use of a culture medium comprising at least one precursor;
   ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
   iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
   iv) Adding at least one precursor feed in a continuous manner to the culture medium over the course of 1 day, 2 days, 3 days, 4 days, 5 days by means of a feeding solution;
   v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.
56. Method according to any one of embodiments 23 to 25, 28 to 54, the method comprising at least one of the following steps:
   i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
   ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.
57. Method according to any one of embodiments 28 to 53, 55, the method comprising at least one of the following steps:
   i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
   ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.
58. Method according to any one of embodiment 56 or 57, wherein the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.
59. Method according to any one of embodiment 56 to 58, wherein said lactose feed is accomplished by adding lactose to the cultivation in a concentration, such, that throughout the production phase of the cultivation a lactose concentration of at least 5 mM, preferably 10 mM or 30 mM is obtained.
60. Method according to any one of embodiments 23 to 25, 28 to 59, wherein the cells are cultivated for at least about 60, 80, 100, or about 120 hours or in a continuous manner.
61. Method according to any one of embodiments 23 to 25, 28 to 60, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
62. Method according to any one of embodiments 23 to 25, 28 to 61, wherein the culture medium contains at least one precursor selected from the group comprising lactose, galactose, fucose, sialic acid, GlcNAc, N-acetylgalactosamine (GalNAc), LNB and N-acetyllactosamine (LacNAc).
63. Method according to any one of embodiments 23 to 25, 28 to 62, wherein a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.
64. Method according to any one of embodiments 23 to 25, 28 to 62, wherein a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.
65. Method according to any one of embodiments 23 to 25, 28 to 64, wherein the cell produces a mixture of negatively charged, preferably sialylated, and/or neutral di- and oligosaccharides comprising at least one of said fucosylated compound.
66. Method according to any one of embodiments 23 to 25, 28 to 65, wherein the cell produces a mixture of negatively charged, preferably sialylated, and/or neutral oligosaccharides comprising at least one of said fucosylated compound.
67. Method according to any one of embodiments 28 to 66, wherein the cell produces a mixture of negatively charged, preferably sialylated, and/or neutral di- and oligosaccharides comprising 3-FL.
68. Method according to any one of embodiments 28 to 67, wherein the cell produces a mixture of negatively charged, preferably sialylated, and/or neutral oligosaccharides comprising 3-FL.
69. Method according to any one of previous embodiments, wherein said method comprises separation and wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.
70. Method according to any one of previous embodiments, further comprising purification of said fucosylated compound or said 3-FL, respectively.
71. Method according to embodiment 70, wherein said purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
72. A cell metabolically engineered for the production of a fucosylated compound, said fucosylated compound comprising a fucosylated version of a saccharide substrate comprising Gal-β1,m-GlcNAc-β1,n-Gal-β1,4-Glc wherein said m is 3 or 4 and said n is 3 or 6, wherein said cell is capable to express, preferably expresses, a fucosyltransferase, characterized in that said fucosyltransferase (a) has alpha-1,3-fucosyltransferase activity on the N-acetylglucosamine (GlcNAc) and/or the glucose (Glc) residue of Gal-β1,m-GlcNAc-β1,n-Gal-β1,4-Glc wherein said m is 3 or 4 and said n is 3 or 6, of a saccharide substrate comprising said Gal-β1,m-GlcNAc-β1,n-Gal-β1,4-Glc, optionally, said saccharide substrate is linked to a peptide, a protein and/or a lipid, and (b):
   comprises a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
   is a polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
   comprises a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31, or
   comprises a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31.
73. Cell according to embodiment 72, wherein said fucosylated compound is a saccharide comprising Gal-β1,m-[Fuc-α1,3]-GlcNAc-β1,n-Gal-β1,4-Glc, wherein said m is 3 or 4 and said n is 3 or 6, optionally said saccharide is linked to a peptide, a protein and/or a lipid.
74. Cell according to embodiment 72, wherein said fucosylated compound is a saccharide comprising Gal-β1,m-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
75. Cell according to embodiment 72, wherein said fucosylated compound is a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
76. Cell according to any one of embodiment 72 or 73, wherein said fucosylated compound is an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation of at least six, preferably at least seven, optionally said oligosaccharide is linked to a peptide, a protein and/or a lipid.
77. Cell according to any one of embodiment 72 or 74, wherein said fucosylated compound is an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation of at least six, preferably at least seven, optionally said oligosaccharide is linked to a peptide, a protein and/or a lipid.
78. Cell according to any one of embodiment 72 or 75, wherein said fucosylated compound is an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation of at least seven, preferably at least eight, optionally said oligosaccharide is linked to a peptide, a protein and/or a lipid.
79. Cell according to any one of embodiments 72 to 78, wherein said fucosylated compound is an oligosaccharide, preferably said oligosaccharide is a mammalian milk oligosaccharide (MMO), more preferably a human milk oligosaccharide (HMO).
80. Cell according to any one of embodiments 72 to 79, wherein said fucosylated compound is a negatively charged, preferably sialylated, molecule or a neutral molecule, preferably, wherein said fucosylated compound is a negatively charged, preferably sialylated, oligosaccharide or a neutral oligosaccharide.
81. Cell according to any one of embodiments 72 to 80, wherein said fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH II).
82. Cell according to any one of embodiments 72 to 81, wherein said saccharide substrate is an oligosaccharide comprising Gal-β1,m-GlcNAc-β1,n-Gal-β1,4-Glc wherein said m is 3 or 4 and said n is 3 or 6 and having a degree of polymerisation of at least five, preferably at least six, optionally said oligosaccharide is linked to a peptide, a protein and/or a lipid.
83. Cell according to any one of embodiments 72 to 82, wherein said saccharide substrate is an oligosaccharide, preferably said oligosaccharide is a mammalian milk oligosaccharide (MMO), more preferably a human milk oligosaccharide (HMO).
84. Cell according to any one of embodiments 72 to 83, wherein said saccharide substrate is a negatively charged, preferably sialylated, molecule or a neutral molecule, preferably, wherein said fucosylated compound is a negatively charged, preferably sialylated, oligosaccharide or a neutral oligosaccharide.
85. Cell according to any one of embodiments 72 to 84, wherein said saccharide substrate is chosen from the list comprising Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).
86. Cell according to any one of embodiments 72 to 85, wherein said fucosyltransferase has additional alpha-1,3-fucosyltransferase activity on
   a) a monosaccharide residue of said saccharide substrate excluding the GlcNAc and Glc residues of said Gal-β1,m-GlcNAc-β1,n-Gal-β1,4-Glc of said saccharide substrate, and/or
   b) a compound that is different from said saccharide substrate, said compound being chosen from the list comprising a monosaccharide, a disaccharide and an oligosaccharide, optionally said compound is linked to a peptide, a protein and/or a lipid.
87. Cell according to any one of embodiments 72 to 86, wherein said fucosyltransferase has alpha-1,4-fucosyltransferase activity on said saccharide substrate and/or on a compound that is different from said saccharide substrate, said compound being chosen from the list comprising a monosaccharide, a disaccharide and an oligosaccharide, optionally said compound is linked to a peptide, a protein and/or a lipid.
88. Cell according to any one of embodiments 72 to 87, wherein said fucosyltransferase has alpha-1,3-fucosyltransferase activity on the GlcNAc residue of LNnT and
   comprises a polypeptide according to any one of SEQ ID NO 06, 19, 23, 14, 22, 21, 16, 04, 13, 15, 05, 07, 26, 31, 11, 29, 17, 25, 24, 28, 08, 09 or 10, or
   is a polypeptide comprising or consisting of an amino acid sequence having 72.50% or more sequence identity to the full-length amino acid sequence of any one of SEQ ID NO 06, 19, 23, 14, 22, 21, 16, 04, 13, 15, 05, 07, 26, 31, 11, 29, 17, 25, 24, 28, 08, 09 or 10, or
   comprises a functional fragment of a polypeptide according to any one of SEQ ID NO 06, 19, 23, 14, 22, 21, 16, 04, 13, 15, 05, 07, 26, 31, 11, 29, 17, 25, 24, 28, 08, 09 or 10, or
   comprises a functional fragment comprising an amino acid sequence of at least 10 consecutive amino acid residues from any one of SEQ ID NO 06, 19, 23, 14, 22, 21, 16, 04, 13, 15, 05, 07, 26, 31, 11, 29, 17, 25, 24, 28, 08, 09 or 10.
89. Cell according to any one of embodiments 72 to 87, wherein said fucosyltransferase has alpha-1,3-fucosyltransferase activity on the Glc residue of LNnT and
   comprises a polypeptide according to any one of SEQ ID NO 03, 01, 12, 18, 02, 20, 09, 10, 08, 07, 31, 28, 05, 19 or 26, or
   is a polypeptide comprising or consisting of an amino acid sequence having 50.50% or more sequence identity to the full-length amino acid sequence of any one of SEQ ID NO 03, 01, 12, 18, 02, 20, 09, 10, 08, 07, 31, 28, 05, 19 or 26, or
   comprises a functional fragment of a polypeptide according to any one of SEQ ID NO 03, 01, 12, 18, 02, 20, 09, 10, 08, 07, 31, 28, 05, 19 or 26, or
   comprises a functional fragment comprising an amino acid sequence of at least 10 consecutive amino acid residues from any one of SEQ ID NO 03, 01, 12, 18, 02, 20, 09, 10, 08, 07, 31, 28, 05, 19 or 26.
90. Cell according to any one of embodiments 72 to 87, wherein said fucosyltransferase has alpha-1,3-fucosyltransferase activity on the GlcNAc residue and on the Glc residue of LNnT and
   comprises a polypeptide according to any one of SEQ ID NO 10, 09, 08, 07, 28, 31, 19, 26 or 05, or
   is a polypeptide comprising or consisting of an amino acid sequence having 50.50% or more sequence identity to the full-length amino acid sequence of any one of SEQ ID NO 10, 09, 08, 07, 28, 31, 19, 26 or 05, or
   comprises a functional fragment of a polypeptide according to any one of SEQ ID NO 10, 09, 08, 07, 28, 31, 19, 26 or 05, or
   comprises a functional fragment comprising an amino acid sequence of at least 10 consecutive amino acid residues from any one of SEQ ID NO 10, 09, 08, 07, 28, 31, 19, 26 or 05.
91. Cell according to any one of embodiments 72 to 87, wherein said fucosyltransferase has alpha-1,3-fucosyltransferase activity on the Glc residue of LNT and
   comprises a polypeptide according to any one of SEQ ID NO 03, 09, 01, 31, 28, 07, 10, 18, 26, 02, 05, 08, 30, 06, 04, 27 or 11, or
   is a polypeptide comprising or consisting of an amino acid sequence having 50.50% or more sequence identity to the full-length amino acid sequence of any one of SEQ ID NO 03, 09, 01, 31, 28, 07, 10, 18, 26, 02, 05, 08, 30, 06, 04, 27 or 11, or
   comprises a functional fragment of a polypeptide according to any one of SEQ ID NO 03, 09, 01, 31, 28, 07, 10, 18, 26, 02, 05, 08, 30, 06, 04, 27 or 11, or
   comprises a functional fragment comprising an amino acid sequence of at least 10 consecutive amino acid residues from any one of SEQ ID NO 03, 09, 01, 31, 28, 07, 10, 18, 26, 02, 05, 08, 30, 06, 04, 27 or 11.
92. Cell according to any one of embodiments 72 to 87, wherein said fucosyltransferase has alpha-1,3-fucosyltransferase activity on the Glc residue of LNT and alpha-1,4-fucosyltransferase activity on the GlcNAc residue of LNT, and
   comprises a polypeptide according to any one of SEQ ID NO 03, 09, 08, 01, 10, 31, 28, 07, 18, 26, 02, 05, 30, 06, 04, 27 or 11, or
   is a polypeptide comprising or consisting of an amino acid sequence having 50.50% or more sequence identity to the full-length amino acid sequence of any one of SEQ ID NO 03, 09, 08, 01, 10, 31, 28, 07, 18, 26, 02, 05, 30, 06, 04, 27 or 11, or
   comprises a functional fragment of a polypeptide according to any one of SEQ ID NO 03, 09, 08, 01, 10, 31, 28, 07, 18, 26, 02, 05, 30, 06, 04, 27 or 11, or
   comprises a functional fragment comprising an amino acid sequence of at least 10 consecutive amino acid residues from any one of SEQ ID NO 03, 09, 08, 01, 10, 31, 28, 07, 18, 26, 02, 05, 30, 06, 04, 27 or 11.
93. A cell metabolically engineered for the production of 3-FL, wherein said cell is capable to express, preferably expresses, a fucosyltransferase, characterized in that said fucosyltransferase has alpha-1,3-fucosyltransferase activity on the glucose (Glc) residue of lactose, and:
   comprises a polypeptide according to any one of SEQ ID NO 27, 28, 29 or 31, or
   is a polypeptide comprising or consisting of an amino acid sequence having 50.0% or more sequence identity to the full-length amino acid sequence of any one of SEQ ID NO 27, 28, 29 or 31, or
   comprises a functional fragment of a polypeptide according to any one of SEQ ID NO 27, 28, 29 or 31, or
   comprises a functional fragment comprising an amino acid sequence of at least 10 consecutive amino acid residues from any one of SEQ ID NO 27, 28, 29 or 31.
94. Cell according to any one of embodiments 72 to 93, wherein said cell is modified with one or more gene expression modules, characterized in that the expression from any of said expression modules is either constitutive or is created by a natural inducer.
95. Cell according to any one of embodiments 72 to 94, wherein said cell is modified in the expression or activity of any one of said fucosyltransferases.
96. Cell according to any one of embodiments 72 to 95, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose.
97. Cell according to any one of embodiments 72 to 96, wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.
98. Cell according to any one of embodiments 72 to 97, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, said fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is an alpha-1,3-N-acetylgalactosaminyltransferase,
   preferably, said cell is modified in the expression or activity of any one of said glycosyltransferases.
99. Cell according to any one of embodiments 72 to 98, wherein said cell is using one or more precursor(s) for the production of said fucosylated compound and/or said 3-FL, said precursor(s) being fed to the cell from the cultivation medium.
100. Cell according to any one of embodiments 72 to 99, wherein said cell is producing one or more precursor(s) for the production of said fucosylated compound and/or said 3-FL.
101. Cell according to any one of embodiment 99 or 100, wherein said precursor for the production of said fucosylated compound or 3-FL is completely converted into said fucosylated compound or 3-FL, respectively.
102. Cell according to any one of embodiments 72 to 101, wherein said cell is capable to produce said saccharide substrate.
103. Cell according to any one of embodiments 72 to 102, wherein said cell is capable to produce lactose.
104. Cell according to any one of embodiments 72 to 103, wherein said cell produces said fucosylated compound and/or 3-FL intracellularly and wherein a fraction or substantially all of said produced fucosylated compound and/or 3-FL, respectively, remains intracellularly and/or is excreted outside said cell via passive or active transport.
105. Cell according to any one of embodiments 72 to 104, wherein said cell expresses a membrane transporter protein or a polypeptide having transport activity hereby transporting compounds across the outer membrane of the cell wall, preferably, said cell is modified in the expression or activity of said membrane transporter protein or polypeptide having transport activity.
106. Cell according to embodiment 105, wherein said membrane transporter protein or polypeptide having transport activity is chosen from the list comprising porters, P—P-bond-hydrolysis-driven transporters, β-barrel porins, auxiliary transport proteins, and phosphotransfer-driven group translocators,
   preferably, said porters comprise MFS transporters, sugar efflux transporters and siderophore exporters,
   preferably, said P—P-bond-hydrolysis-driven transporters comprise ABC transporters and siderophore exporters.
107. Cell according to any one of embodiment 105 or 106, wherein said membrane transporter protein or polypeptide having transport activity controls the flow over the outer membrane of the cell wall of said fucosylated compound and/or of one or more precursor(s) to be used in said production of said fucosylated compound and/or said 3-FL and/or of one or more precursor(s) to be used in said production of 3-FL.
108. Cell according to any one of embodiments 105 to 107, wherein said membrane transporter protein or polypeptide having transport activity provides improved production and/or enabled and/or enhanced efflux of said fucosylated compound and/or said 3-FL.
109. Cell according to any one of embodiments 72 to 108, wherein said cell comprises multiple copies of the same coding DNA sequence encoding for one protein.
110. Cell according to any one of embodiments 72 to 109, wherein the cell comprises a catabolic pathway for selected mono-, di- or oligosaccharides which is at least partially inactivated, the mono-, di-, or oligosaccharides being involved in and/or required for said production of said fucosylated compound and/or said 3-FL.
111. Cell according to any one of embodiments 72 to 110, wherein said cell resists the phenomenon of lactose killing when grown in an environment in which lactose is combined with one or more other carbon source(s).
112. Cell according to any one of embodiments 72 to 111, wherein said cell produces 90 g/L or more of said fucosylated compound and/or 3-FL in the whole broth and/or supernatant and/or wherein said fucosylated compound in the whole broth and/or supernatant has a purity of at least 80% measured on the total amount of said fucosylated compound and its precursor(s) in the whole broth and/or supernatant, respectively, and/or wherein said 3-FL in the whole broth and/or supernatant has a purity of at least 80% measured on the total amount of said 3-FL and its precursor(s) in the whole broth and/or supernatant, respectively.
113. Cell according to any one of embodiments 72 to 112, wherein said cell is a bacterium, fungus, yeast, a plant cell, an animal cell, or a protozoan cell,
   preferably said bacterium is an Escherichia coli strain, more preferably an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
114. Cell according to embodiment 113, wherein said cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.
115. Cell according to any one of embodiments 72 to 114, wherein the cell produces a mixture of negatively charged, preferably sialylated, and/or neutral di- and oligosaccharides comprising at least one of said fucosylated compound.
116. Cell according to any one of embodiments 72 to 115, wherein the cell produces a mixture of negatively charged, preferably sialylated, and/or neutral oligosaccharides comprising at least one of said fucosylated compound.
117. Cell according to any one of embodiments 72 to 116, wherein the cell produces a mixture of negatively charged, preferably sialylated, and/or neutral di- and oligosaccharides comprising 3-FL.
118. Cell according to any one of embodiments 72 to 117, wherein the cell produces a mixture of negatively charged, preferably sialylated, and/or neutral oligosaccharides comprising 3-FL.
119. Use of a cell according to any one of embodiments 72 to 118 or method according to any one of embodiments 1 to 71 for the production of a fucosylated compound comprising a fucosylated version of a saccharide substrate comprising Gal-β1,m-GlcNAc-β1,n-Gal-β1,4-Glc wherein said m is 3 or 4 and said n is 3 or 6.
120. Use of a cell according to any one of embodiments 93 to 118 or method according to any one of embodiments 26 to 71 for the production of 3-FL.
121. Use of a fucosylated compound obtained by the method according to any one of embodiments 1 to 71 for the manufacture of a preparation, preferably a nutritional composition, more preferably a medicinal formulation, a dietary supplement, a dairy drink or an infant formula.
122. Use of 3-FL obtained by the method according to any one of embodiments 26 to 71 for the manufacture of a preparation, preferably a nutritional composition, more preferably a medicinal formulation, a dietary supplement, a dairy drink or an infant formula.
123. A spray-dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
124. A spray-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of embodiments 1 to 71.
125. A spray-dried powder according to any one of embodiment 123 or 124, wherein said at least one fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH II).
126. A spray-dried powder according to any one of embodiment 123 or 124, wherein said powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH II).
127. A spray-dried powder comprising a mixture of mammalian milk oligosaccharides (MMOs), preferably at least one negatively charged and/or at least one neutral MMO, wherein said mixture comprises, consists of or consists essentially of at least one of LNFP-III, LNFP-VI, LNnDFH II, LNFP-V and LNDFH II, preferably wherein said LNFP-III, LNFP-VI, LNnDFH II, LNFP-V and/or LNDFH II is/are obtainable, preferably obtained, by the method according to any one of embodiments 1 to 71.
128. A spray-dried powder according to embodiment 127, wherein said mixture of MMOs comprises, consists of or consists essentially of at least one MMO chosen from the group comprising LNFP-1, LNFP-II, LNFP-III, LNFP-V, LNFP-VI, LNDFH-1, LNDFH II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.
129. A drum-dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
130. A drum-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of embodiments 1 to 71.
131. A drum-dried powder according to any one of embodiment 129 or 130, wherein said at least one fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH II).
132. A drum-dried powder according to any one of embodiment 129 or 130, wherein said powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH II).
133. A drum-dried powder comprising a mixture of mammalian milk oligosaccharides (MMOs), preferably at least one negatively charged and/or at least one neutral MMO, wherein said mixture comprises, consists of or consists essentially of at least one of LNFP-III, LNFP-VI, LNnDFH II, LNFP-V and LNDFH II, preferably wherein said LNFP-III, LNFP-VI, LNnDFH II, LNFP-V and/or LNDFH II is/are obtainable, preferably obtained, by the method according to any one of embodiments 1 to 71.
134. A drum-dried powder according to embodiment 133, wherein said mixture of MMOs comprises, consists of or consists essentially of at least one MMO chosen from the group comprising LNFP-1, LNFP-II, LNFP-III, LNFP-V, LNFP-VI, LNDFH-1, LNDFH II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.
135. A roller-dried powder comprising at least one fucosylated compound chosen from the list comprising a saccharide comprising, consisting of or consisting essentially of Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
136. A roller-dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of embodiments 1 to 71.
137. A roller-dried powder according to any one of embodiment 135 or 136, wherein said at least one fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH II).
138. A roller-dried powder according to any one of embodiment 135 or 136, wherein said powder comprises consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH II).
139. A roller-dried powder comprising a mixture of mammalian milk oligosaccharides (MMOs), preferably at least one negatively charged and/or at least one neutral MMO, wherein said mixture comprises, consists of or consists essentially of at least one of LNFP-III, LNFP-VI, LNnDFH II, LNFP-V and LNDFH II, preferably wherein said LNFP-III, LNFP-VI, LNnDFH II, LNFP-V and/or LNDFH II is/are obtainable, preferably obtained, by the method according to any one of embodiments 1 to 71.
140. A roller-dried powder according to embodiment 139, wherein said mixture of MMOs comprises, consists of or consists essentially of at least one MMO chosen from the group comprising LNFP-1, LNFP-II, LNFP-III, LNFP-V, LNFP-VI, LNDFH-1, LNDFH II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.
141. A preparation comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of embodiments 1 to 71.
142. A preparation comprising a spray-dried powder according to any one of embodiments 123 to 128.
143. A preparation comprising a drum-dried powder according to any one of embodiments 129 to 134.
144. A preparation comprising a roller-dried powder according to any one of embodiments 135 to 140.
145. A preparation comprising a mixture of mammalian milk oligosaccharides (MMOs), preferably at least one negatively charged MMO and/or at least one neutral MMO, wherein said mixture comprises at least one of LNFP-III, LNFP-VI, LNnDFH II, LNFP-V and LNDFH II, preferably wherein said LNFP-III, LNFP-VI, LNnDFH II, LNFP-V and/or LNDFH II is/are obtainable, preferably obtained, by the method according to any one of embodiments 1 to 71.
146. A preparation according to embodiment 145, wherein the at least one negatively charged MMO is a sialylated MMO, preferably chosen from the group comprising 3′-sialyllactose, 6′-sialyllactose, sialyllacto-N-tetraose (LST-a, LST-b, LST-c, LSTd) and disialyllacto-N-tetraose.
147. A preparation according to embodiment 145, wherein the at least one neutral MMO is chosen from the list comprising fucosylated neutral MMOs and non-fucosylated neutral MMOs, preferably chosen from the group comprising 2′-fucosyllactose, 3-fucosyllactose, lacto-N-tetraose, lacto-N-neotetraose, lacto-N-fucopentaose I, LNFP-II, LNFP-III, LNFP-V, LNFP-VI, LNDFH-1, LNDFH II and LNnDFH II.
148. A preparation according to any one of embodiment 145 to 147, wherein said mixture of MMOs comprises, consists of or consists essentially of LNFP-III, LNFP-VI, LNnDFH II, LNFP-V and LNDFH II.
149. A preparation according to any one of embodiments 141 to 148, wherein said preparation further comprises at least one probiotic microorganism.
150. A preparation according to any one of embodiments 141 to 149, wherein said preparation is a nutritional composition, preferably a medicinal formulation, a dietary supplement, a dairy drink or an infant formula.

Moreover, the present invention relates to the following preferred specific embodiments:
1. A method for the production of a fucosylated compound, said method comprising the steps of:
   a) providing
      i) GDP-fucose,
      ii) a saccharide substrate comprising Gal-β1,m-GlcNAc-β1,n-Gal-β1,4-Glc wherein said m is 3 or 4 and said n is 3 or 6, optionally said saccharide is linked to a peptide, a protein and/or a lipid, and
      iii) a fucosyltransferase, wherein said fucosyltransferase has alpha-1,3-fucosyltransferase activity on the N-acetylglucosamine (GlcNAc) and/or the glucose (Glc) residue of said Gal-β1,m-GlcNAc-β1,n-Gal-β1,4-Glc of said saccharide substrate, and:
         comprises a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
         is a polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
         comprises a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
         comprises a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51,
   b) contacting said fucosyltransferase and GDP-fucose with said saccharide substrate under conditions where the fucosyltransferase catalyses the transfer of a fucose residue from said GDP-fucose to the GlcNAc and/or Glc residue of said Gal-β1,m-GlcNAc-β1,n-Gal-β1,4-Glc of said saccharide substrate in an alpha-1,3-glycosidic linkage resulting in the production of said fucosylated compound,
   c) preferably, separating said produced fucosylated compound.
2. Method according to preferred embodiment 1, wherein said fucosylated compound is:
   a saccharide comprising Gal-β1,m-[Fuc-α1,3]-GlcNAc-β1,n-Gal-β1,4-Glc, wherein said m is 3 or 4 and said n is 3 or 6, optionally said saccharide is linked to a peptide, a protein and/or a lipid, or
   a saccharide comprising Gal-β1,m-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, or
   a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
3. Method according to any one of preferred embodiment 1 or 2, wherein said fucosylated compound is:
   an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation of at least six, preferably at least seven, optionally said oligosaccharide is linked to a peptide, a protein and/or a lipid, or
   an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation of at least six, preferably at least seven, optionally said oligosaccharide is linked to a peptide, a protein and/or a lipid, or
   an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation of at least seven, preferably at least eight, optionally said oligosaccharide is linked to a peptide, a protein and/or a lipid.
4. Method according to any one of previous preferred embodiments, wherein said fucosylated compound is:
   an oligosaccharide, preferably said oligosaccharide is a mammalian milk oligosaccharide (MMO), more preferably a human milk oligosaccharide (HMO),
   a negatively charged, preferably sialylated, molecule or a neutral molecule, preferably, a negatively charged, preferably sialylated, oligosaccharide or a neutral oligosaccharide, and/or
   chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II).
5. Method according to any one of previous preferred embodiments, wherein said saccharide substrate is:
   an oligosaccharide comprising Gal-β1,m-GlcNAc-β1,n-Gal-β1,4-Glc wherein said m is 3 or 4 and said n is 3 or 6 and having a degree of polymerisation of at least five, preferably at least six, optionally said oligosaccharide is linked to a peptide, a protein and/or a lipid,
   an oligosaccharide, preferably said oligosaccharide is a mammalian milk oligosaccharide (MMO), more preferably a human milk oligosaccharide (HMO),
   a negatively charged, preferably sialylated, molecule or a neutral molecule, preferably, a negatively charged, preferably sialylated, oligosaccharide or a neutral oligosaccharide, and/or
   chosen from the list comprising Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).
6. Method according to any one of previous preferred embodiments, wherein said fucosyltransferase has additional alpha-1,3-fucosyltransferase activity on
   a) a monosaccharide residue of said saccharide substrate excluding the GlcNAc and Glc residues of said Gal-β1,m-GlcNAc-β1,n-Gal-β1,4-Glc of said saccharide substrate, and/or
   b) a compound that is different from said saccharide substrate, said compound being chosen from the list comprising a monosaccharide, a disaccharide and an oligosaccharide, optionally said compound is linked to a peptide, a protein and/or a lipid.
7. Method according to any one of previous preferred embodiments, wherein said fucosyltransferase has alpha-1,4-fucosyltransferase activity on said saccharide substrate and/or on a compound that is different from said saccharide substrate, said compound being chosen from the list comprising a monosaccharide, a disaccharide and an oligosaccharide, optionally said compound is linked to a peptide, a protein and/or a lipid.
8. Method according to any one of previous preferred embodiments, wherein said fucosyltransferase has alpha-1,3-fucosyltransferase activity on the GlcNAc residue of LNnT and
   comprises a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   is a polypeptide comprising or consisting of an amino acid sequence having 72.50% or more sequence identity to the full-length amino acid sequence of any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   comprises a functional fragment of a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   comprises a functional fragment comprising an amino acid sequence of at least 10 consecutive amino acid residues from any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50.
9. Method according to any one of preferred embodiments 1 to 7, wherein said fucosyltransferase has:
   a) alpha-1,3-fucosyltransferase activity on the Glc residue of LNnT and
      comprises a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or
      is a polypeptide comprising or consisting of an amino acid sequence having 50.50% or more sequence identity to the full-length amino acid sequence of any one of SEQ ID NO 03, 01, 12, 18, 02, 50, 20, 09, 48, 10, 36, 08, 33, 32, 49, 35, 34, 07, 31, 28, 05, 19 or 26, or
      comprises a functional fragment of a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or
      comprises a functional fragment comprising an amino acid sequence of at least 10 consecutive amino acid residues from any one of SEQ ID NO 03, 01, 12, 18, 02, 50, 20, 09, 48, 10, 36, 08, 33, 32, 49, 35, 34, 07, 31, 28, 05, 19 or 26, or
   b) alpha-1,3-fucosyltransferase activity on the GlcNAc residue and on the Glc residue of LNnT and
      comprises a polypeptide according to any one of SEQ ID NO 10, 09, 34, 36, 08, 07, 28, 31, 48, 35, 32, 19, 49, 26, 05 or 33, or
      is a polypeptide comprising or consisting of an amino acid sequence having 50.50% or more sequence identity to the full-length amino acid sequence of any one of SEQ ID NO 10, 09, 34, 36, 08, 07, 28, 31, 48, 35, 32, 19, 49, 26, 05 or 33, or
      comprises a functional fragment of a polypeptide according to any one of SEQ ID NO 10, 09, 34, 36, 08, 07, 28, 31, 48, 35, 32, 19, 49, 26, 05 or 33, or
      comprises a functional fragment comprising an amino acid sequence of at least 10 consecutive amino acid residues from any one of SEQ ID NO 10, 09, 34, 36, 08, 07, 28, 31, 48, 35, 32, 19, 49, 26, 05 or 33, or
   c) alpha-1,3-fucosyltransferase activity on the Glc residue of LNT and
      comprises a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or
      is a polypeptide comprising or consisting of an amino acid sequence having 50.50% or more sequence identity to the full-length amino acid sequence of any one of SEQ ID NO 48, 34, 32, 50, 03, 09, 33, 35, 01, 31, 28, 07, 49, 10, 18, 26, 02, 05, 08, 30, 06, 04, 27 or 11, or
      comprises a functional fragment of a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or
      comprises a functional fragment comprising an amino acid sequence of at least 10 consecutive amino acid residues from any one of SEQ ID NO 48, 34, 32, 50, 03, 09, 33, 35, 01, 31, 28, 07, 49, 10, 18, 26, 02, 05, 08, 30, 06, 04, 27 or 11, or
   d) alpha-1,3-fucosyltransferase activity on the Glc residue of LNT and alpha-1,4-fucosyltransferase activity on the GlcNAc residue of LNT, and
      comprises a polypeptide according to any one of SEQ ID NO 03, 09, 33, 35, 08, 01, 10, 31, 28, 07, 18, 26, 02, 05, 30, 06, 04, 27 or 11, or
      is a polypeptide comprising or consisting of an amino acid sequence having 50.50% or more sequence identity to the full-length amino acid sequence of any one of SEQ ID NO 03, 09, 33, 35, 08, 01, 10, 31, 28, 07, 18, 26, 02, 05, 30, 06, 04, 27 or 11, or
      comprises a functional fragment of a polypeptide according to any one of SEQ ID NO 03, 09, 33, 35, 08, 01, 10, 31, 28, 07, 18, 26, 02, 05, 30, 06, 04, 27 or 11, or
      comprises a functional fragment comprising an amino acid sequence of at least 10 consecutive amino acid residues from any one of SEQ ID NO 03, 09, 33, 35, 08, 01, 10, 31, 28, 07, 18, 26, 02, 05, 30, 06, 04, 27 or 11.
10. Method according to any one of previous preferred embodiments, wherein said fucosylated compound is produced in a cell-free system or by a cell, preferably a single cell, preferably said cell is a metabolically engineered cell, preferably wherein said cell is metabolically engineered for the production of said fucosylated compound.
11. Method according to preferred embodiment 10, wherein said method comprises the steps of:
   i. providing a cell expressing said fucosyltransferase, and
   ii. providing GDP-fucose, optionally said GDP-fucose is produced by said cell, and
   iii. providing said saccharide substrate comprising Gal-β1,m-GlcNAc-β1,n-Gal-β1,4-Glc, optionally said saccharide substrate is produced by said cell, and
   iv. cultivating and/or incubating said cell under conditions permissive to express said fucosyltransferase, optionally permissive to produce said GDP-fucose and/or said saccharide substrate, resulting in the production of said fucosylated compound,
   v. preferably, separating said fucosylated compound from said cultivation.
12. A method for the production of a 3-fucosyllactose (3-FL), said method comprising the steps of:
   a) providing GDP-fucose, lactose and a fucosyltransferase, wherein said fucosyltransferase has alpha-1,3-fucosyltransferase activity on the glucose (Glc) residue of said lactose, and:
      comprises a polypeptide according to any one of SEQ ID NO 36, 32, 34, 28, 31, 37, 50, 27, 38, 29, 41, 49, 35, 39, 45, 43, 40 or 51, or
      is a polypeptide comprising or consisting of an amino acid sequence having 50.0% or more sequence identity to the full-length amino acid sequence of any one of SEQ ID NO 36, 32, 34, 28, 31, 37, 50, 27, 38, 29, 41, 49, 35, 39, 45, 43, 40 or 51, or
      comprises a functional fragment of a polypeptide according to any one of SEQ ID NO 36, 32, 34, 28, 31, 37, 50, 27, 38, 29, 41, 49, 35, 39, 45, 43, 40 or 51, or
      comprises a functional fragment comprising an amino acid sequence of at least 10 consecutive amino acid residues from any one of SEQ ID NO 36, 32, 34, 28, 31, 37, 50, 27, 38, 29, 41, 49, 35, 39, 45, 43, 40 or 51,
   b) contacting said fucosyltransferase and GDP-fucose with said lactose under conditions where the fucosyltransferase catalyses the transfer of a fucose residue from said GDP-fucose to the Glc residue of said lactose in an alpha-1,3-glycosidic linkage resulting in the production of said 3-FL,
   c) preferably, separating said produced 3-FL.
13. Method according to preferred embodiment 12, wherein said 3-FL is produced in a cell-free system or by a cell, preferably a single cell, preferably said cell is a metabolically engineered cell, preferably wherein said cell is metabolically engineered for the production of said 3-FL.
14. Method according to preferred embodiment 13, wherein said method comprises the steps of:
   i. providing a cell expressing said fucosyltransferase, and
   ii. providing GDP-fucose, optionally said GDP-fucose is produced by said cell, and
   iii. providing lactose, optionally said lactose is produced by said cell, and
   iv. cultivating and/or incubating said cell under conditions permissive to express said fucosyltransferase, optionally permissive to produce said GDP-fucose and/or said lactose, resulting in the production of said 3-FL,
   v. preferably, separating said 3-FL from said cultivation.
15. Method according to any one of preferred embodiments 10, 11, 13 or 14, wherein said cell is modified in the expression or activity of any one of said fucosyltransferases.
16. Method according to any one of preferred embodiments 10, 11, 13 to 15, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose and/or wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.
17. Method according to any one of preferred embodiments 10, 11, 13 to 16, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, said fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is an alpha-1,3-N-acetylgalactosaminyltransferase,
   preferably, said cell is modified in the expression or activity of any one of said glycosyltransferases.
18. Method according to any one of preferred embodiments 10, 11, 13 to 17, wherein said cell is using one or more precursor(s) for the production of said fucosylated compound and/or said 3-FL, said precursor(s) being fed to the cell from the cultivation medium and/or wherein said cell is producing one or more precursor(s) for the production of said fucosylated compound and/or said 3-FL.
19. Method according to preferred embodiment 18, wherein said precursor for the production of said fucosylated compound or 3-FL is completely converted into said fucosylated compound or 3-FL, respectively.
20. Method according to any one of preferred embodiments 10, 11, 13 to 19, wherein said cell is capable to produce, preferably produces, said saccharide substrate and/or lactose.
21. Method according to any one of preferred embodiments 10, 11, 13 to 20, wherein said cell produces said fucosylated compound and/or 3-FL intracellularly and wherein a fraction or substantially all of said produced fucosylated compound and/or 3-FL, respectively, remains intracellularly and/or is excreted outside said cell via passive or active transport.
22. Method according to any one of preferred embodiments 10, 11, 13 to 21, wherein said cell expresses a membrane transporter protein or a polypeptide having transport activity hereby transporting compounds across the outer membrane of the cell wall,
   preferably, said cell is modified in the expression or activity of said membrane transporter protein or polypeptide having transport activity.
23. Method according to preferred embodiment 22, wherein said membrane transporter protein or polypeptide having transport activity is chosen from the list comprising porters, P—P-bond-hydrolysis-driven transporters, β-barrel porins, auxiliary transport proteins, and phosphotransfer-driven group translocators,
   preferably, said porters comprise MFS transporters, sugar efflux transporters and siderophore exporters,
   preferably, said P—P-bond-hydrolysis-driven transporters comprise ABC transporters and siderophore exporters.
24. Method according to any one of preferred embodiment 22 or 23, wherein said membrane transporter protein or polypeptide having transport activity controls the flow over the outer membrane of the cell wall of said fucosylated compound and/or of one or more precursor(s) to be used in said production of said fucosylated compound and/or said 3-FL and/or of one or more precursor(s) to be used in said production of 3-FL.
25. Method according to any one of preferred embodiments 22 to 24, wherein said membrane transporter protein or polypeptide having transport activity provides improved production and/or enabled and/or enhanced efflux of said fucosylated compound and/or said 3-FL.
26. Method according to any one of preferred embodiments 10, 11, 13 to 25, wherein the cell comprises a catabolic pathway for selected mono-, di- or oligosaccharides which is at least partially inactivated, the mono-, di-, or oligosaccharides being involved in and/or required for said production of said fucosylated compound and/or said 3-FL.
27. Method according to any one of preferred embodiments 10, 11, 13 to 26, wherein said cell produces 90 g/L or more of said fucosylated compound and/or 3-FL in the whole broth and/or supernatant and/or wherein said fucosylated compound in the whole broth and/or supernatant has a purity of at least 80% measured on the total amount of said fucosylated compound and its precursor(s) in the whole broth and/or supernatant, respectively, and/or wherein said 3-FL in the whole broth and/or supernatant has a purity of at least 80% measured on the total amount of said 3-FL and its precursor(s) in the whole broth and/or supernatant, respectively.
28. Method according to any one of preferred embodiments 10, 11, 13 to 27, wherein said cell is a bacterium, fungus, yeast, a plant cell, an animal cell, or a protozoan cell,
   preferably, said bacterium belongs to a phylum chosen from the group comprising Proteobacteria, Firmicutes, Cyanobacteria, Deinococcus-Thermus and Actinobacteria; more preferably, said bacterium belongs to a family chosen from the group comprising Enterobacteriaceae, Bacillaceae, Lactobacillaceae, Corynebacteriaceae and Vibrionaceae; even more preferably, said bacterium is chosen from the list comprising an Escherichia coli strain, a Bacillus subtilis strain, a Vibrio natriegens strain; even more preferably said Escherichia coli strain is a K-12 strain, most preferably said Escherichia coli K-12 strain is E. coli MG1655,
   preferably, said fungus belongs to a genus chosen from the group comprising Rhizopus, Dictyostelium, Penicillium, Mucor or Aspergillus,
   preferably, said yeast belongs to a genus chosen from the group comprising Saccharomyces, Zygosaccharomyces, Pichia, Komagataella, Hansenula, Yarrowia, Starmerella, Kluyveromyces, Debaromyces, Candida, Schizosaccharomyces, Schwanniomyces or Torulaspora; more preferably, said yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
   preferably, said plant cell is an algal cell or is derived from tobacco, alfalfa, rice, tomato, cotton, rapeseed, soy, maize, or corn plant,
   preferably, said animal cell is derived from insects, amphibians, reptiles, invertebrates, fish, birds or mammalian cells excluding human embryonic stem cells, more preferably said mammalian cell is chosen from the list comprising an epithelial cell, an embryonic kidney cell, a fibroblast cell, a COS cell, a Chinese hamster ovary (CHO) cell, a murine myeloma cell, an NIH-3T3 cell, a lactocyte derived from mammalian induced pluripotent stem cells, more preferably said mammalian induced pluripotent stem cells are human induced pluripotent stem cells, a post-parturition mammary epithelium cell, a polarized mammary cell, more preferably said polarized mammary cell is selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably, said protozoan cell is a Leishmania tarentolae cell.
29. Method according to any one of preferred embodiments 10, 11, 13 to 28, wherein said cell is stably cultured in a medium.
30. Method according to any one of preferred embodiments 10, 11, 13 to 29, wherein said conditions comprise:
   use of a culture medium comprising at least one precursor for the production of said fucosylated compound and/or said 3-FL, and/or
   adding to the culture medium at least one precursor feed for the production of said fucosylated compound and/or said 3-FL.
31. Method according to any one of preferred embodiments 10, 11, 13 to 30, the method comprising at least one of the following steps:
   i) Use of a culture medium comprising at least one precursor;
   ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
   iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
   iv) Adding at least one precursor feed in a continuous manner to the culture medium over the course of 1 day, 2 days, 3 days, 4 days, 5 days by means of a feeding solution;
   v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.
32. Method according to any one of preferred embodiments 13 to 30, the method comprising at least one of the following steps:
   i) Use of a culture medium comprising at least one precursor;
   ii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed;
   iii) Adding to the culture medium in a reactor at least one precursor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor feed and wherein preferably, the pH of said precursor feed is set between 3.0 and 7.0 and wherein preferably, the temperature of said precursor feed is kept between 20° C. and 80° C.;
   iv) Adding at least one precursor feed in a continuous manner to the culture medium over the course of 1 day, 2 days, 3 days, 4 days, 5 days by means of a feeding solution;
   v) Adding at least one precursor feed in a continuous manner to the culture medium over the course of 1 day, 2 days, 3 days, 4 days, 5 days by means of a feeding solution and wherein preferably, the pH of said feeding solution is set between 3.0 and 7.0 and wherein preferably, the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.
33. Method according to any one of preferred embodiments 10, 11, 13 to 31, the method comprising at least one of the following steps:
   i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
   ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said fucosylated compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.
34. Method according to any one of preferred embodiments 13 to 30, 32, the method comprising at least one of the following steps:
   i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
   ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3.0 and 7.0 and wherein preferably the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, g/L, more preferably 450 g/L, more preferably 500 g/L, even more preferably, 550 g/L, most preferably 600 g/L; and wherein preferably the pH of said feeding solution is set between 3.0 and 7.0 and wherein preferably the temperature of said feeding solution is kept between 20° C. and 80° C.;
   said method resulting in said 3-FL with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.
35. Method according to any one of preferred embodiment 33 or 34, wherein the lactose feed is accomplished by:
   adding lactose from the beginning of the cultivation in a concentration of at least 5 mM, preferably in a concentration of 30, 40, 50, 60, 70, 80, 90, 100, 150 mM, more preferably in a concentration >300 mM, and/or
   adding lactose to the cultivation in a concentration, such, that throughout the production phase of the cultivation a lactose concentration of at least 5 mM, preferably 10 mM or 30 mM is obtained.
36. Method according to any one of preferred embodiments 10, 11, 13 to 35, wherein said cell is cultivated:
   for at least about 60, 80, 100, or about 120 hours or in a continuous manner, and/or
   in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate and/or wherein the culture medium contains at least one precursor selected from the group comprising lactose, galactose, fucose, sialic acid, GlcNAc, N-acetylgalactosamine (GalNAc), LNB and N-acetyllactosamine (LacNAc).
37. Method according to any one of preferred embodiments 10, 11, 13 to 36, wherein a first phase of exponential cell growth is provided by adding a carbon-based substrate, preferably glucose or sucrose, to the culture medium comprising a precursor, preferably lactose, followed by a second phase wherein:
   only a carbon-based substrate, preferably glucose or sucrose, is added to the culture medium, or
   a carbon-based substrate, preferably glucose or sucrose, and a precursor, preferably lactose, are added to the culture medium.
38. Method according to any one of preferred embodiments 10, 11, 13 to 37, wherein the cell produces:
   a mixture of negatively charged, preferably sialylated, and/or neutral di- and oligosaccharides comprising at least one of said fucosylated compound, or
   a mixture of negatively charged, preferably sialylated, and/or neutral oligosaccharides comprising at least one of said fucosylated compound.
39. Method according to any one of preferred embodiments 13 to 38, wherein the cell produces:
   a mixture of negatively charged, preferably sialylated, and/or neutral di- and oligosaccharides comprising 3-FL, or
   a mixture of negatively charged, preferably sialylated, and/or neutral oligosaccharides comprising 3-FL.
40. Method according to any one of previous preferred embodiments, wherein said method comprises separation and wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.
41. Method according to any one of previous preferred embodiments, further comprising purification of said fucosylated compound or said 3-FL, respectively, preferably wherein said purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
42. A cell metabolically engineered for the production of a fucosylated compound, said fucosylated compound comprising a fucosylated version of a saccharide substrate comprising Gal-β1,m-GlcNAc-β1,n-Gal-β1,4-Glc wherein said m is 3 or 4 and said n is 3 or 6, wherein said cell is capable to express, preferably expresses, a fucosyltransferase, characterized in that said fucosyltransferase (a) has alpha-1,3-fucosyltransferase activity on the N-acetylglucosamine (GlcNAc) and/or the glucose (Glc) residue of Gal-β1,m-GlcNAc-β1,n-Gal-β1,4-Glc wherein said m is 3 or 4 and said n is 3 or 6, of a saccharide substrate comprising said Gal-β1,m-GlcNAc-β1,n-Gal-β1,4-Glc, optionally, said saccharide substrate is linked to a peptide, a protein and/or a lipid, and (b):
   comprises a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
   is a polypeptide comprising or consisting of an amino acid sequence having 72.50% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
   comprises a functional fragment of a polypeptide according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
   comprises a functional fragment comprising an amino acid sequence of at least 10 consecutive amino acid residues from any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51.
43. Cell according to preferred embodiment 42, wherein said fucosylated compound is:
   a saccharide comprising Gal-β1,m-[Fuc-α1,3]-GlcNAc-β1,n-Gal-β1,4-Glc, wherein said m is 3 or 4 and said n is 3 or 6, optionally said saccharide is linked to a peptide, a protein and/or a lipid,
   a saccharide comprising Gal-β1,m-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, or
   a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid.
44. Cell according to any one of preferred embodiment 42 or 43, wherein said fucosylated compound is:
   an oligosaccharide comprising Gal-β1,m-[Fuc-α1,3]-GlcNAc-β1,n-Gal-β1,4-Glc wherein said m is 3 or 4 and said n is 3 or 6 and having a degree of polymerisation of at least six, preferably at least seven, optionally said oligosaccharide is linked to a peptide, a protein and/or a lipid, or
   an oligosaccharide comprising Gal-β1,m-GlcNAc-β1,n-Gal-β1,4-[Fuc-α1,3]-Glc wherein said m is 3 or 4 and said n is 3 or 6 and having a degree of polymerisation of at least six, preferably at least seven, optionally said oligosaccharide is linked to a peptide, a protein and/or a lipid, or
   an oligosaccharide comprising Gal-β1,m-[Fuc-α1,3]-GlcNAc-β1,n-Gal-β1,4-[Fuc-α1,3]-Glc wherein said m is 3 or 4 and said n is 3 or 6 and having a degree of polymerisation of at least seven, preferably at least eight, optionally said oligosaccharide is linked to a peptide, a protein and/or a lipid.
45. Cell according to any one of preferred embodiments 42 to 44, wherein said fucosylated compound is:
   an oligosaccharide, preferably said oligosaccharide is a mammalian milk oligosaccharide (MMO), more preferably a human milk oligosaccharide (HMO),
   a negatively charged, preferably sialylated, molecule or a neutral molecule, preferably, a negatively charged, preferably sialylated, oligosaccharide or a neutral oligosaccharide, and/or
   chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II).
46. Cell according to any one of preferred embodiments 42 to 45, wherein said saccharide substrate is:
   an oligosaccharide comprising Gal-β1,m-GlcNAc-β1,n-Gal-β1,4-Glc wherein said m is 3 or 4 and said n is 3 or 6 and having a degree of polymerisation of at least five, preferably at least six, optionally said oligosaccharide is linked to a peptide, a protein and/or a lipid,
   an oligosaccharide, preferably said oligosaccharide is a mammalian milk oligosaccharide (MMO), more preferably a human milk oligosaccharide (HMO),
   a negatively charged, preferably sialylated, molecule or a neutral molecule, preferably, a negatively charged, preferably sialylated, oligosaccharide or a neutral oligosaccharide, and/or
   chosen from the list comprising Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).
47. Cell according to any one of preferred embodiments 42 to 46, wherein said fucosyltransferase has additional alpha-1,3-fucosyltransferase activity on
   a) a monosaccharide residue of said saccharide substrate excluding the GlcNAc and Glc residues of said Gal-β1,m-GlcNAc-β1,n-Gal-β1,4-Glc of said saccharide substrate, and/or
   b) a compound that is different from said saccharide substrate, said compound being chosen from the list comprising a monosaccharide, a disaccharide and an oligosaccharide, optionally said compound is linked to a peptide, a protein and/or a lipid.
48. Cell according to any one of preferred embodiments 42 to 47, wherein said fucosyltransferase has alpha-1,4-fucosyltransferase activity on said saccharide substrate and/or on a compound that is different from said saccharide substrate, said compound being chosen from the list comprising a monosaccharide, a disaccharide and an oligosaccharide, optionally said compound is linked to a peptide, a protein and/or a lipid.
49. Cell according to any one of preferred embodiments 42 to 48, wherein said fucosyltransferase has alpha-1,3-fucosyltransferase activity on the GlcNAc residue of LNnT and
   comprises a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   is a polypeptide comprising or consisting of an amino acid sequence having 72.50% or more sequence identity to the full-length amino acid sequence of any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   comprises a functional fragment of a polypeptide according to any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
   comprises a functional fragment comprising an amino acid sequence of at least 10 consecutive amino acid residues from any one of SEQ ID NO 37, 41, 45, 40, 38, 39, 51, 36, 43, 44, 34, 42, 47, 46, 06, 32, 19, 35, 23, 14, 22, 21, 16, 04, 13, 15, 05, 07, 26, 31, 11, 29, 17, 25, 24, 28, 08, 09, 10, 33, 48, 49 or 50, or
50. Cell according to any one of preferred embodiments 42 to 48, wherein said fucosyltransferase has:
   a) alpha-1,3-fucosyltransferase activity on the Glc residue of LNnT and
      comprises a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or
      is a polypeptide comprising or consisting of an amino acid sequence having 50.50% or more sequence identity to the full-length amino acid sequence of any one of SEQ ID NO 03, 01, 12, 18, 02, 50, 20, 09, 48, 10, 36, 08, 33, 32, 49, 35, 34, 07, 31, 28, 05, 19 or 26, or
      comprises a functional fragment of a polypeptide according to any one of SEQ ID NO 03, 01, 12, 18, 02, 50, 20, 09, 48, 10, 36, 08, 33, 32, 49, 35, 34, 07, 31, 28, 05, 19 or 26, or
      comprises a functional fragment comprising an amino acid sequence of at least 10 consecutive amino acid residues from any one of SEQ ID NO 03, 01, 12, 18, 02, 50, 20, 09, 48, 10, 36, 08, 33, 32, 49, 35, 34, 07, 31, 28, 05, 19 or 26, or
   b) alpha-1,3-fucosyltransferase activity on the GlcNAc residue and on the Glc residue of LNnT and
      comprises a polypeptide according to any one of SEQ ID NO 10, 09, 34, 36, 08, 07, 28, 31, 48, 35, 32, 19, 49, 26, 05 or 33, or
      is a polypeptide comprising or consisting of an amino acid sequence having 50.50% or more sequence identity to the full-length amino acid sequence of any one of SEQ ID NO 10, 09, 34, 36, 08, 07, 28, 31, 48, 35, 32, 19, 49, 26, 05 or 33, or
      comprises a functional fragment of a polypeptide according to any one of SEQ ID NO 10, 09, 34, 36, 08, 07, 28, 31, 48, 35, 32, 19, 49, 26, 05 or 33, or
      comprises a functional fragment comprising an amino acid sequence of at least 10 consecutive amino acid residues from any one of SEQ ID NO 10, 09, 34, 36, 08, 07, 28, 31, 48, 35, 32, 19, 49, 26, 05 or 33, or
   c) alpha-1,3-fucosyltransferase activity on the Glc residue of LNT and
      comprises a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or
      is a polypeptide comprising or consisting of an amino acid sequence having 50.50% or more sequence identity to the full-length amino acid sequence of any one of SEQ ID NO 48, 34, 32, 50, 03, 09, 33, 35, 01, 31, 28, 07, 49, 10, 18, 26, 02, 05, 08, 30, 06, 04, 27 or 11, or
      comprises a functional fragment of a polypeptide according to any one of SEQ ID NO 48, 34, 32, 50, 03, 09, 33, 35, 01, 31, 28, 07, 49, 10, 18, 26, 02, 05, 08, 30, 06, 04, 27 or 11, or
      comprises a functional fragment comprising an amino acid sequence of at least 10 consecutive amino acid residues from any one of SEQ ID NO 48, 34, 32, 50, 03, 09, 33, 35, 01, 31, 28, 07, 49, 10, 18, 26, 02, 05, 08, 30, 06, 04, 27 or 11, or
   d) alpha-1,3-fucosyltransferase activity on the Glc residue of LNT and alpha-1,4-fucosyltransferase activity on the GlcNAc residue of LNT, and
      comprises a polypeptide according to any one of SEQ ID NO 03, 09, 33, 35, 08, 01, 10, 31, 28, 07, 18, 26, 02, 05, 30, 06, 04, 27 or 11, or
      is a polypeptide comprising or consisting of an amino acid sequence having 50.50% or more sequence identity to the full-length amino acid sequence of any one of SEQ ID NO 03, 09, 33, 35, 08, 01, 10, 31, 28, 07, 18, 26, 02, 05, 30, 06, 04, 27 or 11, or
      comprises a functional fragment of a polypeptide according to any one of SEQ ID NO 03, 09, 33, 35, 08, 01, 10, 31, 28, 07, 18, 26, 02, 05, 30, 06, 04, 27 or 11, or
      comprises a functional fragment comprising an amino acid sequence of at least 10 consecutive amino acid residues from any one of SEQ ID NO 03, 09, 33, 35, 08, 01, 10, 31, 28, 07, 18, 26, 02, 05, 30, 06, 04, 27 or 11.
51. A cell metabolically engineered for the production of 3-FL, wherein said cell is capable to express, preferably expresses, a fucosyltransferase, characterized in that said fucosyltransferase has alpha-1,3-fucosyltransferase activity on the glucose (Glc) residue of lactose, and:
   comprises a polypeptide according to any one of SEQ ID NO 36, 32, 34, 28, 31, 37, 50, 27, 38, 29, 41, 49, 35, 39, 45, 43, 40 or 51, or
   is a polypeptide comprising or consisting of an amino acid sequence having 50.0% or more sequence identity to the full-length amino acid sequence of any one of SEQ ID NO 36, 32, 34, 28, 31, 37, 50, 27, 38, 29, 41, 49, 35, 39, 45, 43, 40 or 51, or
   comprises a functional fragment of a polypeptide according to any one of SEQ ID NO 36, 32, 34, 28, 31, 37, 50, 27, 38, 29, 41, 49, 35, 39, 45, 43, 40 or 51, or
   comprises a functional fragment comprising an amino acid sequence of at least 10 consecutive amino acid residues from any one of SEQ ID NO 36, 32, 34, 28, 31, 37, 50, 27, 38, 29, 41, 49, 35, 39, 45, 43, 40 or 51.
52. Cell according to any one of preferred embodiments 42 to 51, wherein said cell is modified in the expression or activity of any one of said fucosyltransferases.
53. Cell according to any one of preferred embodiments 42 to 52, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-rhamnose and UDP-xylose and/or wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.
54. Cell according to any one of preferred embodiments 42 to 53, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, said fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is an alpha-1,3-N-acetylgalactosaminyltransferase,
   preferably, said cell is modified in the expression or activity of any one of said glycosyltransferases.
55. Cell according to any one of preferred embodiments 42 to 54, wherein said cell is using one or more precursor(s) for the production of said fucosylated compound and/or said 3-FL, said precursor(s) being fed to the cell from the cultivation medium and/or wherein said cell is producing one or more precursor(s) for the production of said fucosylated compound and/or said 3-FL.
56. Cell according to preferred embodiment 55, wherein said precursor for the production of said fucosylated compound or 3-FL is completely converted into said fucosylated compound or 3-FL, respectively.
57. Cell according to any one of preferred embodiments 42 to 56, wherein said cell is capable to produce, preferably produces, said saccharide substrate and/or lactose.
58. Cell according to any one of preferred embodiments 42 to 57, wherein said cell produces said fucosylated compound and/or 3-FL intracellularly and wherein a fraction or substantially all of said produced fucosylated compound and/or 3-FL, respectively, remains intracellularly and/or is excreted outside said cell via passive or active transport.
59. Cell according to any one of preferred embodiments 42 to 58, wherein said cell expresses a membrane transporter protein or a polypeptide having transport activity hereby transporting compounds across the outer membrane of the cell wall, preferably, said cell is modified in the expression or activity of said membrane transporter protein or polypeptide having transport activity.
60. Cell according to preferred embodiment 59, wherein said membrane transporter protein or polypeptide having transport activity is chosen from the list comprising porters, P—P-bond-hydrolysis-driven transporters, b-barrel porins, auxiliary transport proteins, and phosphotransfer-driven group translocators,
   preferably, said porters comprise MFS transporters, sugar efflux transporters and siderophore exporters,
   preferably, said P—P-bond-hydrolysis-driven transporters comprise ABC transporters and siderophore exporters.
61. Cell according to any one of preferred embodiment 59 or 60, wherein said membrane transporter protein or polypeptide having transport activity:
   controls the flow over the outer membrane of the cell wall of said fucosylated compound and/or of one or more precursor(s) to be used in said production of said fucosylated compound and/or said 3-FL and/or of one or more precursor(s) to be used in said production of 3-FL, and/or
   provides improved production and/or enabled and/or enhanced efflux of said fucosylated compound and/or said 3-FL.
62. Cell according to any one of preferred embodiments 42 to 61, wherein the cell comprises a catabolic pathway for selected mono-, di- or oligosaccharides which is at least partially inactivated, the mono-, di-, or oligosaccharides being involved in and/or required for said production of said fucosylated compound and/or said 3-FL.
63. Cell according to any one of preferred embodiments 42 to 62, wherein said cell produces 90 g/L or more of said fucosylated compound and/or 3-FL in the whole broth and/or supernatant and/or wherein said fucosylated compound in the whole broth and/or supernatant has a purity of at least 80% measured on the total amount of said fucosylated compound and its precursor(s) in the whole broth and/or supernatant, respectively, and/or wherein said 3-FL in the whole broth and/or supernatant has a purity of at least 80% measured on the total amount of said 3-FL and its precursor(s) in the whole broth and/or supernatant, respectively.
64. Cell according to any one of preferred embodiments 42 to 63, wherein said cell is a bacterium, fungus, yeast, a plant cell, an animal cell, or a protozoan cell,
   preferably, said bacterium belongs to a phylum chosen from the group comprising Proteobacteria, Firmicutes, Cyanobacteria, Deinococcus-Thermus and Actinobacteria; more preferably, said bacterium belongs to a family chosen from the group comprising Enterobacteriaceae, Bacillaceae, Lactobacillaceae, Corynebacteriaceae and Vibrionaceae; even more preferably, said bacterium is chosen from the list comprising an Escherichia coli strain, a Bacillus subtilis strain, a Vibrio natriegens strain; even more preferably said Escherichia coli strain is a K-12 strain, most preferably said Escherichia coli K-12 strain is E. coli MG1655,
   preferably, said fungus belongs to a genus chosen from the group comprising Rhizopus, Dictyostelium, Penicillium, Mucor or Aspergillus,
   preferably, said yeast belongs to a genus chosen from the group comprising Saccharomyces, Zygosaccharomyces, Pichia, Komagataella, Hansenula, Yarrowia, Starmerella, Kluyveromyces, Debaromyces, Candida, Schizosaccharomyces, Schwanniomyces or Torulaspora; more preferably, said yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
   preferably, said plant cell is an algal cell or is derived from tobacco, alfalfa, rice, tomato, cotton, rapeseed, soy, maize, or corn plant,
   preferably, said animal cell is derived from insects, amphibians, reptiles, invertebrates, fish, birds or mammalian cells excluding human embryonic stem cells, more preferably said mammalian cell is chosen from the list comprising an epithelial cell, an embryonic kidney cell, a fibroblast cell, a COS cell, a Chinese hamster ovary (CHO) cell, a murine myeloma cell, an NIH-3T3 cell, a lactocyte derived from mammalian induced pluripotent stem cells, more preferably said mammalian induced pluripotent stem cells are human induced pluripotent stem cells, a post-parturition mammary epithelium cell, a polarized mammary cell, more preferably said polarized mammary cell is selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably, said protozoan cell is a Leishmania tarentolae cell.
65. Cell according to any one of preferred embodiments 42 to 64, wherein the cell produces:
   a mixture of negatively charged, preferably sialylated, and/or neutral di- and oligosaccharides comprising at least one of said fucosylated compound, or
   a mixture of negatively charged, preferably sialylated, and/or neutral oligosaccharides comprising at least one of said fucosylated compound.
66. Cell according to any one of preferred embodiments 42 to 65, wherein the cell produces:
   a mixture of negatively charged, preferably sialylated, and/or neutral di- and oligosaccharides comprising 3-FL or
   a mixture of negatively charged, preferably sialylated, and/or neutral oligosaccharides comprising 3-FL.
67. Use of a cell according to any one of preferred embodiments 42 to 66 for the production of a fucosylated compound comprising a fucosylated version of a saccharide substrate comprising Gal-β1,m-GlcNAc-β1,n-Gal-β1,4-Glc wherein said m is 3 or 4 and said n is 3 or 6.
68. Use of a method according to any one of preferred embodiments 1 to 41 for the production of a fucosylated compound comprising a fucosylated version of a saccharide substrate comprising Gal-β1,m-GlcNAc-β1,n-Gal-β1,4-Glc wherein said m is 3 or 4 and said n is 3 or 6.
69. Use of a cell according to any one of preferred embodiments 51 to 66 for the production of 3-FL.
70. Use of a method according to any one of preferred embodiments 12 to 41 for the production of 3-FL.
71. Use of a fucosylated compound obtained by the method according to any one of preferred embodiments 1 to 41 for the manufacture of a preparation, preferably a nutritional composition, more preferably a medicinal formulation, a dietary supplement, a dairy drink or an infant formula.
72. Use of 3-FL obtained by the method according to any one of preferred embodiments 12 to 41 for the manufacture of a preparation, preferably a nutritional composition, more preferably a medicinal formulation, a dietary supplement, a dairy drink or an infant formula.
73. A dried powder comprising, consisting of or consisting essentially of at least one fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, preferably wherein said powder is dried by any one of spray-drying, drum-drying or roller-drying.
74. A dried powder comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of preferred embodiments 1 to 41, preferably wherein said powder is dried by any one of spray-drying, drum-drying or roller-drying.
75. A dried powder according to any one of preferred embodiment 73 or 74, wherein said at least one fucosylated compound is chosen from the list comprising Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II).
76. A dried powder according to any one of preferred embodiment 73 or 74, wherein said powder comprises, consists of or consists essentially of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and/or Gal-β1,3-[Fuc-α1,4]-GlcNAc-β1,3-Gal-β1,4-[Fuc-α1,3]-Glc (lacto-N-difucohexaose II, LNDFH-II).
77. A dried powder comprising, consisting of, or consisting essentially of a mixture of mammalian milk oligosaccharides (MMOs), preferably at least one negatively charged and/or at least one neutral MMO, wherein said mixture comprises, consists of or consists essentially of at least one of LNFP-III, LNFP-VI, LNnDFH II, LNFP-V and LNDFH-II, preferably wherein said LNFP-III, LNFP-VI, LNnDFH II, LNFP-V and/or LNDFH-II is/are obtainable, preferably obtained, by the method according to any one of preferred embodiments 1 to 41, preferably wherein said powder is dried by any one of spray-drying, drum-drying or roller-drying.
78. A dried powder according to preferred embodiment 77, wherein said mixture of MMOs comprises, consists of or consists essentially of at least one MMO chosen from the group comprising LNFP-1, LNFP-II, LNFP-III, LNFP-V, LNFP-VI, LNDFH-1, LNDFH-II, LNnDFH II, 2′-FL, 3-FL, LN3, LNT, LNnT, 3′SL, 6′SL, sialyllacto-N-tetraose (LST-a, LST-b, LST-c, LSTd) and disialyllacto-N-tetraose.
79. A dried powder comprising at least 50% w/w of a fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, preferably wherein said powder is dried by any one of spray-drying, drum-drying or roller-drying.
80. A dried powder comprising at least 50% w/w of a fucosylated compound chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, preferably wherein said fucosylate compound is obtainable, preferably obtained, by the method according to any one of preferred embodiments 1 to 41, preferably wherein said powder is dried by any one of spray-drying, drum-drying or roller-drying.
81. A dried powder comprising, consisting of, or consisting essentially of a mixture of MMOs, wherein said mixture comprises 0.1 to 30% w/w, preferably 0.1 to 20% w/w, more preferably 1 to 10% w/w, of one or more fucosylated compound(s) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, preferably wherein said powder is dried by any one of spray-drying, drum-drying or roller-drying.
82. A dried powder comprising, consisting of, or consisting essentially of a mixture of MMOs, wherein said mixture comprises 0.1 to 30% w/w, preferably 0.1 to 20% w/w, more preferably 1 to 10% w/w, of one or more fucosylated compound(s) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid, preferably wherein said fucosylate compound is obtainable, preferably obtained, by the method according to any one of preferred embodiments 1 to 41, preferably wherein said powder is dried by any one of spray-drying, drum-drying or roller-drying.
83. A preparation comprising, consisting of or consisting essentially of at least one fucosylated compound 1) chosen from the list comprising a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein said m is 3 or 4 and said n is 3 or 6, optionally said saccharide is linked to a peptide, a protein and/or a lipid and 2) obtainable, preferably obtained, by the method according to any one of preferred embodiments 1 to 41.
84. A preparation comprising, consisting of, or consisting essentially of a dried powder according to any one of preferred embodiments 73 to 82.
85. A preparation comprising, consisting of, or consisting essentially of a mixture of mammalian milk oligosaccharides (MMOs), preferably at least one negatively charged MMO and/or at least one neutral MMO, wherein said mixture comprises, consists of, or consists essentially of at least one of LNFP-III, LNFP-VI, LNnDFH II, LNFP-V and LNDFH-II, preferably wherein said LNFP-III, LNFP-VI, LNnDFH II, LNFP-V and/or LNDFH-II is/are obtainable, preferably obtained, by the method according to any one of preferred embodiments 1 to 41.
86. A preparation according to preferred embodiment 85, wherein:
   the at least one negatively charged MMO is a sialylated MMO, preferably chosen from the group comprising 3′-sialyllactose, 6′-sialyllactose, sialyllacto-N-tetraose (LST-a, LST-b, LST-c, LSTd) and disialyllacto-N-tetraose, and/or
   the at least one neutral MMO is chosen from the list comprising fucosylated neutral MMOs and non-fucosylated neutral MMOs, preferably chosen from the group comprising 2′-fucosyllactose, 3-fucosyllactose, lacto-N-tetraose, lacto-N-neotetraose, lacto-N-fucopentaose I, LNFP-II, LNFP-III, LNFP-V, LNFP-VI, LNDFH-1, LNDFH-II and LNnDFH II.
87. A preparation according to any one of preferred embodiment 85 or 86, wherein said mixture of MMOs comprises, consists of or consists essentially of LNFP-III, LNFP-VI, LNnDFH II, LNFP-V and LNDFH-II.
88. A preparation according to any one of preferred embodiments 83 to 87, wherein said preparation further comprises at least one probiotic microorganism.
89. A preparation according to any one of preferred embodiments 83 to 88, wherein said preparation is a nutritional composition, preferably a medicinal formulation, a dietary supplement, a dairy drink or an infant formula.

The invention will be described in more detail in the examples. The following examples will serve as further illustration and clarification of the present invention and are not intended to be limiting.

### EXAMPLES

### Example 1. Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e., spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity (i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

### Example 2. Materials and Methods—General

### Heterologous and Homologous Expression

Genes that needed to be expressed, be it from a plasmid or from the genome were synthetically synthetized with one of the following companies: IDT or Twist Bioscience. Proteins described in present disclosure are summarized in Tables 1 and 2. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Overview of proteins with corresponding SEQ ID NOs
as described in the present invention

| SEQ | | | Country of |
|---|---|---|---|
| ID | | | origin of |
| NO | Organism | Origin | digital |
| 01 | Chitinophaga eiseniae | Synthetic | South Korea |
| 03 | Chitinophaga arvensicola | Synthetic | Japan |
| 04 | Azospirillum lipoferum 4B | Synthetic | Unknown |
| 05 | Azospirillum oryzae A2P | Synthetic | Japan |
| 06 | Azospirillum brasiliense LMG4388 | Synthetic | Belgium |
| | | | (Flanders) |
| 07 | Basilea psittacipulmonis JF4266 | Synthetic | Switzerland |
| 08 | Porphyromonas catoniae | Synthetic | Unknown |
| 09 | Porphyromonas sp. COT-239 OH1446 | Synthetic | United |
| | | | Kingdom |
| 10 | Selenomonas infelix | Synthetic | Unknown |
| 11 | Helicobacter bilis strain AAQJH | Synthetic | United States |
| 12 | Kingella denitrificans | Synthetic | Unknown |
| 13 | Planctopirus limnophila | Synthetic | Germany |
| 14 | Polynucleobacter asymbioticus | Synthetic | Austria |
| 15 | Helicobacter bilis ATCC 43879 | Synthetic | United States |
| 16 | Devosia insulae DS-56 | Synthetic | South Korea |
| 17 | delta proteobacterium NaphS2 | Synthetic | Germany |
| 18 | Pedobacter glucosidilyticus | Synthetic | Germany |
| 19 | Azospirillum sp. B510 | Synthetic | Japan |
| 20 | Paludibacter propionicigenes | Synthetic | Japan |
| 21 | Azospirillum sp. TSH58 | Synthetic | Japan |
| 22 | Azospirillum brasilense Sp7 | Synthetic | United States |
| 23 | Azospirillum sp. TSH64 | Synthetic | Japan |
| 24 | Azospirillum sp. TSH100 | Synthetic | Japan |
| 25 | Azospirillum brasiliense LMG 04385 | Synthetic | Belgium |
| | | | (Flanders) |
| 26 | [Haemophilus] felis | Synthetic | Sweden |
| | strain CCUG 35220 | | |
| 27 | Psittacicella hinzii strain B96_3 | Synthetic | Unknown |
| 28 | Methylovirgula ligni strain BW863 | Synthetic | Netherlands |
| 29 | Lewinella xylanilytica strain | Synthetic | South Korea |
| 30 | Unknown (seawater metagenome | Synthetic | United States |
| | SRR7054517 - ERZ650666) | | |
| 31 | Unknown (Unidentified | Synthetic | Unknown |
| | organism present in human buccal | | |
| | mucosa, HMP project: | | |
| | SRS 024347) | | |
| 32 | Bacteroides | Synthetic | Unknown |
| | bouchesdurhonensis isolate | | |
| | MGYG-HGUT-00098 | | |
| 33 | Butyrivibrio sp. TB | Synthetic | Unknown |
| 34 | Lewinella lacunae | Synthetic | South Korea |
| 35 | Porphyromonas catoniae | Synthetic | Unknown |
| 36 | Porphyromonas sp. | Synthetic | United States |
| 37 | Polynucleobacter sp. 86C-FISCH | Synthetic | Austria |
| 38 | Polynucleobacter sp. MG-5-Ahmo-C2 | Synthetic | Finland |
| 39 | Polynucleobacter sp. GWA2_45_21 | Synthetic | United States |
| 40 | Polynucleobacter sp. UK-Pondora-W15 | Synthetic | Finland |
| 41 | Polynucleobacter sp. UB-Tiil-W10 | Synthetic | Finland |
| 42 | Helicobacter cinaedi | Synthetic | Unknown |
| 43 | Helicobacter bilis | Synthetic | United States |
| 44 | Helicobacter bilis | Synthetic | United States |
| 45 | Campylobacter sp. P146 | Synthetic | United |
| | | | Kingdom |
| 46 | Devosia sp. | Synthetic | Denmark |
| 47 | Hyphomicrobiales bacterium | Synthetic | United States |
| 48 | Arcticibacter pallidicorallinus | Synthetic | Unknown |
| 49 | Parapedobacter defluvii | Synthetic | Unknown |
| 50 | Pedobacter schmidteae | Synthetic | Unknown |
| 51 | Helicobacter bilis | Synthetic | Italy |

TABLE 2
Overview of proteins with corresponding UniProt IDs
(sequence version 01, UniProt Database 2021_03
of 09 Jun. 2021) as described in the present invention

| | | | | Country of origin of |
|---|---|---|---|---|
| | | | | digital sequence |
| UniProt ID | Name | Organism | Origin | information |
| P27550 | acs | Escherichia coli | Synthetic | USA |
| | | K-12 MG1655 | | |
| A7LVG6 | AGE | Bacteroides ovatus | Synthetic | USA |
| P94526 | AraL | Bacillus subtilis | Synthetic | USA |
| A0ZZH6 | BaSP | Bifidobacterium | Synthetic | Germany |
| | | adolescentis | | |
| B7GPD4 | Blon_2475 | Bifidobacterium | Synthetic | Germany |
| | | longum subsp. infantis | | |
| Q8NFW8 | CMAS | Homo sapiens | Synthetic | Unknown |
| E0IXR1 | cscB | Escherichia coli W | Synthetic | USA |
| Q61420 | CST | Mus musculus | Synthetic | USA |
| P24077 | entS | Escherichia coli K-12 | Synthetic | USA |
| | | MG1655 | | |
| A0A378GQ13 | entS | Kluyvera ascorbata | Synthetic | USA |
| A0A6Y2K4E8 | entS | Salmonella enterica | Synthetic | USA |
| | | subsp. arizonae | | |
| P32055 | fcl | Escherichia coli K-12 | Synthetic | USA |
| | | MG1655 | | |
| SUV40286.1 | fkp | Bacteroides fragilis | Synthetic | United Kingdom |
| Q03417 | Frk | Zymomonas mobilis | Synthetic | United Kingdom |
| P11551 | fucP | Escherichia coli K-12 | Synthetic | USA |
| | | MG1655 | | |
| P09147 | galE | Escherichia coli K-12 | Synthetic | USA |
| | | MG1655 | | |
| O49213 | GER1 | Arabidopsis thaliana | Synthetic | USA |
| Q06210 | GFPT1 | Homo sapiens | Synthetic | Unknown |
| Q13630 | GFUS | Homo sapiens | Synthetic | Unknown |
| P0AC88 | gmd | Escherichia coli K-12 | Synthetic | USA |
| | | MG1655 | | |
| P31120* | glmM | Escherichia coli K-12 | Synthetic | USA |
| | | MG1655 | | |
| P17169** | glmS | Escherichia coli K-12 | Synthetic | USA |
| | | MG1655 | | |
| P0CI73 | glmS | Bacillus subtilis | Synthetic | USA |
| Q8NND3*** | glmS | Corynebacterium | Synthetic | USA |
| | | glutamicum | | |
| P0ACC7 | glmU | Escherichia coli K-12 | Synthetic | USA |
| | | MG1655 | | |
| Q96EK6 | GNA1 | Homo sapiens | Synthetic | Unknown |
| P43577 | GNA1 | Saccharomyces | Synthetic | USA |
| | | cerevisiae | | |
| Q9Y223 | GNE | Homo sapiens | Synthetic | Unknown |
| Q91WG8 | GNE | Mus musculus | Synthetic | USA |
| O30511 | HpFucT | Helicobacter pylori | Synthetic | Canada |
| | | UA1234 | | |
| Q9X435 | HpFutC | Helicobacter pylori | Synthetic | Canada |
| | | UA1234 | | |
| F8X274 | a1,2- | Dysgonomonas mossii | Synthetic | USA |
| | fucosyltransferase | | | |
| A0A1B8TNT0 | a1,2- | Polaribacter vadi | Synthetic | South Korea |
| | fucosyltransferase | | | |
| Q316B5 | a1,2- | Desulfovibrio | Synthetic | USA |
| | fucosyltransferase | alaskensis | | |
| A0A2N8IVT4 | a1,2- | Akkermansia | Synthetic | Netherlands |
| | fucosyltransferase | muciniphila | | |
| D4B8A6 | iceT | Citrobacter youngae | Synthetic | USA |
| A0A024L207 | iceT | Escherichia coli K-12 | Synthetic | USA |
| | | MG1655 | | |
| Q9SEE5 | KIN1 | Arabidopsis thaliana | Synthetic | USA |
| P07921 | LAC12 | Kluyveromyces lactis | Synthetic | USA |
| P02920 | LacY | Escherichia coli K-12 | Synthetic | USA |
| | | MG1655 | | |
| Q9JXQ6 | LgtA | Neisseria meningitidis | Synthetic | United Kingdom |
| | | MC58 | | |
| Q51116**** | LgtB | Neisseria meningitidis | Synthetic | United Kingdom |
| | | MC58 | | |
| A0A1V0NEL4 | ImrA | Lactococcus lactis | Synthetic | Serbia |
| | | subsp. lactis bv. | | |
| | | diacetylactis | | |
| P00946 | manA | Escherichia coli K-12 | Synthetic | USA |
| | | MG1655 | | |
| P24175 | manB | Escherichia coli K-12 | Synthetic | USA |
| | | MG1655 | | |
| P24174 | manC | Escherichia coli K-12 | Synthetic | USA |
| | | MG1655 | | |
| A0A2T7ANQ9 | MdfA | Cronobacter | Synthetic | USA |
| | | muytjensii | | |
| D4BC23 | MdfA | Citrobacter youngae | Synthetic | USA |
| P0AEY8 | MdfA | Escherichia coli K-12 | Synthetic | USA |
| | | MG1655 | | |
| G9Z5F4 | MdfA | Yokenella | Synthetic | Unknown |
| | | regensburgei | | |
| Q8A712 | NANP | Bacteroides | Synthetic | Unknown |
| | | thetaiotaomicron | | |
| KPA15328.1 | NANP | Candidatus | Synthetic | Germany |
| | | Magnetomorum sp. | | |
| | | HK-1 | | |
| Q9NR45 | NANS | Homo sapiens | Synthetic | Unknown |
| K9NPH9 | NANS | Pseudomonas sp. | Synthetic | Unknown |
| | | UW4 | | |
| B1EFH1 | nanT | Escherichia albertii | Synthetic | USA |
| P41036 | nanT | Escherichia coli K-12 | Synthetic | USA |
| | | MG1655 | | |
| Q8FD59 | nanT | Escherichia coli | Synthetic | USA |
| | | O6:H1 | | |
| Q93MP7 | NeuA | Campylobacter jejuni | Synthetic | USA |
| Q99KK2 | NeuA | Mus musculus | Synthetic | USA |
| A0A849CI62 | NeuA | Pasteurella multocida | Synthetic | USA |
| Q93MP9 | NeuB | Campylobacter jejuni | Synthetic | USA |
| E0NCD4 | NeuB | Neisseria meningitidis | Synthetic | United Kingdom |
| Q93MP8 | NeuC | Campylobacter jejuni | Synthetic | USA |
| P77737 | oppF | Escherichia coli K-12 | Synthetic | USA |
| | | MG1655 | | |
| O66375 | PdST6 | Photobacterium | Synthetic | Japan |
| | | damselae | | |
| A8QYL1 | P-JT-ISH-224-ST6 | Photobacterium sp. | Synthetic | Japan |
| | | JT-ISH-224 | | |
| Q64689 | a2,8- | Mus musculus | Synthetic | USA |
| | sialyltransferase | | | |
| O95394 | PGM3 | Homo sapiens | Synthetic | Unknown |
| Q9CNC4 | PmultST2 | Pasteurella multocida | Synthetic | Unknown |
| | | subsp. multocida str. | | |
| | | Pm70 | | |
| Q9CLP3 | PmultST3 | Pasteurella multocida | Synthetic | USA |
| A0A078LM16 | SetA | Citrobacter koseri | Synthetic | Unknown |
| P31675 | SetA | Escherichia coli K-12 | Synthetic | USA |
| | | MG1655 | | |
| P33026 | SetB | Escherichia coli K-12 | Synthetic | USA |
| | | MG1655 | | |
| P31436 | SetC | Escherichia coli K-12 | Synthetic | USA |
| | | MG1655 | | |
| Q11203 | ST3GAL3 | Homo sapiens | Synthetic | Unknown |
| P13721 | ST6 | Rattus norvegicus | Synthetic | USA |
| P0A884 | thyA | Escherichia coli K-12 | Synthetic | USA |
| | | MG1655 | | |
| Q16222 | UAP1 | Homo sapiens | Synthetic | Unknown |
| Q9C5I1 | USP | Arabidopsis thaliana | Synthetic | USA |
| D3QY14 | wbgO | Escherichia coli | Synthetic | Germany |
| | | O55:H7 | | |

*Sequence version 03 (23 Jan. 2007) as present in the UniProt Database 2021_03 of 09 Jun. 2021
**Sequence version 04 (23 Jan. 2007) as present in the UniProt Database 2021_03 of 09 Jun. 2021
***Sequence version 02 (23 Jan. 2007) as present in the UniProt Database 2021_03 of 09 Jun. 2021
**Sequence version 02 (01 Dec. 2000) as present in the UniProt Database 2021_03 of 09 Jun. 2021

### Analytical Analysis

Standards such as but not limited to sucrose, lactose, lacto-N-biose (LNB, Gal-β1,3-GlcNAc), fucosylated LNB (2′FLNB, 4-FLNB), N-acetyllactosamine (LacNAc, Gal-β1,4-GlcNAc), fucosylated LacNAc (2′FLacNAc, 3-FLacNAc), lacto-N-triose II (LN3), lacto-N-tetraose (LNT), lacto-N-neo-tetraose (LNnT), LNFP-I, LNFP-II, LNFP-III, LNFP-V, LNFP-VI, were purchased from Carbosynth (UK), Elicityl (France) and IsoSep (Sweden). Other compounds were analysed with in-house made standards.

Neutral oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 m) column with an Acquity UPLC BEH Amide VanGuard column, 130 Å, 2.1×5 mm. The column temperature was 50° C. The mobile phase consisted of a ¼ water and % acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 am). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Protein Quantification

For protein quantification a method is used that is compatible with reducing agents, such as reducing sugars or oligosaccharides with a reducing end. To this end, a Bradford assay (Thermo Scientific, Pierce) was used with a linear range between 1 and 1500 μg/mL. The assay was calibrated with a standard curve of BSA. The protein content of dried oligosaccharide products was quantified by dissolving a pre-weighed quantify in 18.2 MO-cm (Millipore, Bedford, MA, USA) de-ionized water (DIW) up to a quantity of 50% (m/v). The amount of protein is measured at 595 nm and converted to concentration with the calibration curve based on BSA.

### DNA Quantification

Production host specific DNA residue is quantified by RT-qPCR, for which specific primers on the host are designed so that residual DNA of the production host is amplified. The RT-qPCR was performed according to the standard protocol of a kit obtained from Sigma and was based on SYBR Green detection.

Total DNA is measured by means of a Threshold assay (Molecular Devices), based on an immunoassay allowing to measure as low as 2 μg of DNA in a sample in solution. Double stranded DNA is measured by means of SpectraMax® Quant™ AccuBlue™ Pico dsDNA Assay Kit (Molecular Devices) having a linear range between 5 μg and 3 ng of dsDNA.

### Example 3. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4.7H2O, 30 g/L sucrose or 30 g/L glycerol, 1 mL/L vitamin solution, 100 μl/L molybdate solution, and 1 mL/L selenium solution. As specified in the respective examples, 0.30 g/L sialic acid, 0.30 g/L GlcNAc, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4.7H2O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 0.30 g/L GlcNAc, 20 g/L lactose, 20 g/L LacNAc and/or 20 g/L LNB were additionally added to the medium as precursor(s). Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZAM15, Δ(lacZYA-argF) U169, deoR, recA1, endA1, hsdR17(rk⁻, mk⁺), phoA, supE44, lambda-, thi-1, gyrA96, re/A1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645).

In an example for GDP-fucose production, the mutant strain was derived from E. coli K12 MG1655 comprising knock-outs of the E. coli wcaJ and thyA genes and genomic knock-ins of constitutive transcriptional units containing a sucrose transporter like e.g. CscB from E. coli W (UniProt ID E0IXR1), a fructose kinase like e.g. Frk originating from Zymomonas mobilis (UniProt ID Q03417) and a sucrose phosphorylase like e.g. BaSP originating from Bifidobacterium adolescentis (UniProt ID A0ZZH6). GDP-fucose production can further be optimized in the mutant E. coli strain by genomic knock-outs of any one or more of the E. coli genes comprising glgC, agp, pfkA, pfkB, pgi, arcA, iclR, pgi and/on as described in WO2016075243 and WO2012007481. GDP-fucose production can additionally be optimized comprising genomic knock-ins of constitutive transcriptional units for a mannose-6-phosphate isomerase like e.g. manA from E. coli (UniProt ID P00946), a phosphomannomutase like e.g. manB from E. coli (UniProt ID P24175), a mannose-1-phosphate guanylyltransferase like e.g. manC from E. coli (UniProt ID P24174), a GDP-mannose 4,6-dehydratase like e.g. gmd from E. coli (UniProt ID P0AC88) and a GDP-L-fucose synthase like e.g. fcl from E. coli (UniProt ID P32055). GDP-fucose production can also be obtained by genomic knock-outs of the E. coli fucK and fuc/genes and genomic knock-ins of constitutive transcriptional units containing a fucose permease like e.g. fucP from E. coli (UniProt ID P11551) and a bifunctional enzyme with fucose kinase/fucose-1-phosphate guanylyltransferase activity like e.g. fkp from Bacteroides fragilis (UniProt ID SUV40286.1). All mutant strains can be additionally modified with genomic knock-outs of the E. coli LacZ, LacY and LacA genes and with a genomic knock-in of a constitutive transcriptional unit for a lactose permease like e.g. the E. coli LacY (UniProt ID P02920).

For production of fucosylated oligosaccharides, the mutant GDP-fucose production strain was additionally modified with expression plasmids comprising constitutive transcriptional units for a fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 and/or an alpha-1,2-fucosyltransferase like e.g. a polypeptide chosen from the list comprising UniProt IDs F8X274, A0A1B8TNT0 and Q316B5 and with a constitutive transcriptional unit for the E. coli thyA (UniProt ID P0A884) as selective marker. Additionally, and/or alternatively, the constitutive transcriptional units of the fucosyltransferase genes could be present in the mutant E. coli strain via genomic knock-ins.

Alternatively, and/or additionally, GDP-fucose and/or fucosylated oligosaccharide production can further be optimized in the mutant E. coli strains with genomic knock-ins of constitutive transcriptional units comprising a membrane transporter protein like e.g. MdfA from Cronobacter muytjensii (UniProt ID A0A2T7ANQ9), MdfA from Citrobacter youngae (UniProt ID D4BC23), MdfA from E. coli (UniProt ID POAEY8), MdfA from Yokenella regensburgei (UniProt ID G9Z5F4), iceT from E. coli (UniProt ID A0A024L207) or iceT from Citrobacter youngae (UniProt ID D4B8A6).

In an example for production of N-acetyllactosamine (LacNAc, Gal-β1,4-GlcNAc) the strains were modified with genomic knock-ins or expression plasmids comprising constitutive transcriptional units for a glucosamine 6-phosphate N-acetyltransferase like e.g. GNA1 from S. cerevisiae (UniProt ID P43577) and an N-acetylglucosamine beta-1,4-galactosyltransferase like e.g. IgtB from Neisseria meningitidis (Uniprot ID Q51116, sequence version 02, 1 Dec. 2000).

In an example to produce lacto-N-triose (LN3, GlcNAc-b1,3-Gal-b1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like e.g. the E. coli LacY (UniProt ID P02920) and a galactoside beta-1,3-N-acetylglucosaminyltransferase like e.g. IgtA (UniProt ID Q9JXQ6) from N. meningitidis.

In an example for production of LN3 derived oligosaccharides like lacto-N-tetraose (LNT, Gal-b1,3-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g. wbgO (Uniprot ID D3QY14) from E. coli O55:H7.

In an example for production of LN3 derived oligosaccharides like lacto-N-neotetraose (LNnT, Gal-b1,4-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,4-galactosyltransferase like e.g. LgtB (Uniprot ID Q51116, sequence version 02, 1 Dec. 2000) from Neisseria meningitidis.

LacNAc, LN3, LNT and/or LNnT production can further be optimized in the mutant E. coli strains with genomic knock-outs of the E. coli genes comprising any one or more of galT, ushA, ldhA and agp.

The mutant LacNAc, LN3, LNT and/or LNnT producing strains can also be optionally modified for enhanced UDP-GlcNAc production with a genomic knock-in of a constitutive transcriptional unit for an L-glutamine-D-fructose-6-phosphate aminotransferase like e.g. the mutant glmS*54 from E. coli (differing from the wild-type E. coli glmS protein, having UniProt ID P17169, sequence version 04, 23 Jan. 2007, by an A39T, an R250C and an G472S mutation as described by Deng et al. (Biochimie 2006, 88: 419-429).

The mutant E. coli strains can also optionally be adapted with a genomic knock-in of a constitutive transcriptional unit for an UDP-glucose-4-epimerase like e.g. galE from E. coli (UniProt ID P09147), a phosphoglucosamine mutase like e.g. glmM from E. coli (UniProt ID P31120, sequence version 03, 23 Jan. 2007) and an N-acetylglucosamine-1-phosphate uridylyltransferase/glucosamine-1-phosphate acetyltransferase like e.g. glmU from E. coli (UniProt ID P0ACC7).

The mutant LacNAc, LN3, LNT and/or LNnT producing E. coli strains can also optionally be adapted for growth on sucrose via genomic knock-ins of constitutive transcriptional units containing a sucrose transporter like e.g. CscB from E. coli W (UniProt ID E0IXR1), a fructose kinase like e.g. Frk originating from Zymomonas mobilis (UniProt ID Q03417) and a sucrose phosphorylase like e.g. BaSP originating from Bifidobacterium adolescentis (UniProt ID A0ZZH6).

Alternatively, and/or additionally, production of LacNAc, LN3, LNT, LNnT and oligosaccharides derived thereof can further be optimized in the mutant E. coli strains with genomic knock-ins of constitutive transcriptional units comprising a membrane transporter protein like e.g. MdfA from Cronobacter muytjensii (UniProt ID A0A2T7ANQ9), MdfA from Citrobacter youngae (UniProt ID D4BC23), MdfA from E. coli (UniProt ID POAEY8), MdfA from Yokenella regensburgei (UniProt ID G9Z5F4), iceT from E. coli (UniProt ID A0A024L207) or iceT from Citrobacter youngae (UniProt ID D4B8A6). In an example for sialic acid production, the mutant strain was derived from E. coli K12 MG1655 comprising genomic knock-ins of constitutive transcriptional units containing one or more copies of a glucosamine 6-phosphate N-acetyltransferase like e.g. GNA1 from Saccharomyces cerevisiae (UniProt ID P43577), an N-acetylglucosamine 2-epimerase like e.g. AGE from Bacteroides ovatus (UniProt ID A7LVG6) and an N-acetylneuraminate synthase like e.g. from Neisseria meningitidis (UniProt ID E0NCD4) or Campylobacter jejuni (UniProt ID Q93MP9).

Alternatively, and/or additionally, sialic acid production can be obtained by genomic knock-ins of constitutive transcriptional units containing an UDP-N-acetylglucosamine 2-epimerase like e.g. NeuC from C. jejuni (UniProt ID Q93MP8) and an N-acetylneuraminate synthase like e.g. from Neisseria meningitidis (UniProt ID E0NCD4) or Campylobacter jejuni (UniProt ID Q93MP9).

Alternatively and/or additionally, sialic acid production can be obtained by genomic knock-ins of constitutive transcriptional units containing a phosphoglucosamine mutase like e.g. glmM from E. coli (UniProt ID P31120, sequence version 03, 23 Jan. 2007), an N-acetylglucosamine-1-phosphate uridyltransferase/glucosamine-1-phosphate acetyltransferase like e.g. glmU from E. coli (UniProt ID P0ACC7), an UDP-N-acetylglucosamine 2-epimerase like e.g. NeuC from C. jejuni (UniProt ID Q93MP8) and an N-acetylneuraminate synthase like e.g. from Neisseria meningitidis (UniProt ID E0NCD4) or Campylobacter jejuni (UniProt ID Q93MP9).

Alternatively, and/or additionally, sialic acid production can be obtained by genomic knock-ins of constitutive transcriptional units containing a bifunctional UDP-GlcNAc 2-epimerase/N-acetylmannosamine kinase like e.g. from Mus musculus (strain C57BL/6J) (UniProt ID Q91WG8), an N-acylneuraminate-9-phosphate synthetase like e.g. from Pseudomonas sp. UW4 (UniProt ID K9NPH9) and an N-acylneuraminate-9-phosphatase like e.g. from Candidatus Magnetomorum sp. HK-1 (UniProt ID KPA15328.1) or from Bacteroides thetaiotaomicron (UniProt ID Q8A712).

Alternatively, and/or additionally, sialic acid production can be obtained by genomic knock-ins of constitutive transcriptional units containing a phosphoglucosamine mutase like e.g. glmM from E. coli (UniProt ID P31120, sequence version 03, 23 Jan. 2007), an N-acetylglucosamine-1-phosphate uridyltransferase/glucosamine-1-phosphate acetyltransferase like e.g. glmU from E. coli (UniProt ID P0ACC7), a bifunctional UDP-GlcNAc 2-epimerase/N-acetylmannosamine kinase like e.g. from M. musculus (strain C57BL/6J) (UniProt ID Q91WG8), an N-acylneuraminate-9-phosphate synthetase like e.g. from Pseudomonas sp. UW4 (UniProt ID K9NPH9) and an N-acylneuraminate-9-phosphatase like e.g. from Candidatus Magnetomorum sp. HK-1 (UniProt ID KPA15328.1) or from Bacteroides thetaiotaomicron (UniProt ID Q8A712).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of nanT, poxB, ldhA, adhE, aldB, pf/A, pflC, ybiY, ackA and/or pta and with genomic knock-ins of constitutive transcriptional units comprising one or more copies of an L-glutamine-D-fructose-6-phosphate aminotransferase like e.g. the mutant glmS*54 from E. coli (differing from the wild-type E. coli glmS, having UniProt ID P17169, sequence version 04, 23 Jan. 2007, by an A39T, an R250C and an G472S mutation as described by Deng et al. (Biochimie 88, 419-29 (2006)), preferably a phosphatase like any one of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225 and an acetyl-CoA synthetase like e.g. acs from E. coli (UniProt ID P27550).

For sialylated oligosaccharide production, said sialic acid production strains were further modified to express an N-acylneuraminate cytidylyltransferase like e.g. the NeuA enzyme from C. jejuni (UniProt ID Q93MP7) or the NeuA enzyme from Pasteurella multocida (UniProt ID A0A849C162) and to express one or more copies of a beta-galactoside alpha-2,3-sialyltransferase like e.g. PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity, or PmultST2 from P. multocida subsp. multocida str. Pm70 (UniProt ID Q9CNC4), a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g. from M. musculus (UniProt ID Q64689). Constitutive transcriptional units of the N-acylneuraminate cytidylyltransferase and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g. E. coli LacY (UniProt ID P02920). All mutant strains producing sialic acid, CMP-sialic acid and/or sialylated oligosaccharides could optionally be adapted for growth on sucrose via genomic knock-ins of constitutive transcriptional units containing a sucrose transporter like e.g. CscB from E. coli W (UniProt ID E0IXR1), a fructose kinase like e.g. Frk originating from Z. mobilis (UniProt ID Q03417) and a sucrose phosphorylase like e.g. BaSP from B. adolescentis (UniProt ID A0ZZH6).

Alternatively, and/or additionally, sialic acid and/or sialylated oligosaccharide production can further be optimized in the mutant E. coli strains with genomic knock-ins of constitutive transcriptional units comprising a membrane transporter protein like e.g. a sialic acid transporter like e.g. nanT from E. coli K-12 MG1655 (UniProt ID P41036), nanT from E. coli O6:H1 (UniProt ID Q8FD59), nanT from E. albertii (UniProt ID B1EFH1) or a porter like e.g. EntS from E. coli (UniProt ID P24077), EntS from Kluyvera ascorbata (UniProt ID A0A378GQ13) or EntS from Salmonella enterica subsp. arizonae (UniProt ID A0A6Y2K4E8), MdfA from Cronobacter muytjensii (UniProt ID A0A2T7ANQ9), MdfA from Citrobacter youngae (UniProt ID D4BC23), MdfA from E. coli (UniProt ID POAEY8), MdfA from Yokenella regensburgei (UniProt ID G9Z5F4), iceT from E. coli (UniProt ID A0A024L207), iceT from Citrobacter youngae (UniProt ID D4B8A6), SetA from E. coli (UniProt ID P31675), SetB from E. coli (UniProt ID P33026) or SetC from E. coli (UniProt ID P31436) or an ABC transporter like e.g. oppF from E. coli (UniProt ID P77737), ImrA from Lactococcus lactis subsp. lactis bv. diacetylactis (UniProt ID A0A1V0NEL4), or Blon_2475 from Bifidobacterium longum subsp. infantis (UniProt ID B7GPD4).

Preferably but not necessarily, any one or more of the glycosyltransferases, the proteins involved in nucleotide-activated sugar synthesis and/or the membrane transporter protein were N- and/or C-terminally fused to a solubility enhancer tag like e.g. a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fmicb.2014.00063; Fox et al., Protein Sci. 2001, 10(3), 622-630; Jia and Jeaon, Open Biol. 2016, 6: 160196).

Optionally, the mutant E. coli strains were modified with a genomic knock-ins of a constitutive transcriptional unit encoding a chaperone protein like e.g. DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcal, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP.

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360).

All strains were stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor (having a 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2S04 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Endotoxin Measurement

Endotoxin in the liquid was measured by means of a LAL test.

### Example 4. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2p yeast on and the Ura3 selection marker for selection and maintenance in yeast. This plasmid is further modified with constitutive transcriptional units for a lactose permease like e.g. LAC12 from K. lactis (UniProt ID P07921), a GDP-mannose 4,6-dehydratase like e.g. gmd from E. coli (UniProt ID P0AC88) and a GDP-L-fucose synthase like e.g. fcl from E. coli (UniProt ID P32055). The yeast expression plasmid p2a_2μ_Fuc2 can be used as an alternative expression plasmid of the p2a_2μ_Fuc plasmid comprising next to the ampicillin resistance gene, the bacterial ori, the 2p yeast on and the Ura3 selection marker constitutive transcriptional units for a lactose permease like e.g. LAC12 from K. lactis (UniProt ID P07921), a fucose permease like e.g. fucP from E. coli (UniProt ID P11551) and a bifunctional enzyme with fucose kinase/fucose-1-phosphate guanylyltransferase activity like e.g. fkp from Bacteroides fragilis (UniProt ID SUV40286.1). To further produce fucosylated oligosaccharides, the p2a_2μ_Fuc and its variant the p2a_2μ_Fuc2, additionally contained a constitutive transcriptional unit for a fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 and/or an alpha-1,2-fucosyltransferase like e.g. a polypeptide chosen from the list comprising UniProt IDs F8X274, A0A1B8TNT0 and Q316B5 or HpFutC from H. pylori (UniProt ID Q9X435).

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g. galE from E. coli (UniProt ID P09147). This plasmid can be further modified with constitutive transcriptional units for a lactose permease like e.g. LAC12 from K. lactis (UniProt ID P07921) and a galactoside beta-1,3-N-acetylglucosaminyltransferase activity like e.g. IgtA from N. meningitidis (UniProt ID Q9JXQ6) to produce LN3. In an example to further produce LN3-derived oligosaccharides like LNT, the mutant LN3 producing strains were further modified with a constitutive transcriptional unit for an N-acetylglucosamine beta-1,3-galactosyltransferase like e.g. WbgO (Uniprot ID D3QY14) from E. coli O55:H7.

In an example for production of LN3 derived oligosaccharides like lacto-N-neotetraose (LNnT, Gal-β1,4-GlcNAc-β1,3-Gal-β1,4-Glc), the mutant LN3 producing strain were further modified with a constitutive transcriptional unit for an N-acetylglucosamine beta-1,4-galactosyltransferase like e.g. LgtB (Uniprot ID Q51116, sequence version 02, 1 Dec. 2000) from Neisseria meningitidis.

In an example for production of N-acetyllactosamine (LacNAc, Gal-β1,4-GlcNAc), a yeast expression plasmid can be derived from the pRS420-plasmid series (Christianson et al., 1992, Gene 110: 119-122) containing the TRP1 selection marker and constitutive transcriptional units for one or more copies of an L-glutamine-D-fructose-6-phosphate aminotransferase like e.g. the mutant glmS*54 from E. coli (differing from the wild-type E. coli glmS, having UniProt ID P17169 (sequence version 04, 23 Jan. 2007), by an A39T, an R250C and an G472S mutation as described by Deng et al. (Biochimie 88, 419-29 (2006)), a phosphatase like any one of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225, a glucosamine 6-phosphate N-acetyltransferase like e.g. GNA1 from S. cerevisiae (UniProt ID P43577) and an N-acetylglucosamine beta-1,4-galactosyltransferase like e.g. LgtB from N. meningitidis (Uniprot ID Q51116, sequence version 02, 1 Dec. 2000).

Preferably but not necessarily, any one or more of the glycosyltransferase and/or the proteins involved in nucleotide-activated sugar synthesis were N- and/or C-terminally fused to a SUMOstar tag (e.g. obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g. Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275). Plasmids were maintained in the host E. coli DH5alpha (F-, phi80dlacZdeltaM15, delta(lacZYA-argF)U169, deoR, recA1, endA1, hsdR17(rk⁻, mk⁺), phoA, supE44, lambda⁻, thi-1, gyrA96, re/A1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by Blazeck (Biotechnology and Bioengineering, Vol. 109, No. 11, 2012).

### Example 5. Production of LNFP-III with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 04, 05, 06, 07, 11, 13, 14, 15, 16, 17, 19, 21, 22, 23, 24, 25, 26, 28, 29 or 31. The novel strains were evaluated in a growth experiment for production of LNFP-III (Gal-β1,4-[Fuc-α1,3]-GlcNAc-β1,3-Gal-β1,4-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured LNFP-III concentration was averaged over all biological replicates, and then normalized to the averaged LNFP-III concentration of a reference strain expressing the fucosyltransferase with SEQ ID NO 07. As demonstrated in Table 3, all novel strains demonstrated to produce LNFP-III.

TABLE 3
Relative production of LNFP-III (Gal-β1,4-[Fuc-α1,3]-
GlcNAc-β1,3-Gal-β1,4-Glc) (%) in mutant E. coli strains
expressing a fucosyltransferase with SEQ ID NO
04, 05, 06, 11, 13, 14, 15, 16, 17, 19, 21, 22,
23, 24, 25, 26, 28, 29 or 31 and producing GDP-fucose and
LNnT, when evaluated in a growth experiment
according to the culture conditions provided in Example 3,
in which the culture medium contained 30 g/L sucrose as carbon source
and 20 g/L lactose, and compared to a reference strain expressing the
fucosyltransferase with SEQ ID NO 07.

| Strain | Fucosyltransferase expressed | LNFP-III (%) |
|---|---|---|
| 1 | SEQ ID NO 07 | 100 |
| 2 | SEQ ID NO 04 | 147 |
| 3 | SEQ ID NO 05 | 105 |
| 4 | SEQ ID NO 06 | 378 |
| 5 | SEQ ID NO 11 | 60.6 |
| 6 | SEQ ID NO 13 | 120 |
| 7 | SEQ ID NO 14 | 184 |
| 8 | SEQ ID NO 15 | 109 |
| 9 | SEQ ID NO 16 | 149 |
| 10 | SEQ ID NO 17 | 31.0 |
| 11 | SEQ ID NO 19 | 216 |
| 12 | SEQ ID NO 21 | 159 |
| 13 | SEQ ID NO 22 | 173 |
| 14 | SEQ ID NO 23 | 188 |
| 15 | SEQ ID NO 24 | 18.5 |
| 16 | SEQ ID NO 25 | 26.9 |
| 17 | SEQ ID NO 26 | 72.5 |
| 18 | SEQ ID NO 28 | 0.91 |
| 19 | SEQ ID NO 29 | 57.9 |
| 20 | SEQ ID NO 31 | 69.3 |

### Example 6. Production of LNFP-VI with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 01, 02, 03, 07, 08, 09, 10, 12, 18, 20, 28 or 31. The novel strains were evaluated in a growth experiment for production of LNFP-VI (Gal-β1,4-GlcNAc-β1,3-Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured LNFP-VI concentration was averaged over all biological replicates, and then normalized to the averaged LNFP-VI concentration of a reference strain expressing the fucosyltransferase with SEQ ID NO 07. As demonstrated in Table 4, all novel strains demonstrated to produce LNFP-VI.

TABLE 4
Relative production of LNFP-VI (Gal-β1,4-GlcNAc-β1,3-Gal-β1,4-[Fuc-α1,3]-Glc) (%) in
mutant E. coli strains expressing a fucosyltransferase with SEQ ID NO 01, 02, 03, 08, 09,
10, 12, 18, 20, 28 or 31 and producing GDP-fucose and LNnT, when evaluated in a growth
experiment according to the culture conditions provided in Example 3,
in which the culture medium contained 30 g/L sucrose as carbon
source and 20 g/L lactose, and compared to a reference strain expressing
the fucosyltransferase with SEQ ID NO 07.

| Strain | Fucosyltransferase expressed | LNFP-VI (%) |
|---|---|---|
| 1 | SEQ ID NO 07 | 100 |
| 21 | SEQ ID NO 01 | 1012 |
| 22 | SEQ ID NO 02 | 498 |
| 23 | SEQ ID NO 03 | 1428 |
| 24 | SEQ ID NO 08 | 271 |
| 25 | SEQ ID NO 09 | 340 |
| 26 | SEQ ID NO 10 | 281 |
| 27 | SEQ ID NO 12 | 802 |
| 28 | SEQ ID NO 18 | 656 |
| 29 | SEQ ID NO 20 | 372 |
| 18 | SEQ ID NO 28 | 3.81 |
| 20 | SEQ ID NO 31 | 61.8 |

### Example 7. Production of LNnDFH II with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 05, 07, 08, 09, 10, 19, 26, 28 or 31. The novel strains were evaluated in a growth experiment for production of LNnDFH II (Gal-β1,4-[Fuc-α1,3]-GlcNAc-β1,3-Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured LNnDFH II concentration was averaged over all biological replicates, and then normalized to the averaged LNnDFH II concentration of a reference strain expressing the fucosyltransferase with SEQ ID NO 07. As demonstrated in Table 5, all novel strains demonstrated to produce LNnDFH II.

TABLE 5
Relative production of LNnDFH II (Gal-β1,4-[Fuc-α1,3]-GlcNAc-β1,3-
Gal-β1,4-[Fuc-α1,3]-Glc) (%) in mutant E. coli strains expressing a fucosyltransferase with
SEQ ID NO 05, 08, 09, 10, 19, 26, 28 or 31 and producing GDP-fucose and LNnT,
when evaluated in a growth experiment according to the culture conditions provided in Example
3, in which the culture medium contained 30 g/L sucrose as carbon source and 20 g/L lactose,
and compared to a reference strain expressing the fucosyltransferase with SEQ ID NO 07.

| Strain | Fucosyltransferase expressed | LNnDFH II (%) |
|---|---|---|
| 1 | SEQ ID NO 07 | 100 |
| 3 | SEQ ID NO 05 | 8.90 |
| 24 | SEQ ID NO 08 | 104 |
| 25 | SEQ ID NO 09 | 166 |
| 26 | SEQ ID NO 10 | 207 |
| 11 | SEQ ID NO 19 | 41.5 |
| 17 | SEQ ID NO 26 | 13.0 |
| 18 | SEQ ID NO 28 | 94.1 |
| 20 | SEQ ID NO 31 | 69.2 |

### Example 8. Production of an Oligosaccharide Mixture Comprising LNFP-III, LNFP-VI and LNnDFH II with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 07, 28 or 31. The novel strains were evaluated in a growth experiment for production of LNFP-III, LNFP-VI and LNnDFH II according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, each of the measured LNFP-III, LNFP-VI and LNnDFH II concentration was averaged over all biological replicates, and then normalized to the averaged LNFP-III, LNFP-VI and LNnDFH II concentration, respectively, of a reference strain expressing the fucosyltransferase with SEQ ID NO 07. As demonstrated in Table 6, all novel strains demonstrated to produce an oligosaccharide mixture comprising LNFP-III, LNFP-VI and LNnDFH II.

TABLE 6
Relative production of LNFP-III, LNFP-VI and LNnDFH II (%) in mutant E. coli strains
expressing a fucosyltransferase with SEQ ID NO 28 or 31 and producing GDP-fucose and LNnT,
when evaluated in a growth experiment according to the culture conditions provided in Example 3,
in which the culture medium contained 30 g/L sucrose as carbon source and 20 g/L lactose,
and compared to a reference strain expressing the fucosyltransferase with SEQ ID NO 07.

| Strain | Fucosyltransferase expressed | LNFP-III (%) | LNFP-VI (%) | LNnDFH II (%) |
|---|---|---|---|---|
| 1 | SEQ ID NO 07 | 100 | 100 | 100 |
| 18 | SEQ ID NO 28 | 0.91 | 3.81 | 94.1 |
| 20 | SEQ ID NO 31 | 69.3 | 61.8 | 69.2 |

### Example 9. Production of LNFP-V and LNDFH-II with a Modified E. coli Strain

A mutant E. coli K12 MG1655 strain modified for production of GDP-fucose and LNT (Gal-β1,3-GlcNAc-β1,3-Gal-β1,4-Glc) as described in Example 3 was transformed with an expression plasmid comprising a constitutive transcriptional unit for a fucosyltransferase selected from SEQ ID NO 01, 02, 03, 04, 05, 06, 07, 08, 09, 10, 11, 18, 26, 27, 28, 30 or 31. The novel strains were evaluated in a growth experiment for production of LNFP-V (Gal-β1,3-GlcNAc-β1,3-Gal-β1,4-[Fuc-α1,3]-Glc) and LNDFH-II (Gal-β1,3-[Fuc-α1,4]-GlcNAc-β1,3-Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, each of the measured LNFP-V and LNDFH-II concentration was averaged over all biological replicates, and then normalized to the averaged LNFP-V and LNDFH-II concentration, respectively, of a reference strain expressing the fucosyltransferase with SEQ ID NO 07. As demonstrated in Table 7, all novel strains demonstrated to produce LNFP-V and LNDFH-II.

TABLE 7
Relative production of LNFP-V (Gal-β1,3-GlcNAc-β1,3-Gal-β1,4-[Fuc-α1,3]-Glc)
(%) and LNDFH-II (Gal-β1,3-[Fuc-α1,4]-GlcNAc-β1,3-Gal-β1,4-[Fuc-α1,3]-Glc) (%) in mutant
E. coli strains expressing a fucosyltransferase with SEQ ID NO 01, 02, 03, 04, 05, 06, 08,
09, 10, 11, 18, 26, 27, 28, 30 or 31 and producing GDP-fucose and LNT, when evaluated in a
growth experiment according to the culture conditions provided in Example 3, in which the
culture medium contained 30 g/L sucrose as carbon source and 20 g/L lactose, and
compared to a reference strain expressing the fucosyltransferase with SEQ ID NO 07.

| Strain | Fucosyl transferase expressed | LNFP-V (%) | [LNFP-V + LNDFH-II] (%) |
|---|---|---|---|
| 30 | SEQ ID NO 07 | 100 | 100 |
| 31 | SEQ ID NO 01 | 197 | 167 |
| 32 | SEQ ID NO 02 | 48.1 | 40.8 |
| 33 | SEQ ID NO 03 | 297 | 252 |
| 34 | SEQ ID NO 04 | 21.9 | 18.6 |
| 35 | SEQ ID NO 05 | 46.4 | 39.4 |
| 36 | SEQ ID NO 06 | 23.2 | 19.7 |
| 37 | SEQ ID NO 08 | 40.3 | 188 |
| 38 | SEQ ID NO 09 | 252 | 214 |
| 39 | SEQ ID NO 10 | 83.6 | 137 |
| 40 | SEQ ID NO 11 | 14.1 | 12.0 |
| 41 | SEQ ID NO 18 | 66.4 | 56.3 |
| 42 | SEQ ID NO 26 | 54.3 | 46.1 |
| 43 | SEQ ID NO 27 | 20.1 | 17.1 |
| 44 | SEQ ID NO 28 | 118 | 100 |
| 45 | SEQ ID NO 30 | 24.9 | 21.1 |
| 46 | SEQ ID NO 31 | 152 | 129 |

### Example 10. Production of 3-FL with a Modified E. Coli Strain

A mutant E. coli K12 MG1655 strain modified for production of GDP-fucose as described in Example 3 was transformed with an expression plasmid comprising a constitutive transcriptional unit for a fucosyltransferase selected from SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30 or 31. The novel strains were evaluated in a growth experiment for production of 3-FL (Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured 3-FL concentration was averaged over all biological replicates, and then normalized to the averaged 3-FL concentration of a reference strain expressing the fucosyltransferase with SEQ ID NO 07. As demonstrated in Table 8, the novel strains expressing a fucosyltransferase with SEQ ID NO 03, 04, 05, 06, 07, 08, 09, 10, 13, 18, 19, 21, 22, 23, 24, 27, 28, 29 or 31 demonstrated to produce 3-FL.

TABLE 8
Relative production of 3-FL (Gal-β1,4-[Fuc-α1,3]-Glc) (%) in mutant E. coli strains
expressing a fucosyltransferase with SEQ ID NO 01, 02, 03, 04, 05, 06, 08, 09, 10, 11, 12, 13,
14, 15, 16, 17, 18, 19, 20, 21, 22, 23, 24, 25, 26, 27, 28, 29, 30 or 31 and producing GDP-fucose,
when evaluated in a growth experiment according to the culture conditions provided in Example
3, in which the culture medium contained 30 g/L sucrose as carbon source and 20 g/L lactose,
and compared to a reference strain expressing the fucosyltransferase with SEQ ID NO 07.

| Strain | Fucosyltransferase expressed | 3-FL (%) |
|---|---|---|
| 47 | SEQ ID NO 07 | 100 |
| 48 | SEQ ID NO 01 | 0 |
| 49 | SEQ ID NO 02 | 0 |
| 50 | SEQ ID NO 03 | 0.37 |
| 51 | SEQ ID NO 04 | 98.0 |
| 52 | SEQ ID NO 05 | 101 |
| 53 | SEQ ID NO 06 | 73.9 |
| 54 | SEQ ID NO 08 | 94.3 |
| 55 | SEQ ID NO 09 | 116 |
| 56 | SEQ ID NO 10 | 97.3 |
| 57 | SEQ ID NO 11 | 0 |
| 58 | SEQ ID NO 12 | 0 |
| 59 | SEQ ID NO 13 | 16.4 |
| 60 | SEQ ID NO 14 | 0 |
| 61 | SEQ ID NO 15 | 0 |
| 62 | SEQ ID NO 16 | 0 |
| 63 | SEQ ID NO 17 | 0 |
| 64 | SEQ ID NO 18 | 9.75 |
| 65 | SEQ ID NO 19 | 30.0 |
| 66 | SEQ ID NO 20 | 0 |
| 67 | SEQ ID NO 21 | 1.24 |
| 68 | SEQ ID NO 22 | 6.12 |
| 69 | SEQ ID NO 23 | 10.5 |
| 70 | SEQ ID NO 24 | 36.4 |
| 71 | SEQ ID NO 25 | 0 |
| 72 | SEQ ID NO 26 | 0 |
| 73 | SEQ ID NO 27 | 26.3 |
| 74 | SEQ ID NO 28 | 50.6 |
| 75 | SEQ ID NO 29 | 22.6 |
| 76 | SEQ ID NO 30 | 0 |
| 77 | SEQ ID NO 31 | 42.1 |

### Example 11. Production of an Oligosaccharide Mixture Comprising 3-FL, LNFP-III, LNFP-VI and LNnDFH II with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 07, 28 or 31. The novel strains were evaluated in a growth experiment for production of 3-FL, LNFP-III, LNFP-VI and LNnDFH II according to the culture conditions provided in Example 3, in which the strains were cultivated in minimal medium with 30 g/L sucrose and 20 g/L lactose. The strains were grown in four biological replicates in a 96-well plate. All novel strains demonstrated to produce an oligosaccharide mixture comprising 3-FL, LNFP-III, LNFP-VI and LNnDFH II.

### Example 12. Production of 3-FLacNAc with a Modified E. coli Strain

A mutant E. coli K12 MG1655 strain modified for production of GDP-fucose and N-acetyllactosamine (LacNAc, Gal-β1,4-GlcNAc) as described in Example 3 was transformed with an expression plasmid comprising a constitutive transcriptional unit for a fucosyltransferase selected from SEQ ID NO 03, 04, 05, 06, 07, 08, 09, 10, 11, 13, 14, 16, 19, 21, 22, 23 or 25. The novel strains were evaluated in a growth experiment for production of 3-FLacNAc (Gal-β1,4-[Fuc-α1,3]-GlcNAc) according to the culture conditions provided in Example 3, in which the strains were cultivated in minimal medium with 30 g/L sucrose. The strains were grown in four biological replicates in a 96-well plate. After 72 h of incubation, the culture broth was harvested, and the sugars were analysed on UPLC. For each strain with a particular fucosyltransferase tested, the measured 3-FLacNAc concentration was averaged over all biological replicates, and then normalized to the averaged 3-FLacNAc concentration of a reference strain expressing the fucosyltransferase with SEQ ID NO 07. As demonstrated in Table 9, all novel strains demonstrated to produce 3-FLacNAc.

TABLE 9
Relative production of 3-FLacNAc (Gal-β1,4-[Fuc-α1,3]-GlcNAc) (%) in mutant E. coli
strains expressing a fucosyltransferase with SEQ ID NO 03, 04, 05, 06, 08, 09, 10, 11, 13, 14,
16, 19, 21, 22, 23 or 25 and producing GDP-fucose and LacNAc (Gal-β1,4-GlcNAc), when
evaluated in a growth experiment according to the culture conditions provided in Example
3, in which the culture medium contained 30 g/L sucrose as carbon source, and compared to
a reference strain expressing the fucosyltransferase with SEQ ID NO 07.

| Strain | Fucosyltransferase expressed | 3-FLacNAc (%) |
|---|---|---|
| 78 | SEQ ID NO 07 | 100 |
| 79 | SEQ ID NO 03 | 6.56 |
| 80 | SEQ ID NO 04 | 56.8 |
| 81 | SEQ ID NO 05 | 49.9 |
| 82 | SEQ ID NO 06 | 75.2 |
| 83 | SEQ ID NO 08 | 93.0 |
| 84 | SEQ ID NO 09 | 111 |
| 85 | SEQ ID NO 10 | 82.4 |
| 86 | SEQ ID NO 11 | 14.1 |
| 87 | SEQ ID NO 13 | 24.6 |
| 88 | SEQ ID NO 14 | 6.49 |
| 89 | SEQ ID NO 16 | 18.4 |
| 90 | SEQ ID NO 19 | 39.3 |
| 91 | SEQ ID NO 21 | 2.05 |
| 92 | SEQ ID NO 22 | 5.49 |
| 93 | SEQ ID NO 23 | 19.8 |
| 94 | SEQ ID NO 25 | 0.49 |

### Example 13. Evaluation of Alpha-1,3-Fucosyltransferase Activity In Vitro

Another example provides the evaluation of alpha-1,3-fucosyltransferase activity of the enzymes with SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of the present invention in an in vitro enzymatic assay. These enzymes can be produced in a cell-free expression system such as but not limited to the PURExpress system (NEB), or in a host organism such as but not limited to Escherichia coli or Saccharomyces cerevisiae, after which the above listed enzymes can be isolated and optionally further purified. Each of the above enzyme extracts or purified enzymes are added to a reaction mixture together with GDP-fucose and a buffering component such as Tris-HCl or HEPES and a substrate like e.g. lactose, N-acetyllactosamine (LacNAc), lacto-N-tetraose (LNT) or lacto-N-neotetraose (LNnT). Said reaction mixture is then incubated at a certain temperature (for example 37° C.) for a certain amount of time (for example 8 hours, 16 hours, 24 hours), during which the lactose, LacNAc, LNT or LNnT will be converted by the enzyme using GDP-fucose to 3-FL, 3-FLacNAc, LNFP-V, LNDFH-II, LNFP-III, LNFP-VI and/or LNnDFH II, respectively. The oligosaccharides are then separated from the reaction mixture by methods known in the art. Further purification of 3-FL, 3-FLacNAc, LNFP-V, LNDFH-II, LNFP-III, LNFP-VI and/or LNnDFH II can be performed if preferred. At the end of the reaction or after separation and/or purification, the production of 3-FL, 3-FLacNAc, LNFP-V, LNDFH-II, LNFP-III, LNFP-VI and/or LNnDFH II is measured via analytical methods as described in Example 3 and known by the person skilled in the art. In vitro enzymatic assays were performed to test the alpha-1,3 fucosyltransferase activity of the fucosyltransferases with SEQ ID NO 01, 03 or 08 on either LNT or LNnT as saccharide substrate. The enzymes were isolated from E. coli host cultures expressing each time one of said enzymes. The reaction mixtures were incubated for 24 h at 37° C., after which the sugars were analysed on HPLC. For every enzyme tested, i.e. the polypeptide with SEQ ID NO 01, 03 and 08 a significant amount of either 1) LNFP-V and LNDFH-II or 2) LNFP-VI was produced in an in vitro enzymatic reaction with 1) LNT or with 2) LNnT as saccharide substrate, respectively. The enzyme with SEQ ID NO 08 also showed a significant production of LNnDFH II in the in vitro enzymatic reaction with LNnT as saccharide substrate.

### Example 14. Production of an Oligosaccharide Mixture Comprising LNFP-V, LNDFH-II and LNFP-VI with a Modified E. coli Host

A mutant E. coli K12 MG1655 strain modified for production of GDP-fucose, LNT and LNnT as described in Example 3 is transformed with an expression plasmid comprising a constitutive transcriptional unit for a fucosyltransferase selected from SEQ ID NO 01, 02, 03 and 18. The novel strains are evaluated for production of an oligosaccharide mixture comprising LNFP-V, LNDFH-II and LNFP-VI in whole broth samples in a growth experiment according to the culture conditions provided in Example 3, in which the culture medium contains sucrose as carbon source and lactose as precursor.

### Example 15. Production of an Oligosaccharide Mixture Comprising LNFP-II, LNFP-III, LNFP-V and LNDFH-II with a Modified E. coli Host

A mutant E. coli K12 MG1655 strain modified for production of GDP-fucose, LNT and LNnT as described in Example 3 is transformed with an expression plasmid comprising a constitutive transcriptional unit for a fucosyltransferase selected from SEQ ID NO 04, 05 and 06. The novel strains are evaluated for production of an oligosaccharide mixture comprising LNFP-II (Gal-β1,3-[Fuc-α1,4]-GlcNAc-β1,3-Gal-β1,4-Glc), LNFP-III, LNFP-V and LNDFH-II in whole broth samples in a growth experiment according to the culture conditions provided in Example 3, in which the culture medium contains sucrose as carbon source and lactose as precursor.

### Example 16. Production of an Oligosaccharide Mixture Comprising LNFP-II, LNFP-III and 3-FL with a Modified E. coli Host

A mutant E. coli K12 MG1655 strain modified for production of GDP-fucose and LNnT as described in Example 3 is transformed with an expression plasmid comprising a constitutive transcriptional unit for the fucosyltransferase with SEQ ID NO 29. The novel strain is evaluated for production of an oligosaccharide mixture comprising LNFP-II (Gal-β1,3-[Fuc-α1,4]-GlcNAc-β1,3-Gal-β1,4-Glc), LNFP-III and 3-FL in whole broth samples in a growth experiment according to the culture conditions provided in Example 3, in which the culture medium contains sucrose as carbon source and lactose as precursor.

### Example 17. Production of an Oligosaccharide Mixture Comprising LNFP-II, LNFP-III, LNFP-V, LNDFH-II, LNFP-VI, LNnDFH II and 3-FL with a Modified E. coli Host

A mutant E. coli K12 MG1655 strain modified for production of GDP-fucose, LNT and LNnT as described in Example 3 is transformed with an expression plasmid comprising a constitutive transcriptional unit for the fucosyltransferase with SEQ ID NO 31. The novel strain is evaluated for production of an oligosaccharide mixture comprising LNFP-II (Gal-β1,3-[Fuc-α1,4]-GlcNAc-β1,3-Gal-β1,4-Glc), LNFP-III, LNFP-V, LNDFH-II, LNFP-VI, LNnDFH II and 3-FL in whole broth samples in a growth experiment according to the culture conditions provided in Example 3, in which the culture medium contains sucrose as carbon source and lactose precursor.

### Example 18. Production of an Oligosaccharide Mixture Comprising 2′FL, DiFL, LNFP-I, LNFP-V and LNDFH-II with a Modified E. coli Host

The mutant E. coli K12 MG1655 strains modified for production of GDP-fucose, LNT, LNFP-V and LNDFH-II as described in Example 9 are further transformed with an expression plasmid comprising a constitutive transcriptional unit for the alpha-1,2-fucosyltransferase (HpFutC) from H. pylori (UniProt ID Q9X435). The novel strains are evaluated for production of an oligosaccharide mixture comprising 2′FL, DiFL, LNFP-I, LNFP-V and LNDFH-II in whole broth samples in a growth experiment according to the culture conditions provided in Example 3, in which the culture medium contains sucrose as carbon source and lactose as precursor.

### Example 19. Production of an Oligosaccharide Mixture Comprising 2′FL, DiFL, LNFP-I, LNFP-II, LNFP-III, LNFP-V, LNDFH-II, LNFP-VI, LNnDFH II and 3-FL with a Modified E. coli Host

The mutant E. coli K12 MG1655 strain modified for production of GDP-fucose, LNT and LNnT and for expression of the fucosyltransferase with SEQ ID NO 31 as described in Example 17 is further transformed with an expression plasmid comprising a constitutive transcriptional unit for the alpha-1,2-fucosyltransferase (HpFutC) from H. pylori (UniProt ID Q9X435). The novel strain is evaluated for production of an oligosaccharide mixture comprising 2′FL, DiFL, LNFP-1, LNFP-II, LNFP-III, LNFP-V, LNDFH-II, LNFP-VI, LNnDFH II and 3-FL in whole broth samples in a growth experiment according to the culture conditions provided in Example 3, in which the culture medium contains sucrose as carbon source and lactose as precursor.

### Example 20. Production of an Oligosaccharide Mixture Comprising Fucosylated and Sialylated Oligosaccharide Structures with a Modified E. coli Host

A mutant E. coli K12 MG1655 strain modified for production of GDP-fucose and LNT as described in Example 3 is further modified with genomic knock-outs of the E. coli nagA, nagB, nanA, nanE and nanK genes and genomic knock-ins of constitutive expression cassettes for glmS*54 from E. coli (differing from the wild-type E. coli glmS (UniProt ID P17169, sequence version 04, 23 Jan. 2007) by an A39T, an R250C and an G472S mutation), the UDP-N-acetylglucosamine 2-epimerase (neuC) from C. jejuni (UniProt ID Q93MP8) and the N-acetylneuraminate (Neu5Ac) synthase (neuB) of N. meningitidis (UniProt ID E0NCD4). In a next step, the novel strain is transformed with two compatible expression plasmids wherein a first plasmid comprises a constitutive transcriptional unit for a fucosyltransferase selected from SEQ ID NO 01, 02, 03, 04, 05, 06, 07, 08, 09, 10, 11, 18, 26, 27, 28, 30 or 31, and for the alpha-1,2-fucosyltransferase (HpFutC) from H. pylori (UniProt ID Q9X435) and wherein a second plasmid comprises constitutive expression units for the alpha-2,3-sialyltransferase PmultST3 from P. multocida (UniProt ID Q9CLP3), the alpha-2,6-sialyltransferase PdST6 from Photobacterium damselae (UniProt ID 066375) and the N-acylneuraminate cytidylyltransferase (NeuA) from P. multocida (UniProt ID A0A849C162). The novel strains are evaluated for production of an oligosaccharide mixture comprising fucosylated and sialylated structures such as 2′FL, DiFL, LNFP-1, LNFP-V, LNDFH-II, 3′SL, 6′SL, LSTa, LN3, LNT, 3'S-LN3 and 6'S-LN3 in whole broth samples, in a growth experiment according to the culture conditions provided in Example 3 in which the cultivation contains sucrose as carbon source and lactose as precursor.

### Example 21. Production of an Oligosaccharide Mixture Comprising Fucosylated and Sialylated Oligosaccharide Structures with a Modified E. coli Host

A mutant E. coli K12 MG1655 strain modified for production of GDP-fucose and LNnT as described in Example 3 is further modified with genomic knock-outs of the E. coli nagA, nagB, nanA, nanE and nanK genes and genomic knock-ins of constitutive expression cassettes for glmS*54 from E. coli (differing from the wild-type E. coli glmS (UniProt ID P17169, sequence version 04, 23 Jan. 2007) by an A39T, an R250C and an G472S mutation), the UDP-N-acetylglucosamine 2-epimerase (neuC) from C. jejuni (UniProt ID Q93MP8) and the N-acetylneuraminate (Neu5Ac) synthase (neuB) of N. meningitidis (UniProt ID E0NCD4). In a next step, the novel strain is transformed with two compatible expression plasmids wherein a first plasmid comprises a constitutive transcriptional unit for a fucosyltransferase selected from SEQ ID NO 04, 05, 06, 07, 11, 13, 14, 15, 16, 17, 19, 21, 22, 23, 24, 25, 26, 28, 29 or 31, and for the alpha-1,2-fucosyltransferase (HpFutC) from H. pylori (UniProt ID Q9X435) and wherein a second plasmid comprises constitutive expression units for the alpha-2,3-sialyltransferase PmultST3 from P. multocida (UniProt ID Q9CLP3), the alpha-2,6-sialyltransferase PdST6 from Photobacterium damselae (UniProt ID 066375) and the N-acylneuraminate cytidylyltransferase (NeuA) from P. multocida (UniProt ID A0A849C162). The novel strains are evaluated for production of an oligosaccharide mixture comprising fucosylated and sialylated structures such as 2′FL, DiFL, LNFP-III, LSTc, LSTd, 3′SL, 6′SL, LNnT, LN3, 3'S-LN3 and 6'S-LN3 in whole broth samples, in a growth experiment according to the culture conditions provided in Example 3 in which the cultivation contains sucrose as carbon source and lactose as precursor.

### Example 22. Production of an Oligosaccharide Mixture Comprising Fucosylated and Sialylated Oligosaccharide Structures with a Modified E. coli Host

A mutant E. coli K12 MG1655 strain modified for production of GDP-fucose, LNT and LNnT as described in Example 3 is further modified with genomic knock-outs of the E. coli nagA, nagB, nanA, nanE and nanK genes and genomic knock-ins of constitutive expression cassettes for glmS*54 from E. coli (differing from the wild-type E. coli glmS (UniProt ID P17169, sequence version 04, 23 Jan. 2007) by an A39T, an R250C and an G472S mutation), the UDP-N-acetylglucosamine 2-epimerase (neuC) from C. jejuni (UniProt ID Q93MP8) and the N-acetylneuraminate (Neu5Ac) synthase (neuB) of N. meningitidis (UniProt ID E0NCD4). In a next step, the novel strain is transformed with two compatible expression plasmids wherein a first plasmid comprises a constitutive transcriptional unit for the fucosyltransferase with SEQ ID NO 31 and for the alpha-1,2-fucosyltransferase (HpFutC) from H. pylori (UniProt ID Q9X435) and wherein a second plasmid comprises constitutive expression units for the alpha-2,3-sialyltransferase PmultST3 from P. multocida (UniProt ID Q9CLP3), the alpha-2,6-sialyltransferase PdST6 from Photobacterium damselae (UniProt ID 066375) and the N-acylneuraminate cytidylyltransferase (NeuA) from P. multocida (UniProt ID A0A849C162). The novel strains are evaluated for production of an oligosaccharide mixture comprising fucosylated and sialylated structures such as 2′FL, DiFL, 3-FL, LNFP-1, LNFP-II, LNFP-III, LNFP-V, LNDFH-II, LNFP-VI, LNnDFH II, LSTa, LSTc, LSTd, 3′SL, 6′SL, LN3, 3'S-LN3 and 6'S-LN3, LNT and LNnT in whole broth samples, in a growth experiment according to the culture conditions provided in Example 3 in which the cultivation contains sucrose as carbon source and lactose as precursor.

### Example 23. Production of an Oligosaccharide Mixture Comprising Fucosylated and Sialylated Oligosaccharide Structures with a Modified E. coli Host

A mutant E. coli K12 MG1655 strain modified for production of GDP-fucose, LNT and LNnT as described in Example 3 is further modified with genomic knock-outs of the E. coli nagA, nagB, nanA, nanE and nanK genes and genomic knock-ins of constitutive expression cassettes for glmS*54 from E. coli (differing from the wild-type E. coli glmS (UniProt ID P17169, sequence version 04, 23 Jan. 2007) by an A39T, an R250C and an G472S mutation), the UDP-N-acetylglucosamine 2-epimerase (neuC) from C. jejuni (UniProt ID Q93MP8) and the N-acetylneuraminate (Neu5Ac) synthase (neuB) of N. meningitidis (UniProt ID E0NCD4). In a next step, the novel strain is transformed with two compatible expression plasmids wherein a first plasmid comprises a constitutive transcriptional unit for the fucosyltransferase with SEQ ID NO 29 and for the alpha-1,2-fucosyltransferase (HpFutC) from H. pylori (UniProt ID Q9X435) and wherein a second plasmid comprises constitutive expression units for the alpha-2,3-sialyltransferase PmultST3 from P. multocida (UniProt ID Q9CLP3), the alpha-2,6-sialyltransferase PdST6 from Photobacterium damselae (UniProt ID 066375) and the N-acylneuraminate cytidylyltransferase (NeuA) from P. multocida (UniProt ID A0A849C162). The novel strains are evaluated for production of an oligosaccharide mixture comprising fucosylated and sialylated structures such as 2′FL, DiFL, 3-FL, LNFP-1, LNFP-II, LNFP-III, LSTa, LSTc, LSTd, 3′SL, 6′SL, LN3, 3'S-LN3 and 6'S-LN3, LNT and LNnT in whole broth samples, in a growth experiment according to the culture conditions provided in Example 3 in which the cultivation contains sucrose as carbon source and lactose as precursor.

### Example 24. Evaluation of Mutant E. coli Strains in Fed-Batch Fermentations

In another experiment, the mutant E. coli strains 31, 33 and 37 as described in Example 9 were evaluated in a fed-batch fermentation process. Fed-batch fermentations at bioreactor scale were performed as described in Example 3. Sucrose was used as a carbon source and lactose was added in the batch medium as precursor to the fermentation process. In contrast to the cultivation experiments that are described herein and wherein only end samples were taken at the end of cultivation (i.e., 72 hours as described herein), regular broth samples were taken at several time points during the fermentation process and the production of oligosaccharides was measured using UPLC as described in Example 3. Fermentations with mutant strains expressing a fucosyltransferase with SEQ ID NO 01, 03 or 08 demonstrated to produce an oligosaccharide mixture comprising LN3, LNT and LNFP-V in whole broth samples taken in fed-batch phase. The mutant E. coli strain 37, expressing the fucosyltransferase with SEQ ID NO 08, additionally showed production of LNFP-II and LNDFH-II. Results of relative production of LN3(%), LNT (%), LNFP-V (%), LNFP-II (%) and LNDFH-II (%) measured in whole broth samples are summarized in Table 10. For each strain, the relative production of LN3, LNT, LNFP-V, LNFP-II and LNDFH-II was calculated by dividing the production titres of LN3, LNT, LNFP-V, LNFP-II or LNDFH-II by the total sum of the production of LN3, LNT, LNFP-V, LNFP-II and LNDFH-II produced by that strain.

TABLE 10
Relative production of LN3 (%), LNT (%), LNFP-V (%), LNFP-II (%)
and LNDFH-II (%) measured in whole broth samples of mutant E. coli
strains adapted for production of GDP-fucose and LNT and for expression
of a fucosyltransferase with SEQ ID NO 01, 03 or 08, and cultivated
in a fed-batch fermentation run performed at bioreactor scale using
sucrose and lactose as described in Example 3.

| | Fucosyl- | | | | | |
|---|---|---|---|---|---|---|
| | transferase | LN3 | LNT | LNFP-V | LNFP-II | LNDFH-II |
| Strain | expressed | (%) | (%) | (%) | (%) | (%) |
| 31 | SEQ ID NO 01 | 15.3 | 8.90 | 75.8 | 0 | 0 |
| 33 | SEQ ID NO 03 | 12.6 | 22.2 | 65.2 | 0 | 0 |
| 37 | SEQ ID NO 08 | 19.1 | 21.6 | 15.0 | 4.80 | 39.5 |

### Example 25. Evaluation of Mutant E. coli Strains in Fed-Batch Fermentations

In another experiment, the mutant E. coli strains 07 and 13 as described in Example 5 were evaluated in a fed-batch fermentation process. Fed-batch fermentations at bioreactor scale were performed as described in Example 3. Sucrose was used as a carbon source and lactose was added in the batch medium as precursor to the fermentation process. In contrast to the cultivation experiments that are described herein and wherein only end samples were taken at the end of cultivation (i.e., 72 hours as described herein), regular broth samples were taken at several time points during the fermentation process and the production of oligosaccharides was measured using UPLC as described in Example 3. Fermentations with the mutant strains expressing a fucosyltransferase with SEQ ID NO 14 or 22 demonstrated to produce an oligosaccharide mixture comprising LN3, LNnT and LNFP-III in whole broth samples taken in fed-batch phase. Results of relative production of LN3(%), LNnT (%) and LNFP-III (%) measured in whole broth samples are summarized in Table 11. For each strain, the relative production of LN3, LNnT and LNFP-III was calculated by dividing the production titres of LN3, LNnT or LNFP-III by the total sum of the production of LN3, LNnT and LNFP-III produced by that strain.

TABLE 11
Relative production of LN3 (%), LNnT (%) and
LNFP-III (%) measured in whole broth samples of mutant
E. coli strains adapted for production of
GDP-fucose and LNnT and for expression
of a fucosyltransferase with SEQ ID NO
14 or 22, and cultivated in a fed-batch
fermentation run performed at bioreactor scale
using sucrose and lactose as described in Example 3.

| | Fucosyltransferase | | | |
|---|---|---|---|---|
| Strain | expressed | LN3 (%) | LNnT (%) | LNFP-III (%) |
| 07 | SEQ ID NO 14 | 45.3 | 24.9 | 29.9 |
| 13 | SEQ ID NO 22 | 13.3 | 7.80 | 78.9 |

### Example 26. Evaluation of Mutant E. coli Strains in Fed-Batch Fermentations

In another experiment, the mutant E. coli strains as described in Examples 05 to 12 and 14 to 19 are evaluated in a fed-batch fermentation process. Fed-batch fermentations at bioreactor scale are performed as described in Example 3. Sucrose is used as a carbon source and lactose is added in the batch medium as precursor to the fermentation process. In contrast to the cultivation experiments that are described herein and wherein only end samples were taken at the end of cultivation (i.e., 72 hours as described herein), regular broth samples are taken at several time points during the fermentation process and the production of neutral fucosylated and non-fucosylated oligosaccharides at each of said time points is evaluated using UPLC as described in Example 3.

### Example 27. Evaluation of Mutant E. coli Strains in Fed-Batch Fermentations

In another experiment, the mutant E. coli strains as described in Examples 20 to 23 are evaluated in a fed-batch fermentation process. Fed-batch fermentations at bioreactor scale are performed as described in Example 3. Sucrose is used as a carbon source and lactose is added in the batch medium as precursor to the fermentation process. In contrast to the cultivation experiments that are described herein and wherein only end samples were taken at the end of cultivation (i.e., 72 hours as described herein), regular broth samples are taken at several time points during the fermentation process and the production of sialylated and neutral oligosaccharides like at each of said time points is evaluated using UPLC as described in Example 3.

### Example 28. Composition Determination of Fermentation Broth

For the fermentation broths obtained in Examples 24 to 27 the composition was determined by measuring the cell dry mass of the broth, the ash content of the supernatant and the broth, and the total dry solids in the broth in accordance with the methods described below. The oligosaccharide content of the supernatant and the broth was determined using UPLC as described in Example 3. For all samples the total oligosaccharide content was below 80% on total dry solids. The oligosaccharide mixture purity in the broth ranged from 30% to 77%.

### Biomass Dry Mass Content (Cell Dry Mass)

Cell dry weight was obtained by centrifugation (15 min, 5000 g) of 20 g broth in pre-dried (70° C. overnight) and weighted falcons. The pellets were subsequently washed once with 20 mL physiological solution (9 g/L NaCl) and dried at 70° C. to a constant weight. The final weight was corrected for the added sodium chloride to the sample.

### Ash Content

The ash content is a measure of the total amount of minerals present within a food or ingredients such as oligosaccharides, whereas the mineral content is a measure of the amount of specific inorganic components present within a food, such as Ca, Na, K, Mg, phosphate, sulphate and Cl. Determination of the ash and mineral content of foods or oligosaccharides is important for a number of reasons: Nutritional labeling. The concentration and type of minerals present must often be stipulated on the label of a food or ingredient such as oligosaccharides. The quality of many foods depends on the concentration and type of minerals they contain, including their taste, appearance, texture and stability. Microbiological stability. High mineral contents are sometimes used to retard the growth of certain microorganisms. Nutrition. Some minerals are essential to a healthy diet (e.g., calcium, phosphorous, potassium and sodium) whereas others can be toxic (e.g., lead, mercury, cadmium and aluminium). Processing. It is often important to know the mineral content of foods/products during processing because this affects the physicochemical properties of foods or ingredient such as oligosaccharides.

Ash is the inorganic residue remaining after the water and organic matter have been removed by heating in the presence of oxidizing agents, which provides a measure of the total amount of minerals within a food. Analytical techniques for providing information about the total mineral content are based on the fact that the minerals (the analyte) can be distinguished from all the other components (the matrix) within a food or ingredient in some measurable way. The most widely used methods are based on the fact that minerals are not destroyed by heating, and that they have a low volatility compared to other food components. The three main types of analytical procedure used to determine the ash content of foods are based on this principle: dry ashing, wet ashing and low temperature plasma dry ashing. The method chosen for a particular analysis depends on the reason for carrying out the analysis, the type of food or ingredient analyzed and the equipment available. Ashing may also be used as the first step in preparing samples for analysis of specific minerals, by atomic spectroscopy or the various traditional methods described below.

For the sample preparation a sample whose composition represents that of the ingredient is selected to ensure that its composition does not change significantly prior to analysis. For instance, a dry oligosaccharide sample is generally hygroscopic and the selected sample should be kept under dry conditions avoiding the absorption of water. Typically, samples of 1-10 g are used in the analysis of ash content. Solid ingredients are finely ground and then carefully mixed to facilitate the choice of a representative sample. Before carrying out an ash analysis, samples that are high in moisture or in solution are generally dried to prevent spattering during ashing. Other possible problems include contamination of samples by minerals in grinders, glassware or crucibles which come into contact with the sample during the analysis. For the same reason, deionized water is used when preparing samples and the same is used in the blank sample.

Dry ashing procedures use a high temperature muffle furnace capable of maintaining temperatures of between 50° and 600° C. Water and other volatile materials are vaporized and organic substances are burned in the presence of the oxygen in air to CO₂, H₂O and N₂. Most minerals are converted to oxides, sulphates, phosphates, chlorides or silicates. Although most minerals have fairly low volatility at these high temperatures, some are volatile and may be partially lost, e.g., iron, lead and mercury, for these minerals ICP-MS analysis of the product is more appropriate for quantification.

The food sample is weighed before and after ashing to determine the concentration of ash present. The ash content can be expressed on dry basis is calculated by dividing the mass of the ashed material, ingredient, or food by the mass of the dry material, ingredient, or food before ashing. Multiplied with 100, this gives the percentage of ash in the material, ingredient, or food. In a similar way the wet ash percentage can be determined for liquid products, wherein the mass of the liquid before and after ashing is used instead of the mass of the dry material, ingredient, or food.

### Heavy Metal Determination

A robust general inductively coupled plasma-mass spectrometry (ICP-MS) based method was used for the detection and quantitation for each of the following elements: arsenic (As), selenium (Se), cadmium (Cd), tin (Sn), lead (Pb), silver (Ag), palladium (Pd), platinum (Pt), mercury (Hg), molybdenum (Mo), sodium (Na), potassium (K), Calcium (Ca), Magnesium (Mg), Iron (Fe), zinc (Zn), manganese (Mn), Phosphorus (P), selenium (Se).

Nitric acid (≥65%, Sigma-Aldrich) was used for microwave digestion and standard/sample preparation. All dilutions were done using 18.2 MO-cm (Millipore, Bedford, MA, USA) de-ionized water (DIW). About 0.2 g of each oligosaccharide, ingredient, sample were digested in 5 mL of HNO3 using the microwave digestion (CEM, Mars 6) program 15 minutes (min) ramping time and 15 min holding time at 100 W and 50° C. followed by 15 min ramping time and 20 min holding time at 1800 W and 210° C. The samples were cooled after digestion for 30 minutes. 1. The fully digested samples were then diluted to 50 mL with DIW. Analyses were carried out using a standard Agilent 7800 ICP-MS, which includes the fourth-generation ORS cell system for effective control of polyatomic interferences using helium collision mode (He mode). The ORS controls polyatomic interferences using He to reduce the transmission of all common matrix-based polyatomic interferences. Smaller, faster analyte ions are separated from larger, slower interference-ions using kinetic energy discrimination (KED). All elements, except Se, were measured in He mode with a flow rate of 5 mL/min. Se was measured in High Energy He (HEHe) mode, using a cell gas flow rate of 10 mL/min. The 7800 ICP-MS was configured with the standard sample introduction system consisting of a MicroMist glass concentric nebulizer, quartz spray chamber, quartz torch with 2.5 mm i.d. injector, and nickel interface cones. The ICP-MS operating conditions are: 1550 W RF power, 8 mm sampling depth, 1.16 I/min nebulizing gas, autotuned lens tuning, 5 or 10 mL/min helium gas flow, 5 V KED.

### Dry Matter/Dry Solid and Moisture Content Quantification

Sartorius MA150 Infrared Moisture Analyzer is used to determine the dry matter content of the oligosaccharides. 0.5 g of oligosaccharide is weighed on an analytical balance and is dried in the infrared moisture analyzer until the weight of the sample is stable. The mass of the dried sample divided by the mass of the sample before drying gives the dry matter content (in percent) of the oligosaccharides or sample including oligosaccharides. In a similar way a liquid sample is weighed, however, the amount of liquid weighed is adapted to the expected amount of dry matter in the liquid, so the mass of the dry matter is properly measurable on an analytical balance.

A moisture analyser measures the dry matter, but not the water content. Karl Fisher titration is used to determine the amount of water present in a powder, ingredient of food. The KF titration is carried out with a Karl Fischer titrator DL31 from Mettler Toledo using the two-component technique with Hydra-Point Solvent G and Hydra-Point titrant (5 mg H₂O/mL), both purchased from J.T. Baker (Deventer, Holland). The polarising current for bipotentiometric end-point determination was 20 microA and the stop voltage 100 mV. The end-point criterion was the drift stabilisation (15 micro gram H₂O min⁻¹) or maximum titration time (10 min).

The moisture content (MC) of sample was calculated using the following equation:

MC=V_KF W_eq 100/W_sample; where V_KF is the consumption of titrant in mL, W_eq the titre of titrant in mg H₂O/mL and W_sample the weight of sample in mg.

### Example 29. Purification of an Oligosaccharide and/or an Oligosaccharide Mixture from Fermentation Broth

### Cell Lysis

In many of the above-described mutant strains the product is readily excreted from the cell. Larger molecules however tend to be released more difficult during the fermentation process. Therefore, an additional step is optionally introduced to release the product from the cell. The broths from the fermentation processes of Examples 24 to 27 are used in a cell lysis experiment.

A soft release of the product was established by heating the broth for 1 hour to a temperature between 60° C. and 80° C. The higher the temperature, the more release was obtained, but colour formation increased. The product release was most optimal at a pH below 6.5 and above 3.0. The least monosaccharide formation was found at a pH of above 3.9. The release of the product is quantified by the measurement of the total oligosaccharide pool, as described in Example 3, in the broth before and after treatment. When observing an increase in oligosaccharide concentration, the product is released from the cells.

To disrupt the cell integrity even more other methods are also commonly used, such as, freeze thawing and/or shear stress through sonication, mixing, a homogenizer and/or French press.

### Broth Clarification

The broth originating from the cultivation or fermentation and, as the case may be, lysis step, are further clarified through microfiltration. For filtration several types of microfiltration membranes have been used to clarify the fermentation broth with a pore size ranging between 0.1 to 10 μm (ceramic, PES, PVDF membranes). The membrane types were first used as dead-end filtration and further optimization was performed in cross flow filtration. The cross-flow microfiltration was followed by diafiltration to increase product yield after this purification step. The membranes are capable of separating large, suspended solids such as colloids, particulates, fat, bacteria, yeasts, fungi, cells, while allowing sugars, proteins, salts, and low molecular weight molecules pass through the membrane.

The particle concentration in the filtrate was measured with a spectrophotometer at light adsorption at nm. This method allows the validation of particle removal and filtration optimization.

Alternative to microfiltration membranes, ultrafiltration membranes are used. Ultrafiltration membranes with a cut-off between 1000 Da and 10 kDa were tested (microdyne Nadir (3 kDa PES), Synder (3 kDa, PES), Synder Filtration MT (5 kDa, PES) and Synder Filtration ST (10 kDa, PES)). Alternative membranes with larger cut-offs will also work for broth clarification. The membranes were used in cross flow mode, and diafiltrations were applied similar to the microfiltration operation described above to increase product yield. The filtration efficiency is evaluated based on the particle concentration of the filtrate. Apart from cells and cell debris, membranes below 10 kDa efficiently remove DNA, protein and endotoxins, which were measured with the methods described in Example 3. Higher cut-off membranes between 10 and kDa remove cell mass efficiently, but do not retain smaller molecular weight products as efficiently, therefore requiring an additional Ultrafiltration step with a molecular weight cut-off below 10 kDa. A final recovery through ultrafiltration for broth clarification of above 95% was obtained.

To enhance broth clarification through centrifugation, flocculants/coagulants have been used. Generally, gypsum, alum, calcium hydroxide, polyaluminium chloride, aluminium chlorohydrate, are used as good flocculation agents. These flocculants were applied at a pH >7.0 and at temperatures between 4° C. and 20° C., more preferably between 4° C. and 10° C. pH<7.0 released toxic cations which are removed further through cation exchange. Alternative flocculants tested are based on polyacrylamide or biopolymer (chitosan), Floquant (SNF inc), Superfloc (Kemira) or hyperfloc (Hychem inc), Tramfloc. These flocculants were used in different concentrations: 0.05, 0.1 and 0.2 v/v % after diluting the broth 1:1 with RO-water, they were directly added to the broth and gently mixed for 10 minutes at room temperature. pH was kept at neutral conditions, between pH 6.0 and 7.0. At higher pH some degradation of the flocculant occurs, leading to compounds that are removed by means of ion exchange.

To test flocculation efficiency centrifugation was performed at 4000 g and the pellet strength and supernatant turbidity was evaluated after different centrifugation times. The oligosaccharide yield was measured by measuring the oligosaccharide supernatant concentration and the total supernatant volume. The pellet was washed several times to increase the release of oligosaccharides. A final oligosaccharide recovery between 90 and 98% was obtained.

### Ultrafiltration

Ultrafiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). The membrane to further remove DNA, protein and endotoxin was a 10 kDa membrane based on PES (Synder), used in crossflow. After filtration, the DNA, protein and endotoxin content was measured in the filtrate as described in Example 2. The protein content was below 100 mg per kg dry solid, the DNA content below 10 ng per gram dry solid and the endotoxin was below 10000 EU per gram dry solid. No DNA from the production hosts could be detected in the filtrate.

Although in this example a polysulfon based membrane was used, other membrane materials will perform equally, these membrane materials can be a ceramic or made of a synthetic or natural polymer, e.g. polypropylene, cellulose acetate or polylactic acid from suppliers such as Synder, Tami, TriSep, Microdyn Nadir, GE.

### Ion and Disaccharide Removal Through Nanofiltration

Tangential flow nanofiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). Clarified liquid treated with ultrafiltration was further subjected to nanofiltration and sequential diafiltrations. To this end a polyamide base membrane with a cut off between 300 and 500 Da was used (TriSep XN-45 (TriSep Corporation, USA) at 40° C. The diafiltrations were done with deionized water with a total volume of five times the volume of the oligosaccharide mixture concentrate. This step reduced the disaccharide fraction on dry solid below 5% and reduced the total ash content of the liquid with 50%. The concentration of the oligosaccharide mixture was increased to about 200 g/L.

### Ion Removal Through Electrodialysis

The ED equipment used is a PCCell ED 64004 lab-scale ED stack, fitted with 5 cell pairs of the PC SA and PC SK standard ion-exchange membranes. The initial diluted and concentrated both consisted of 1.5 L of the feed stream obtained after the clarification and ultrafiltration steps. The liquids obtained contained oligosaccharides and cations and anions with an ash content above 10% on dry solid. The desalination was done against a concentration gradient. Both streams are recirculated while a constant voltage of 7.5V is applied and the current and conductivity are monitored. Samples are taken at the beginning and end and periodically during the experiment. Water transport across the membranes is monitored by measuring the volume of all streams at the end of the experiment. To ensure efficient transfer of the current to the stack, an electrolyte solution of 60 g/L NaNO3 is recirculated at the electrodes.

The ED experiment was maintained until a stabilization of the current and conductivity was noticed. This indicates the point where desalination becomes slow and more inefficient. The conductivity decreases from 3.79 mS/cm in the feed to 0.88 mS/cm at the end of the experiment, indicating an overall desalination of 77%. The multivalent anions were removed up to 90%. The final oligosaccharide recovery was between 90 and 99%. The ash content on dry solid after electrodialysis was about 2.5% on dry solid.

### Ion Removal Through Ion Exchange

To remove ions from the broth to an ash content <1%, first a cation exchange and second an anion exchange step was performed. Depending on the mixture of oligosaccharides different anion exchange resins were selected to enhance the yield of the purification step.

Clarified broths containing neutral fucosylated and/or non-fucosylated oligosaccharides were first passed through a strong acid cation exchange resin containing column (1 L of Amberlite IR120) in the proton form at a temperature of 10° C., resulting in exchange of all cations with a proton in the liquid. The liquid resulting from the cation exchange step was subjected to a weak base anion exchange resin containing column (1 L of Amberlite IR400) in the hydroxide form at a temperature of 10° C., exchanging the anions in the liquid for hydroxide ions. After both cation and anion exchange, the pH was set to a pH between 6.0 and 7.0. The oligosaccharide recovery was between 95 and 98%. Alternative cation and anion exchange resins are Amberlite IR100, Amberlite IR120, Amberlite FPC22, Dowex 50WX, Finex CS16GC, Finex CS13GC, Finex CS12GC, Finex CS11GC, Lewatit S, Diaion SK, Diaion UBK, Amberjet 1000, Amberjet 1200 and Amberjet 4200, Amberjet 4600, Amberlite IR400, Amberlite IR410, Amberlite IR458, Diaion SA, Diaion UBA120, Lewatit MonoPlus M, Lewatit S7468. The cation and anion exchange treated liquids were then tested on ash, oligosaccharide content and heavy metal content. The ash content after treatment was below 0.5% (on total dry solid), the lead content was lower than 0.1 mg/kg dry solid, the arsenic content was lower than 0.2 mg/kg dry solid, the cadmium content was lower than 0.1 mg/kg dry solid and the mercury content was lower than 0.5 mg/kg dry solid.

For clarified broths containing negatively charged oligosaccharides specific anion exchange resins were used that do not retain the negatively charged oligosaccharides (containing a sialyl group). These resins are characterized to have a moisture content of 30-48% and preferably a gel type anion exchanger. Examples of such resins are DIAION SA20A, Diaion WA20A (Mitsubishi), XA4023 (Applexion), Dowex 1-X8 (Dow). In a first step the liquid was first passed through a strong acid cation exchange resin containing column (1 L of Amberlite IR120) in the proton form at a temperature of 10° C., resulting in exchange of all cations with a proton in the liquid. This was then passed immediately through an anion exchange resin column (1 L of XA4023), exchanging salts like phosphates and sulphates for hydroxide ions. The resulting liquid was set to a pH between 5.0 and 7.0. The ash content corrected for the sodium counter ions for the sialylated oligosaccharides was below 1% (on total dry solid) after ion exchange treatment, the lead content was lower than 0.1 mg/kg dry solid, the arsenic content was lower than 0.2 mg/kg dry solid, the cadmium content was lower than 0.1 mg/kg dry solid and the mercury content was lower than 0.5 mg/kg dry solid.

An alternative to sequential cation and anion exchange steps is mixed bed ion exchange. The resins are mixed in a ratio typically within the range of 35:65 and 65:35 volume percentage. Typically, a mixed bed ion exchange step is introduced in the process after a first de-ionization step such as a nanofiltration step, an electrodialysis step or ion exchange step but is also used as sole ion exchange step.

The clarified broth obtained after ultrafiltration of the oligosaccharide mixtures containing neutral fucosylated and/or non-fucosylated oligosaccharides was subjected to a mixed bed column of Amberlite FPC 22H and Amberlite FPA51 mixed in a ratio 1:1.3 on a 1 L column. The mixed bed step was performed at a temperature between 4° C. and 10° C. Finally, the liquid was set to a pH between 5.0 and 7.0 and the ash content of the solution was measured to be below 1%. The oligosaccharide recovery was between 95 and 98%. The clarified broths obtained after ultrafiltration of the oligosaccharide mixtures containing negatively charged oligosaccharides was subjected a mixed bed column of Diaion SA20A and Amberlite FPC 22H mixed in a ratio 1,3:1 on a 1 L column. Similar to the above the mixed bed step was performed at a temperature between 4° C. and 10° C. Finally, the liquid was set to a pH between 5.0 and 7.0 and the ash content of the solution was measured to be below 1%. None of the sialylated oligosaccharides were retained in this step, retaining the mixture composition, the oligosaccharide recovery was between 95 and 98%.

### Concentration Through Nanofiltration

Nanofiltration was carried out with an NF-2540 membrane (DOW) with a cut-off of 200 Da to concentrate the de-ionized solutions after ion exchange, electrodialysis or nanofiltration up to 25 Brix. During the filtration process a pressure across the membrane in the range of 20-25 bar was used and a process temperature of 45° C. The solution was continuous recirculated over the membrane for concentration, leading to a dry matter content of the concentrate up to 25% Brix.

### Colour Removal

To achieve decolourization, several samples throughout the process were subjected to activated charcoal treatment with Norit SX PLUS activated charcoal (0.5% m/v). Colour removal was measured with a spectrophotometer at 420 nm. In all samples the colour intensity at 420 nm was reduced 50 to 100-fold. The activated charcoal is filtered of by means of a plate filter or chamber filter press preferably at elevated temperatures.

### Example 30. Spray-Drying of a Solution Comprising a Single Oligosaccharide or of a Solution Comprising a Mixture of Di- and/or Oligosaccharides

In a next example, a solution comprising a single oligosaccharide or a solution comprising a mixture of di- and/or oligosaccharides can be dried by means of for example spray drying. Said mixture may comprise negatively charged and/or neutral di- and/or oligosaccharides. Said negatively charged and/or neutral di- and/or oligosaccharides may comprise fucosylated and/or non-fucosylated di- and/or oligosaccharides. Said mixture can e.g. comprise seven structurally different oligosaccharides such as e.g. 2′-FL, 3-FL, LNT, LNnT, LNFP-III, 3′-SL and 6′-SL. Said mixture can also e.g. comprise oligosaccharides such as 2′-FL, 3-FL, DiFL, LNT, LNnT, LNFP-I, LNFP-II, LNFP-III, LNFP-V, LNFP-VI, LNDFH-1, LNDFH-II, LNnDFH II, 3′-SL and 6′-SL. Before spray drying a solution, the Brix value of said solution, i.e. an approximation of the sugar content of said solution, is measured by e.g. the use of an appropriately calibrated refractometer, and the temperature of the solution is set at about 2 to about 60° C., preferably set at about 2 to about 30° C., more preferably at about 20 to about 30° C., even more preferably at about 20° C. Spray drying can be performed using a co-current spray dryer or in a counter-current spray dryer. The spray drying can comprise an atomizer wheel, a rotary disk, a high-pressure nozzle or a two-fluid nozzle.

In an example, a solution of a single oligosaccharide, like e.g. LNFP-III, dissolved in water can be spray dried using an SPX Anhydro CSD Type 70 (vol.=^{˜}43 m³) co-current spray dryer equipped with an atomizer wheel, applying the following parameters: inlet temperature: 135° C., outlet temperature 104° C., air inlet flow 3000 m³/h, atomizer speed 25 136 rpm. The solution is fed into the spray dryer at an initial rate of 80 kg/h, which is increased to 110 kg/h over the course of the first 3 hours of spray drying.

In another example, a solution comprising a mixture of oligosaccharides, like e.g. a mixture of LNT, LNFP-V and LNDFH-II, dissolved in water can be spray dried using a GEA Niro Production Minor (vol.=1.24 m3) co-current spray dryer equipped with an atomizer wheel, applying the following parameters: inlet temperature: 180° C., outlet temperature 104° C., air inlet flow 360 kg/h, atomizer speed 25 500 rpm. The solution is fed into the spray dryer at a rate of 5-15 kg/h.

The spray-dry process of the product can be repeated for multiple times, e.g., three to four times. The spray dryer can be attached to an external fluid bed. The spray-dried powder is then collected from the separation cyclone on the fluid bed and undergoes a second round of drying. When an agglomerated product is desired, the fluid bed provides a humid environment that causes small particles to clump together, thereby creating a dried powder with larger particle size. Said powder is less dusty and flows more readily, which makes it easier to handle. Water can be added by e.g. two fluid nozzles to create the agglomeration.

After each spray-dry process the bulk density of 100 g powder can be measured using a Jolting Stampfvolumeter (STAV 203, J. Engelsmann AG), a 250 mL measuring cylinder and a technical weighing scale. Bulk density is the weight of the particles of a particulate solid (such as a powder) in a given volume and expressed in grams per litre (g/L). Said bulk density of a powder comprises loose bulk density and tapped bulk density like e.g., a 100× tapped bulk density, a 625× tapped bulk density and a 1250× tapped bulk density. Loose bulk density (also known in the art as “freely settled” or “poured” bulk density) is the weight of a particulate solid divided by its volume where the particulate solid has been allowed to settle into that volume of its own accord (e.g. a powder poured into a container). Tapped bulk density (also known in the art as “tamped” bulk density) is the weight of a particulate solid divided by its volume where the particulate solid has been tapped and allowed to settle into the volume of a precise number of times. A “100× tapped bulk density” refers to the bulk density of the particulate solid after it has been tapped times. A “625× tapped bulk density” refers to the bulk density of the particulate solid after it has been tapped 625 times. A “1250× tapped bulk density” refers to the bulk density of the particulate solid after it has been tapped 1250 times.

Moisture content of the spray-dried powder can be measured using Karl-Fischer titration, wherein the quantity of Karl-Fischer solution absorbed by a sample indicates the amount of water in the sample, or via gravimetric methods, wherein a sample is dried and weight loss due to evaporation of solvent is measured at intervals. In an example, the moisture content of the spray-dried powder is less than 9% (by weight) water. In another example, the moisture content of the spray-dried powder is no greater than 5% (by weight) water. In another example, the spray drying process is operated to achieve a moisture content of from about 3.0 to 5.0% (by weight) water. In another example, the moisture content of the spray-dried powder is less than 3% (by weight) water.

The morphology of the spray-dried powder can be analysed using wide angle X-ray powder diffraction by an X-ray diffractometer like e.g. the X-ray diffractometer Empyrean (Panalytical, Almelo, the Netherlands). Differential Scanning Calorimetry can be used to determine thermal events of the spray-dried powder like e.g., glass transition temperature, further exo- and endothermic events. For this, 25 mg of the spray-dried powder is analysed in crimped Aluminium crucibles (Mettler Toledo). The samples are cooled to 0° C. with 10 K/min and reheated to 100° C. with a scanning rate of 10 K/min. After cooling down the samples to 0° C. again, the samples are heated to 150° C. in a second heating cycle. The midpoint of the endothermic shift of the baseline during a heating scan is taken as the glass transition temperature. Exothermic and endothermic peaks are reported by means of the peak temperature and the normalized energy of the event.

The powder particle size can be assessed by laser diffraction. The system detects scattered and diffracted light by an array of concentrically arranged sensor elements. The software-algorithm is then approximating the particle counts by calculating the z-values of the light intensity values, which arrive at the different sensor elements. The analysis can be executed using a SALD-7500 Aggregate Sizer (Shimadzu Corporation, Kyoto, Japan) quantitative laser diffraction system (qLD). A small amount (spatula tip) of each sample can be dispersed in 2 mL isooctane and homogenized by ultrasonication for five minutes. The dispersion will then be transferred into a batch cell filled with isooctane and analyzed in manual mode. Data acquisition settings can be as follows: Signal Averaging Count per Measurement: 128, Signal Accumulation Count: 3, and Interval: 2 seconds. Prior to measurement, the system can be blanked with isooctane. Each sample dispersion will be measured three times and the mean values and the standard deviation will be reported. Data can be evaluated using software WING SALD II version V3.1. When the refractive index of the sample is unknown, the refractive index of sugar (disaccharide) particles (1.530) can be used for determination of size distribution profiles. Size values for mean and median diameter are reported. The mean particle sizes for all samples are very similar due to the spray dryer settings used. In addition, the particle size distribution will show the presence of one main size population for all of the samples.

### Example 31. Spray Drying of an Oligosaccharide Mixture

A mixture of oligosaccharides at different concentrations was spray dried with pilot spray dry equipment. The equipment had an evaporation capacity of 25 kg/h.

For spray drying the liquid was heated to a temperature between 5° and 100° C., to lower the viscosity. The pH of the liquid was set to a pH of 4.0 to 6.0. More preferably the pH is set to 4.0 to 5.0 and temperatures are kept between 5° and 70° C.

The oligosaccharide concentration in the feed is between 20% and 80% Brix. These concentrations were obtained by rotary evaporation or wiped film evaporation. The concentrated liquids were fed to the spray dryer at a rate between 50% and 90%. The higher the percentage Brix, the faster the feed rate.

The used inlet temperature ranged between 120° C. and 280° C. The outlet temperature ranged between 100° C. and 180° C. The atomizer wheel rotation speed was set between 10000 and 28000 rpm. In one specific test the inlet temperature was set at 184° C., outlet temperature was set at 110° C. and atomizer rate was set at 21500 rpm.

The obtained powder had a white to off-white colour and after redissolving water at a concentration of 10%, the pH was between 4.0 and 6.0. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the lead content was lower than 0.1 mg/kg dry solid, the arsenic content was lower than 0.2 mg/kg dry solid, the cadmium content was lower than 0.1 mg/kg dry solid and the mercury content was lower than 0.5 mg/kg dry solid.

### Example 32. Stepwise Purification of a Fucosylated Oligosaccharide Mixture

The broth of each fermentation described in Examples 5 to 12, 14 to 19, 24 and 25 was clarified by first applying microfiltration with a 0.45 μm pore sized membrane, removing biomass at 60° C. and a pH of 4.0 to 5.0. The filtrate of the microfiltration step was in a second step subjected to ultrafiltration in which a PES membrane of 10 kDa was used, removing protein, endotoxin and DNA. The resulting filtrate was further concentrated by nanofiltration, partially removing salts and disaccharides from the liquid with a polyamide membrane of 300 to 500 Da at 40° C. In the nanofiltration step the oligosaccharide mixture was concentrated to a concentration of about 200 g/L or 20 Brix. The resulting concentrate was further decoloured by means of activated charcoal and de-ionized with a cation exchange step and an anion exchange step resulting in an ash content below 1% on dry mass. This de-ionized liquid was set to a pH between 5.0 and 7.0 and concentrated by means of evaporation to about 50 brix. The final solution was spray dried with an inlet temperature of 160° C., outlet temperature of 75° C., an airflow of 600 L/h and a feed-rate of 8 mL/min on a Procept spray dryer. The obtained powder had a white to off white colour and after redissolving water at a concentration of 10%, the pH was between 4.0 and 6.0. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the lead content was lower than 0.1 mg/kg dry solid, the arsenic content was lower than 0.2 mg/kg dry solid, the cadmium content was lower than 0.1 mg/kg dry solid and the mercury content was lower than 0.5 mg/kg dry solid. The oligosaccharides present in the powder obtained e.g. from the fermentation of the strains as described in Example 24 are LN3, LNnT and LNFP-III.

### Example 33. Stepwise Purification of a Sialylated and Fucosylated Oligosaccharide Mixture

The broth of each fermentation described in Examples 20 to 23 was clarified by first applying microfiltration with a 0.45 μm pore sized membrane, removing biomass at 60° C. and a pH of 4.0 to 5.0. The filtrate of the microfiltration step was in a second step subjected to ultrafiltration in which a PES membrane of 10 kDa was used, removing protein, endotoxin and DNA. The resulting filtrate was further concentrated by nanofiltration, partially removing salts and disaccharides from the liquid with a polyamide membrane of 300 to 500 Da at 40° C. In the nanofiltration step the oligosaccharide mixture was concentrated to a concentration of about 200 g/L or 20 Brix. The resulting concentrate was further decoloured by means of activated charcoal and de-ionized with a cation exchange step and an anion exchange step resulting in an ash content below 1% on dry mass. This de-ionized liquid was set to a pH between 5.0 and 7.0 and concentrated by means of evaporation to about 50 brix. The final solution heated to 70° C. and was spray dried with an inlet temperature of 184° C., outlet temperature of 104° C., atomizer speed 21800 rpm, at a feed rate of 66% on an Anhydro spray dryer. The obtained powder had a white to off white colour and after redissolving water at a concentration of 10%, the pH was between 4.0 and 7.0. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 1% (on total dry solid), the lead content was lower than 0.1 mg/kg dry solid, the arsenic content was lower than 0.2 mg/kg dry solid, the cadmium content was lower than 0.1 mg/kg dry solid and the mercury content was lower than 0.5 mg/kg dry solid. The oligosaccharides present in the powder obtained e.g. from the fermentation of the strains as described in Example 20 are 2′FL, DiFL, LNFP-I, LNFP-V, LNDFH-II, 3′SL, 6′SL, LSTa, LN3, LNT, 3'S-LN3 and 6'S-LN3.

### Example 34. Production of LNFP-III with a Modified S. cerevisiae Strain

An S. cerevisiae strain is adapted for production of GDP-fucose and LNnT and for expression of a fucosyltransferase as described in Example 4 with a first yeast expression plasmid comprising constitutive transcriptional units for the lactose permease LAC12 from K. lactis (UniProt ID P07921), the GDP-mannose 4,6-dehydratase gmd from E. coli (UniProt ID P0AC88), the GDP-L-fucose synthase fcl from E. coli (UniProt ID P32055) and a fucosyltransferase chosen from the list comprising SEQ ID NO 04, 05, 06, 07, 11, 13, 14, 15, 16, 17, 19, 21, 22, 23, 24, 25, 26, 28, 29, 31, 32, 34, 35, 36, 37, 38, 39, 40, 41, 42, 43, 44, 45, 46, 47 and 51 and with a second yeast expression plasmid comprising constitutive transcriptional units for the UDP-glucose 4-epimerase galE from E. coli (UniProt ID P09147), the galactoside beta-1,3-N-acetylglucosaminyltransferase IgtA from N. meningitidis (UniProt ID Q9JXQ6) and the N-acetylglucosamine beta-1,4-galactosyltransferase IgtB from N. meningitidis (UniProt ID Q51116, sequence version 02, 1 Dec. 2000). The novel strains are evaluated in a growth experiment for the production of LNnT and LNFP-III according to the culture conditions provided in Example 4, in which the SD CSM-Ura-His drop-out medium comprises glucose as carbon source and lactose as precursor. The strains are grown in four biological replicates in a 96-well plate. After 72 h of incubation, the culture broth is harvested, and the sugars are analysed on UPLC.

### Example 35. Production of LNFP-VI with a Modified S. cerevisiae Strain

An S. cerevisiae strain is adapted for production of GDP-fucose and LNnT and for expression of a fucosyltransferase as described in Example 4 with a first yeast expression plasmid comprising constitutive transcriptional units for the lactose permease LAC12 from K. lactis (UniProt ID P07921), the GDP-mannose 4,6-dehydratase gmd from E. coli (UniProt ID P0AC88), the GDP-L-fucose synthase fcl from E. coli (UniProt ID P32055) and a fucosyltransferase chosen from the list comprising SEQ ID NO 01, 02, 03, 07, 08, 09, 10, 12, 18, 20, 28, 31, 32, 33, 34, 35, 36, 48, 49 and 50 and with a second yeast expression plasmid comprising constitutive transcriptional units for the UDP-glucose 4-epimerase galE from E. coli (UniProt ID P09147), the galactoside beta-1,3-N-acetylglucosaminyltransferase IgtA from N. meningitidis (UniProt ID Q9JXQ6) and the N-acetylglucosamine beta-1,4-galactosyltransferase IgtB from N. meningitidis (UniProt ID Q51116, sequence version 02, 1 Dec. 2000). The novel strains are evaluated in a growth experiment for the production of LNnT and LNFP-VI according to the culture conditions provided in Example 4, in which the SD CSM-Ura-His drop-out medium comprises glucose as carbon source and lactose as precursor. The strains are grown in four biological replicates in a 96-well plate. After 72 h of incubation, the culture broth is harvested, and the sugars are analysed on UPLC.

### Example 36. Production of LNFP-V and LNDFH-II with a Modified S. cerevisiae Strain

An S. cerevisiae strain is adapted for production of GDP-fucose and LNT and for expression of a fucosyltransferase as described in Example 4 with a first yeast expression plasmid comprising constitutive transcriptional units for the lactose permease LAC12 from K. lactis (UniProt ID P07921), the GDP-mannose 4,6-dehydratase gmd from E. coli (UniProt ID P0AC88), the GDP-L-fucose synthase fcl from E. coli (UniProt ID P32055) and a fucosyltransferase chosen from the list comprising SEQ ID NO 01, 02, 03, 04, 05, 06, 07, 08, 09, 10, 11, 18, 26, 27, 28, 30, 31, 33 and 35 and with a second yeast expression plasmid comprising constitutive transcriptional units for the UDP-glucose 4-epimerase galE from E. coli (UniProt ID P09147), the galactoside beta-1,3-N-acetylglucosaminyltransferase IgtA from N. meningitidis (UniProt ID Q9JXQ6) and the N-acetylglucosamine beta-1,3-galactosyltransferase WbgO from E. coli O55:H7 (UniProt ID D3QY14). The novel strains are evaluated in a growth experiment for the production of LNT, LNFP-V and LNDFH-II according to the culture conditions provided in Example 4, in which the SD CSM-Ura-His drop-out medium comprises glucose as carbon source and lactose as precursor. The strains are grown in four biological replicates in a 96-well plate. After 72 h of incubation, the culture broth is harvested, and the sugars are analysed on UPLC.

### Example 37. Production of 3-FL with a Modified S. cerevisiae Strain

An S. cerevisiae strain is adapted for production of GDP-fucose and for expression of selected fucosyltransferases as described in Example 4 with a yeast expression plasmid (a variant of p2a_2μ_Fuc) comprising constitutive transcriptional units for LAC12 from K. lactis (UniProt ID P07921), gmd from E. coli (UniProt ID P0AC88), fcl from E. coli (UniProt ID P32055) and a fucosyltransferase selected from the list comprising SEQ ID NO 27, 28, 29, 31, 32, 34, 35, 36, 37, 38, 39, 40, 41, 43, 45, 49, 50 and 51. The mutant yeast strains are evaluated for production of 3-FL in a growth experiment according to the culture conditions described in Example 4 using SD CSM-Ura drop-out medium comprising lactose as precursor.

### Example 38. Production of an Oligosaccharide Mixture Comprising 2′FL, DiFL, LNFP-I, LNFP-II, LNFP-III, LNFP-V, LNDFH-II, LNFP-VI, LNnDFH II and 3-FL with a Modified S. cerevisiae Strain

An S. cerevisiae strain is adapted for production of GDP-fucose, LNT and LNnT and for expression of selected fucosyltransferases as described in Example 4 with a first yeast expression plasmid (a variant of p2a_2μ_Fuc) comprising constitutive transcriptional units for LAC12 from K. lactis (UniProt ID P07921), gmd from E. coli (UniProt ID P0AC88), fcl from E. coli (UniProt ID P32055), the fucosyltransferase with SEQ ID NO 31 and the a-1,2-fucosyltransferase from H. pylori (UniProt ID Q9X435) and with a second yeast expression plasmid comprising constitutive transcriptional units for the UDP-glucose 4-epimerase galE from E. coli (UniProt ID P09147), the galactoside beta-1,3-N-acetylglucosaminyltransferase IgtA from N. meningitidis (UniProt ID Q9JXQ6), the N-acetylglucosamine beta-1,3-galactosyltransferase WbgO from E. coli O55:H7 (UniProt ID D3QY14) and the N-acetylglucosamine beta-1,4-galactosyltransferase IgtB from N. meningitidis (UniProt ID Q51116, sequence version 02, 1 Dec. 2000). The mutant yeast strain is evaluated for production of an oligosaccharide mixture comprising 2′FL, DiFL, LNFP-1, LNFP-II, LNFP-III, LNFP-V, LNDFH-II, LNFP-VI, LNnDFH II and 3-FL in a growth experiment according to the culture conditions provided in Example 4, in which the SD CSM-Ura-His drop-out medium comprises glucose as carbon source and lactose as precursor. The strains are grown in four biological replicates in a 96-well plate. After 72 h of incubation, the culture broth is harvested, and the sugars are analysed on UPLC.

### Example 39. Production of an Oligosaccharide Mixture Comprising Fucosylated and Sialylated Oligosaccharide Structures with a Modified S. cerevisiae Strain

An S. cerevisiae strain is adapted for production of GDP-fucose, CMP-sialic acid and LNT and for expression of selected fucosyltransferases and sialyltransferases as described in Example 4 with a first yeast expression plasmid (a variant of p2a_2μ_Fuc) comprising constitutive transcriptional units for LAC12 from K. lactis (UniProt ID P07921), gmd from E. coli (UniProt ID P0AC88), fcl from E. coli (UniProt ID P32055), a fucosyltransferase with SEQ ID NO NO 01, 02, 03, 04, 05, 06, 07, 08, 09, 10, 11, 18, 26, 27, 28, 30 or 31 and the alpha-1,2-fucosyltransferase (HpFutC) from H. pylori (UniProt ID Q9X435) and with a second yeast expression plasmid (a pRS420-plasmid variant) comprising constitutive transcriptional units for the mutant glmS*54 from E. coli (differing from the wild-type E. coli glmS (UniProt ID P17169, sequence version 04, 23 Jan. 2007) by an A39T, an R250C and an G472S mutation), the phosphatase yqaB from E. coli (UniProt ID NP_417175.1), AGE from B. ovatus (UniProt ID A7LVG6), neuB from N. meningitidis (UniProt ID E0NCD4), neuA from P. multocida (UniProt ID A0A849C162), the alpha-2,3-sialyltransferase PmultST3 from P. multocida (UniProt ID Q9CLP3) and the alpha-2,6-sialyltransferase PdST6 from Photobacterium damselae (UniProt ID 066375), and with a third yeast expression plasmid comprising constitutive transcriptional units for the UDP-glucose 4-epimerase galE from E. coli (UniProt ID P09147), the galactoside beta-1,3-N-acetylglucosaminyltransferase IgtA from N. meningitidis (UniProt ID Q9JXQ6) and the N-acetylglucosamine beta-1,3-galactosyltransferase WbgO from E. coli O55:H7 (UniProt ID D3QY14). The mutant yeast strains are evaluated for production of an oligosaccharide mixture comprising 2′FL, DiFL, LNFP-1, LNFP-V, LNDFH-II, 3′SL, 6′SL, LSTa, LN3, LNT, 3'S-LN3 and 6'S-LN3, in a growth experiment according to the culture conditions described in Example 4 using SD CSM-Ura-Trp-His drop-out medium comprising lactose as precursor.

### Example 40. Material and Methods Bacillus subtilis

### Media

Two different media are used, namely a rich Luria Broth (LB) and a minimal medium for shake flask (MMsf). The minimal medium uses a trace element mix.

Trace element mix consisted of 0.735 g/L CaCl2·2H2O, 0.1 g/L MnCl2·2H2O, 0.033 g/L CuCl2·2H2O, 0.06 g/L CoCl2·6H2O, 0.17 g/L ZnCl2, 0.0311 g/L H3BO4, 0.4 g/L Na2EDTA·2H2O and 0.06 g/L Na2MoO4. The Fe-citrate solution contained 0.135 g/L FeCl3·6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH4)2SO4, 7.5 g/L KH2PO4, 17.5 g/L K2HPO4, 1.25 g/L Na-citrate, 0.25 g/L MgSO4.7H2O, 0.05 g/L tryptophan, from 10 up to 30 g/L glucose or another carbon source including but not limited to fructose, maltose, sucrose, glycerol and maltotriose when specified in the examples, 10 mL/L trace element mix and 10 mL/L Fe-citrate solution. The medium was set to a pH of 7 with 1M KOH. Depending on the experiment lactose, GlcNAc, LNB or LacNAc could be added as a precursor.

Complex medium, e.g. LB, was sterilized by autoclaving (121° C., 21′) and minimal medium by filtration (0.22 μm Sartorius). When necessary, the medium was made selective by adding an antibiotic (e.g. zeocin (20 mg/L)).

### Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., September 2008, p5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for the production of LacNAc, Bacillus subtilis mutant strains are modified with genomic knock-ins comprising constitutive transcriptional units for the mutant glmS*54 from E. coli (differing from the wild-type E. coli glmS, having UniProt ID P17169 (sequence version 04, 23 Jan. 2007), by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577), one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, DOG1 from S. cerevisiae and AraL from Bacillus subtilis as described in WO18122225 and an N-acetylglucosamine beta-1,4-galactosyltransferase LgtB from N. meningitidis (UniProt ID Q51116, sequence version 02, 1 Dec. 2000).

In an example for the production of lactose-based oligosaccharides, Bacillus subtilis mutant strains are created to contain a gene coding for a lactose importer (such as e.g. the E. coli lacY with UniProt ID P02920).

In an example for the production of lacto-N-triose (LNT-II, LN3, GlcNAc-β1,3-Gal-β1,4-Glc), the B. subtilis strain is modified with a genomic knock-in of constitutive transcriptional units comprising a lactose importer (such as e.g. the E. coli lacY with UniProt ID P02920) and a galactoside beta-1,3-N-acetylglucosaminyltransferase like e.g. LgtA from N. meningitidis (UniProt ID Q9JXQ6). For LNT production, the LN3 producing strain is further modified with a constitutive transcriptional unit for an N-acetylglucosamine beta-1,3-galactosyltransferase like e.g. WbgO from E. coli O55:H7 (UniProt ID D3QY14). For LNnT production, the LN3 producing strain is further modified with a constitutive transcriptional unit for an N-acetylglucosamine beta-1,4-galactosyltransferase like e.g. LgtB from N. meningitidis (UniProt ID Q51116, sequence version 02, 1 Dec. 2000). The N-acetylglucosamine beta-1,3-galactosyltransferase and the N-acetylglucosamine beta-1,4-galactosyltransferase can be delivered to the strain either via genomic knock-in or from an expression plasmid.

To further produce fucosylated oligosaccharides, the mutant B. subtilis strains are further modified with a constitutive transcriptional unit for a fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 and/or an alpha-1,2-fucosyltransferase like e.g. a polypeptide chosen from the list comprising UniProt IDs F8X274, A0A1B8TNT0 and Q316B5 or HpFutC from H. pylori (UniProt ID Q9X435).

In an example for sialic acid production, a mutant B. subtilis strain is created by overexpressing a fructose-6-P-aminotransferase like the native fructose-6-P-aminotransferase (UniProt ID P0CI73) to enhance the intracellular glucosamine-6-phosphate pool. Further on, the enzymatic activities of the genes nagA, nagB and gamA are disrupted by genetic knockouts and a glucosamine-6-P-aminotransferase like e.g. from S. cerevisiae (UniProt ID P43577), an N-acetylglucosamine-2-epimerase like e.g. from B. ovatus (UniProt ID A7LVG6) and an N-acetylneuraminate synthase like e.g. from N. meningitidis (UniProt ID E0NCD4) are overexpressed on the genome. To allow sialylated oligosaccharide production, the sialic acid producing strain is further modified with a constitutive transcriptional unit comprising an N-acylneuraminate cytidylyltransferase like e.g. the NeuA enzyme from P. multocida (UniProt ID A0A849C162), and one or more copies of a beta-galactoside alpha-2,3-sialyltransferase like e.g. PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity or PmultST2 from P. multocida subsp. multocida str. Pm70 (UniProt ID Q9CNC4), a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g. from M. musculus (UniProt ID Q64689).

For growth on sucrose, the mutant strains can additionally be modified with genomic knock-ins of constitutive transcriptional units comprising the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6).

### Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Example 41. Production of LNFP-V and LNDFH-II with a Modified B. subtilis Strain

A B. subtilis strain is first modified for LN3 production and growth on sucrose by genomic knock-out of the nagB, glmS and gamA genes and genomic knock-ins of constitutive transcriptional units comprising genes encoding the lactose permease (LacY) from E. coli (UniProt ID P02920), the native fructose-6-P-aminotransferase (UniProt ID P0CI73), the galactoside beta-1,3-N-acetylglucosaminyltransferase LgtA from N. meningitidis (UniProt ID Q9JXQ6), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is further modified with a genomic knock-in of a constitutive transcriptional unit comprising the N-acetylglucosamine beta-1,3-galactosyltransferase WbgO from E. coli O55:H7 (UniProt ID D3QY14) to produce LNT. In a subsequent step, the LNT producing strain is transformed with an expression plasmid comprising constitutive transcriptional units for a fucosyltransferase chosen from the list comprising SEQ ID NO 01, 02, 03, 04, 05, 06, 07, 08, 09, 10, 11, 18, 26, 27, 28, 30, 31, 33 and 35. The novel strains are evaluated for the production of an oligosaccharide mixture comprising LNFP-V and LNDFH-II in a growth experiment on MMsf medium comprising sucrose as carbon source and lactose as precursor according to the culture conditions provided in Example 40. After 72 h of incubation, the culture broth is harvested, and the sugars are analysed on UPLC.

### Example 42. Production of 3-FL with a Modified B. subtilis Strain

A B. subtilis strain is modified by genomic knock-out of the nagB, glmS and gamA genes and genomic knock-ins of constitutive transcriptional units comprising genes encoding the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is further modified with a genomic knock-in of a constitutive transcriptional unit comprising the lactose permease (LacY) from E. coli (UniProt ID P02920) and is transformed with an expression plasmid comprising constitutive transcriptional units for a fucosyltransferase chosen from the list comprising SEQ ID NO 27, 28, 29, 31, 32, 34, 35, 36, 37, 38, 39, 40, 41, 43, 45, 49, 50 and 51. The novel strains are evaluated for the production of 3-FL in a growth experiment on MMsf medium comprising sucrose as carbon source and lactose as precursor according to the culture conditions provided in Example 40. After 72 h of incubation, the culture broth is harvested, and the sugars are analysed on UPLC.

### Example 43. Production of an Oligosaccharide Mixture Comprising Fucosylated and Sialylated Oligosaccharides with a Modified B. subtilis Strain

A B. subtilis strain is modified by genomic knock-out of the nagA, nagB, glmS and gamA genes and genomic knock-ins of constitutive transcriptional units for the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417), the sucrose phosphorylase (BaSP) from B. adolescentis (UniProt ID A0ZZH6), the galactoside beta-1,3-N-acetylglucosaminyltransferase LgtA from N. meningitidis (UniProt ID Q9JXQ6), the N-acetylglucosamine beta-1,4-galactosyltransferase like e.g. LgtB from N. meningitidis (UniProt ID Q51116, sequence version 02, 1 Dec. 2000), the native fructose-6-P-aminotransferase (UniProt ID P0CI73), GNA1 from S. cerevisiae (UniProt ID P43577), the mutant glmS*54 from E. coli (differing from the wild-type E. coli glmS protein, having UniProt ID P17169 (sequence version 04, 23 Jan. 2007), by an A39T, an R250C and an G472S mutation as described by Deng et al. (Biochimie 2006, 88: 419-429)), a phosphatase like e.g. a phosphatase chosen from the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae or BsAraL from B. subtilis as described in WO18122225, AGE from B. ovatus (UniProt ID A7LVG6), the N-acetylneuraminate synthase (NeuB) from N. meningitidis (UniProt ID E0NCD4) and the N-acylneuraminate cytidylyltransferase NeuA from P. multocida (UniProt ID A0A849C162). In a next step, the strain is transformed with an expression plasmid comprising constitutive transcriptional units for the alpha-2,3-sialyltransferase PmultST3 from P. multocida (UniProt ID Q9CLP3) and the alpha-2,6-sialyltransferase PdST6 from Photobacterium damselae (UniProt ID 066375). In a further step, the mutant strain is transformed with a second compatible expression plasmid comprising constitutive transcriptional units for the alpha-1,2-fucosyltransferase HpFutC from H. pylori (UniProt ID Q9X435) and a fucosyltransferase with SEQ ID NO 04, 05, 06, 07, 11, 13, 14, 15, 16, 17, 19, 21, 22, 23, 24, 25, 26, 28, 29 or 31. The novel strain is evaluated for the production of an oligosaccharide mixture comprising 2′FL, DiFL, LNFP-III, LSTc, LSTd, 3′SL, 6′SL, LNnT, LN3, 3'S-LN3 and 6'S-LN3 in a growth experiment on MMsf medium comprising lactose according to the culture conditions provided in Example 40. After 72 h of incubation, the culture broth is harvested, and the sugars are analysed on UPLC.

### Example 44. Material and Methods Corynebacterium glutamicum

### Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl2, 10 g/L FeSO4·7H2O, 10 g/L MnSO4·H2O, 1 g/L ZnSO4·7H2O, 0.2 g/L CuSO4, 0.02 g/L NiCl2·6H2O, 0.2 g/L biotin (pH 7.0) and 0.03 g/L protocatechuic acid.

The minimal medium for the shake flasks (MMsf) experiments contained 20 g/L (NH4)2SO4, 5 g/L urea, 1 g/L KH2PO4, 1 g/L K2HPO4, 0.25 g/L MgSO4.7H2O, 42 g/L MOPS, from 10 up to 30 g/L glucose or another carbon source including but not limited to fructose, maltose, sucrose, glycerol and maltotriose when specified in the examples and 1 mL/L trace element mix. Depending on the experiment lactose, GlcNAc, LNB or LacNAc could be added as a precursor.

The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added.

Complex medium, e.g. TY, was sterilized by autoclaving (121° C., 21′) and minimal medium by filtration (0.22 μm Sartorius). When necessary, the medium was made selective by adding an antibiotic (e.g. kanamycin, ampicillin).

### Strains and Mutations

Corynebacterium glutamicum ATCC 13032, available at the American Type Culture Collection. Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for the production of LacNAc, the C. glutamicum strain is modified with a genomic knock-in of constitutive expression units comprising the mutant glmS*54 from E. coli (differing from the wild-type E. coli glmS, having UniProt ID P17169 (sequence version 04, 23 Jan. 2007), by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577), one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225 and an N-acetylglucosamine beta-1,4-galactosyltransferase LgtB from Neisseria meningitidis (UniProt ID Q51116, sequence version 02, 1 Dec. 2000).

In an example for the production of lactose-based oligosaccharides, C. glutamicum mutant strains are created to contain a gene coding for a lactose importer (such as e.g. the E. coli lacY with UniProt ID P02920).

In an example for the production of lacto-N-triose (LNT-II, LN3, GlcNAc-b1,3-Gal-b1,4-Glc), the C. glutamicum strain is modified with a genomic knock-in of constitutive expression units comprising a lactose importer (such as e.g. the E. coli lacY with UniProt ID P02920) and a galactoside beta-1,3-N-acetylglucosaminyltransferase like e.g. LgtA from N. meningitidis (UniProt ID Q9JXQ6). For LNT production, the LN3 producing strain is further modified with a constitutive transcriptional unit for an N-acetylglucosamine beta-1,3-galactosyltransferase like e.g. WbgO from E. coli O55:H7 (UniProt ID D3QY14). For LNnT production, the LN3 producing strain is further modified with a constitutive transcriptional unit for an N-acetylglucosamine beta-1,4-galactosyltransferase like e.g. LgtB from N. meningitidis (UniProt ID Q51116, sequence version 02, 1 Dec. 2000). The N-acetylglucosamine beta-1,3-galactosyltransferase and the N-acetylglucosamine beta-1,4-galactosyltransferase can be delivered to the strain either via genomic knock-in or from an expression plasmid.

To further produce fucosylated oligosaccharides, the mutant C. glutamicum strains are further modified with a constitutive transcriptional unit for a fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 and/or an alpha-1,2-fucosyltransferase like e.g. a polypeptide chosen from the list comprising UniProt IDs F8X274, A0A1B8TNT0 and Q316B5 or HpFutC from H. pylori (UniProt ID Q9X435).

In an example for sialic acid production, a mutant C. glutamicum strain is created by overexpressing a fructose-6-P-aminotransferase like the native fructose-6-P-aminotransferase (UniProt ID Q8NND3, sequence version 02, 23 Jan. 2007) to enhance the intracellular glucosamine-6-phosphate pool. Further on, the enzymatic activities of the genes nagA, nagB and gamA are disrupted by genetic knockouts and a glucosamine-6-P-aminotransferase like e.g. from S. cerevisiae (UniProt ID P43577), an N-acetylglucosamine-2-epimerase like e.g. from B. ovatus (UniProt ID A7LVG6) and an N-acetylneuraminate synthase like e.g. from N. meningitidis (UniProt ID E0NCD4) are overexpressed on the genome. To allow sialylated oligosaccharide production, the sialic acid producing strain is further modified with a constitutive transcriptional unit comprising an N-acylneuraminate cytidylyltransferase like e.g. the NeuA enzyme from P. multocida (UniProt ID A0A849C162), and one or more copies of a beta-galactoside alpha-2,3-sialyltransferase like e.g. PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity or PmultST2 from P. multocida subsp. multocida str. Pm70 (GenBank No. AAK02592.1), a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g. from M. musculus (UniProt ID Q64689). For growth on sucrose, the mutant strains can additionally be modified with genomic knock-ins of constitutive transcriptional units comprising the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g. LNFP-III concentrations, measured in the whole broth by the biomass, in relative percentages compared to the reference strain. The biomass is empirically determined to be approximately ⅓rd of the optical density measured at 600 nm.

### Example 45. Production of LNFP-V and LNDFH-II with a Modified C. glutamicum Strain

A C. glutamicum strain is first modified for LN3 production and growth on sucrose by genomic knock-out of the Idh, cgl2645 and nagB genes and genomic knock-ins of constitutive transcriptional units comprising genes encoding the lactose permease (LacY) from E. coli (UniProt ID P02920), the native fructose-6-P-aminotransferase (UniProt ID P0CI73), the galactoside beta-1,3-N-acetylglucosaminyltransferase LgtA from N. meningitidis (UniProt ID Q9JXQ6), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is further modified with a genomic knock-in of a constitutive transcriptional unit comprising the N-acetylglucosamine beta-1,3-galactosyltransferase WbgO from E. coli O55:H7 (UniProt ID D3QY14) to produce LNT. In a subsequent step, the LNT producing strain is transformed with an expression plasmid comprising constitutive transcriptional units for a fucosyltransferase chosen from the list comprising SEQ ID NO 01, 02, 03, 04, 05, 06, 07, 08, 09, 10, 11, 18, 26, 27, 28, 30, 31, 33 and 35. The novel strains are evaluated for the production of LNFP-V and LNDFH-II in a growth experiment on MMsf medium comprising sucrose as carbon source and lactose as precursor according to the culture conditions provided in Example 44. After 72 h of incubation, the culture broth is harvested, and the sugars are analysed on UPLC.

### Example 46. Production of 3-FL with a Modified C. glutamicum Strain

A C. glutamicum strain is modified by genomic knock-out of the ldh, cgl2645 and nagB genes and genomic knock-ins of constitutive transcriptional units comprising genes encoding the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is transformed with an expression plasmid comprising a constitutive transcriptional unit for a fucosyltransferase chosen from the list comprising SEQ ID NO 27, 28, 29, 31, 32, 34, 35, 36, 37, 38, 39, 40, 41, 43, 45, 49, 50 and 51. The novel strains are evaluated for the production of 3-FL in a growth experiment on MMsf medium comprising sucrose and lactose according to the culture conditions provided in Example 44. After 72 h of incubation, the culture broth is harvested, and the sugars are analysed on UPLC.

### Example 47. Production of LNFP-III with a Modified C. glutamicum Strain

A C. glutamicum strain is first modified for LN3 production and growth on sucrose by genomic knock-out of the Idh, cgl2645 and nagB genes and genomic knock-ins of constitutive transcriptional units comprising genes encoding the lactose permease (LacY) from E. coli (UniProt ID P02920), the native fructose-6-P-aminotransferase (UniProt ID P0CI73), the galactoside beta-1,3-N-acetylglucosaminyltransferase LgtA from N. meningitidis (UniProt ID Q9JXQ6), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is further modified with a genomic knock-in of a constitutive transcriptional unit comprising the N-acetylglucosamine beta-1,4-galactosyltransferase LgtB from N. meningitidis (UniProt ID Q51116, sequence version 02, 1 Dec. 2000) to produce LNnT. In a subsequent step, the LNnT producing strain is transformed with an expression plasmid comprising constitutive transcriptional units for a fucosyltransferase chosen from the list comprising SEQ ID NO 04, 05, 06, 07, 11, 13, 14, 15, 16, 17, 19, 21, 22, 23, 24, 25, 26, 28, 29, 31, 32, 34, 35, 36, 37, 38, 39, 40, 41, 42, 43, 44, 45, 46, 47 and 51. The novel strains are evaluated for the production of LNFP-III in a growth experiment on MMsf medium comprising sucrose as carbon source and lactose as precursor according to the culture conditions provided in Example 44. After 72 h of incubation, the culture broth is harvested, and the sugars are analysed on UPLC.

### Example 48. Production of an Oligosaccharide Mixture Comprising Fucosylated and Sialylated Oligosaccharide Structures with a Modified C. glutamicum Strain

A C. glutamicum strain is modified as described in Example 44 for LN3 production and growth on sucrose by genomic knock-out of the Idh, cgl2645, nagB, gamA and nagA genes and genomic knock-ins of constitutive transcriptional units comprising genes encoding the lactose permease (LacY) from E. coli (UniProt ID P02920), the native fructose-6-P-aminotransferase (UniProt ID Q8NND3, sequence version 02, 23 Jan. 2007), the galactoside beta-1,3-N-acetylglucosaminyltransferase LgtA from N. meningitidis (UniProt ID Q9JXQ6), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the mutant strain is further modified with a genomic knock-in of a constitutive transcriptional unit comprising the N-acetylglucosamine beta-1,3-galactosyltransferase WbgO from E. coli O55:H7 (UniProt ID D3QY14) to produce LNT. In a subsequent step, the LNT producing strain is transformed with an expression plasmid comprising a constitutive transcriptional unit for a fucosyltransferase with SEQ ID NO 01, 02, 03, 04, 05, 06, 07, 08, 09, 10, 11, 18, 26, 27, 28, 30 or 31 and for the alpha-1,2-fucosyltransferase HpFutC from H. pylori (UniProt ID Q9X435). In a next step, the mutant strain is further modified with a genomic knock-in of a constitutive transcriptional unit comprising GNA1 from S. cerevisiae (UniProt ID P43577), AGE from B. ovatus (UniProt ID A7LVG6), and the N-acetylneuraminate synthase from N. meningitidis (UniProt ID E0NCD4) to produce sialic acid. In a next step, the novel strain is transformed with an expression plasmid comprising constitutive transcriptional units for the NeuA enzyme from P. multocida (UniProt ID A0A849C162) and the beta-galactoside alpha-2,6-sialyltransferase PdST6 from P. damselae (UniProt ID 066375). The novel strain is evaluated for production of an oligosaccharide mixture comprising 2′FL, DiFL, LNFP-1, LNFP-V, LNDFH-II, 3′SL, 6′SL, LSTa, LN3, LNT, 3'S-LN3 and 6'S-LN3 in a growth experiment on MMsf medium comprising sucrose and lactose according to the culture conditions provided in Example 44. After 72 h of incubation, the culture broth is harvested, and the sugars are analysed on UPLC.

### Example 49. Materials and Methods Chlamydomonas reinhardtii

### Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na2EDTA·H2O (Titriplex III), 22 g/L ZnSO4·7H2O, 11.4 g/L H3B03, 5 g/L Mnl2·4H2O, 5 g/L FeSO4·7H2O, 1.6 g/L CoCl2·6H2O, 1.6 g/L CuSO4·5H2O and 1.1 g/L (NH4)6MoO3.

The TAP medium contained 2.42 g/L Tris (tris(hydroxymethyl)aminomethane), 25 mg/L salt stock solution, 0.108 g/L K2HPO4, 0.054 g/L KH2PO4 and 1.0 mL/L glacial acetic acid. The salt stock solution consisted of 15 g/L N4H4CL, 4 g/L MgSO4.7H2O and 2 g/L CaCl2·2H2O. As precursor for saccharide synthesis, precursors like e.g. galactose, glucose, fructose, fucose, GlcNAc, LNB and/or LacNAc could be added. Medium was sterilized by autoclaving (121° C., 21′). For stock cultures on agar slants TAP medium was used containing 1% agar (of purified high strength, 1000 g/cm2).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (https://www.chlamycollection.org), University of Minnesota, U.S.A.

Expression plasmids originated from pS1103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g. Scranton et al. (Algal Res. 2016, 15: 135-142). Targeted gene modification (like gene knock-out or gene replacement) can be carried using the Crispr-Cas technology as described e.g. by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×107 cells/mL. Then, the cells were inoculated into fresh liquid TAP medium in a concentration of 1.0×106 cells/mL and grown under continuous light for 18-20 h until the cell density reached 4.0×106 cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×107 cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575 Ω, 50 μFD). After electroporation, the cuvette was immediately placed on ice for 10 min. Finally, the cell suspension was transferred into a 50 mL conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analysed 5-7 days later.

In an example for production of UDP-galactose, C. reinhardtii cells are modified with transcriptional units comprising the gene encoding the galactokinase from Arabidopsis thaliana (KIN, UniProt ID Q9SEE5) and the gene encoding the UDP-sugar pyrophosphorylase (USP) from A. thaliana (UniProt ID Q9C511). In an example for production of LacNAc, C. reinhardtii cells modified for UDP-galactose production are further modified with an expression plasmid comprising a transcriptional unit for the N-acetylglucosamine beta-1,4-galactosyltransferase LgtB from N. meningitidis (UniProt ID Q51116, sequence version 02, 1 Dec. 2000).

In an example for LN3 production, a constitutive transcriptional comprising a galactoside beta-1,3-N-acetylglucosaminyltransferase like e.g. IgtA from N. meningitidis (UniProt ID Q9JXQ6). In an example for LNT production, the LN3 producing strain is further modified with a constitutive transcriptional unit comprising an N-acetylglucosamine beta-1,3-galactosyltransferase like e.g. WbgO from E. coli O55:H7 (UniProt ID D3QY14). In an example for LNnT production, the LN3 producing strain is further modified with a constitutive transcriptional unit comprising an N-acetylglucosamine beta-1,4-galactosyltransferase like e.g. LgtB from N. meningitidis (UniProt ID Q51116, sequence version 02, 1 Dec. 2000).

In an example for CMP-sialic acid synthesis, C. reinhardtii cells are modified with constitutive transcriptional units for an UDP-N-acetylglucosamine-2-epimerase/N-acetylmannosamine kinase like e.g. GNE from Homo sapiens (UniProt ID Q9Y223) or a mutant form of the human GNE polypeptide comprising the R263L mutation, an N-acylneuraminate-9-phosphate synthetase like e.g. NANS from Homo sapiens (UniProt ID Q9NR45) and an N-acylneuraminate cytidylyltransferase like e.g. CMAS from Homo sapiens (UniProt ID Q8NFW8). In an example for production of sialylated oligosaccharides, C. reinhardtii cells are modified with a CMP-sialic acid transporter like e.g. CST from Mus musculus (UniProt ID Q61420), and a Golgi-localised sialyltransferase chosen from species like e.g. Homo sapiens, Mus musculus, Rattus norvegicus.

In an example for production of GDP-fucose, C. reinhardtii cells are modified with a transcriptional unit for a GDP-fucose synthase like e.g. from Arabidopsis thaliana (GER1, UniProt ID 049213).

In an example for fucosylation, C. reinhardtii cells can be modified with an expression plasmid comprising a constitutive transcriptional unit for a fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 and/or an alpha-1,2-fucosyltransferase like e.g. HpFutC from H. pylori (UniProt ID Q9X435).

### Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analysed after 5 to 7 days of cultivation. For high-density cultures, cells could be cultivated in closed systems like e.g. vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 50. Production of LNFP-V and LNDFH-II in Modified C. reinhardtii Cells

C. reinhardtii cells are engineered as described in Example 49 for production of UDP-Gal and GDP-Fuc with genomic knock-ins of constitutive transcriptional units comprising the galactokinase from A. thaliana (KIN, UniProt ID Q9SEE5), the UDP-sugar pyrophosphorylase (USP) from A. thaliana (UniProt ID Q9C511) and the GDP-fucose synthase from Arabidopsis thaliana (GER1, UniProt ID 049213). In a next step, the mutant cells are transformed with an expression plasmid comprising transcriptional units comprising the galactoside beta-1,3-N-acetylglucosaminyltransferase IgtA from N. meningitis (UniProt ID Q9JXQ6), the N-acetylglucosamine beta-1,3-galactosyltransferase WbgO from E. coli O55:H7 (UniProt ID D3QY14) and a fucosyltransferase chosen from the list comprising SEQ ID NO 01, 02, 03, 04, 05, 06, 07, 08, 09, 10, 11, 18, 26, 27, 28, 30, 31, 33 and 35. The novel strains are evaluated in a cultivation experiment on TAP-agar plates comprising fucose, galactose, glucose and GlcNAc as precursors according to the culture conditions provided in Example 49. After 5 days of incubation, the cells are harvested, and the production of LNFP-V and LNDFH-II is analysed on UPLC.

### Example 51. Production of LNFP-III in Modified C. reinhardtii Cells

C. reinhardtii cells are engineered as described in Example 49 for production of UDP-Gal and GDP-Fuc with genomic knock-ins of constitutive transcriptional units comprising the galactokinase from A. thaliana (KIN, UniProt ID Q9SEE5), the UDP-sugar pyrophosphorylase (USP) from A. thaliana (UniProt ID Q9C511) and the GDP-fucose synthase from Arabidopsis thaliana (GER1, UniProt ID 049213). In a next step, the mutant cells are transformed with an expression plasmid comprising transcriptional units comprising the galactoside beta-1,3-N-acetylglucosaminyltransferase IgtA from N. meningitis (UniProt ID Q9JXQ6), the N-acetylglucosamine beta-1,4-galactosyltransferase IgtB from N. meningitidis (Uniprot ID Q51116, sequence version 02, 1 Dec. 2000) and a fucosyltransferase chosen from the list comprising SEQ ID NO 04, 05, 06, 07, 11, 13, 14, 15, 16, 17, 19, 21, 22, 23, 24, 25, 26, 28, 29, 31, 32, 34, 35, 36, 37, 38, 39, 40, 41, 42, 43, 44, 45, 46, 47 and 51. The novel strains are evaluated in a cultivation experiment on TAP-agar plates comprising fucose, galactose, glucose and GlcNAc as precursors according to the culture conditions provided in Example 49. After 5 days of incubation, the cells are harvested, and the production of LNFP-III and LNDFH-II is analysed on UPLC.

### Example 52. Production of 3-FL in Modified C. reinhardtii Cells

C. reinhardtii cells are engineered as described in Example 49 for production of UDP-Gal and GDP-Fuc with genomic knock-ins of constitutive transcriptional units comprising the galactokinase from A. thaliana (KIN, UniProt ID Q9SEE5), the UDP-sugar pyrophosphorylase (USP) from A. thaliana (UniProt ID Q9C511) and the GDP-fucose synthase from Arabidopsis thaliana (GER1, UniProt ID 049213). In a next step, the mutant cells are transformed with an expression plasmid comprising a transcriptional unit comprising a fucosyltransferase chosen from the list comprising SEQ ID NO 27, 28, 29, 31, 32, 34, 35, 36, 37, 38, 39, 40, 41, 43, 45, 49, 50 and 51. The novel strains are evaluated in a cultivation experiment on TAP-agar plates comprising fucose, galactose and glucose as precursors according to the culture conditions provided in Example 49. After 5 days of incubation, the cells are harvested, and the production of 3-FL is analysed on UPLC.

### Example 53. Materials and Methods Animal Cells

Isolation of Mesenchymal Stem Cells from Adipose Tissue of Different Mammals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% foetal bovine serum), and 1% antibiotics. The culture medium is subsequently replaced with 10% FBS (foetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% foetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

### Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 pg/mL insulin. At confluence, cells were fed with growth medium supplemented with 2% foetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

### Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling is performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO21067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/mL EGF, and 5 pg/mL hydrocortisone. Completed lactation media includes high glucose DMEM/F12, 1% NEAA, 1% pen/strep, 1% ITS-X, 1% F-Glu, 10 ng/mL EGF, 5 pg/mL hydrocortisone, and 1 pg/mL prolactin (5 ug/mL in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example the use of nickel resins to remove His-tagged growth factors, to further reduce the levels of contaminants in the lactated product.

### Example 54. Production of an Oligosaccharide Mixture Comprising Fucosylated Oligosaccharides in a Non-Mammary Adult Stem Cell

Isolated mesenchymal cells and re-programmed into mammary-like cells as described in Example 53 are modified via CRISPR-CAS to over-express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase (UniProt ID Q16222), the galactoside beta-1,3-N-acetylglucosaminyltransferase LgtA from N. meningitidis (UniProt ID Q9JXQ6), the N-acetylglucosamine beta-1,3-galactosyltransferase WbgO from E. coli O55:H7 (UniProt ID D3QY14), the GDP-fucose synthase GFUS from Homo sapiens (UniProt ID Q13630), the alpha-1,2-fucosyltransferase (HpFutC) from H. pylori (UniProt ID Q9X435) and a codon-optimized fucosyltransferase chosen from the list comprising SEQ ID NO 01, 02, 03, 04, 05, 06, 07, 08, 09, 10, 11, 18, 26, 27, 28, 30 and 31. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 53, cells are subjected to UPLC to analyse for production of an oligosaccharide mixture comprising 2′FL, DiFL, LNFP-1, LNFP-V and LNDFH-II.

### Example 55. Production of an Oligosaccharide Mixture Comprising Fucosylated and Sialylated Oligosaccharides in a Non-Mammary Adult Stem Cell

Isolated mesenchymal cells and re-programmed into mammary-like cells as described in Example 52 are modified via CRISPR-CAS to over-express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase (UniProt ID Q16222), the galactoside beta-1,3-N-acetylglucosaminyltransferase LgtA from N. meningitidis (UniProt ID Q9JXQ6), the N-acetylglucosamine beta-1,4-galactosyltransferase IgtB from N. meningitidis (Uniprot ID Q51116, sequence version 02, 1 Dec. 2000), the GDP-fucose synthase GFUS from Homo sapiens (UniProt ID Q13630), the alpha-1,2-fucosyltransferase (HpFutC) from H. pylori (UniProt ID Q9X435), a codon-optimized fucosyltransferase chosen from the list comprising SEQ ID NO 04, 05, 06, 07, 11, 13, 14, 15, 16, 17, 19, 21, 22, 23, 24, 25, 26, 28, 29 or 31, the N-acylneuraminate cytidylyltransferases from Mus musculus (UniProt ID Q99KK2), the CMP-N-acetylneuraminate-beta-1,4-galactoside alpha-2,3-sialyltransferase ST3GAL3 from Homo sapiens (UniProt ID Q11203) and the alpha-2,6-sialyltransferase (UniProt ID P13721) from Rattus norvegicus. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 53, cells are subjected to UPLC to analyse for production of an oligosaccharide mixture comprising 2′FL, DiFL, LNFP-III, LSTc, LSTd, 3′SL, 6′SL, LNnT, LN3, 3'S-LN3 and 6'S-LN3.

### Example 56. Production of 3-FL in a Non-Mammary Adult Stem Cell

Isolated mesenchymal cells and re-programmed into mammary-like cells as described in Example 52 are modified via CRISPR-CAS to over-express the GDP-fucose synthase GFUS from Homo sapiens (UniProt ID Q13630) and a codon-optimized fucosyltransferase chosen from the list comprising SEQ ID NO 27, 28, 29, 31, 32, 34, 35, 36, 37, 38, 39, 40, 41, 43, 45, 49, 50 and 51. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 53, cells are subjected to UPLC to analyse for production of 3-FL.

### Example 57. Production of LNFP-III, LNFP-VI and LNnDFH II with a Modified E. coli Strain

A mutant E. coli K12 MG1655 strain modified for production of GDP-fucose and LNnT (Gal-β1,4-GlcNAc-β1,3-Gal-β1,4-Glc) as described in Example 3 was transformed with an expression plasmid comprising a constitutive transcriptional unit for a fucosyltransferase selected from SEQ ID NO 08, 32, 33, 34, 35, 36, 37, 38, 39, 40, 41, 42, 43, 44, 45, 46, 47, 48, 49, 50 or 51. The novel strains were evaluated in a growth experiment for production of LNFP-III (Gal-β1,4-[Fuc-α1,3]-GlcNAc-β1,3-Gal-β1,4-Glc), LNFP-VI (Gal-β1,4-GlcNAc-β1,3-Gal-β1,4-[Fuc-α1,3]-Glc) and LNnDFH II (Gal-β1,4-[Fuc-α1,3]-GlcNAc-β1,3-Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured LNFP-III, LNFP-VI and LNnDFH II concentration was averaged over all biological replicates, and then normalized to the averaged concentrations of a reference strain expressing the fucosyltransferase with SEQ ID NO 08. As demonstrated in Table 12, all novel strains demonstrated to produce either LNFP-III, LNFP-VI, LNnDFH II or a combination of these sugar molecules.

TABLE 2
Relative production of LNFP-III
(Gal-β1,4-[Fuc-α1,3]-GlcNAc-β1,3-Gal-β1,4-Glc), LNFP-VI
(Gal-β1,4-GlcNAc-β1,3-Gal-β1,4-[Fuc-α1,3]-Glc) and/or
LNnDFH II (Gal-β1,4-[Fuc-α1,3]-GlcNAc-β1,3-Gal-β1,4-
[Fuc-α1,3]-Glc) (%) in mutant E. coli strains
expressing a fucosyltransferase
with SEQ ID NO 08, 32, 33, 34,
35, 36, 37, 38, 39, 40, 41, 42, 43, 44, 45, 46, 47, 48, 49, 50
or 51 and producing GDP-fucose and LNnT, when
evaluated in a growth experiment according
to the culture conditions provided in
Example 3, in which the culture medium
contained 30 g/L sucrose as carbon source and 20 g/L lactose, and
compared to a reference strain expressing
the fucosyltransferase with SEQ ID NO 08.

| | Fucosyltransferase | | | |
|---|---|---|---|---|
| Strain | expressed | LNFP-III (%) | LNFP-VI (%) | LNnDFH II (%) |
| 95 | SEQ ID NO 08 | 100 | 100 | 100 |
| 96 | SEQ ID NO 32 | 228 | 235 | 51 |
| 97 | SEQ ID NO 33 | 0 | 262 | 7 |
| 98 | SEQ ID NO 34 | 596 | 168 | 131 |
| 99 | SEQ ID NO 35 | 205 | 192 | 57 |
| 100 | SEQ ID NO 36 | 785 | 276 | 115 |
| 10 | SEQ ID NO 37 | 2027 | 0 | 0 |
| 102 | SEQ ID NO 38 | 1223 | 0 | 0 |
| 103 | SEQ ID NO 39 | 1208 | 0 | 0 |
| 104 | SEQ ID NO 40 | 1340 | 0 | 0 |
| 10 | SEQ ID NO 41 | 1683 | 0 | 0 |
| 106 | SEQ ID NO 42 | 564 | 0 | 0 |
| 107 | SEQ ID NO 43 | 748 | 0 | 0 |
| 108 | SEQ ID NO 44 | 671 | 0 | 0 |
| 109 | SEQ ID NO 45 | 1609 | 0 | 0 |
| 110 | SEQ ID NO 46 | 518 | 0 | 0 |
| 111 | SEQ ID NO 47 | 562 | 0 | 0 |
| 112 | SEQ ID NO 48 | 0 | 308 | 61 |
| 113 | SEQ ID NO 49 | 0 | 219 | 21 |
| 114 | SEQ ID NO 50 | 0 | 383 | 0 |
| 115 | SEQ ID NO 51 | 978 | 0 | 0 |

### Example 58. Production of 3-FL with a Modified E. coli Strain

A mutant E. coli K12 MG1655 strain modified for production of GDP-fucose as described in Example 3 was transformed with an expression plasmid comprising a constitutive transcriptional unit for a fucosyltransferase selected from SEQ ID NO 08, 32, 33, 34, 35, 36, 37, 38, 39, 40, 41, 42, 43, 44, 45, 46, 47, 48, 49, 50 or 51. The novel strains were evaluated in a growth experiment for production of 3-FL (Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, the measured 3-FL concentration was averaged over all biological replicates, and then normalized to the averaged 3-FL concentration of a reference strain expressing the fucosyltransferase with SEQ ID NO 08. As demonstrated in Table 13, the novel strains expressing a fucosyltransferase with SEQ ID NO 08, 32, 33, 34, 35, 36, 37, 38, 39, 40, 41, 43, 45, 49, 50 or 51 demonstrated to produce 3-FL.

TABLE 3
Relative production of 3-FL (Gal-β1,4-[Fuc-α1,3]-Glc)
(%) in mutant E. coli strains expressing a fucosyltransferase
with SEQ ID NO 08, 32, 33, 34, 35, 36, 37, 38,
39, 40, 41, 42, 43, 44, 45, 46, 47, 48,
49, 50 or 51 and producing GDP-fucose,
when evaluated in a growth experiment
according to the culture conditions
provided in Example 3, in which the culture
medium contained 30 g/L sucrose as
carbon source and 20 g/L lactose, and compared
to a reference strain expressing the
fucosyltransferase with SEQ ID NO 08.

| Strain | Fucosyltransferase expressed | 3-FL (%) |
|---|---|---|
| 116 | SEQ ID NO 08 | 100 |
| 117 | SEQ ID NO 32 | 133 |
| 118 | SEQ ID NO 33 | 325 |
| 119 | SEQ ID NO 34 | 62 |
| 120 | SEQ ID NO 35 | 11 |
| 121 | SEQ ID NO 36 | 243 |
| 122 | SEQ ID NO 37 | 34 |
| 123 | SEQ ID NO 38 | 23 |
| 124 | SEQ ID NO 39 | 10 |
| 125 | SEQ ID NO 40 | 3 |
| 126 | SEQ ID NO 41 | 17 |
| 127 | SEQ ID NO 42 | 0 |
| 128 | SEQ ID NO 43 | 5 |
| 129 | SEQ ID NO 44 | 0 |
| 130 | SEQ ID NO 45 | 7 |
| 131 | SEQ ID NO 46 | 0 |
| 132 | SEQ ID NO 47 | 0 |
| 133 | SEQ ID NO 48 | 0 |
| 134 | SEQ ID NO 49 | 12 |
| 135 | SEQ ID NO 50 | 29 |
| 136 | SEQ ID NO 51 | 2 |

### Example 59. Production of LNFP-V and LNDFH-II with a Modified E. coli Strain

A mutant E. coli K12 MG1655 strain modified for production of GDP-fucose and LNT (Gal-β1,3-GlcNAc-β1,3-Gal-β1,4-Glc) as described in Example 3 was transformed with an expression plasmid comprising a constitutive transcriptional unit for a fucosyltransferase selected from SEQ ID NO 08, 32, 33, 34, 35, 48, 49 or 50. The novel strains were evaluated in a growth experiment for production of LNFP-V (Gal-β1,3-GlcNAc-β1,3-Gal-β1,4-[Fuc-α1,3]-Glc) and LNDFH-II (Gal-β1,3-[Fuc-α1,4]-GlcNAc-β1,3-Gal-β1,4-[Fuc-α1,3]-Glc) according to the culture conditions provided in Example 3, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. For each strain with a particular fucosyltransferase tested, each of the measured LNFP-V and LNDFH-II concentration was averaged over all biological replicates, and then normalized to the averaged LNFP-V and LNDFH-II concentration, respectively, of a reference strain expressing the fucosyltransferase with SEQ ID NO 08. As demonstrated in Table 14, all novel strains demonstrated to produce LNFP-V and LNDFH-II.

TABLE 4
Relative production of LNFP-V (Gal-β1,3-
GlcNAc-β1,3-Gal-β1,4-[Fuc-α1,3]-Glc) (%) and LNDFH-II
(Gal-β1,3-[Fuc-α1,4]-GlcNAc-β1,3-Gal-β1,4-[Fuc-α1,3]-Glc) (%)
in mutant E. coli strains expressing a
fucosyltransferase with SEQ ID NO 08, 32, 33, 34, 35,
48, 49 or 50 and producing GDP-fucose and LNT, when
evaluated in a growth experiment according to the
culture conditions provided in Example 3,
in which the culture medium contained 30 g/L
sucrose as carbon source and 20 g/L lactose,
and compared to a reference strain expressing the
fucosyltransferase with SEQ ID NO 08.

| Strain | Fucosyltransferase expressed | LNFP-V (%) | LNDFH-II (%) |
|---|---|---|---|
| 137 | SEQ ID NO 08 | 100 | 100 |
| 138 | SEQ ID NO 32 | 397 | 0 |
| 139 | SEQ ID NO 33 | 245 | 7 |
| 140 | SEQ ID NO 34 | 434 | 0 |
| 141 | SEQ ID NO 35 | 236 | 66 |
| 142 | SEQ ID NO 48 | 757 | 0 |
| 143 | SEQ ID NO 49 | 99 | 0 |
| 144 | SEQ ID NO 50 | 391 | 0 |

### Example 60. Evaluation of a Mutant E. coli Strain in Fed-Batch Fermentations

In another experiment, the mutant E. coli strain 18 as described in Example 5 was evaluated in a fed-batch fermentation process. Fed-batch fermentations at bioreactor scale were performed as described in Example 3. Sucrose was used as a carbon source and lactose was added in the batch medium as precursor to the fermentation process. In contrast to the cultivation experiments that are described herein and wherein only end samples were taken at the end of cultivation (i.e., 72 hours as described herein), regular broth samples were taken at several time points during the fermentation process and the production of oligosaccharides was measured using UPLC as described in Example 3. Fermentations with the mutant strains expressing a fucosyltransferase with SEQ ID NO 28 demonstrated to produce an oligosaccharide mixture comprising LNnDFH II, and intermediates LN3, LNnT LNFP-III and LNFP-VI in whole broth samples taken in fed-batch phase. Results of relative production of LN3(%), LNnT (%), LNFP-III (%), LNFP-VI (%) and LNnDFH II (%) measured in whole broth samples are summarized in Table 15. For each strain, the relative production of LN3, LNnT, LNFP-III, LNFP-VI and LNnDFH II was calculated by dividing the production titres of LN3, LNnT, LNFP-III, LNFP-VI or LNnDFH II by the total sum of the production of LN3, LNnT, LNFP-III, LNFP-VI and LNnDFH II produced by that strain.

TABLE 5
Relative production of LN3 (%), LNnT (%), LNFP-III (%), LNFP-VI
(%) and LNnDFH II (%) measured in whole broth samples of mutant
E. coli strains adapted for production of GDP-fucose and LNnT
and for expression of a fucosyltransferase with SEQ ID NO 28,
and cultivated in a fed-batch fermentation performed at bioreactor
scale using sucrose and lactose as described in Example 3.

| | Fucosyl- | | | LNFP- | LNFP- | LNnDFH |
|---|---|---|---|---|---|---|
| | transferase | LN3 | LNnT | III | VI | II |
| Strain | expressed | (%) | (%) | (%) | (%) | (%) |
| 18 | SEQ ID NO 28 | 17.6 | 9.39 | 4.1 | 7.9 | 61.0 |

### Example 61. Evaluation of a Mutant E. coli Strain Producing an Oligosaccharide Mixture in Fed-Batch Fermentations

A mutant E. coli strain, engineered as described in Example 3, optimized for GDP-fucose, LNT and LNnT production, and expressing the a1,2-fucosyltransferase with UniProt ID A0A2N8IVT4 and the fucosyltransferase with SEQ ID NO 06 was evaluated in a fed-batch fermentation process. Fed-batch fermentations at bioreactor scale were performed as described in Example 3. Sucrose was used as a carbon source and lactose was added in the batch medium as precursor to the fermentation process. Regular broth samples were taken at several time points during the fermentation process and the sugar content was analysed on UPLC as described in Example 3. The concentration (in g/L) of each sugar was divided by the sum of the concentrations measured of all detected sugars, including the remaining lactose substrate concentration. Whole broth samples taken at the end of the fermentation showed production of 15% 2′FL, 1% 3FL, 5% DiFL, 5% LN3, 14% LNT, 17% LNnT, 1% pLNnH, 14% LNFP-1, 2% LNFP-III, 2% LNnFP-I and 7% multifucosylated LNT and/or LNnT (LNDFH). About 46% of the total sugar pool was fucosylated. Half of the fucosylated sugars consisted of (multi) fucosylated LNT and LNnT core structures, and the other half of small fucosylated structures like 2′FL, 3FL and DiFL.

1. A method for producing a fucosylated compound, said method comprising:
a) providing
i) GDP-fucose,
ii) a saccharide substrate comprising Gal-β1,m-GlcNAc-β1,n-Gal-β1,4-Glc wherein m is 3 or 4 and n is 3 or 6, optionally said saccharide substrate is linked to a peptide, a protein and/or a lipid, and
iii) a fucosyltransferase, wherein said fucosyltransferase has alpha-1,3-fucosyltransferase activity on N-acetylglucosamine (GlcNAc) and/or a glucose (Glc) residue of said Gal-β1,m-GlcNAc-β1,n-Gal-β1,4-Glc of said saccharide substrate, and:
comprises a polypeptide according to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
is a polypeptide comprising an amino acid sequence having 72.50% or more sequence identity to a full-length amino acid sequence of any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
comprises a functional fragment of a polypeptide according to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51, or
comprises a functional fragment comprising an amino acid sequence of at least 10 consecutive amino acid residues from any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51,
b) contacting said fucosyltransferase and GDP-fucose with said saccharide substrate under conditions where the fucosyltransferase catalyzes transfer of a fucose residue from said GDP-fucose to the GlcNAc and/or Glc residue of said Gal-β1,m-GlcNAc-β1,n-Gal-β1,4-Glc of said saccharide substrate in an alpha-1,3-glycosidic linkage resulting in the production of said fucosylated compound, and
c) optionally, separating said produced fucosylated compound.

2. The method according to claim 1, wherein said fucosylated compound comprises:
a saccharide comprising Gal-β1,m-[Fuc-α1,3]-GlcNAc-β1,n-Gal-β1,4-Glc, or
a saccharide comprising Gal-β1,m-GlcNAc-β1,n-Gal-β1,4-[Fuc-α1,3]-Glc, or
a saccharide comprising Gal-β1,m-[Fuc-α1,3]-GlcNAc-β1,n-Gal-β1,4-[Fuc-α1,3]-Glc.

3. The method according to claim 1, wherein said fucosylated compound comprises:
an oligosaccharide comprising Gal-β1,m-[Fuc-α1,3]-GlcNAc-β1,n-Gal-β1,4-Glc having a degree of polymerization of at least six,
an oligosaccharide comprising Gal-β1,m-GlcNAc-β1,n-Gal-β1,4-[Fuc-α1,3]-Glc having a degree of polymerization of at least six, or
an oligosaccharide comprising Gal-β1,m-[Fuc-α1,3]-GlcNAc-β1,n-Gal-β1,4-[Fuc-α1,3]-Glc having a degree of polymerization of at least seven.

4. The method according to claim 1, wherein said fucosylated compound is:
an oligosaccharide, a mammalian milk oligosaccharide (MMO), or a human milk oligosaccharide (HMO),
a negatively charged molecule, a sialylated molecule, a neutral molecule, a negatively charged oligosaccharide, a sialylated oligosaccharide, or a neutral oligosaccharide, and/or
selected from the group consisting of Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI), Gal-β1,4-[Fuc-α1,3]-GlcNAc-β1,3-Gal-β1,4-[Fuc-α1,3]-Glc (lacto-N-neodifucohexaose II, LNnDFH II), Gal-β1,3-GlcNAc-β1,3-Gal-β1,4-[Fuc-α1,3]-Glc (lacto-N-fucopentaose V, LNFP-V) and Gal-β1,3-[Fuc-α1,4]-GlcNAc-β1,3-Gal-β1,4-[Fuc-α1,3]-Glc (lacto-N-difucohexaose II, LNDFH-II).

5. The method according to claim 1, wherein said saccharide substrate comprises:
an oligosaccharide comprising Gal-β1,m-GlcNAc-β1,n-Gal-β1,4-Glc having a degree of polymerization of at least five,
an oligosaccharide, a mammalian milk oligosaccharide (MMO), and/or a human milk oligosaccharide (HMO),
a negatively charged molecule, a sialylated molecule, a neutral molecule, a negatively charged oligosaccharide, a sialylated oligosaccharide, or a neutral oligosaccharide, and/or
selected from the group consisting of Gal-β1,4-GlcNAc-β1,3-Gal-β1,4-Glc (lacto-N-neotetraose, LNnT), Gal-β1,3-GlcNAc-β1,3-Gal-β1,4-Glc (lacto-N-tetraose, LNT), Gal-β1,4-[Fuc-α1,3]-GlcNAc-β1,3-Gal-β1,4-Glc (lacto-N-fucopentaose III, LNFP-III), Gal-β1,4-GlcNAc-β1,3-Gal-β1,4-[Fuc-α1,3]-Glc (lacto-N-neofucopentaose V, LNFP-VI) and Gal-β1,3-[Fuc-α1,4]-GlcNAc-β1,3-Gal-β1,4-Glc (lacto-N-fucopentaose II, LNFP II).

6. The method according to claim 1, wherein said fucosyltransferase has additional alpha-1,3-fucosyltransferase activity on
a) a monosaccharide residue of said saccharide substrate excluding the GlcNAc and Glc residues of said Gal-β1,m-GlcNAc-β1,n-Gal-β1,4-Glc of said saccharide substrate, and/or
b) a compound that is different from said saccharide substrate, said compound being selected from the group consisting of a monosaccharide, a disaccharide and an oligosaccharide, optionally said compound is linked to a peptide, a protein and/or a lipid.

7. The method according to claim 1, wherein said fucosyltransferase has alpha-1,4-fucosyltransferase activity on said saccharide substrate and/or on a compound that is different from said saccharide substrate, said compound being selected from the group consisting of a monosaccharide, a disaccharide and an oligosaccharide, optionally said compound is linked to a peptide, a protein and/or a lipid.

8. The method according to claim 1, wherein said fucosyltransferase has alpha-1,3-fucosyltransferase activity on the GlcNAc residue of LNnT and
comprises the polypeptide according to any one of SEQ ID NO: 37, 41, 45, 40, 38, 39, 51, 36, 43, 44, 34, 42, 47, 46, 6, 32, 19, 35, 23, 14, 22, 21, 16, 4, 13, 15, 5, 7, 26, 31, 11, 29, 17, 25, 24, 28, 8, 9, 10, 33, 48, 49 or 50,
is a polypeptide comprising an amino acid sequence having 72.50% or more sequence identity to a full-length amino acid sequence of any one of SEQ ID NO: 37, 41, 45, 40, 38, 39, 51, 36, 43, 44, 34, 42, 47, 46, 6, 32, 19, 35, 23, 14, 22, 21, 16, 4, 13, 15, 5, 7, 26, 31, 11, 29, 17, 25, 24, 28, 8, 9, 10, 33, 48, 49 or 50,
comprises a functional fragment of a polypeptide according to any one of SEQ ID NO: 37, 41, 45, 40, 38, 39, 51, 36, 43, 44, 34, 42, 47, 46, 6, 32, 19, 35, 23, 14, 22, 21, 16, 4, 13, 15, 5, 7, 26, 31, 11, 29, 17, 25, 24, 28, 8, 9, 10, 33, 48, 49 or 50, or
comprises a functional fragment comprising an amino acid sequence of at least 10 consecutive amino acid residues from any one of SEQ ID NO: 37, 41, 45, 40, 38, 39, 51, 36, 43, 44, 34, 42, 47, 46, 6, 32, 19, 35, 23, 14, 22, 21, 16, 4, 13, 15, 5, 7, 26, 31, 11, 29, 17, 25, 24, 28, 8, 9, 10, 33, 48, 49 or 50.

9. The method according to claim 1, wherein said fucosyltransferase has:
a) alpha-1,3-fucosyltransferase activity on the Glc residue of LNnT and
comprises a polypeptide according to any one of SEQ ID NO: 3, 1, 12, 18, 2, 50, 20, 9, 48, 10, 36, 8, 33, 32, 49, 35, 34, 7, 31, 28, 5, 19 or 26, or
is a polypeptide comprising an amino acid sequence having 50.50% or more sequence identity to a full-length amino acid sequence of any one of SEQ ID NO: 3, 1, 12, 18, 2, 50, 20, 9, 48, 10, 36, 8, 33, 32, 49, 35, 34, 7, 31, 28, 5, 19, or 26,
comprises a functional fragment of a polypeptide according to any one of SEQ ID NO: 3, 1, 12, 18, 2, 50, 20, 9, 48, 10, 36, 8, 33, 32, 49, 35, 34, 7, 31, 28, 5, 19, or 26, or
comprises a functional fragment comprising at least 10 consecutive amino acid residues from any one of SEQ ID NO: 3, 1, 12, 18, 2, 50, 20, 9, 48, 10, 36, 8, 33, 32, 49, 35, 34, 7, 31, 28, 5, 19, or 26, or
b) alpha-1,3-fucosyltransferase activity on the GlcNAc residue and on the Glc residue of LNnT and
comprises a polypeptide according to any one of SEQ ID NO: 10, 9, 34, 36, 8, 7, 28, 31, 48, 35, 32, 19, 49, 26, 5, or 33,
is a polypeptide comprising an amino acid sequence having 50.50% or more sequence identity to a full-length amino acid sequence of any one of SEQ ID NO: 10, 9, 34, 36, 8, 7, 28, 31, 48, 35, 32, 19, 49, 26, 5, or 33,
comprises a functional fragment of a polypeptide according to any one of SEQ ID NO: 10, 9, 34, 36, 8, 7, 28, 31, 48, 35, 32, 19, 49, 26, 5, or 33, or
comprises a functional fragment comprising an amino acid sequence of at least 10 consecutive amino acid residues from any one of SEQ ID NO: 10, 9, 34, 36, 8, 7, 28, 31, 48, 35, 32, 19, 49, 26, 5, or 33, or
c) alpha-1,3-fucosyltransferase activity on the Glc residue of LNT and
comprises a polypeptide according to any one of SEQ ID NO: 48, 34, 32, 50, 3, 9, 33, 35, 1, 31, 28, 7, 49, 10, 18, 26, 2, 5, 8, 30, 6, 4, 27, or 11,
is a polypeptide comprising an amino acid sequence having 50.50% or more sequence identity to a full-length amino acid sequence of any one of SEQ ID NO: 48, 34, 32, 50, 3, 9, 33, 35, 1, 31, 28, 7, 49, 10, 18, 26, 2, 5, 8, 30, 6, 4, 27, or 11,
comprises a functional fragment of a polypeptide according to any one of SEQ ID NO: 48, 34, 32, 50, 3, 9, 33, 35, 1, 31, 28, 7, 49, 10, 18, 26, 2, 5, 8, 30, 6, 4, 27 or 11,
comprises a functional fragment comprising an amino acid sequence of at least 10 consecutive amino acid residues from any one of SEQ ID NO: 48, 34, 32, 50, 3, 9, 33, 35, 1, 31, 28, 7, 49, 10, 18, 26, 2, 5, 8, 30, 6, 4, 27, or 11, or
d) alpha-1,3-fucosyltransferase activity on the Glc residue of LNT and alpha-1,4-fucosyltransferase activity on the GlcNAc residue of LNT, and
comprises the polypeptide according to any one of SEQ ID NO: 3, 9, 33, 35, 8, 1, 10, 31, 28, 7, 18, 26, 2, 5, 30, 6, 4, 27, or 11,
is a polypeptide comprising an amino acid sequence having 50.50% or more sequence identity to a full-length amino acid sequence of any one of SEQ ID NO: 3, 9, 33, 35, 8, 1, 10, 31, 28, 7, 18, 26, 2, 5, 30, 6, 4, 27, or 11,
comprises a functional fragment of a polypeptide according to any one of SEQ ID NO: 3, 9, 33, 35, 8, 1, 10, 31, 28, 7, 18, 26, 2, 5, 30, 6, 4, 27, or 11, or
comprises a functional fragment comprising an amino acid sequence of at least 10 consecutive amino acid residues from any one of SEQ ID NO: 3, 9, 33, 35, 8, 1, 10, 31, 28, 7, 18, 26, 2, 5, 30, 6, 4, 27, or 11.

10. The method according to claim 1, wherein said fucosylated compound is produced by a cell.

11. The method according to claim 10, wherein said method comprises:
i) providing a cell expressing said fucosyltransferase,
ii) providing GDP-fucose, optionally said GDP-fucose is produced by said cell,
iii) providing said saccharide substrate comprising Gal-β1,m-GlcNAc-β1,n-Gal-β1,4-Glc, optionally said saccharide substrate is produced by said cell,
iv) cultivating and/or incubating said cell under conditions permissive to express said fucosyltransferase, optionally permissive to produce said GDP-fucose and/or said saccharide substrate, resulting in the production of said fucosylated compound, and
v) optionally, separating said fucosylated compound from said cultivation.

12. A method for producing a 3-fucosyllactose (3-FL), said method comprising:
a) providing GDP-fucose, lactose and a fucosyltransferase, wherein said fucosyltransferase has alpha-1,3-fucosyltransferase activity on a glucose (Glc) residue of said lactose, and:
comprises a polypeptide according to any one of SEQ ID NO: 36, 32, 34, 28, 31, 37, 50, 27, 38, 29, 41, 49, 35, 39, 45, 43, 40, or 51,
is a polypeptide comprising an amino acid sequence having 50.0% or more sequence identity to a full-length amino acid sequence of any one of SEQ ID NO: 36, 32, 34, 28, 31, 37, 50, 27, 38, 29, 41, 49, 35, 39, 45, 43, 40, or 51,
comprises a functional fragment of a polypeptide according to any one of SEQ ID NO: 36, 32, 34, 28, 31, 37, 50, 27, 38, 29, 41, 49, 35, 39, 45, 43, 40, or 51, or
comprises a functional fragment comprising an amino acid sequence of at least 10 consecutive amino acid residues from any one of SEQ ID NO: 36, 32, 34, 28, 31, 37, 50, 27, 38, 29, 41, 49, 35, 39, 45, 43, 40, or 51,
b) contacting said fucosyltransferase and GDP-fucose with said lactose under conditions where the fucosyltransferase catalyzes transfer of a fucose residue from said GDP-fucose to the Glc residue of said lactose in an alpha-1,3-glycosidic linkage resulting in the production of said 3-FL, and
c) optionally, separating said produced 3-FL.

13. The method according to claim 12, wherein said 3-FL is produced by a cell.

14. The method according to claim 13, wherein said method comprises:
i) providing a cell expressing said fucosyltransferase,
ii) providing GDP-fucose, optionally said GDP-fucose is produced by said cell,
iii) providing lactose, optionally said lactose is produced by said cell,
iv) cultivating and/or incubating said cell under conditions permissive to express said fucosyltransferase, optionally permissive to produce said GDP-fucose and/or said lactose, resulting in the production of said 3-FL, and
v) optionally, separating said 3-FL from said cultivation.

15. The method according to claim 10, wherein said cell is modified in expression or activity of any one of said fucosyltransferases.

16. The method according to claim 10, wherein said cell is able to produce one or more nucleotide-activated sugars selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-rhamnose and UDP-xylose and/or wherein said cell expresses one or more polypeptides selected from the group consisting of mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, optionally wherein said cell is modified in the expression or activity of any one of said polypeptides.

17. The method according to claim 10, wherein said cell expresses one or more glycosyltransferases selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

18. The method according to claim 10, wherein said cell is using one or more precursor(s) for the production of said fucosylated compound, said precursor(s) being fed to the cell from a cultivation medium and/or wherein said cell is producing one or more precursor(s) for the production of said fucosylated compound.

19. The method according to claim 18, wherein said precursor for the production of said fucosylated compound is completely converted into said fucosylated compound.

20. The method according to claim 10, wherein said cell is able to produce said saccharide substrate and/or a lactose.

21. The method according to claim 10, wherein said cell produces said fucosylated compound intracellularly and wherein a fraction or substantially all of said produced fucosylated compound remains intracellularly and/or is excreted outside said cell via passive or active transport.

22. The method according to claim 10, wherein said cell expresses a membrane transporter protein or a polypeptide having transport activity hereby transporting compounds across an outer membrane of a cell wall, optionally, said cell is modified in the expression or activity of said membrane transporter protein or polypeptide having transport activity.

23. The method according to claim 22, wherein said membrane transporter protein or polypeptide having transport activity is selected from the group consisting of porters, P—P-bond-hydrolysis-driven transporters, b-barrel porins, auxiliary transport proteins, MFS transporters, sugar efflux transporters, siderophore exporters, and phosphotransfer-driven group translocators.

24. The method according to claim 22, wherein said membrane transporter protein or polypeptide having transport activity controls a flow over the outer membrane of the cell wall of said fucosylated compound and/or of one or more precursor(s) to be used in said production of said fucosylated compound.

25. The method according to claim 22, wherein said membrane transporter protein or polypeptide having transport activity provides improved production and/or enabled and/or enhanced efflux of said fucosylated compound.

26. The method according to claim 10, wherein the cell comprises a catabolic pathway for selected mono-, di- or oligosaccharides, which is at least partially inactivated, the mono-, di-, or oligosaccharides being involved in and/or required for said production of said fucosylated compound.

27. The method according to claim 10, wherein said cell produces 90 g/L or more of said fucosylated compound in the whole broth and/or supernatant and/or wherein said fucosylated compound in the whole broth and/or supernatant has a purity of at least 80% measured on a total amount of said fucosylated compound and its precursor(s) in the whole broth and/or supernatant, respectively.

28. The method according to claim 10, wherein said cell is a bacterium, fungus, yeast, a plant cell, an animal cell, or a protozoan cell.

29. The method according to claim 10, wherein said cell is stably cultured in a medium.

30. The method according to claim 10, wherein said conditions comprise:
use of a culture medium comprising at least one precursor for producing said fucosylated compound with or without at least one precursor feed for producing said fucosylated compound and/or said 3-FL.

31. The method according to claim 10, the method comprising at least one of the following:
i) utilizing a culture medium comprising at least one precursor;
ii) adding to the culture medium in a reactor at least one precursor feed wherein a total reactor volume ranges from 250 mL (milliliter) to 10,000 m³ (cubic meter);
iii) adding to the culture medium in a reactor at least one precursor feed wherein the total reactor volume ranges from 250 mL (milliliter) to 10,000 m³ (cubic meter), optionally in a continuous manner, and optionally so that a final volume of the culture medium is not more than three-fold of the volume of the culture medium before the addition of the precursor feed; and/or
iv) adding at least one precursor feed in a continuous manner to the culture medium over the course of 1 to 5 day(s), by a feeding solution;
by a feeding solution;
said method resulting in said fucosylated compound with a concentration of at least 50 g/L in a final cultivation.

32. The method according to claim 13, the method comprising at least one of the following:
i) utilizing a culture medium comprising at least one precursor;
ii) adding to culture medium in a reactor at least one precursor feed wherein a total reactor volume ranges from 250 mL (milliliter) to 10,000 m³ (cubic meter);
iii) adding to culture medium in a reactor at least one precursor feed wherein total reactor volume ranges from 250 mL (milliliter) to 10,000 m³ (cubic meter), optionally in a continuous manner, and optionally so that a final volume of the culture medium is not more than three-fold of the volume of the culture medium before the addition of the precursor feed; and/or
iv) adding at least one precursor feed in a continuous manner to the culture medium over the course of 1 to 5 day(s), by a feeding solution;
said method resulting in said 3-FL with a concentration of at least 50 g/L in a final cultivation.

33. The method according to claim 10, the method comprising at least one of the following:
i) utilizing a culture medium comprising at least 50 grams of lactose per liter of initial reactor volume wherein the reactor volume ranges from 250 mL to 10,000 m³ (cubic meter);
ii) adding to culture medium a lactose feed comprising at least 50 grams of lactose per liter of initial reactor volume wherein the reactor volume ranges from 250 mL to 10,000 m³ (cubic meter);
iii) adding to culture medium a lactose feed comprising at least 50 grams of lactose per liter of initial reactor volume wherein the reactor volume ranges from 250 mL to 10,000 m³ (cubic meter), optionally in a continuous manner, and optionally so that a final volume of the culture medium is not more than three-fold of the volume of the culture medium before the addition of the precursor feed;
iv) adding a lactose feed in a continuous manner to a culture medium over a course of 1 to 5 day(s), by a feeding solution; and/or
v) adding a lactose feed in a continuous manner to a culture medium over the course of 1 to 5 day(s), by a feeding solution and wherein a concentration of said feeding solution is 50 g/L;
said method resulting in said fucosylated compound with a concentration of at least 50 g/L in a final volume of a cultivation.

34. The method according to claim 13, the method comprising at least one of the following:
i) utilizing a culture medium comprising at least 50 grams of lactose per liter of initial reactor volume wherein the reactor volume ranges from 250 mL to 10,000 m³ (cubic meter);
ii) adding to a culture medium a lactose feed comprising at least 50 grams of lactose per liter of initial reactor volume wherein the reactor volume ranges from 250 mL to 10,000 m³ (cubic meter);
iii) adding to a culture medium a lactose feed comprising at least 50 grams of lactose per liter of initial reactor volume wherein the reactor volume ranges from 250 mL to 10,000 m³ (cubic meter), optionally in a continuous manner, and optionally so that a final volume of the culture medium is not more than three-fold of the volume of the culture medium before the addition of the precursor feed;
iv) adding a lactose feed in a continuous manner to a culture medium over a course of 1 to 5 day(s), by a feeding solution; and/or
v) adding a lactose feed in a continuous manner to a culture medium over the course of 1 to 5 day(s), by a feeding solution and wherein a concentration of said feeding solution is 50 g/L;
said method resulting in said 3-FL with a concentration of at least 50 g/L in a final volume of a cultivation.

35. The method according to claim 33, wherein the lactose feed is accomplished by:
adding lactose from beginning of the cultivation in a concentration of at least 5 mM, and/or
adding lactose to the cultivation in a concentration, such, that throughout a production phase of the cultivation a lactose concentration of at least 5 mM is obtained.

36. The method according to claim 10, wherein said cell is cultivated:
for at least about 60 hours or in a continuous manner, and/or
in a culture medium comprising a carbon source comprising a monosaccharide, disaccharide, oligosaccharide, polysaccharide, polyol, glycerol, a complex medium including molasses, corn steep liquor, peptone, tryptone or yeast extract; optionally, wherein the carbon source is selected from the group consisting of glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate and/or wherein the culture medium contains at least one precursor selected from the group consisting of lactose, galactose, fucose, sialic acid, GlcNAc, N-acetylgalactosamine (GalNAc), LNB and N-acetyllactosamine (LacNAc).

37. The method according to claim 10, wherein a first phase of exponential cell growth is provided by adding a carbon-based substrate to a culture medium comprising a precursor, followed by a second phase wherein:
only a carbon-based substrate is added to the culture medium, or
a carbon-based substrate and a precursor are added to the culture medium.

38. The method according to claim 10, wherein the cell produces:
a mixture of negatively charged, optionally sialylated, and/or neutral di- and oligosaccharides comprising at least one of said fucosylated compound, or
a mixture of negatively charged, optionally sialylated, and/or neutral oligosaccharides comprising at least one of said fucosylated compound.

39. The method according to claim 13, wherein the cell produces:
a mixture of negatively charged, optionally sialylated, and/or neutral di- and oligosaccharides comprising 3-FL, or
a mixture of negatively charged, optionally sialylated, and/or neutral oligosaccharides comprising 3-FL.

40. The method according to claim 1, wherein said method comprises separation and wherein said separation comprises at least one of the following: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, and electrodialysis.

41. The method according to claim 1, further comprising purification of said fucosylated compound.

42.-50. (canceled)

51. A cell metabolically engineered to produce 3-fucosyllactose (3-FL), wherein said cell is able to express a fucosyltransferase, characterized in that said fucosyltransferase has alpha-1,3-fucosyltransferase activity on a glucose (Glc) residue of lactose, and:
comprises a polypeptide according to any one of SEQ ID NO: 36, 32, 34, 28, 31, 37, 50, 27, 38, 29, 41, 49, 35, 39, 45, 43, 40, or 51,
is a polypeptide comprising an amino acid sequence having 50.0% or more sequence identity to a full-length amino acid sequence of any one of SEQ ID NO: 36, 32, 34, 28, 31, 37, 50, 27, 38, 29, 41, 49, 35, 39, 45, 43, 40, or 51,
comprises a functional fragment of a polypeptide according to any one of SEQ ID NO: 36, 32, 34, 28, 31, 37, 50, 27, 38, 29, 41, 49, 35, 39, 45, 43, 40, or 51, or
comprises a functional fragment comprising an amino acid sequence of at least 10 consecutive amino acid residues from any one of SEQ ID NO: 36, 32, 34, 28, 31, 37, 50, 27, 38, 29, 41, 49, 35, 39, 45, 43, 40, or 51.

52.-62. (canceled)

63. A cell metabolically engineered to produce a fucosylated compound comprising a fucosylated version of a saccharide substrate comprising Gal-β1,m-GlcNAc-β1,n-Gal-β1,4-Glc, wherein m is 3 or 4 and n is 3 or 6, wherein the cell expresses a fucosyltransferase, wherein the fucosyltransferase
(a) has α-1,3-fucosyltransferase activity on an N-acetylglucosamine (GlcNAc) and/or a glucose (Glc) residue of Gal-β1,m-GlcNAc-β1,n-Gal-β1,4-Glc of a saccharide substrate comprising Gal-β1,m-GlcNAc-β1,n-Gal-β1,4-Glc, and
(b):
comprises a polypeptide of any one of SEQ ID NOs: 1-51;
is a polypeptide comprising an amino acid sequence having 72.50% or more sequence identity to a full-length amino acid sequence of any one of SEQ ID NOs: 1-51;
comprises a functional fragment of a polypeptide of any one of SEQ ID NOs: 1-51; or
comprises an amino acid sequence of at least 10 consecutive amino acid residues from any one of SEQ ID NOs: 1-51 and having fucosyltransferase activity, wherein said cell produces 90 g/L or more of said fucosylated compound and/or 3-FL in the whole broth and/or supernatant and/or wherein said fucosylated compound in the whole broth and/or supernatant has a purity of at least 80% measured on a total amount of said fucosylated compound and its precursor(s) in the whole broth and/or supernatant, respectively, and/or wherein said 3-FL in the whole broth and/or supernatant has a purity of at least 80% measured on a total amount of said 3-FL and its precursor(s) in the whole broth and/or supernatant, respectively.

64.-78. (canceled)

79. A dried powder comprising at least 50% w/w of a fucosylated compound selected from the group consisting of a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein m is 3 or 4 and n is 3 or 6, optionally said saccharide is linked to a peptide, a protein and/or a lipid.

80. (canceled)

81. A dried powder comprising a mixture of MMOs, wherein said mixture comprises 0.1 to 30% w/w, of one or more fucosylated compound(s) selected from the group consisting of a saccharide comprising Gal-β1,m-[Fuc-α1,3]-GlcNAc-β1,n-Gal-β1,4-Glc, a saccharide comprising Gal-β1,m-GlcNAc-β1,n-Gal-β1,4-[Fuc-α1,3]-Glc and a saccharide comprising Gal-β1,m-[Fuc-α1,3]-GlcNAc-β1,n-Gal-β1,4-[Fuc-α1,3]-Glc, wherein m is 3 or 4 and n is 3 or 6, optionally said saccharide is linked to a peptide, a protein and/or a lipid.

82.-89. (canceled)


SEQUENCE LISTING (51 sequences)

SEQ ID NO: 1 - protein, 319 aa, Chitinophaga eiseniae
MKLNFWNHLFKTDDHYTYNPLENGVEVLNFWPGQASKDLWLYQFVTARFGDILNKKNSLLLASLFGEKSHIRKSPSGTKVFFSGENLTRFPKYADYCTDFVDLAIGFDDIRRPGYIRFPLWLLFLFEPGSTYDTIKARMENISRDIKDIVPGKQQFCSLICSHDKNGNRKKLFNLLSTVDKVDSAGRFLNNTRALKEEYHDNKVDFLRQYRFNICPENTNRNGYVTEKVFEAIMCGCIPVYWGAGNHPEPEILNREAILFYNEKNPQALSKRVKTLQANPQAYTDFAMQDRFQPGAADRIYEMFVALEKGIGEKLLKRR

SEQ ID NO: 2 - protein, 321 aa, Parapedobacter luteus
MHIKRFFQKLGWYRQPAFYQYQPDENHIELLNFWTSQKRDEIWLYQFVRQHFSQYLTTERTLLLSSVFGPRKLITDSHSSIKVFYTGENVRRFHEYRDHCLPDVDLSLGFDDIRHPNYIRFPIWIFYFFGGGQTQRAIQRTLDQFVLPKPISIDARQFCSMVCSHDKNGIRGRLFKALSTVAPIASAGTYLTNTDILQTQYNDDKTHFLQAFQFNICPENTNHPGYVTEKIFQAIQADTVPIYWGSDNNPEPEVLNPDAILFYNGRGSVGTLTQQVEDLYRNPRKYAAFLAQPKFKPTAAEYIAHRIALLRQRLQQLFEGS

SEQ ID NO: 3 - protein, 335 aa, Chitinophaga arvensicola
MAKTSLWSLLKSLSGKFSSRETPYQYQPLASNIEVVNFWPTQRSEDLWLYLFVTSRLGELIDADNTMLLASVFGPRHYISESPSKIKIFYTGENVDRFPKYKDHCLDIVDLSFGFEYLQAPNYMRLPLWPSFVFRANADQREIQTEMDRIMNAIRSTPPASEHFCSLISSHDKSGVRSKLMKSLSGIGHIDSAGKFKNNCNALKEKYNDSKIDFMHNYQFNICPENTNKDGYVTEKVFEAIKAGTIPIYWGSNGRPEPEVLKQEAILFYNGPGSVKGLQQEVARLRQHPKQYEEFRNQDRFQPHAAEYLAEMLHTAEMKIRKIIKEKNALSGKWN

SEQ ID NO: 4 - protein, 325 aa, Azospirillum lipoferum
  source 1..325: note = 4B
MIDPRTSDFLAEFLASANRDPALLDRFLLHGPDRGGRGARPRLKIAFFDFWPDFDFSANFFVEILSSRFEVSVVENDCDLAIVSVFGTRHREARTARALFFTGENVRPPLDGVDMSVSFDRIDDPRHYRLPLYVMHAWEHLREGATPHFCHPVLPPAPPTREEAAKRKFCAFLYKNPNCARRNDFFQMLCARRHVESVGWLLNNTGSVVKMGWLPKIRVFSRYRFAFAFENASYPGYLTEKILDAFQAGAVPLYWGDPGVLRDVAAGSFIDMSRYSSDEEAIDAILAADDDYDTYRRYRGTAPFLGTEDFYFDAYRLAEWIESRL

SEQ ID NO: 5 - protein, 325 aa, Azospirillum oryzae
  source 1..325: note = A2P
MIDQRTSDFLSEFLASSHRDPARLDSFLLHGPGRGARAAKPRLKIAFFDFWPEFDPAANFFVDILSARFDVSVVDNDSDLAIVSVFGTRHREARTARSMFFTGENVRPPLDGVDMSVSFDRIDDPRHYRLPLYVMHAWDHRREGATPHFCQSVLPPVPPTREEAAKRKFCAFLYKNPNCARRNDFFQMLCARRHVESVGWLLNNTGSVVKMGWLPKIRVFSRYRFAFAFENASHPGYLTEKILDAFQAGAVPLYWGDPGVLRDVAAGSFIDVSRYASDEEACDAILAADDDYDTYRRYRSTPPFLGAEDFYFDAFRLAEWIESRL

SEQ ID NO: 6 - protein, 325 aa, Azospirillum brasilense
  source 1..325: note = LMG4388
MLDQRTSAFLEEFLAKPGGDPERLDRFLLHGPYRGRRGGKPRLKLAFHDFWPEFDTGTNFFIEILSGRFDLSVVEDDSDLAIVSVFGGRHRKARSRRTLFFTGENVRPPLDGFDMAVSFDRVEDPRHYRLPLYVMHAYEHMREGAVPHFCSPVLPPVPPTRAAFAERGFCAFLYKNPNGERRNRFFPALDGRRRVDSVGWHLNNTGSVVKMGWLAKIRVFERYRFAFAFENASHPGYLTEKILDVFQAGAVPLYWGDPDLEREVAAGSFIDVSRFATDEEAVEHILAVDDDYDAYCAHRAVAPFLGTEEFYFDAYRLADWIESRL

SEQ ID NO: 7 - protein, 329 aa, Basilea psittacipulmonis
  source 1..329: note = JF4266
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 8 - protein, 342 aa, Porphyromonas catoniae
MPIYDIKAMNTPSKQPLRERLHMMRRRNRVRKRSVIALIKSHLDSSRYQDYNWWDSHASTFWLPRFIDLHLEPKKKINLFSCFQNPLMLIRYYKGVKIFLSGENLTNNEHFGFHPRMLDHRINEVDLALGFEFRKDPKYYRFPLWIYQNEFISPSASLEDICVLVGQINDPSTRRSAKRSRFIGQISSHDKGGMRGRLIDLLSPIGQIDCAGKFRHNTDELLEVYGDDKFKYLANYRFNLCPENSLGEGYITEKVFDSIRAGCIPIYWGAYLEPGILNPKAILRFEEGKEQEFYNRVKELWENEEAYEQFILEPPFVEGAAERIWEILQGLRERLAPLVEEG

SEQ ID NO: 9 - protein, 323 aa, Porphyromonas sp.
  source 1..323: note = COT-239 OH1446
MNAVERVRNILNYCINEVQMYRQCPNSKYYNFWPCDYNNNWFNHFVEHRGLAKERHRLNFFSVFGNPLLPRIIPGKKVFFTGENLADNSIHSIGRAFKKTFPVYDLVLGFDYEVEDSRVNYMRFPLWIAFLIDPTADYQKIKETIERINDPSTRLNASRDRFACLVASHDKTGIRQKLYDVLMPIASVTCPGRFQNNTNELHDLYANDKREYLKLFKFNVCPENSSTPGYITEKLFDSFASGCIPIYFGGGTEEIEPDIVNQGAFIRYWDDGRMDWMDTVRELWESPSAYRAVAEIPPFKEQAADVIYAYMENLHDKLAAIVR

SEQ ID NO: 10 - protein, 316 aa, Selenomonas infelix
MLMRALRKMKRWGRVAFDYTNTTKDGAVCYHNWWPCNYEEEWFHRFVVQNIGTERCYHFFSVFGPRIALTLPTPNKVFFCGENVHNAEWPYKSYQDHALGDVKLALGYDDIQDERYIRFPLWLLYMFDPVVDRYAIRERIEEINHAENTRKYECVLISRHDKWNMRGPIYDALKDHLAISCAGKWKQNTDELWTVYNDDKPRYLKEFKFNICPENFDTPYYVTEKLFEAFRSGTIPIYAGGGDHPEPEIVNRSALLLWERGQSDHSALVQEVIRLARDEIYYDKFVHQVRLLPYTEEFVYEQFSSLKERLLQIRRG

SEQ ID NO: 11 - protein, 414 aa, Helicobacter bilis
  source 1..414: note = strain AAQJH
MNKRVLNMRILDWWNEDNEVNFYSNALIQLLQQKYDVAYSNTPDFILCGPFGYKHLEYDCVRIFYTGENVRPDFNLVDYAISFDYAVFEDRHLHAPLIFFNTYRSKELQNTLKSRIHLSKYKTKFCGFVASNGYMTEMRDNFFEALCAYKNVDSGGKWKNNIGTSVDDKIEWLKSYKFNICFENGSYPGYLTEKLFDAFMSGCVPIYWGDTSLRCGIDKECNETSNKDSKKDISPEVCTSNFFTRNYKYDENLDSNAMGGGGEYGIFDTRIPNIPPYLFDYKINPKAFINAHEFPTFKDLIDEIKRIDDNDKAFRDMLNEPIFLDDFDPQEFYSQRVFHFLDYIVSQGPTCAKRIGRGSYLQRKENVMKTCPYNIDSSITPKFMHYCMKHRNFIENIRKISEFPRDIIRIMRGK

SEQ ID NO: 12 - protein, 218 aa, Kingella denitrificans
MPFDNYYRFPVWVFYSWFKPSVSYGEIKACIREINNKNYRLANKQELKSSANSQFAALLARHDKLTTVRGDLVRLLNQIQPVCCAGKYLHNTDELQTKYQNDKQEYLRQFRFNICPENSDSEGYTTEKIFDAIRAGCIPIYWGSEGCPESEILNQDAILFYDPDNPDALLQQVRRLESDPEYYAEFISRPPFKEDAADKIWQMIDGLRDKLEKVINQH

SEQ ID NO: 13 - protein, 309 aa, Planctopirus limnophila
MQKTFSLRPVPVDAIRWNFANFWSGFDALAFERHLLGVSGKHKFRISEQNPQIVFESVFGTPGKGRERWPKARQVWYTGENVAPPLDQFDKCLSFHRDIKDPRHLRWPYYLLHLASLPMSFNDLVKCQSSVSTWAERPGFCAFIAFNEGCQTRNRFVEKLSRYRRVDCPGRVLNNMTSETLGQRGNLHGKINFLKQYKYAVCFENTSTRGSEGYVTEKLVDAMLAGCIPLYWGDHRVGEDFNENSFINLGVYGNDVNAMVQHVIELDSDERLQNNLFQEPWLPEIKSSEHFSFETSKDAILKLVANVNK

SEQ ID NO: 14 - protein, 314 aa, Polynucleobacter asymbioticus
MRSVRISTTGCPSDYSRGLFPIIAQSLGYQLHWVKPTEADLLILGPFLNGGRKKLSWCPKPLRPTMQSMINLVPARNHEPLRVFHSAENLRHDTVASDYSITFELNITSSNHFRLPYWMEMIDWSHEGVVGNANPRYGHLMKIERLMQPLGERFLEKPKAAAFLSSHIREPRKTLFEAVQKVMPVTGFGAHFNRNIKNHHLSGFLKQELLQEFGFNLCPENGMYPGYYTEKIPEAFFADCLPISWTDENVKVDFNPNAFINLAPMSWCNYTPLAEIINTPKLLEGYSSQALLLKAPSIEPFKNFVKNILEDACS

SEQ ID NO: 15 - protein, 327 aa, Helicobacter bilis
  source 1..327: note = ATCC 43879
MEDNLIIVRVDGGIASQIGFVALGKAFEEKGYQVKYDLSWFETSGKGFYNTINGYDRIYDLTFDMPKAFPQLEMKIASEDEVKRYNKLYFIDDEKVITHKPPLYVGGYLGRHYDIYFARHFATYFSPKEIEQKDAPFYILLQEILNTQSCGIHIRRGDLSQNHIVYGEPTSLTYFERVIQLVAQMNSKSVFYLFSDDVAWVREHIAPLLKDKQFKICDINTPEQGYLDLYLLSRCKVIVASHGSLGAYAKILAPHNPLLIAPRVRNVFFEMENVMLVNWGAKLQITQPCNNVITPPPHCQNLTLRYRLFLYLYNRLRSKLLRKGVIQ

SEQ ID NO: 16 - protein, 333 aa, Devosia insulae
  source 1..333: note = DS-56
MAGPLILFRTPYFRGPVPLAGVPDELRHHFTTDQARLREADAVVFHIPDWRPTQLDDTPKYPGQLWVLWSMESAVNYPRMADPDFLRHFDLSVTYERTADIWSSYLPGRAQWQAALAMPPPPKTETAPLVMFQSALIDGCGRNAFSGALMRQIPVDSYGRVYRNRTLSEDFGRQSKLGTISRYHFCLSLENSLLDDYVTEKLFDPLLAGTVPVYRGAPNAREFAPEHSFIDAEAYGGPRGLADYLRHLLESPGEYAAYFAWRQQPLPDWLEEKLTASATSFWLRLLERVDAARAIRRSGRPALPFGIKAAATARAMRLLRQLGGRRPPPVQLQ

SEQ ID NO: 17 - protein, 321 aa, unidentified
  source 1..321: note = delta proteobacterium NaphS2
MITVGIVKDWDWPDLLRQTPGEKGVWEGIRFVLSPEQTCDFLVVLNNKMKTETRIQCPPDNIWCLMQEPYMKGHTDWMVEKLDSYSKVLTHHVPSTLPKFTLSQPAVSWHVNKTFDQLVALEISAKPKTLSWVVGNAGDLPGHRKRLAFLEYIQKNSSLDIDLFGRAVQFIEDKWDGLAPYKYSLAIENSSSSDYWTEKIADCFLSWSVPIYYGCTNLENYFPPDSFIKINIDSPREAFESVKAIVAGDNWEKRLPALKEARDRVLHQYQLFPHLSRLIRSQPEKDLKPENLVLPAYRRSQKAAINHFFYKVLKFFGFPLQ

SEQ ID NO: 18 - protein, 316 aa, Pedobacter glucosidilyticus
MKYFLLLAKRHKKYLKEKKIFRNSTISFYNFWEIEDYNNFWLQKFIVDRNLNPKNKSINFFSVFGPRYVLKKQKAAINIFFSGETMSRFKKYHDYCLPEVDLALGFDDLQHEKYFRLPLWILDFFEPTVDLEKAKEKLKQLNYYKNNKPIVREKFCSLIARHDENGIRKKIVNTLNPIETVDCAGKLFNNTARLQTEFANNKVKFLENYKFNICPENTNQESYTTEKLFESFAAGCIPIYWGSAQKPEPNIFKPSSIIFFDEFKNTLSEDVERLHKDPKLYLDFISQNPFQDTAAEYIIQTISNLELKLKEIINQA

SEQ ID NO: 19 - protein, 325 aa, Azospirillum sp.
  source 1..325: note = B510
MIDQRTSDFLSEFLASPNRDPAVLDRFLLHGPERGGRAARPRLKIAFFDFWPEFDPSANFFVEILSSRFDVSVVDNDSDLAILSVFGERHREARTARALFFTGENVRPPLDGVDMSVSFDRIDHPRHYRLPLYVMHAWDHRREGATPHFCHPVLPPVPPTREEAAKRKFCAFLYKNPHCARRNDFFQMLCARRHVESVGWLLNNTGSVVKMGWLPKIRVFARYRFAFAFENAAHPGYLTEKILDAFQAGTVPLYWGDSGVLRDVAAGSFIDVSRYASDEEAIEAILAIDDDYDSYRRYRGTAPFLGTEDFYFDAYRLAEWIESRL

SEQ ID NO: 20 - protein, 321 aa, Paludibacter propionicigenes
MNISDSLKLFNFFFNRYSDDTRLNKNGQIRFYNFWDTQLPHEMWFTRFIQHHKLNSNYSAKINFYSVLGPAETLKYRRKGLNIFFSGENMHDERFDEYRLACEKKPFDLSIGFDGDNFGSNIRFPLWILYLFEPESTYEDIKVKVGRLSKKSADNRVRFCSLVASHDWNGIRTEIMDSLQSIDIVSSGGKFRQNTNELYTTYNDNKHEFLKQFKFNICPENSNAEGYITEKIFQSIEAGCIPVYWGANNRPEPEILNQDAIFCWNQGKDNTNLLNVINDLTMDENKYIDFASQQRFLPHAAEVIWSYFETLEQKMRYLTTQ

SEQ ID NO: 21 - protein, 325 aa, Azospirillum sp.
  source 1..325: note = TSH58
MLDQRTSAFLEDFLATPGGDPERLDRFLLHGPYRGRRGGKPRLKLAFHDFWPEFDAGTNFFIEILSSRFDLSVVEDDSDLAIVSVFGGRHRTARSRRTLFFTGENVRPPLDGFDMAVSFDRVDDPRHYRLPLYVMHAYEHMREGAVPHFCSPVLPPVPPTREAFAERNFCAFLYKNPNGERRNRFFPALDGRRRVDSVGWHLNNTGSVVRMGWLAKIRVFQRYRFAFAFENASHPGYLTEKILDVFQAGAVPLYWGDPDLEREVAAGSFIDVSRFASDEEAVEHILAVDDDYEAYRAHRAVAPFLGTEEFHFDAYRLADWIESRL

SEQ ID NO: 22 - protein, 325 aa, Azospirillum brasilense
  source 1..325: note = Sp7
MLDQRTSAFLEDFLAKPGGDPERLDRFLLHGPYRGRRGGRPRLKLAFHDFWPEFDKGTNFFIEILSSRFDLSVVEDDSDLAIVSVFGGRHREAHSRRTLFFTGENVRPPLDGFDMAVSFDRLDDPRHYRLPLYVMHAYEHMREGAVPHFCSPVLPPAPPTRAAFAERGFCAFLYKNPNGERRNRFFPALDGRRRVDSLGWHLNNTGSVVKMGWLAKIRVFERYRFAFAFENASHPGYLTEKILDVFQAGAVPLYWGDPDLEREVAAGSFIDVSRFATDEEAVDHILAVDGDYDAYCAHRAVAPFLGTEEFHFDAYRLADWIESRL

SEQ ID NO: 23 - protein, 325 aa, Azospirillum sp.
  source 1..325: note = TSH64
MIDQRTGVFLSEFLDTRNRDPAVLDRFLLQGPDGGRRGAKPNLKVAFFDFWPEFDPSANFFVEILSARFQVSVVENDSDLAIVSVFGTGPREIRTARSMFFTGENVRPPLDGIDMSVSFDRIDDPRHFRLPLYVVHAYDHLREGAAPYFCQPVLPPVPPTREDAAERKFCAFLYKNPNCARRNDFFHMLGARRHVDSVGWLLNNTGSVVKMGWLPKIRVFSRYRFAFAFENASHPGYLTEKILDAFQAGAVPLYWGDPGVLRDVAAGSFIDVSRYSSDEEAIEAILAIDDDYGAYRRYRSTPPFLGTEDFHFDAYRLAEWIESRL

SEQ ID NO: 24 - protein, 325 aa, Azospirillum sp.
  source 1..325: note = TSH100
MIDRRTSDFLAEFLASANKDPAVLDRFLLHGPDRGGRSAKPRLKIAFFDFWPEFDPAANFFVEILSARFDLSVVDNDSDLAIVSVFGIRHREARTARSLFFTGENVRPPLDGVDMSVSFDRIDDPRHYRLPLYVMHAWDHRREGATRHFCHSVLPPVPPTREEADRRKFCAFLYKNPNCERRNDFFRMLCARRHVESVGWLLNNTGSVVKMGWLPKIRVFSRYRFAFAFENASHPGYLTEKILDAFQAGAVPLYWGDPGVLRDVAAGSFIDVSRYSSDEEAIDAILAIDDDYDTYRRHRSTAPFLGTEDFYFDAFRLAEWIESRL

SEQ ID NO: 25 - protein, 325 aa, Azospirillum brasilense
  source 1..325: note = LMG 04385
MLDQRTSAFLEEFLAKPGGDPERLDRFLLHGPYRGRRGGRPRLKLAFYDFWPEFDTGRNFFIEILSSRFDLSVVEDDSDLAIVSVFGGRHRAARSRRTLFFTGENVRPPLDGFDMAVSFDRVGDPRHYRLPLYVMHAYEHMREGAVPHFCSPVLPPVPPSRAAFAERNFCAFLYKNPNGERRNRFFPALDARRRVDSVGWHLNNTGSVVKMGWLAKIRVFERYRFAFAFENASHPGYLTEKILDVFQAGAVPLYWGDPDVEREVAAGSFIDVSRFATDEEAAEHILALDGDYDAYCAYRAVAPFLGTEEFHFDAYRLADWIESRL

SEQ ID NO: 26 - protein, 350 aa, Haemophilus felis
  source 1..350: note = strain CCUG 35220
MGSRFSEKLTVIRYRIKYALSYYIFEQWKRIGKKPDVYFYNWWNSNREIQWFYRFIKARHIPLERSLNFFSVFGRKAVVSAYSVEGVKIFYTGEDVHKNAFGSYSEYQDYCLDSVDFALGFDKQTNKQTNKQTNKQTNKQTNKQTNYLRFPVWIFNFVDPHWSYEDLKAYIHQINSLENRANKDRACFMSHLARHDANGTRTKLVELFNQIEQVSCAGKLLNNTDELKTAFNDNKRTYLRKFRFNLCPENTSVEGYVTEKCLESIMSGCIPVYWGNDSAEIEPEILNQNAILRYTENNEAELVAKVRELYSSETAYQAFIQQKPFKENAADVIWEKLQQLEKRLTDLARE

SEQ ID NO: 27 - protein, 429 aa, Psittacicella hinzii
  source 1..429: note = strain B96_3
MADINHCGYWLGFSLEKQKSIFLYHKFDLQKLPHNLFLVSGFLRRFWTQSELERRASGYQEKLGWAPLLYHGYPYNAASCLGIPYKLKHGTSERSFYYDQEHSLEFAFSENSQDIYADYLDLALGIYLPEVISTRYCNYYHAPNYLVSQSVGLNTLYGLPTYAQVEAWIESFEQARRQAEVFKRPALSVCISRHDSLSGLRGVRGLIGDLFTEVTKDLHEAPVFYAGYFRNNTSDLHEHFNDDKIAYGKNFVFMICPENSYCPGYVSEKILEAFASGCIPIYWGGIESELDYFNEKAFVYFDPRKPKEFQERIRELVYDQGKLEEMIAQPCFLPGAAARIYLRYIYPIAVAFSNLMDGLNPYQLLKEPVDTTLEQEKAKAEAVLAQLGLVENKYQGLDRLPFKYYVDFEQYNEEEKQRLEYFVRHYNSK

SEQ ID NO: 28 - protein, 327 aa, Methylovirgula ligni
  source 1..327: note = strain BW863
MIFKALEFFTRRSPGARTPATDRQIASEALAICAVDFWPAFSLSGGFWNFVLTESFGQFRVVDEAADADIVLASVFPHEKAQFPEKTIAIIWENIRPNYEFYRFSISSDFDAYQGRNCRVPNWYEELVWNESFRGPKAAFAGAMHGHGHEERVEIERLLAPGEAEPVVRSKFCCLVTSYREPYRALAVEALSQIGPVDIFGNVAGAPLLQSKYEALRDYRFNLCFENSIFPGYYTEKALQAWAAGCIPLYFSDPYFSADFNPKAIINRIDFRELAEFVEAVRRVNDSPDLMAQFHREPLLTSRPNLDSVTAFLRGAAKAITGRDIGG

SEQ ID NO: 29 - protein, 337 aa, Lewinella xylanilytica
  source 1..337: note = strain
MFRPLKQPVKNVVSGLIGEERLTAYNRYARQTAGAYYQHFLLRCYGRGFYNFWRVPSFRNTWFWRFLDRPRLAKLSKDRRIAFISVFGDPRLISRINADLRVFFTGENLDFHPSYRDHLFGSVDLSLGFDQLQRSDYLRFPIWLLLCFDPGMQLADITERLRQWDRNRLDLLQRPAMVASLVASHDPSGSRRRIADLFDQVGTVRYGGAFRNVGERVPPGQAYKHRFIRQYPFHICPENSQRPGYTTEKLFEAVAAGCIPIYRGAEGAVEPDIFNQDAIIAYSPGEETVFRQRLRSLRDDPHQLRDLRGIPPFRPDAAEHIYNFYLRLEEKLFDLLG

SEQ ID NO: 30 - protein, 320 aa, unidentified
  source 1..320: note = seawater metagenome SRR7054517- ERZ650666 from Marineland, Florida, USA
MGLKTNIKNTIADYKRFRKDQRKFCNDSVRYYNFWHLPNSESGWFTQFIKHRNINPTGKKITFLSVVGNPKLSFFIKSPKIFFTGENIDNEEHPSFIFKTHHLKNHNLSLGFSYLNDKKYLRFPLWILYMIPANAGFEDIKNIIKSYNNPEHRLNKNRNNFCSNISRADNNGIRKTIIDLLNPVETVKCAGSFLKNTNDLQEKFGDNKIEYLKTFKFNICPENSDTNGYVTEKLFEAIISGTIPIYWGSENKPDPMIINQDAVLFFDPENPQLLFDKVNLLWNDEKVYEEFCRIKPFQENAAEVIWEWLNSLESKIKSTL

SEQ ID NO: 31 - protein, 327 aa, unidentified
  REGION 1..327: note = Unknown
MIRNIKNKLKNTIHLLKVYINSGQINFYNFWTENNIDDYWLYRFVKSRKINNKISFFSVFGKRFIVDYSFGVGKKVFFTGENLTETNIFFLGYAPKYKNHCLNSVDLSLGFDYIDHPKYMRFPLWIKYLTNGTEQSINDIEEKINNINNPRHRLSKGRNYFASLISRHDEGGTRMKLINLLNPINKVVCAGTFMNNTNNLKEKFNDNKLEFLKQFKFNICPENSRANGYITEKIFEAIISGCIPIYYGGRDTSNTNEAKQSIEPEILNPEAFIFYNGNNEEEVFAKVKELWENQSAYEEFCKIPPFKENAAKVIWSKLQELEDRLKK

SEQ ID NO: 32 - protein, 316 aa, Bacteroides bouchesdurhonensis
  source 1..316: note = isolate MGYG-HGUT-00098
MKLKINFADFWPTFDRHCNFFIELLSKKYEVEVCEDPDYLIYSIFGYENLQYDSCVKIFYVGENITPDFNLCDYAIGFDLMEFGDRYMRLPYYVLYDIEKLATPKIIDAEAVLNRKFCSFVVSNANASPERNRFFHLLSEYKKVDSGGRFENNVGGPVENKREFISQYKFNIAFENSMRDGYTTEKIMEPMLVNSLPIFWGNRKVGLDFNPSSFIDASDYPSLEALVEHIVQLDTNDEEYLSILSEPWLNQDNYLDWKERLVAFFDRIIDKPREEQKYLSPYGYGNNYRMRLRRMLRAYKSKEKLKNIYKPKYWFK

SEQ ID NO: 33 - protein, 343 aa, Butyrivibrio sp.
  source 1..343: note = TB
MNIIHFYARYLRESHNWNREREVTRNGVMTFANWWREDPHKNWFARFIDAGSKDPERRIRFYSIFGPYSKLKEDFDGAKIFFSGENLEQPVYHRILKTDPIEDRIWADRRKLYGNYGAGDVDLAIGFGNREEDSLMGFEGSRKTKYIRFPLWLTYVFDPDCTHDDIKRTIDEINAVRSTGRKDTLLLASHDFWGTRSDILKSLEGVCDVSIAGKWRNNTKELWEDYNNDKNKYLSEFKFNICPENVDAPGYVTEKIFDAFKCGAIPIYQGCLGKPEPDVINTDAVLLWDFDGDNSDTISLIKKLNSDNVYYDNFVSQPKFKPDAAEYVVACMDELRRSFDQLI

SEQ ID NO: 34 - protein, 325 aa, Lewinella lacunae
MKPRGRAIIRAAKQWTKDGLAVARGLQQAQRYGVGYYNFWDLGPVEHSWFYRFLQRPALAALARRRKVGFYSVFGRRRLIGWGRERRKVFFTGENLAHYPAYHDHALGEVELALGFEYLDHPHYLRFPLWMLDQFDPTADYPGLKSQLSVYERAGEDVSARPGFASLVARHDNNGIRGAIGDVAATLGPVNYPGAFRCNQEKGLAEGNAAKLAHLRNYRYNLCPENSNAAGYVTEKVFHAIAAGCIPVYWGADNVPEPEVLRQEAILFYDPARPLALREALEELERKPAALAAFARQPRFQPTAAGFLHQQYLALEKQLLRVLRG

SEQ ID NO: 35 - protein, 352 aa, Porphyromonas catoniae
MLAPYKSPIFVPIYDTKAMNPPTKQPLRERLHMMRRRNRIRKRSVIALIKSHLDSSRYQDYNWWDSHASTFWLPRFIDLHLEPKKRINLFSCFQNPLMLIRYYKGVKIFLSGENLANNEHFGFHPRMLDHRINEVDLALGFEFRKDPKYYRFPLWIYQNEFISPSASLEDIRALLEQINDPSTRRSTGRSRFIGQISSHDKGGMRGRLIDLLNPIGQIDCAGKFRHNTDELLEVYGDDKFKYLANYRFNLCPENSLGEGYITEKVFDSIRAGCIPIYWGAYLEPGILNPKAILRFEEGKEQEFYNRVKELWENEAAYEQFILEPPFVEGAAERIWEILQGLRERLAPLVEEG

SEQ ID NO: 36 - protein, 329 aa, Porphyromonas sp.
MSNRYKSFMQSFWNSDNARTIRSSIRTLLWSYFGKNQYRLVNSWGNDANRFWITQFIEQHFSTNKKVLMCSCFDYPCRYLRFNKAIRIFFTGENLQHSIVPNRTQWYGDHRLKDVDLALGFEHKQESNYIRFPQWIWHRDFISPKATLEDIKQKISNINRLETRLNSNRTRFIAQISSHDLGGIRMSLIDLLDPIGQIDCAGGFNRNTDELQTVYNDKKEDFLSNYRFCICPENSLGDGYITEKLFESIASGCIPIYWGACLEEGILNPKAILRYEKGKEKELFEQVQRLWKDENAYKEFAMQPPFLDGAAEKIWEMLENLKKKLKPLF

SEQ ID NO: 37 - protein, 314 aa, Polynucleobacter sp.
  source 1..314: note = 86C-FISCH
MRSVRISTAGCPSDYSQGLLPIIAQSLGYQLDWVKPNQADLLILGPFTKPDSKKLKWCPKPLRPTVNSILNLTPSRANSPLRVFHSAENLRHDTVPAEYSISFDLNISSEQHFRLPYWMEMIDWSHEGIAGNQNPRYGKLMSIERLMQPLGNRFLEKKQAAAFLSSHIREPRKTLFEAVQKVMPVTGFGAHFDKTIENHHLSGFLKQELLQEFGFNLCPENGMYPGYYTEKIPEAFFADCLPITWTDENVRVDFNPQAFINLAPMTWCNYEPLAEIIHSPQALQSYTAQALLLKAPSILPFRDFVKRILEDATT

SEQ ID NO: 38 - protein, 314 aa, Polynucleobacter sp.
  source 1..314: note = MG-5-Ahmo-C2
MRSIRISTAGSPSDYSHGLLPIIAQSLGYQLDWVKSSHADLLILGPFIKPETKKLKWCPKPLRPSLQSALNLMPLRASPPLRLFHSAENLRHDTILADYSISFDLNVSQANHFRLPYWMEMIDWSHEGITGNHNPRYGQLMKIERLMQPLGNRFLQKKKAAAFLSSHIREPRKTLFEAVQKVMPVTGFGAHFNKNIENHHRSGFLKQELLQEFGFNLCPENGMHPGYYTEKIPEAFFADCLPMTWTDENVKVDFNPNAFINLAPMAWCNYFPLRELVNSPQMLEDYASQALLLETPSILPFRDFVKRVLEDARS

SEQ ID NO: 39 - protein, 314 aa, Polynucleobacter sp.
  source 1..314: note = GWA2_45_21
MRSVRISTIGCPIDYSQSLLPIIAQSLGYQIDWVTPNQADLLILGPFMRPDSKKLRWCPKPLRSTVNSVFKLAPAKNNSALRVFHSAENLRHDTVPADYSISFDLNISSDRYFRLPYWMEMLDWSHEGIVDNRNPRYGKLMDIQRLMQPLGKRFLEKRRVVAFLSSHIREPRKTLYEAVQKVIPIKGFGAHFDKTIKNHHLSNFLKYELLQEYGFNLCPENGMYPGYYTEKIPEAFFAECLPITWTDENVKIDFNPEAFINLGPMSWCNYAPLAEIINSPQRLLAYSSQALLLNAPSILPFREFVKRMLEDATS

SEQ ID NO: 40 - protein, 314 aa, Polynucleobacter sp.
  source 1..314: note = UK-Pondora-W15
MRSVRISTAGCPSDYSQGLLPIIAQSLGYKLDWVKPNQADLLIMGPFMNHHAKKLKWCPKPLRPAAQTLFNSFSSRTDLPLRVFHSAENLRHDTIPADYSISFDLNINSPTHFRLPYWMEMINWSHEGIVGNKNPRYGQLMNIERLMEPLGMHFLDRTKAAAFLSSHLREPRKTLFEAVQNVMPVSGFGAHFDQNIQSHHASGFLKQDILKEFAFNLCPENGMYPGYYTEKIPEAFFAGCLPITWTDQNVRVDFNPNAFINLAPMAWCNFDPLAEIVNSPPHLEAYASEALLLNAPSILPFRDFVKRIMEDVTS

SEQ ID NO: 41 - protein, 314 aa, Polynucleobacter sp.
  source 1..314: note = UB-Tiil-W10
MNSIKIATEGVPHDYQSSLLPMVIQGMGYKIDWVKPGEADLLILGPFCKKNQKPFRWCPKPLRPLLNKSTEAIKTKKSRPLTLFHTQENERHDFVDADYSISFDLGVQSDKHFRFPYWMEMLDWSHEGITTNTNPRYGELLKISTLMSPLGSRYLNRGGSCALLSSHLREPRGSLFKALEKVVPIQGFGAFFNKEIKDHHSSGFLKRGVLKDFSYNLCPENGLYPGYYTEKIPEAFYSGCLPITWADENIGVDFNPSSFLNLQPLFKSDFQDLPEILLNKLRLESYQSEPLLIKKPSLEPLKQFLNKILSEAIS

SEQ ID NO: 42 - protein, 315 aa, Helicobacter cinaedi
MEDNLIIIRVDGGIASQIGFVALGKAFEEKDYQVKYDLSWFETSGKGFYNTTNGYDRIYDLTFDMPKAFPQLEMKIATEDEVKRYNKLYFIDDEKVITHKPPLYVGGYLGRHYDIYFARHFATYFSPKEIEQKDAPFYILLQEILNTQSCGIHIRRGDLSQNHIVYGEPTSLTYFERVIQLVAQMDSKSVFYLFSDDVAWVKEHIAPLLKDKQFKICDINTPEQGYLDLYLLSRCKVIVASHGSLGAYAKILAPHNPLLIAPRVRNVFFEMENVMLVNWGAKPKITQPCNNVITPPPIVKISHCDTDFFSISTIA

SEQ ID NO: 43 - protein, 316 aa, Helicobacter bilis
MKDNLTIIRVDGGIASQIGFVALGKAFEEKGYQVKYDLSWFETSGKGFYNTTNGYDRIYDLTFDMPKAFPQLEMKIATEDEIKRYNKLYFIDDEKVITHKPPLYVGGYLGRHYDIYFARRFATYFNPKEIEQKDAPFYILLQEILNTQSCGIHIRRGDLSQNHIVYGEPTSLTYFERVIQLVAQVNSKSVFYLFSDDVAWVREHIAPLLKDKQFKICDINTPEQGYLDLYLLSRCKVIVASHGSLGAYAKILSPHNPLLIAPRVRNVFFEMENVMLVNWGAKLQITQPCNNVITPPPPFSQTPHFDTTFFLSLQSL

SEQ ID NO: 44 - protein, 316 aa, Helicobacter bilis
MKDNLTIIRVDGGIASQIGFVALGKAFEEKGYQVKYDLSWFETSGKGFYNTTNGYDRIYDLTFDMPKAFPQLEMKIATEDEIKRYNKLYFIDDEKVITHKPPLYVGGYLGRHYDIYFARRFATYFNPKEIEQKDTPFYILLQEILNTQSCGIHIRRGDLSQNHIVYGEPTSLTYFERVIQLVAQVNSKSVFYLFSDDVAWVKEHIAPLLKDKQFKICDINTPEQGYLDLYLLSRCKVIVASHGSLGAYAKILAPHNPLLIAPRVRNVFFEMENVMLVNWGAKPKITQPCNNVITPPPPIVKISHCDTDFFSISTIA

SEQ ID NO: 45 - protein, 317 aa, Campylobacter sp.
  source 1..317: note = P146
MRKDLVIIRVDGGIASQICFVALGLEFESRGYKVKYDLSWFDEFGNGFYNEQNGYDRSYNIAFDIPKAFPDIKCEIASRDEIEIYRKKYFVDNEEVIELKPPLYVGGYLGRHFDFKYRDFFAKHFNPKEIQESDSECKILLDNILSNHSCGVHIRRGDLSKEHVVYGKPLSVEYFLKNIQLVLQIEPKSKFYFFSDDLEWVKKTVIPSLNSIEYEICNVNSVEKGFLDLYLLSRCKIIIASHGSMGKYAKMLAPDTETLLITPNENAPMENMMMINWADSYESSGEKQIQKPLSWRNKIRLKIYKKLRNKLLQKRIL

SEQ ID NO: 46 - protein, 331 aa, Devosia sp.
MTSRRPLILFRTGFFGRSPLDLVPAQIVDHFTTDLSRIGEADAVVFHIPDWRRSLPEDTPKFPGQKWVAWSMESKVNYRQLADPHFLGHFELRMTYEQGADIWMPYLPSTERWQAAMAKDLPVHDEPVPVVMFQSARDDRSGRNAFSAELMRRIAVDSYGRLFNNRRLTVPDRGPETKLATIRRYRFCLGLENALAHDYVTEKLFEPLLAGTVPIYRGAPNVREFAPEHSYIDAEAHDGPAGLAAYLRHLMACPQEYAAYFAWRTRPLPAWLEAKLEAVRVEPFARLLELIADQPRVPGAGRTVLPFGRHKALQTRIRRMLRRARRAVRGR

SEQ ID NO: 47 - protein, 329 aa, Hyphomicrobiales bacterium
MPSNAPLILFWTRYFDKPVDIAEWSQCSVPVEWTNDRRRLADADAVVFHLPNSREIGDARKYPGQLWVAWSMESVVNYPVMADASFMRHFDVAMTYERGSDVWTPYLPRAGWWDAARHEPIPPKVERAPTVHFQSSSFNKSGRDTFVTELSRHIAVDCYGRYRSNRQITGPDLGRRTKLETIGRYPFCLALENSIAPDYVTEKMFDPLAAGTVPVYLGAPNAAEFVPDGCYIDAASYRTPAELAAYLRHLAETPGDYQAYFAWRSRPLPDRLARRLDELDAPAWRRLAELVQRHAQQRSGRLSGRPSLPFGPISFLRTRLRRARADLGR

SEQ ID NO: 48 - protein, 318 aa, Arcticibacter pallidicorallinus
MIDIKRLYQLWVQYNKEVTWCKDSNIKVFNFWNLQKPEDLWLHQFIQHHFGESLSKKNSIALFSVMGDRRKISLNRSKVKVFFTGENTTIWKDYKDHCIQEVDLSMGFEYIDADNYLRFPLWLLYILDPRSDYQAVKKSVHKISVTNKDRQNYPKRFCSLICRHDGNGIRTEMFNLLTSVGQIDSAGPFLNNTSELKEAYNDQKQEYLKNYKFNICPENSNTLGYVTEKLFESISAGCIPLYWGNENPEPEVLNKNAILFYKGVERSHELTTQVRELHNNEKLYGEFISQETFVPTAAEYIWDRLNMLHEHLSRLLKN

SEQ ID NO: 49 - protein, 317 aa, Parapedobacter defluvii
MHVKRLLQKIGWYREPANYTYLPDENHIELLNFWTSQPREEIWLYQFILQHVGSRLSKDNGVLLSSVFGPRNAIKESRSRIKVFYTGENVRRFHEYRDHCLNDVDISLGFDDINHPKYVRFPIWIFYFFGITQTQQEIQYVLDNFSLPPPPERGFCSLVSSHDKNGIRTGLFHALGRIEQVTSAGRLLNNSDVLRTRYNDDKTEMLRAFNFNICPENTDYPGYVTEKLFQCFRASTVPIYWGSGNEPEPDIVNPDAVLFYQGKHSLNQLVRRVEELHRDPEKYRAFLRQPKFKPAAAAHIWERINLLREKLVNLVNG

SEQ ID NO: 50 - protein, 320 aa, Pedobacter schmidteae
MKFKSLLIDLGLYRPKYLPDENHIELLNCWTTDPEKLWQYHFVTGQFSNLLTAQNSLLIGSVFGPKGIFKLSKSKKKLFYTGENVDRFPEYTDYCDGIVDLSIGFDYLDRENYQRFPLWMEYFFAPVMNTKKIHEVFSDFVIKDICRDEDKKFAVMVSRHDEGNSRAAIFNSINQIDQVDSGGQFLKNTNALKEEYNDDKGKFIGQYKFNICPENSNREGYVTEKVFEAIKSNTIPIYWGSNNNPEPDVLNKDAILFYDGPESLPALNRKVEELHRNPKLYREFMAQPKFQPQAVEYVIDAFEGLYSKLHNMLKEDRFKG

SEQ ID NO: 51 - protein, 327 aa, Helicobacter bilis
MEDNLIIVRVDGGIASQIGFVALGKAFEEKGYQVKYDLSWFETSGKGFYNTINGYDRIYDLTFDMPKAFPQLEMKIASEDEVKRYNKLYFIDDEKVITHKPPLYVGGYLGRYYDIYFARHFATYFSPKEIEQKDTPFYILLQEILNTQSCGIHIRRGDLSQNHIVYGEPTSLTYFERVIQLVAQMDSKSVFYLFSDDVAWVREHIAPLLKDKQFKICDINTPEQGYLDLYLLSRCKVIVASHGSLGAYAKILAPHNPLLIAPRVRNVFFEMENVMLVNWGAKPKITQPCNNVITPPPHCQNLTLRYRLFLYLYNRLRSKLLRKGVIQ